(19) United States
(12) Patent Application Publication
(10) Pub. No.: US 2024/0269299 A1
(43) Pub. Date: Aug. 15, 2024

(54) POLYETHYLENE GLYCOL-DRUG CONJUGATE AND USE THEREOF

(71) Applicant: CHONGQING UPGRA BIOTECHNOLOGY CO. LTD., Chongqing (CN)

(72) Inventors: Gaoquan LI, Chongqing (CN); Nian LIU, Chongqing (CN); Yongchen PENG, Chongqing (CN); Xiafan ZENG, Chongqing (CN); Yang GAO, Chongqing (CN); Yuanyuan PENG, Chongqing (CN); Jie LOU, Chongqing (CN); Huiyu CHEN, Chongqing (CN); Kun QIAN, Chongqing (CN); Gang MEI, Chongqing (CN); Sheng GUAN,, Chongqing (CN); Jing LIU, Chongqing (CN); Yongqin WU, Chongqing (CN); Shuai YANG, Chongqing (CN); Xiangwei YANG, Chongqing (CN); Yusong WEI, Chongqing (CN); Dajun LI, Chongqing (CN); Qian ZHANG, Chongqing (CN); Ming RAN, Chongqing (CN)

(21) Appl. No.: 18/561,169

(22) PCT Filed: May 9, 2022
(86) PCT No.: PCT/CN2022/091674

§ 371 Date: Nov. 15, 2023

(30) Foreign Application Priority Data
May 18, 2021 (CN) 202110540872.7

(51) Int. Cl. A61K 47/60; A61P 35/00

(52) U.S. Cl. CPC A61K 47/60; A61P 35/00

(57) ABSTRACT

A polyethylene glycol-drug conjugate and a use thereof, specifically relating to a polyethylene glycol-drug conjugate as shown in formula A, a stereoisomer thereof or a pharmaceutically acceptable salt thereof; an intermediate for the preparation of the polyethylene glycol-drug conjugate, the stereoisomer thereof or the pharmaceutically acceptable salt thereof; a pharmaceutical composition containing the polyethylene glycol-drug conjugate, the stereoisomer thereof or the pharmaceutically acceptable salt thereof; and a use of the polyethylene glycol-drug conjugate, the stereoisomer thereof or the pharmaceutically acceptable salt thereof in the preparation of a drug.

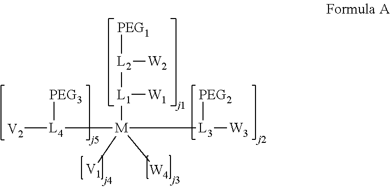

### TECHNICAL FIELD

The present disclosure belongs to the field of medical technology, and in particular, relates to a polyethylene glycol-drug conjugate and use thereof.

### BACKGROUND

Compared with original drugs, PEGylated drugs have great advantages of, for example, increasing water solubility of drug molecules and preventing or reducing drug agglomeration, immunogenicity, and antigenicity. Many drugs can only remain in blood circulation for a few minutes, while polyethylene glycol-drug conjugates as anticancer drugs can last for tens or hundreds of hours or even longer, which is conducive to the “enhanced permeability and retention effect”, namely the EPR effect, caused by the leakage of tumor capillaries. Due to an increase in the hydrodynamic volume, renal elimination of the drugs is lessened, the drugs are prevented from being degraded by enzymes, half-life of the drugs in plasma is prolonged, and bioavailability of the drugs is increased. Through passive targeting of the EPR or active targeting, anticancer drugs are highly enriched in cancerous organs, tissues or cells, thereby greatly reducing the toxic and side effects caused by small molecule anticancer drugs distributed all over the body. Limiting the cellular absorption of the drugs to the endocytic pathway is conducive to drug transport to lysosomes, thus avoiding drug resistance caused by pumping out p-glycoprotein; stimulates or restores immune functions, which is beneficial to killing cancer cells.

US companies such as NEKTAR and ENZON have achieved great success in polyethylene glycol-drug conjugate technology. So far, 26 drugs have been approved by the U.S. FDA for marketing, and drug administrations of South Korea, Russia, India and China have also approved PEGylated drugs to enter their markets. However, all of the above PEGylated drugs are PEGylated mono-drugs.

The inventors of the present application have developed some special urea structure connection methods, which can connect a certain drug to a polyethylene glycol carrier alone or simultaneously with an additional drug or a synergist, and the obtained polyethylene glycol-drug conjugates have significant therapeutic effect.

### SUMMARY

The present disclosure aims to solve one of the technical problems in the related art, at least to a certain extent. To this end, the present disclosure provides a polyethylene glycol-drug conjugate, a stereoisomer thereof or a pharmaceutically acceptable salt thereof, which has excellent tumor inhibitory activity or excellent therapeutic activity for ophthalmic diseases (such as choroidal neovascularization).

According to a first aspect of the present disclosure, the present disclosure provides a polyethylene glycol-drug conjugate represented by Formula A, a stereoisomer thereof or a pharmaceutically acceptable salt thereof,

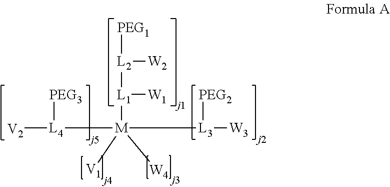

where:
M is selected from

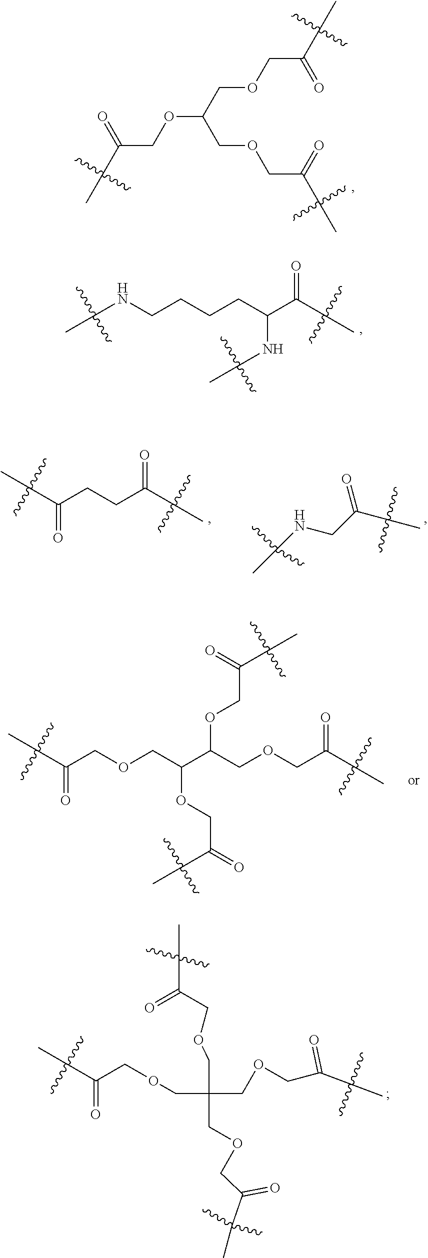 [SMILES: *C(=O)COCC(COCC(*)=O)OCC(*)=O ; *NCCCCC(N*)C(*)=O ; *C(=O)CCC(*)=O ; *NCC(*)=O ; *C(=O)COCC(OCC(*)=O)C(COCC(*)=O)OCC(*)=O ; *C(=O)COCC(COCC(*)=O)(COCC(*)=O)COCC(*)=O]

or
M is selected from

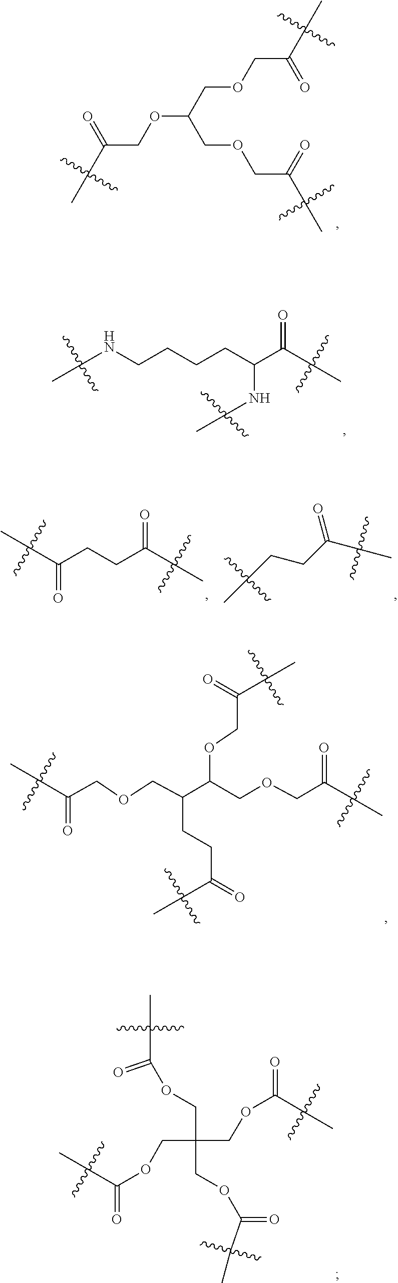 [SMILES: *C(=O)COCC(COCC(*)=O)OCC(*)=O ; *NCCCCC(N*)C(*)=O ; *C(=O)CCC(*)=O ; *CCC(*)=O ; *C(=O)CCC(COCC(*)=O)C(COCC(*)=O)OCC(*)=O ; *C(=O)OCC(COC(*)=O)(COC(*)=O)COC(*)=O]

L₁, L₂, L₃ and L₄ are each independently selected from

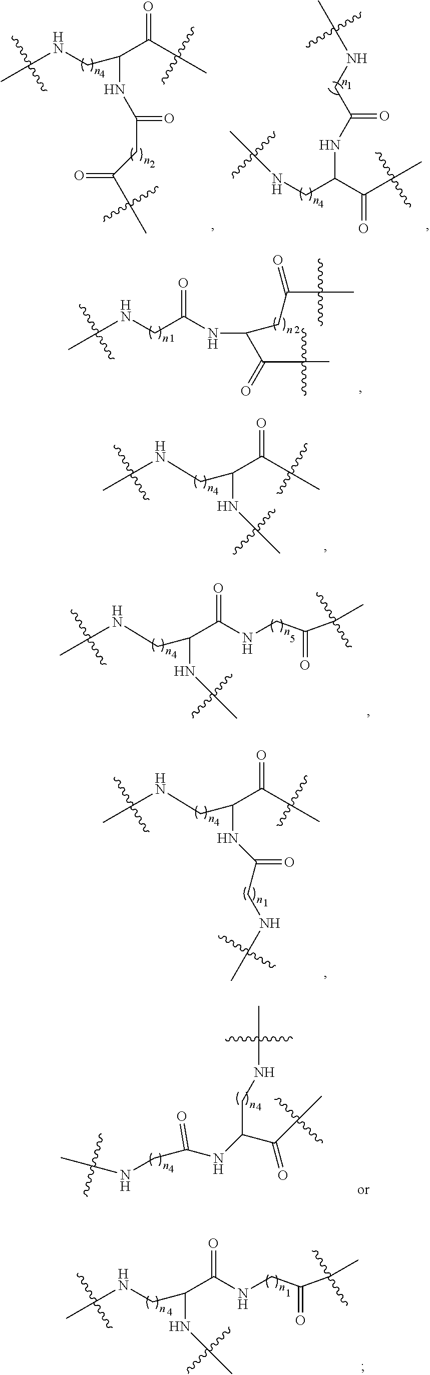

W₁, W₂, W₃ and W₄ are each independently selected from -Q₁,

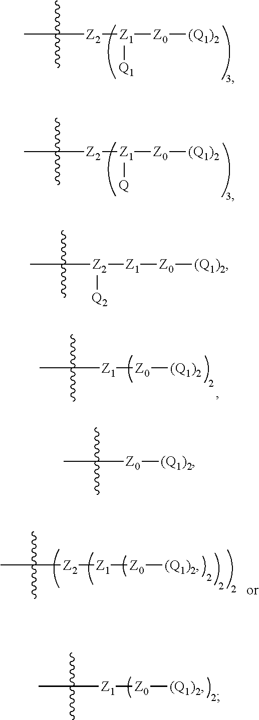

PEG₁, PEG₂ and PEG₃ are each independently a single-armed polyethylene glycol chain
segment, PEG₁ is connected to L₂ through a carbonyl group, PEG₂ is connected to L₃ through a carbonyl group or an amino group, PEG₃ is connected to L₄ through a carbonyl group, and a number-average molecular weight of each of PEG₁, PEG₂ and PEG₃ is 5k to 40k, preferably 5k to 10k or 10k to 40k, and more preferably 10k;
j₁, j₂, j₃, j₄ and j₅ are each independently selected from 0, 1, 2, 3, 4 or 5, and j₁, j₂, j₃, j₄ and j₅ are not 0 at the same time;
Z₂, Z₁ and Z₀ are each independently selected from

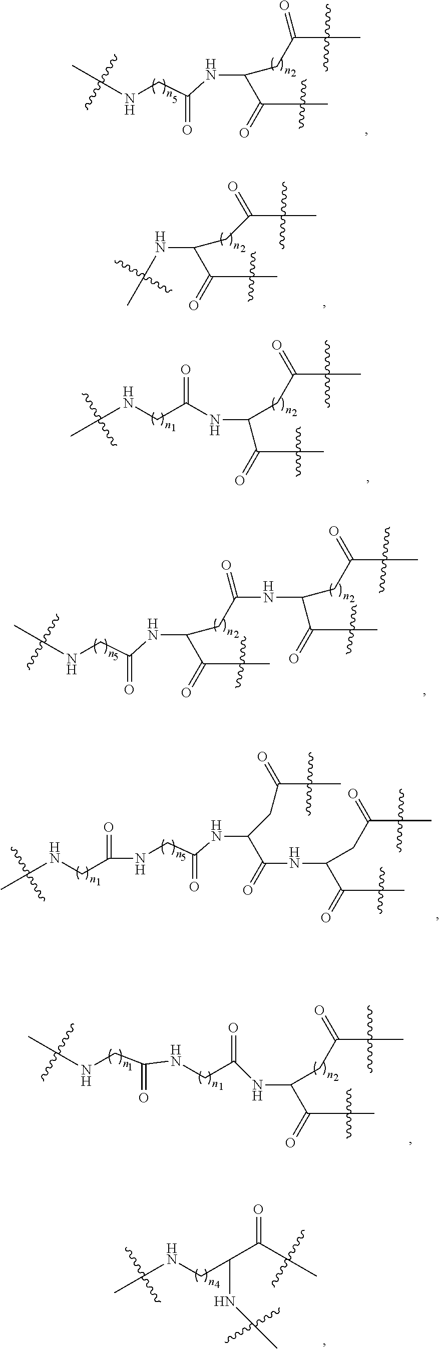

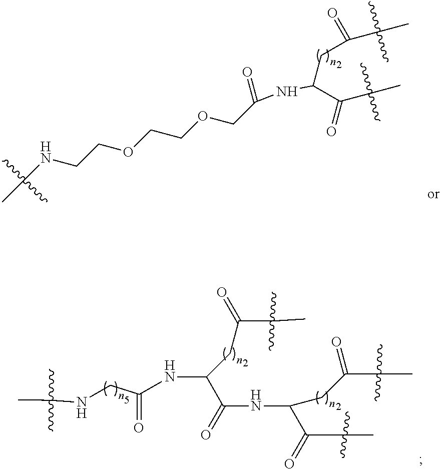

### Q is —N-AC;

Q₁ is —N₁-AC₁;
Q₂ is —N₂-AC₂;
N, N₁ and N₂ are each independently selected from

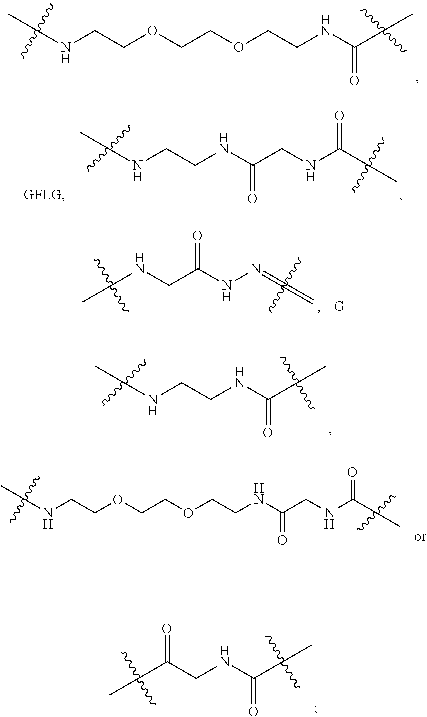

AC, AC₁ and AC₂ are each independently an anticancer drug;
V₁ and V₂ are each independently selected from

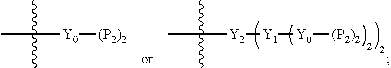

Y₂, Y₁ and Y₀ are each independently selected from

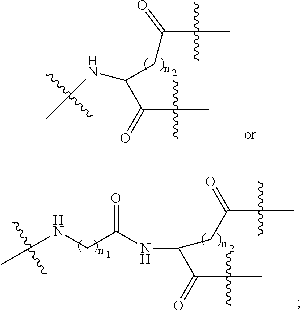

### P is -Lᵥ-T;

Lᵥ is selected from

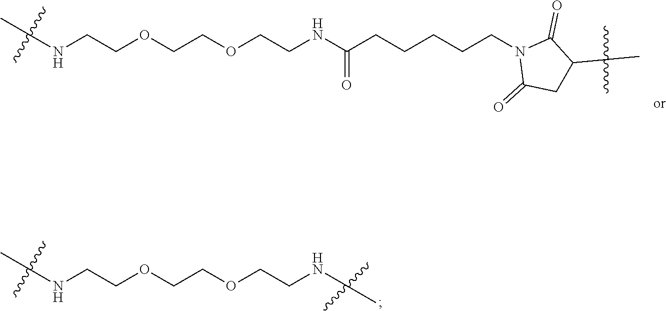 [SMILES: *NCCOCCOCCNC(=O)CCCCCN1C(=O)CC(*)C1=O ; *NCCOCCOCCN*]

T is selected from PPT-iRGD or FA; and
n₁, n₂, n₄ and n₅ are each independently selected from 1, 2, 3, 4, 5, 6, 7 or 8.

In some embodiments, L₁, L₂, L₃ and L₄ are each independently selected from

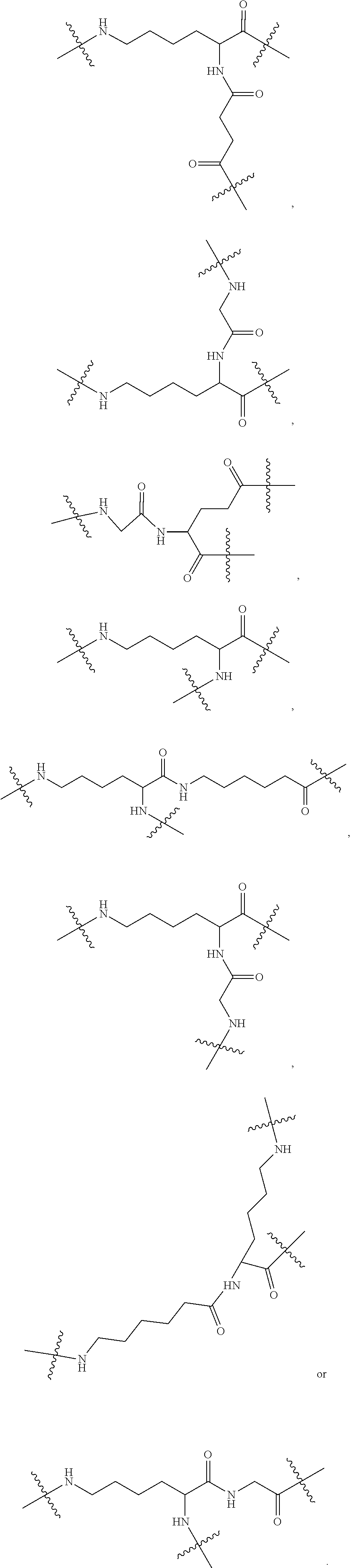

In some embodiments, L₁, L₂, L₃ and L₄ are each independently selected from

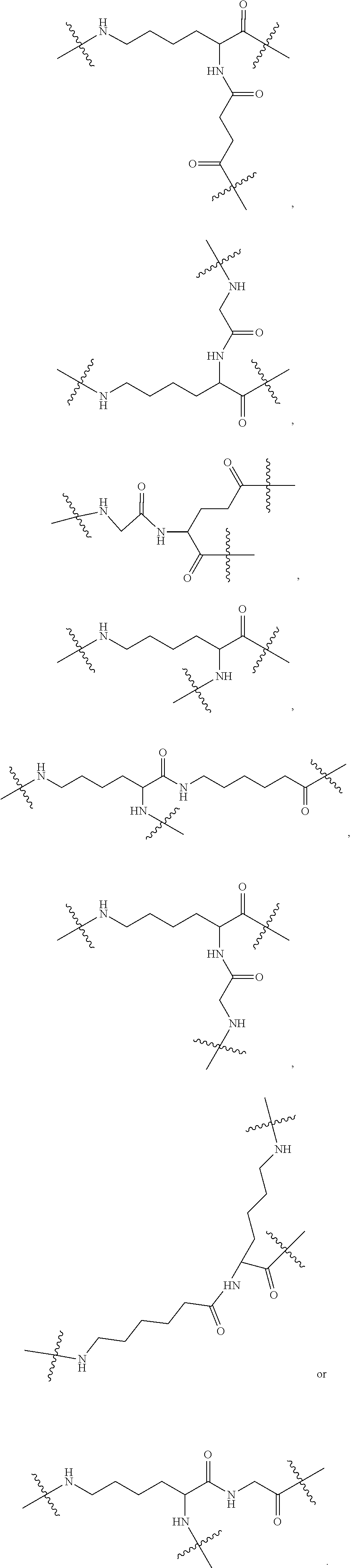

In some embodiments, L₁ is

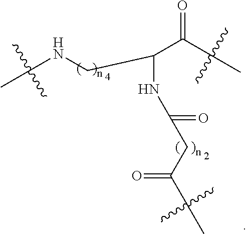

In some embodiments, L₁ is

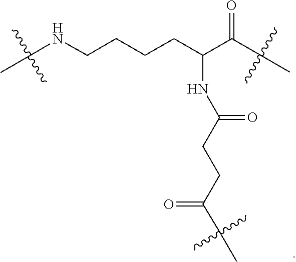 [SMILES: *NCCCCC(NC(=O)CCC(*)=O)C(*)=O]

In some embodiments, L₂ is

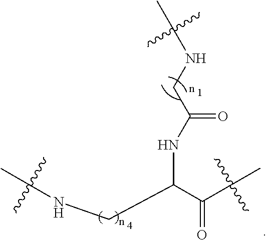

In some embodiments, L₂ is

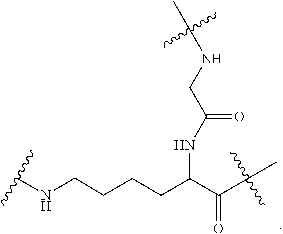 [SMILES: *NCCCCC(NC(=O)CN*)C(*)=O]

In some embodiments, each L₃ is independently selected from

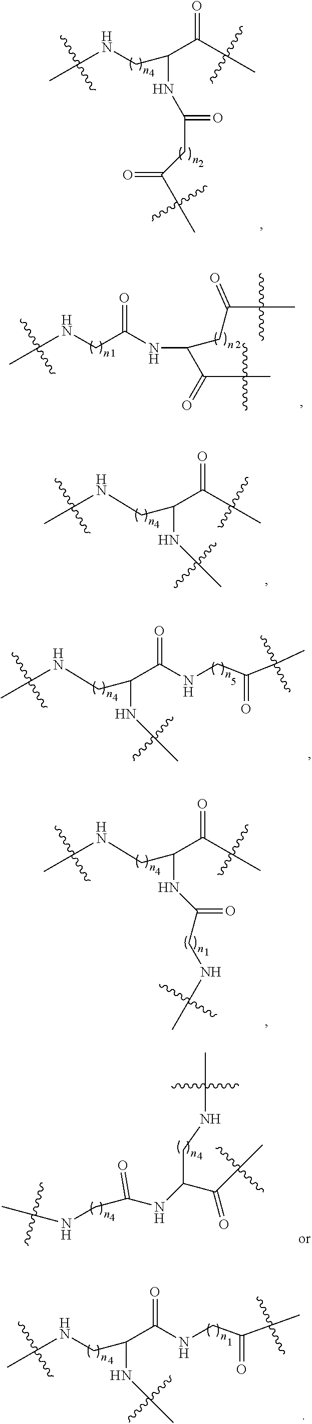

In some embodiments, each L₃ is independently selected from

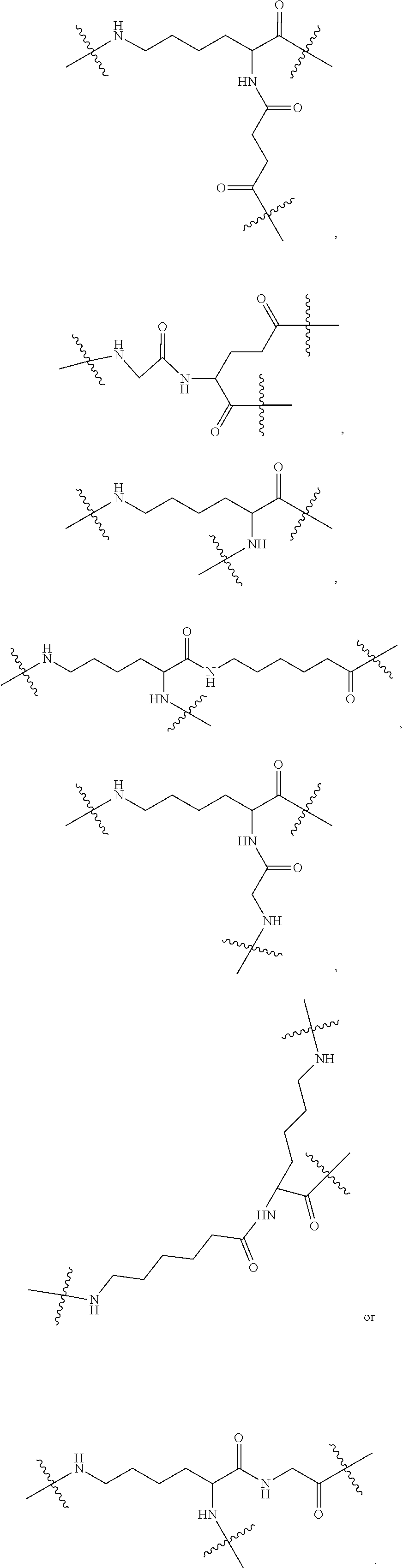

In some embodiments, each L₃ is independently selected from

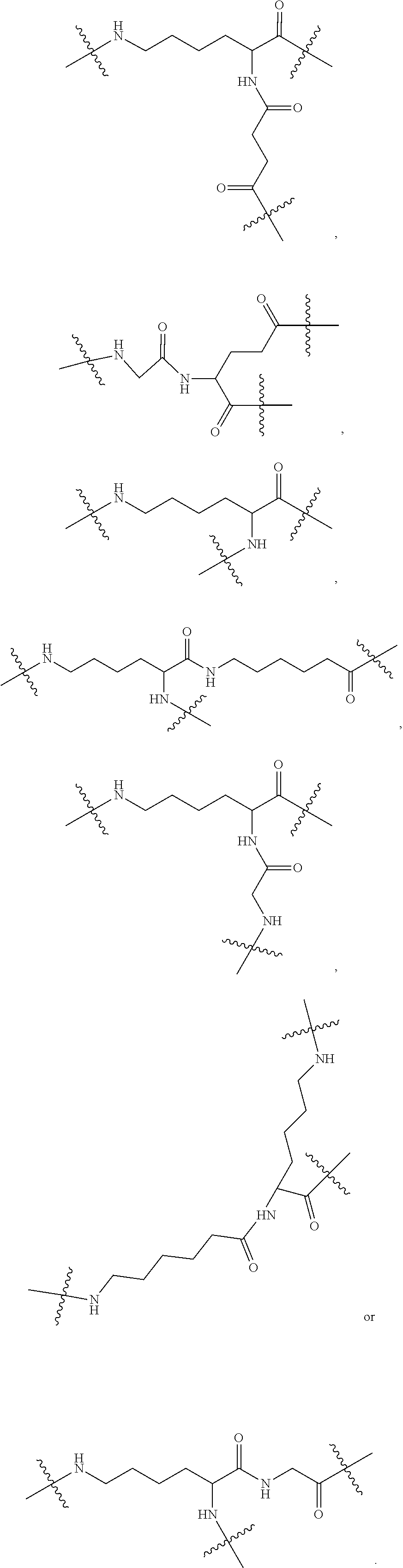

In some embodiments, L₄ is

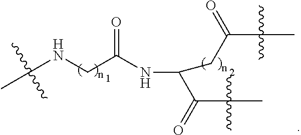

In some embodiments, L₄ is

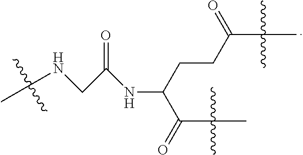 [SMILES: *NCC(=O)NC(CCC(*)=O)C(*)=O]

In some embodiments, W₁ and W₂ are each independently selected from -Q₁ or

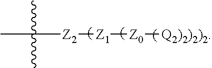

In some embodiments, W₃ and W₄ are each independently selected from

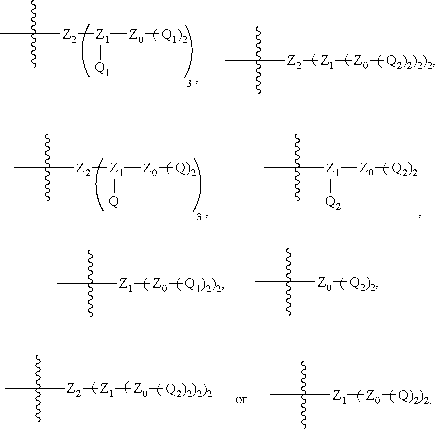

In some embodiments, W₁ is -Q₁.

In some embodiments, W₂ is

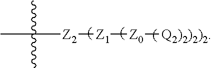

In some embodiments, W₃ is selected from

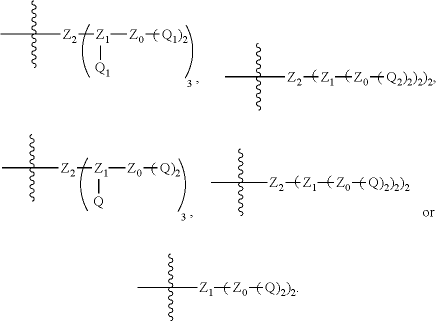

In some embodiments W₄ is selected from

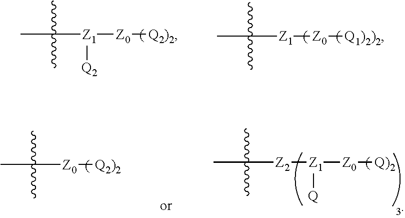

In some embodiments, j₁ is selected from 2, 3 or 4.

In some embodiments, j₁ is 3.

In some embodiments j₂ is selected from 1, 2, 3, 4 or 5.

In some embodiments, j₂ is selected from 1, 2 or 4.

In some embodiments, j₃ is selected from 1, 2 or 3.

In some embodiments, j₃ is 1.

In some embodiments, j₄ is selected from 1, 2 or 3.

In some embodiments, j₄ is 1.

In some embodiments, j₅ is selected from 1, 2 or 3.

In some embodiments, j₅ is 1.

In some embodiments, Z₂, Z₁ and Z₀ are each independently selected from

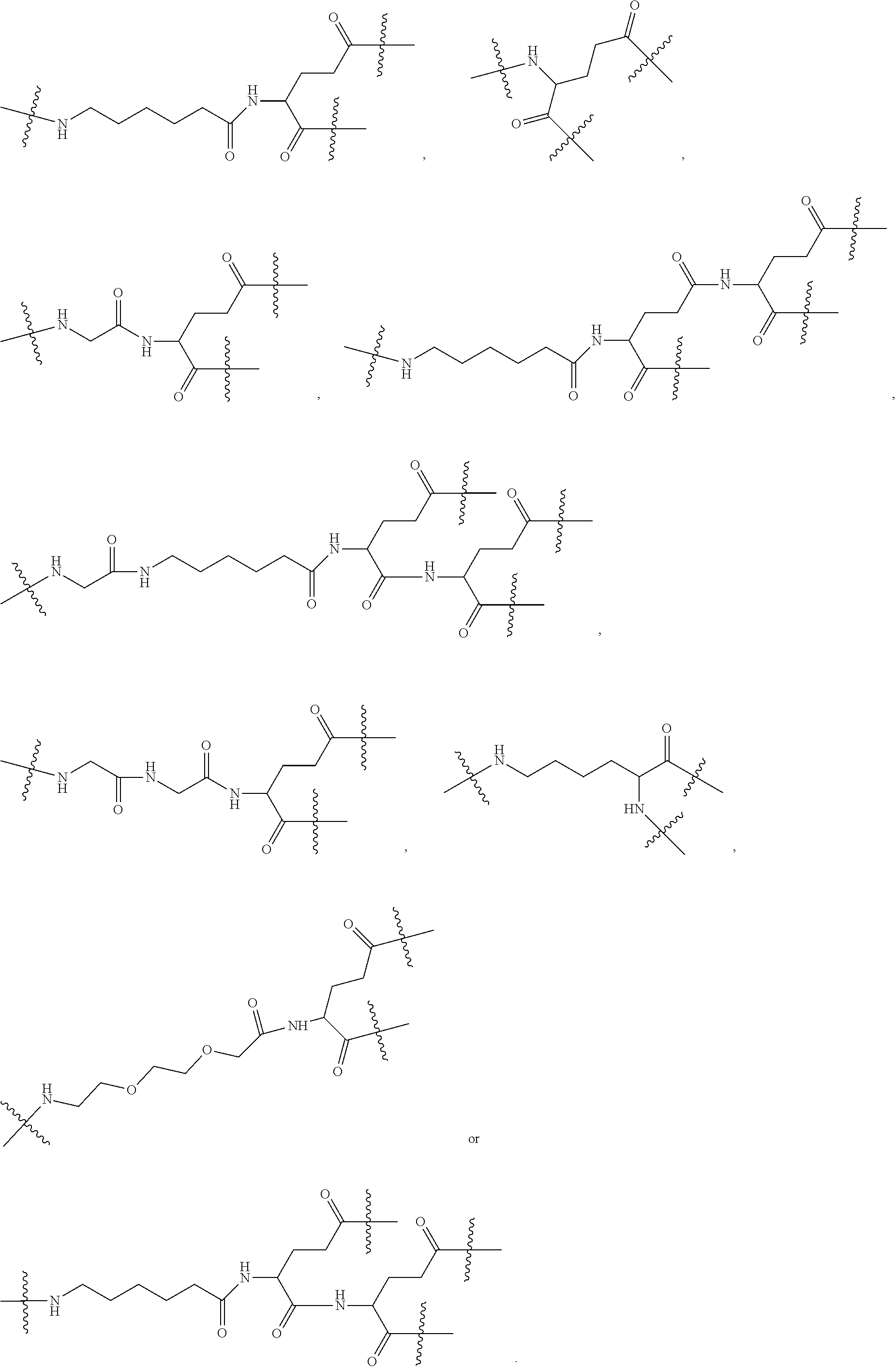

In some embodiments, Z₂ is selected from

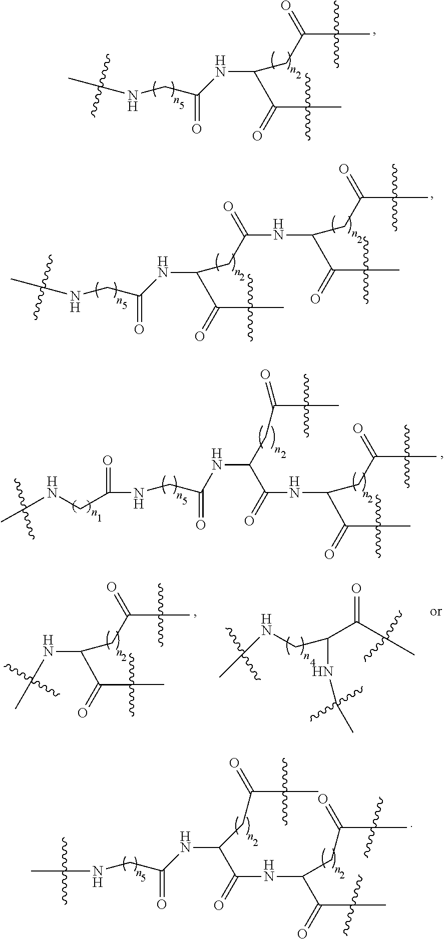

In some embodiments, Z₂ is selected from

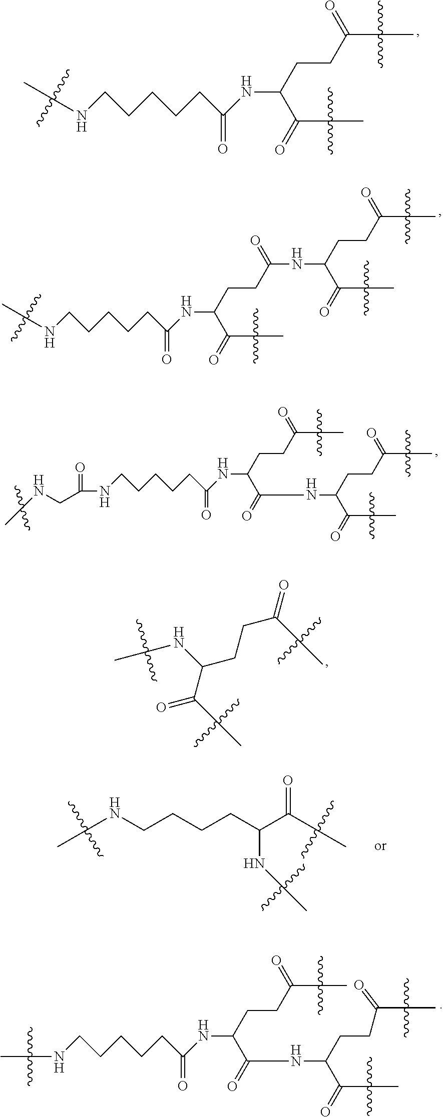 [SMILES: *NCCCCCC(=O)NC(CCC(*)=O)C(*)=O ; *NCCCCCC(=O)NC(CCC(=O)NC(CCC(*)=O)C(*)=O)C(*)=O ; *NCC(=O)NCCCCCC(=O)NC(CCC(*)=O)C(=O)NC(CCC(*)=O)C(*)=O ; *NC(CCC(*)=O)C(*)=O ; *NCCCCC(N*)C(*)=O ; *NCCCCCC(=O)NC(CCC(*)=O)C(=O)NC(CCC(*)=O)C(*)=O]

In some embodiments, Z₁ is selected from

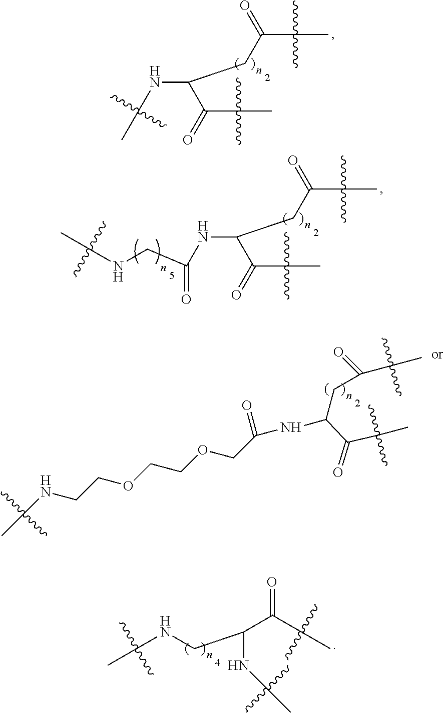

In some embodiments, Z₁ is selected from

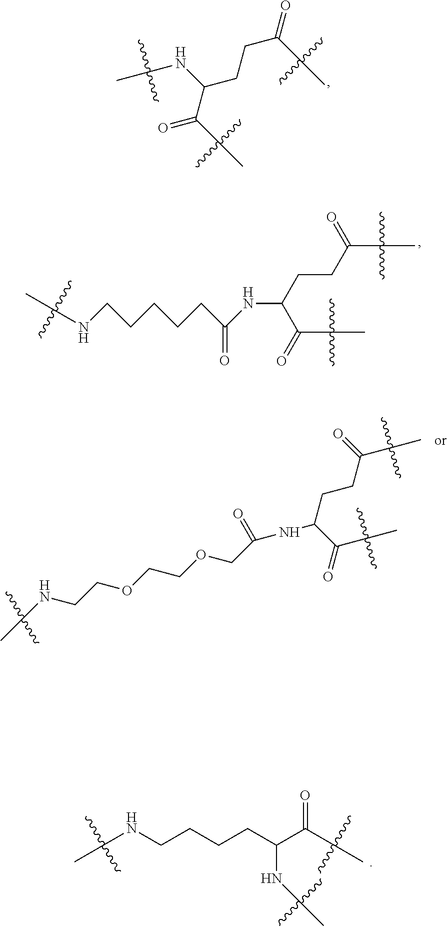 [SMILES: *NC(CCC(*)=O)C(*)=O ; *NCCCCCC(=O)NC(CCC(*)=O)C(*)=O ; *NCCOCCOCC(=O)NC(CCC(*)=O)C(*)=O ; *NCCCCC(N*)C(*)=O]

In some embodiments, Z₀ is selected from

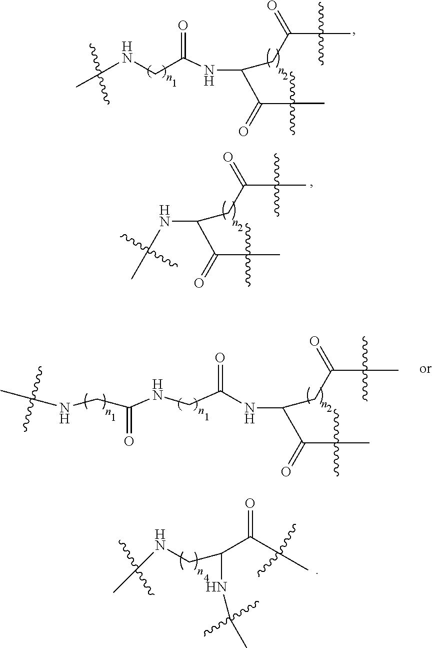

In some embodiments, Z₀ is selected from

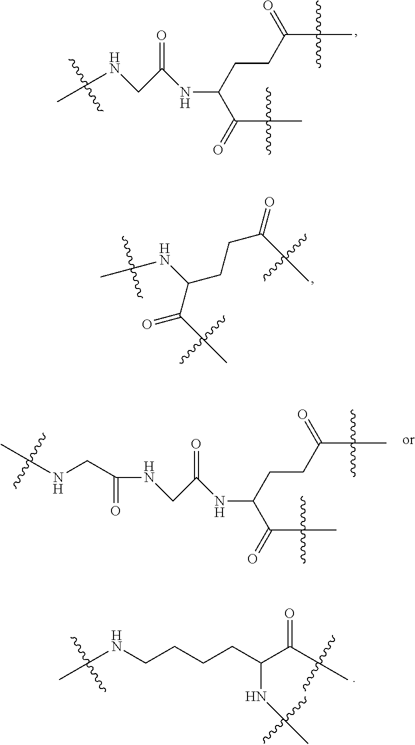 [SMILES: *NCC(=O)NC(CCC(*)=O)C(*)=O ; *NC(CCC(*)=O)C(*)=O ; *NCC(=O)NCC(=O)NC(CCC(*)=O)C(*)=O ; *NCCCCC(N*)C(*)=O]

In some embodiments, N is selected from

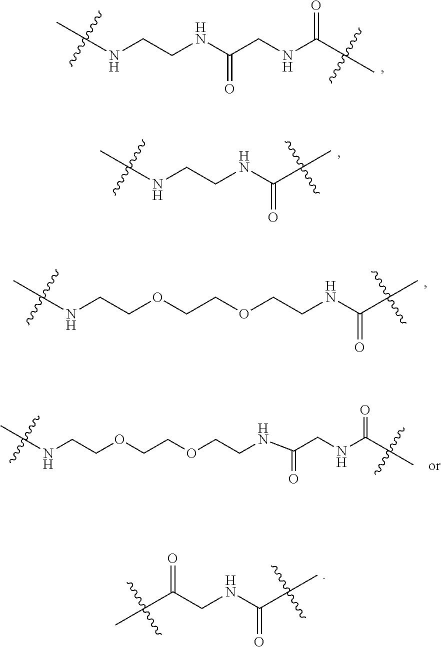 [SMILES: *NCCNC(=O)CNC(*)=O ; *NCCNC(*)=O ; *NCCOCCOCCNC(*)=O ; *NCCOCCOCCNC(=O)CNC(*)=O ; *C(=O)CNC(*)=O]

In some embodiments, N₁ is selected

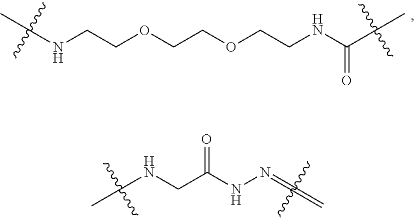 [SMILES: *NCCOCCOCCNC(*)=O ; *NCC(=O)NN=*]

### or G.

In some embodiments, N₂ is selected from GFLG or

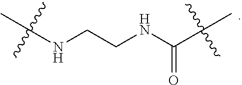 [SMILES: *NCCNC(*)=O]

In some embodiments, AC, AC1 and AC2 are each independently selected from AXT, PCB, 5FU, DXM, SRM, IMQ or LNL.

In some embodiments, AC is selected from 5FU, IMQ, LNL or AXT.

In some embodiments, AC₁ is selected from AXT, DXM or SRM.

In some embodiments AC₂ is selected from PCB or AXT.

In some embodiments, V₁ is

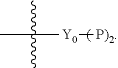

In some embodiments, V₂ is

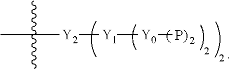

In some embodiments, Y₂, Y₁ and Y₀ are each independently selected from

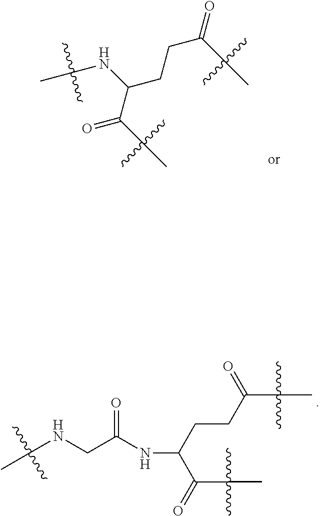 [SMILES: *NC(CCC(*)=O)C(*)=O ; *NCC(=O)NC(CCC(*)=O)C(*)=O]

In some embodiments, Y₂ is

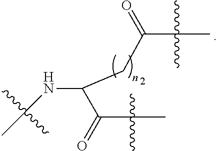

In some embodiments, Y₂ is

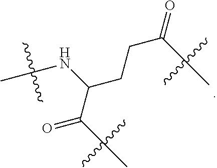 [SMILES: *NC(CCC(*)=O)C(*)=O]

In some embodiments, Y₁ is

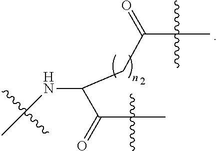

In some embodiments, Y₁ is

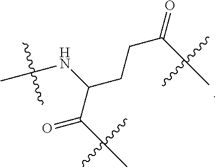 [SMILES: *NC(CCC(*)=O)C(*)=O]

In some embodiments, Y₀ is

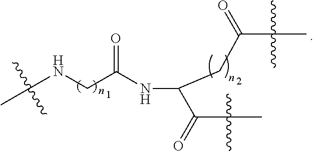

In some embodiments, Y₀ is

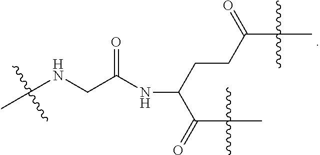 [SMILES: *NCC(=O)NC(CCC(*)=O)C(*)=O]

In some embodiments, n₁ is selected from 1, 2 or 3.

In some embodiments, n₁ is 1.

In some embodiments, n₂ is selected from 1, 2 or 3.

In some embodiments, n₂ is 2.

In some embodiments, n₄ is selected from 3, 4 or 5.

In some embodiments, n₄ is selected from 4 or 5.

In some embodiments, n₄ is 4.

In some embodiments, n₅ is selected from 4, 5 or 6.

In some embodiments, n₅ is 5.

According to a second aspect of the present disclosure, the present disclosure provides a polyethylene glycol-drug conjugate represented by Formula (I), a stereoisomer thereof or a pharmaceutically acceptable salt thereof,

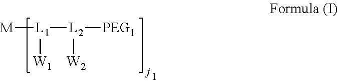

where:

### M is

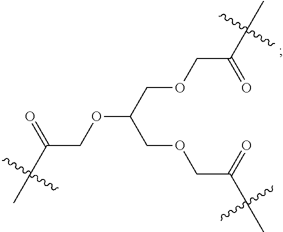 [SMILES: *C(=O)COCC(COCC(*)=O)OCC(*)=O]

### L₁ is

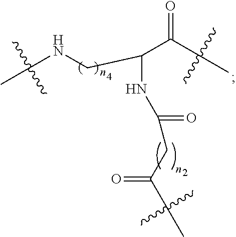

### L₂ is

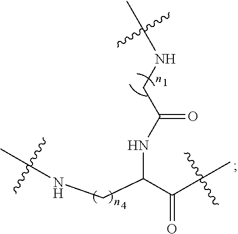

W₁ and W₂ are each independently selected from -Q₁ or

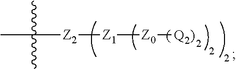

PEG₁ is a single-armed polyethylene glycol chain segment, PEG₁ is connected to L₂ through a carbonyl group, and a number-average molecular weight of PEG₁ is 5k to 40k, preferably 5k to 10k or 10k to 40k, and more preferably 10k;
j₁ is selected from 2, 3 or 4;
Z₂, Z₁ and Z₀ are each independently selected from

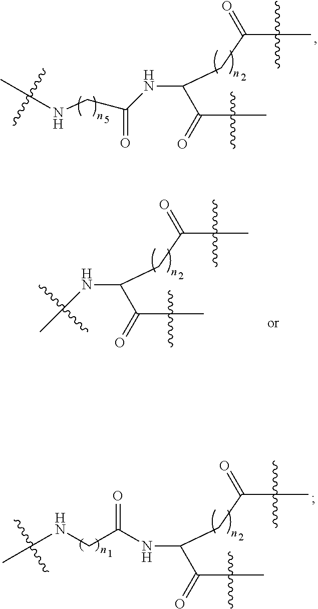

n₁, n₂, n₄ and n₅ are each independently selected from 1, 2, 3, 4, 5, 6, 7 or 8;
Q₁ is —N₁-AC₁;
Q₂ is —N₂-AC₂;
N₁ and N₂ are each independently selected from

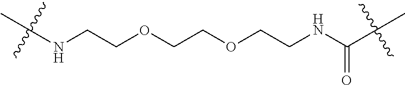 [SMILES: *NCCOCCOCCNC(*)=O]

### or GFLG; and

AC₁ and AC₂ are each independently an anticancer drug.

In some embodiments, L₁ is

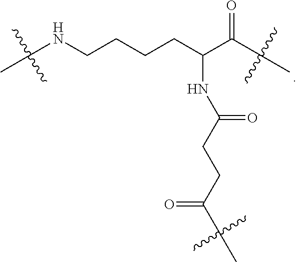 [SMILES: *NCCCCC(NC(=O)CCC(*)=O)C(*)=O]

In some embodiments, L₂ is

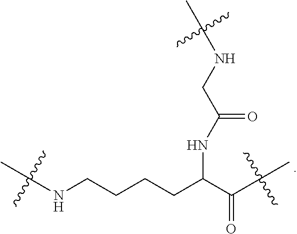 [SMILES: *NCCCCC(NC(=O)CN*)C(*)=O]

In some embodiments, W₁ is -Q₁ and W₂ is

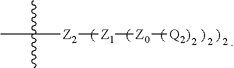

In some embodiments, j₁ is 3.

In some embodiments, Z₂, Z₁ and Z₀ are each independently selected from

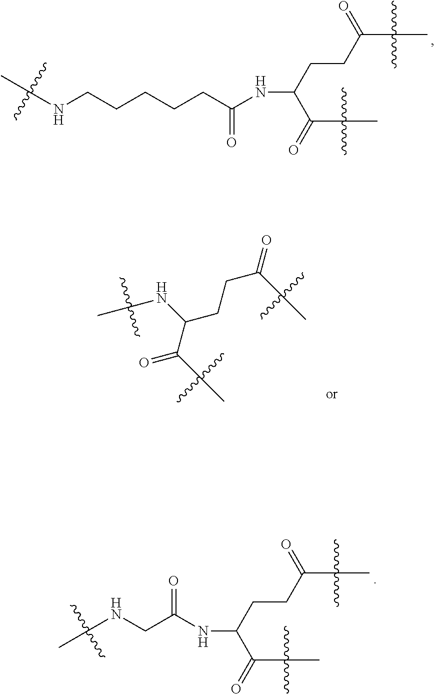 [SMILES: *NCCCCCC(=O)NC(CCC(*)=O)C(*)=O ; *NC(CCC(*)=O)C(*)=O ; *NCC(=O)NC(CCC(*)=O)C(*)=O]

In some embodiments, Z₂ is

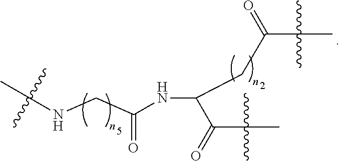

In some embodiments, Z₂ is

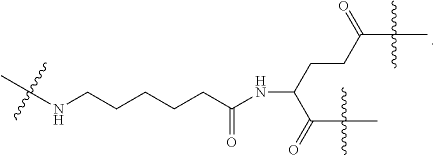 [SMILES: *NCCCCCC(=O)NC(CCC(*)=O)C(*)=O]

In some embodiments, Z₁ is

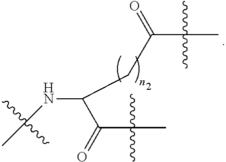

In some embodiments, Z₁ is

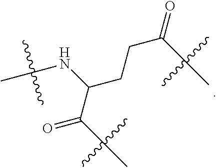 [SMILES: *NC(CCC(*)=O)C(*)=O]

In some embodiments, Z₀ is

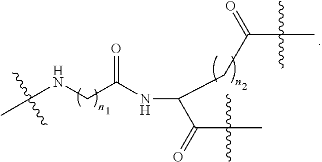

In some embodiments, Z₀ is

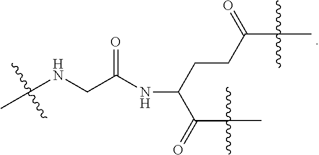 [SMILES: *NCC(=O)NC(CCC(*)=O)C(*)=O]

In some embodiments, n₁ is selected from 1, 2 or 3.

In some embodiments, n₁ is 1.

In some embodiments, n₂ is selected from 1, 2 or 3.

In some embodiments, n₂ is 2.

In some embodiments, n₄ is selected from 3, 4 or 5.

In some embodiments, n₄ is 4.

In some embodiments, n₅ is selected from 4, 5 or 6.

In some embodiments, n₅ is 5.

In some embodiments, N₁ is

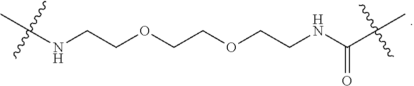 [SMILES: *NCCOCCOCCNC(*)=O]

In some embodiments, N₂ is GFLG.

In some embodiments, AC, AC₁ and AC₂ are each independently selected from AXT, PCB, 5FU, DXM, SRM, IMQ or LNL.

In some embodiments, AC₁ and AC₂ are each independently selected from AXT or PCB.

In some embodiments, AC₁ is AXT.

In some embodiments, AC₂ is PCB.

According to a third aspect of the present disclosure, the present disclosure provides a polyethylene glycol-drug conjugate represented by Formula (II), a stereoisomer thereof or a pharmaceutically acceptable salt thereof,

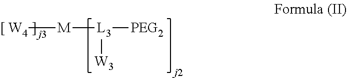

where:

### M is

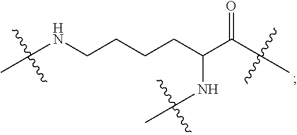 [SMILES: *NCCCCC(N*)C(*)=O]

L₃ is selected from

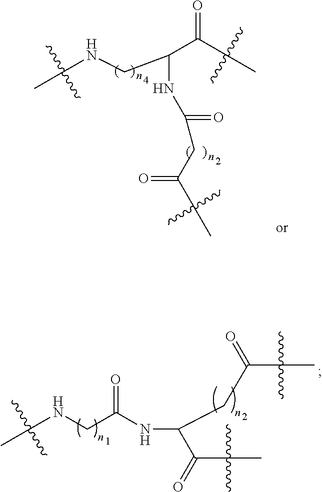

W₃ and W₄ are each independently selected from

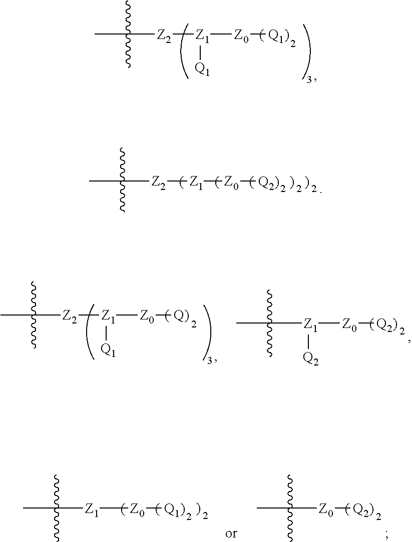

PEG₂ is a single-armed polyethylene glycol chain segment, PEG₂ is connected to L₃ through a carbonyl group, and a number-average molecular weight of PEG₂ is 5k to 40k, preferably 5k to 10k or 10k to 40k, and more preferably 10k;
j₂ and j₃ are each independently selected from 1, 2, 3 or 4;
Z₂, Z₁ and Z₀ are each independently selected from

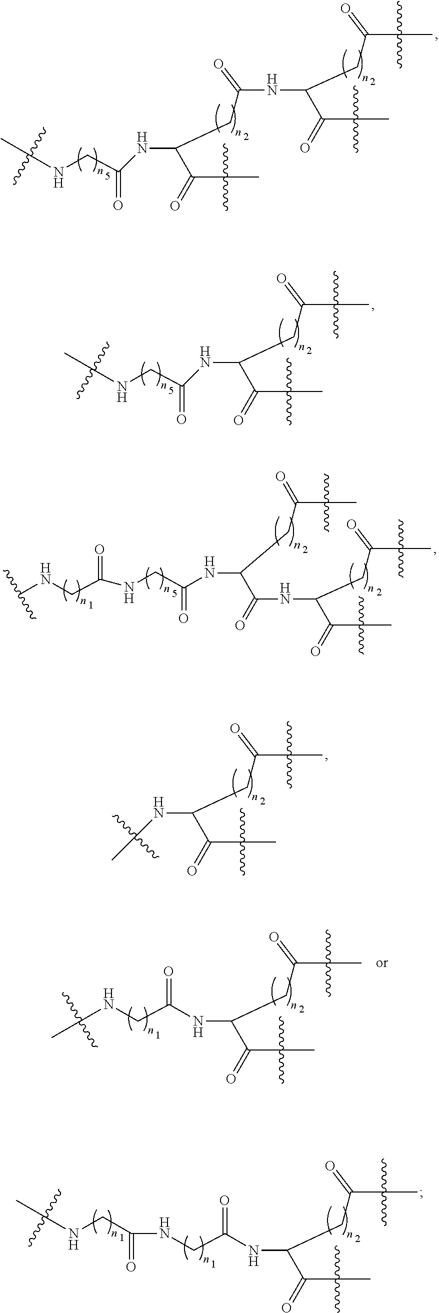

n₁, n₂, n₄ and n₅ are each independently selected from 1, 2, 3, 4, 5, 6, 7 or 8;

### Q is —N-AC;

Q₁ is —N₁-AC₁;
Q₂ is —N₂-AC₂;
N, N₁ and N₂ are each independently selected from

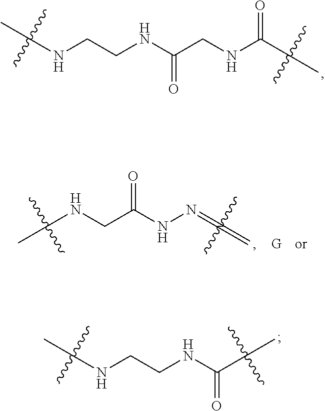 [SMILES: *NCCNC(=O)CNC(*)=O ; *NCC(=O)NN=* ; *NCCNC(*)=O]

and
AC, AC₁ and AC₂ are each independently an anticancer drug.

In some embodiments, L₃ is selected from

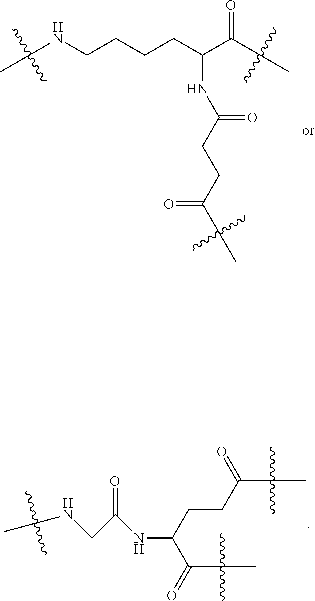 [SMILES: *NCCCCC(NC(=O)CCC(*)=O)C(*)=O ; *NCC(=O)NC(CCC(*)=O)C(*)=O]

In some embodiments W₃ is selected from

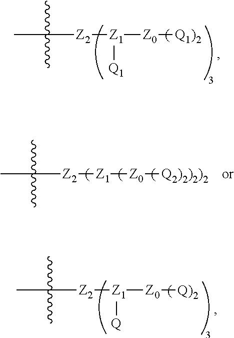

In some embodiments W₄ is selected from

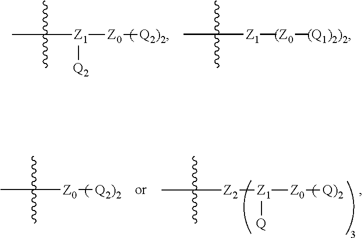

In some embodiments, j₂ is selected from 1, 2 or 3.

In some embodiments, j₂ is 2.

In some embodiments, j₃ is selected from 1, 2 or 3.

In some embodiments, j₃ is 1.

In some embodiments, Z₂, Z₁ and Z₀ are each independently selected from

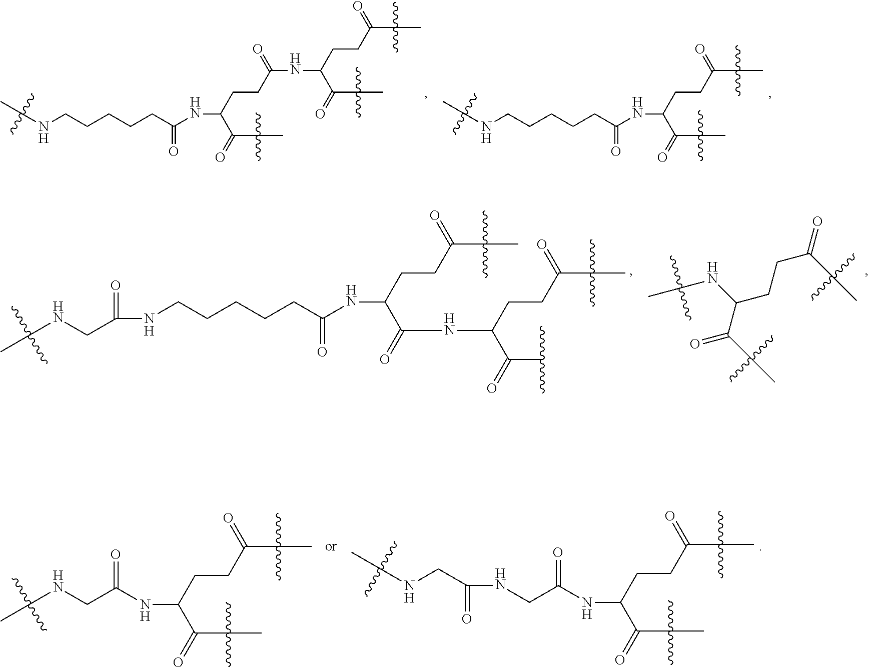 [SMILES: *NCCCCCC(=O)NC(CCC(=O)NC(CCC(*)=O)C(*)=O)C(*)=O ; *NCCCCCC(=O)NC(CCC(*)=O)C(*)=O ; *NCC(=O)NCCCCCC(=O)NC(CCC(*)=O)C(=O)NC(CCC(*)=O)C(*)=O ; *NC(CCC(*)=O)C(*)=O ; *NCC(=O)NC(CCC(*)=O)C(*)=O ; *NCC(=O)NCC(=O)NC(CCC(*)=O)C(*)=O]

In some embodiments, Z₂ is selected from

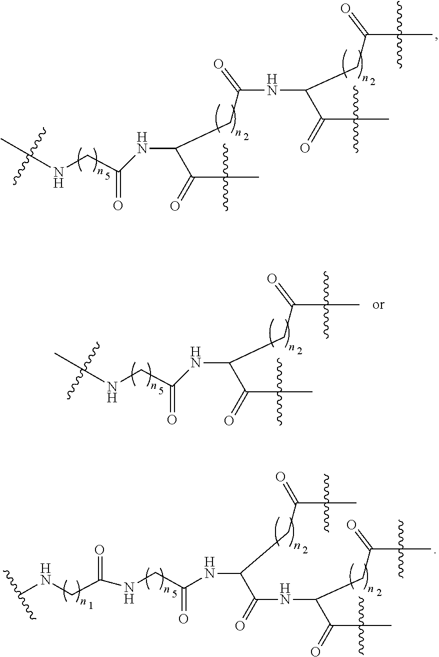

In some embodiments, Z₂ is selected from

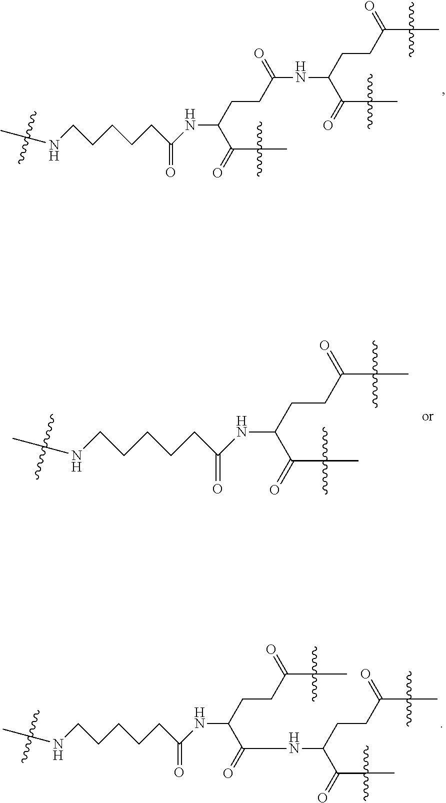 [SMILES: *NCCCCCC(=O)NC(CCC(=O)NC(CCC(*)=O)C(*)=O)C(*)=O ; *NCCCCCC(=O)NC(CCC(*)=O)C(*)=O ; *NCCCCCC(=O)NC(CCC(*)=O)C(=O)NC(CCC(*)=O)C(*)=O]

In some embodiments, Z₁ is selected from

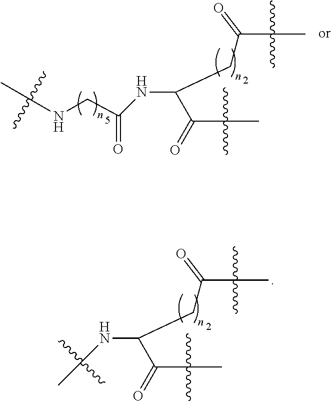

In some embodiments, Z₁ is selected from

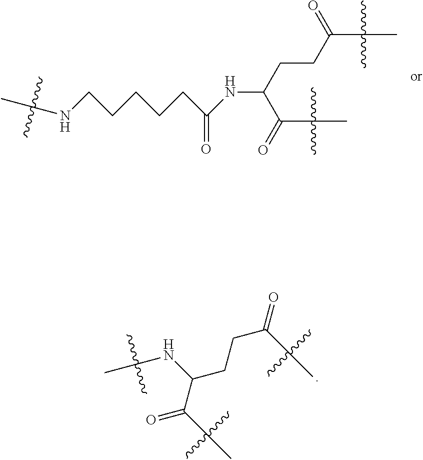 [SMILES: *NCCCCCC(=O)NC(CCC(*)=O)C(*)=O ; *NC(CCC(*)=O)C(*)=O]

In some embodiments, Z₀ is selected from

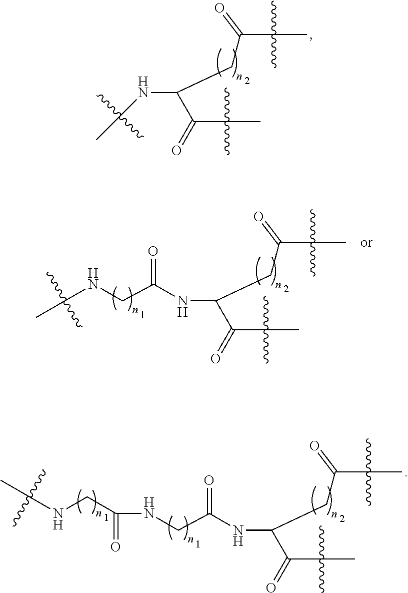

In some embodiments, Z₀ is selected from

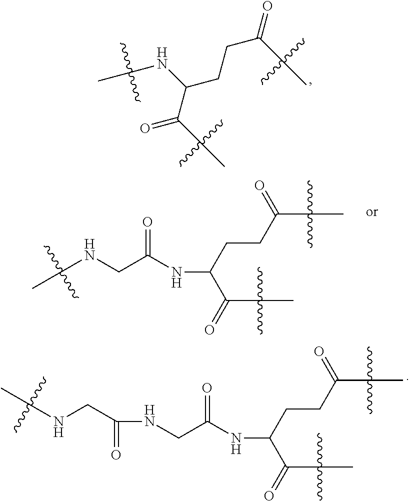 [SMILES: *NC(CCC(*)=O)C(*)=O ; *NCC(=O)NC(CCC(*)=O)C(*)=O ; *NCC(=O)NCC(=O)NC(CCC(*)=O)C(*)=O]

In some embodiments, n₁ is selected from 1, 2 or 3.

In some embodiments, n₁ is 1.

In some embodiments, n₂ is selected from 1, 2 or 3.

In some embodiments, n₂ is 2.

In some embodiments, n₄ is selected from 3, 4 or 5.

In some embodiments, n₄ is 4.

In some embodiments, n₅ is selected from 4, 5 or 6.

In some embodiments, n₅ is 5.

In some embodiments, N is

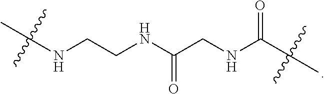 [SMILES: *NCCNC(=O)CNC(*)=O]

In some embodiments, N₁ is selected from

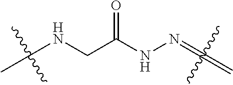 [SMILES: *NCC(=O)NN=*]

### or G.

In some embodiments, N₂ is selected as

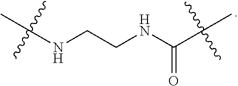 [SMILES: *NCCNC(*)=O]

In some embodiments, AC, AC1 and AC2 are each independently selected from AXT, PCB, 5FU, DXM, SRM, IMQ or LNL.

In some embodiments, AC, AC₁ and AC₂ are each independently selected from 5FU, DXM, SRM or AXT.

In some embodiments, AC is 5FU.

In some embodiments, AC₁ is selected from DXM or SRM.

In some embodiments, AC₂ is AXT.

According to a fourth aspect of the present disclosure, the present disclosure provides a polyethylene glycol-drug conjugate represented by Formula (III), a stereoisomer thereof or a pharmaceutically acceptable salt thereof,

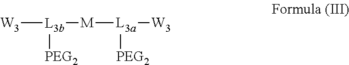

where:

### M is

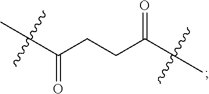 [SMILES: *C(=O)CCC(*)=O]

L₃ₐ and L_{3b} are each independently selected from

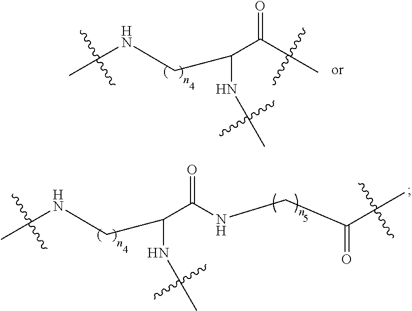

### W₃ is

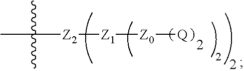

PEG₂ is a single-armed polyethylene glycol chain segment, PEG₂ is connected to L₃ₐ or L_{3b} through a carbonyl group, and a number-average molecular weight of PEG₂ is 5k to 40k, preferably 5k to 10k or 10k to 40k, and more preferably 10k;
Z₂, Z₁ and Z₀ are each independently selected from

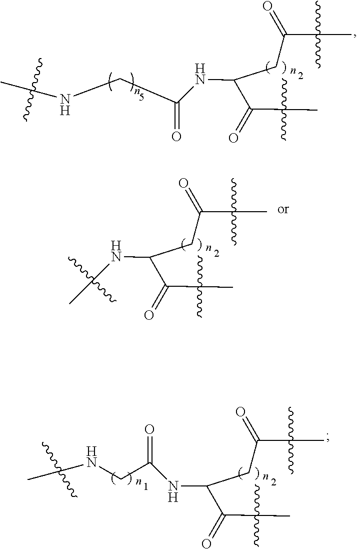

n₁, n₂, n₄ and n₅ are each independently selected from 1, 2, 3, 4, 5, 6, 7 or 8;

### Q is —N-AC;

### N is

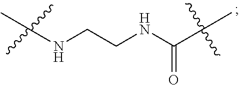 [SMILES: *NCCNC(*)=O]

and
AC is an anticancer drug.

In some embodiments, L₃ₐ and L_{3b} are each independently selected from

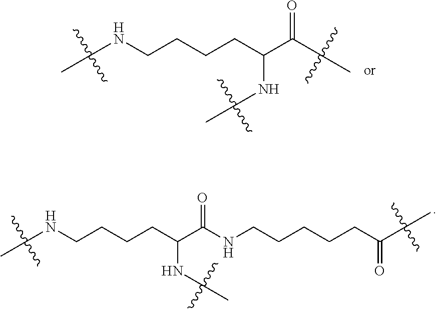 [SMILES: *NCCCCC(N*)C(*)=O ; *NCCCCC(N*)C(=O)NCCCCCC(*)=O]

In some embodiments, L₃ₐ is

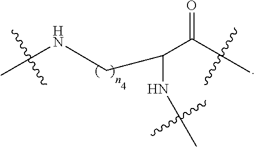 [SMILES: *NCC(N*)C(*)=O]

In some embodiments, L₃ₐ is

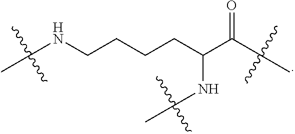 [SMILES: *NCCCCC(N*)C(*)=O]

In some embodiments, L_{3b} is

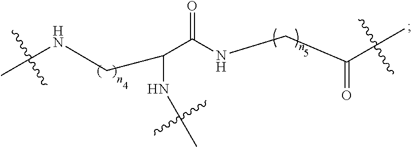

In some embodiments, L_{3b} is

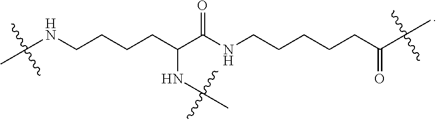 [SMILES: *NCCCCC(N*)C(=O)NCCCCCC(*)=O]

In some embodiments, Z₂, Z₁ and Z₀ are each independently selected from

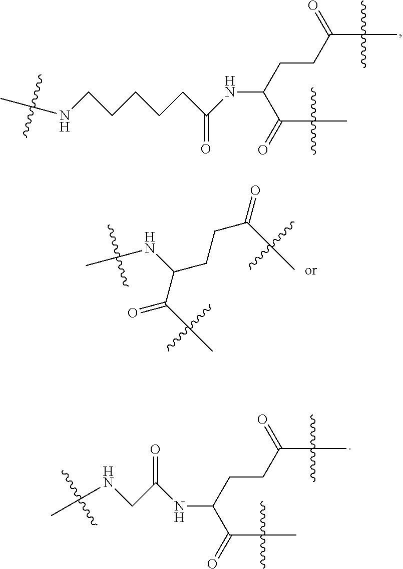

In some embodiments, Z₂ is

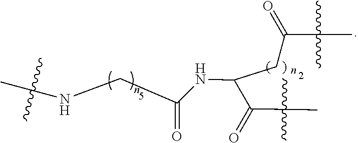

In some embodiments, Z₂ is

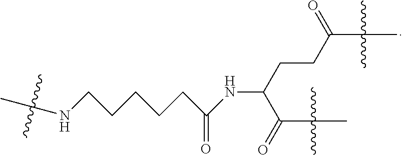 [SMILES: *NCCCCCC(=O)NC(CCC(*)=O)C(*)=O]

In some embodiments, Z₁ is

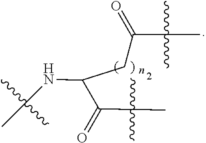 [SMILES: *NC(CC(*)=O)C(*)=O]

In some embodiments, Z₁ is

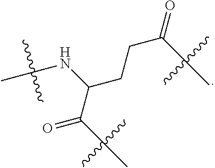 [SMILES: *NC(CCC(*)=O)C(*)=O]

In some embodiments, Z₀ is

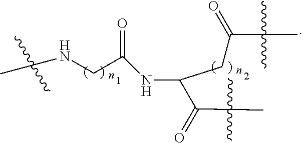 [SMILES: *NCC(=O)NC(CC(*)=O)C(*)=O]

In some embodiments, Z₀ is

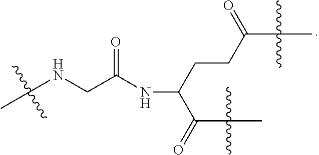 [SMILES: *NCC(=O)NC(CCC(*)=O)C(*)=O]

In some embodiments, n₁ is selected from 1, 2 or 3.

In some embodiments, n₁ is 1.

In some embodiments, n₂ is selected from 1, 2 or 3.

In some embodiments, n₂ is 2.

In some embodiments, n₄ is selected from 3, 4 or 5.

In some embodiments, n₄ is 4.

In some embodiments, n₅ is selected from 4, 5 or 6.

In some embodiments, n₅ is 5.

In some embodiments, AC is selected from AXT, PCB, 5FU, DXM, SRM, IMQ or LNL.

In some embodiments, AC is AXT.

According to a fifth aspect of the present disclosure, the present disclosure provides a polyethylene glycol-drug conjugate represented by Formula (IV), a stereoisomer thereof or a pharmaceutically acceptable salt thereof.

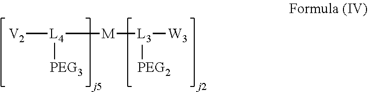

where:

### M is

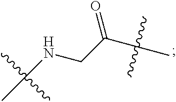 [SMILES: *NCC(*)=O]

L₃ and L₄ are each independently selected from

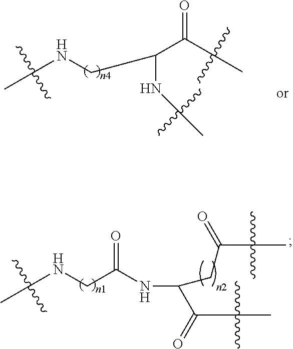

PEG₂ and PEG₃ are each independently a single-armed polyethylene glycol chain segment, PEG₂ is connected to L₃ through a carbonyl group, PEG₃ is connected to L₄ through a carbonyl group, and a number-average molecular weight of each of PEG₂ and PEG₃ is independently 5k to 40k, preferably 5k to 10k or 10k to 40k, and more preferably 10k;
j₂ and j₅ are each independently selected from 1, 2, 3, 4 or 5;

### W₃ is

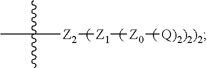

Z₂, Z₁ and Z₀ are each independently selected from

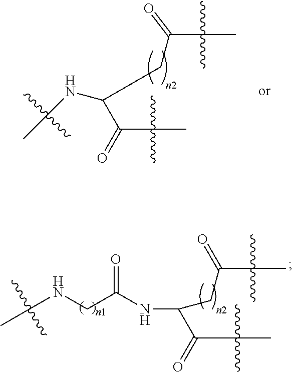

### Q is —N-AC;

### N is

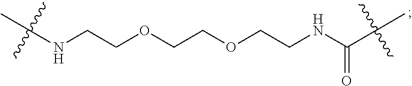 [SMILES: *NCCOCCOCCNC(*)=O]

and
AC is an anticancer drug; and

### V₂ is

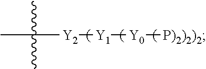

Y₂, Y₁ and Y₀ are each independently selected from

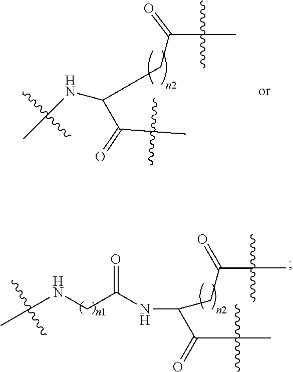

### Lᵥ is

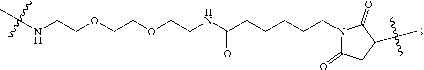 [SMILES: *NCCOCCOCCNC(=O)CCCCCN1C(=O)CC(*)C1=O]

### T is PPT-iRGD; and

n₁, n₂ and n₄ are each independently selected from 1, 2, 3, 4, 5, 6, 7 or 8.

In some embodiments, L₃ and L₄ are each independently selected from

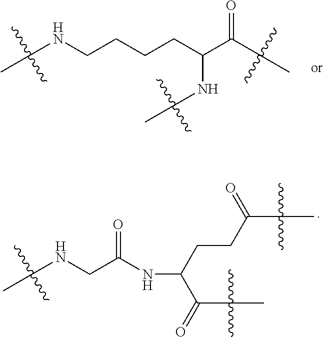 [SMILES: *NCCCCC(N*)C(*)=O ; *NCC(=O)NC(CCC(*)=O)C(*)=O]

In some embodiments, L₃ is

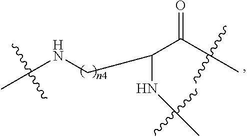

In some embodiments, L₃ is

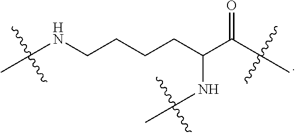 [SMILES: *NCCCCC(N*)C(*)=O]

In some embodiments, L₄ is

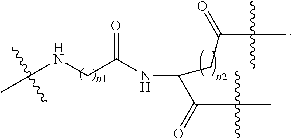

In some embodiments, L₄ is

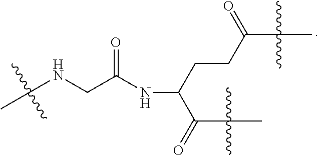 [SMILES: *NCC(=O)NC(CCC(*)=O)C(*)=O]

In some embodiments, j₂ is selected from 1, 2 or 3.

In some embodiments, j₂ is 1.

In some embodiments, j₅ is selected from 1, 2 or 3.

In some embodiments, j₅ is 1.

In some embodiments, Z₂, Z₁ and Z₀ are each independently selected from

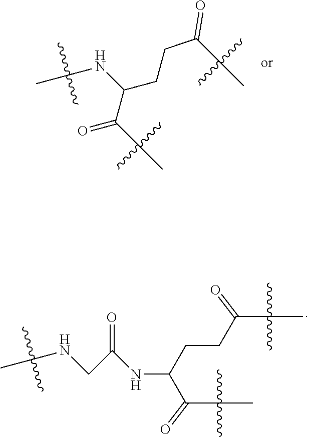 [SMILES: *NC(CCC(*)=O)C(*)=O ; *NCC(=O)NC(CCC(*)=O)C(*)=O]

In some embodiments, Z₂ is

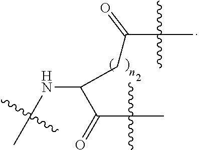

In some embodiments, Z₂ is

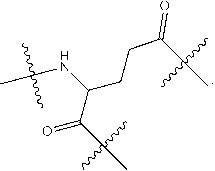 [SMILES: *NC(CCC(*)=O)C(*)=O]

In some embodiments, Z₁ is

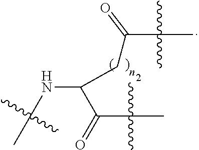

In some embodiments, Z₁ is

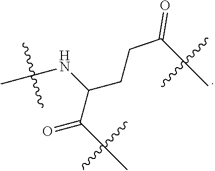 [SMILES: *NC(CCC(*)=O)C(*)=O]

In some embodiments, Z₀ is

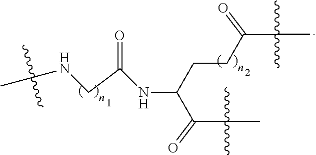

In some embodiments, Z₀ is

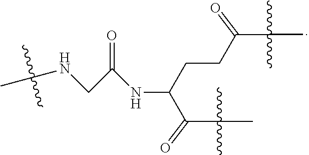 [SMILES: *NCC(=O)NC(CCC(*)=O)C(*)=O]

In some embodiments, AC is selected from AXT, PCB, 5FU, DXM, SRM, IMQ or LNL.

In some embodiments, AC is AXT.

In some embodiments, Y₂, Y₁ and Y₀ are each independently selected from

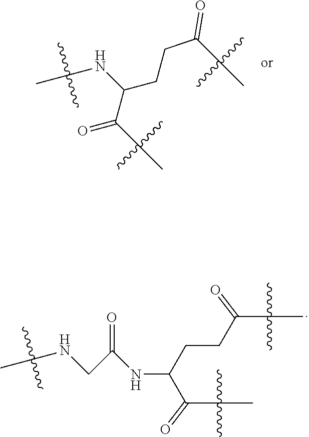 [SMILES: *NC(CCC(*)=O)C(*)=O ; *NCC(=O)NC(CCC(*)=O)C(*)=O]

In some embodiments, Y₂ is

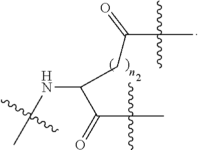

In some embodiments, Y₂ is

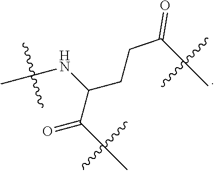 [SMILES: *NC(CCC(*)=O)C(*)=O]

In some embodiments, Y₁ is

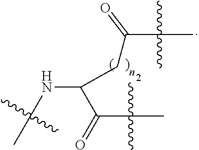

In some embodiments, Y₁ is

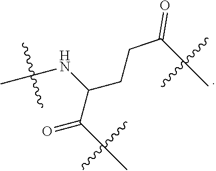 [SMILES: *NC(CCC(*)=O)C(*)=O]

In some embodiments, Y₀ is

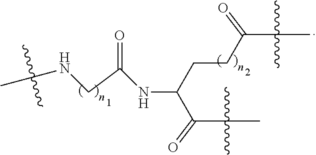

In some embodiments, Y₀ is

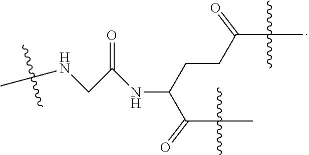 [SMILES: *NCC(=O)NC(CCC(*)=O)C(*)=O]

In some embodiments, n₁ is selected from 1, 2 or 3.

In some embodiments, n₁ is 1.

In some embodiments, n₂ is selected from 1, 2 or 3.

In some embodiments, n₂ is 2.

In some embodiments, n₄ is selected from 3, 4 or 5.

In some embodiments, n₄ is 4.

According to a sixth aspect of the present disclosure, the present disclosure provides a polyethylene glycol-drug conjugate represented by Formula (V), a stereoisomer thereof or a pharmaceutically acceptable salt thereof,

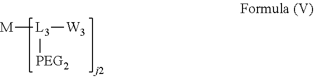

where:
M is selected from

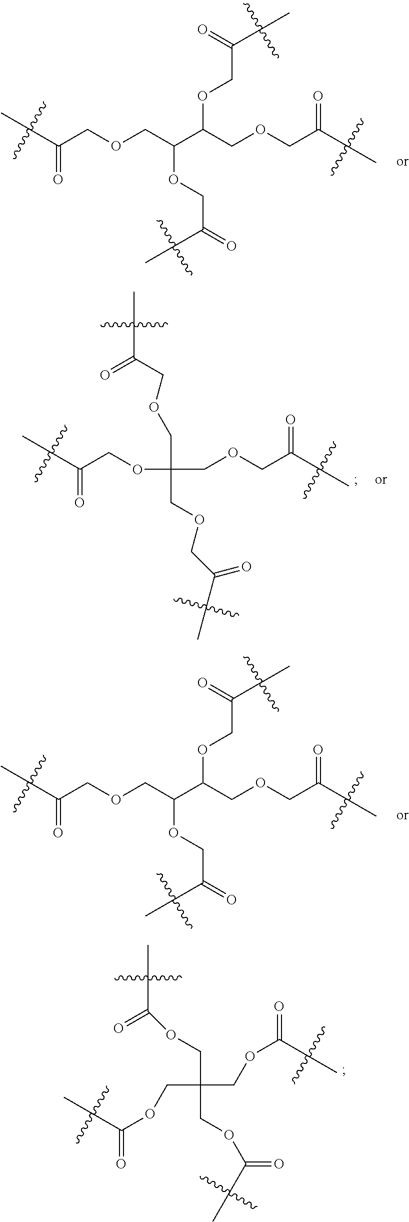 [SMILES: *C(=O)COCC(OCC(*)=O)C(COCC(*)=O)OCC(*)=O ; *C(=O)COCC(COCC(*)=O)(COCC(*)=O)OCC(*)=O ; *C(=O)COCC(OCC(*)=O)C(COCC(*)=O)OCC(*)=O ; *C(=O)OCC(COC(*)=O)(COC(*)=O)COC(*)=O]

M is selected from
and
L₃ is selected from

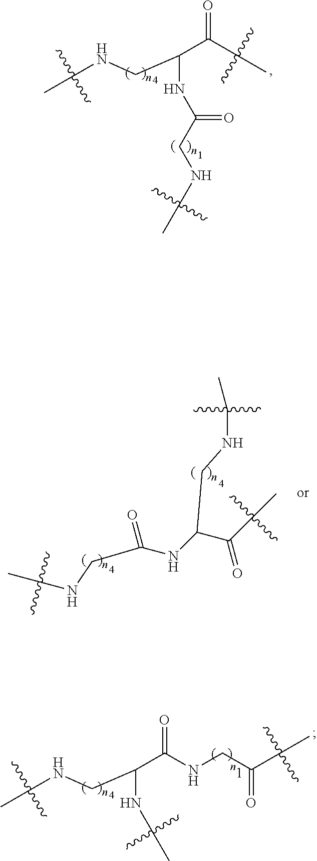

PEG₂ is a single-armed polyethylene glycol chain segment, PEG₂ is connected to L₃ through a carbonyl group or an amino group, and a number-average molecular weight of PEG₂ is 5k to 40k, preferably 5k to 10k or 10k to 40k, and more preferably 10k;
j₂ is selected from 3, 4 or 5;
W₃ is selected from

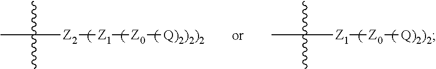

Z₂, Z₁ and Z₀ are each independently selected from

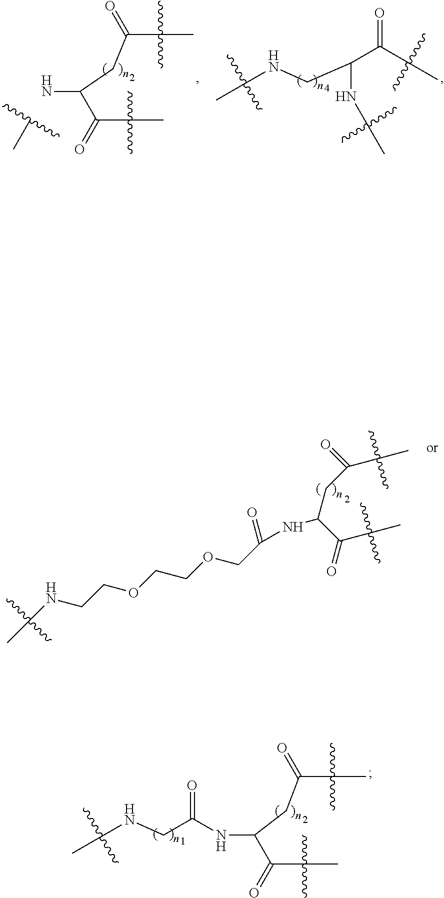

n₁, n₂ and n₄ are each independently selected from 1, 2, 3, 4, 5, 6, 7 or 8;

### Q is —N-AC;

N is selected from

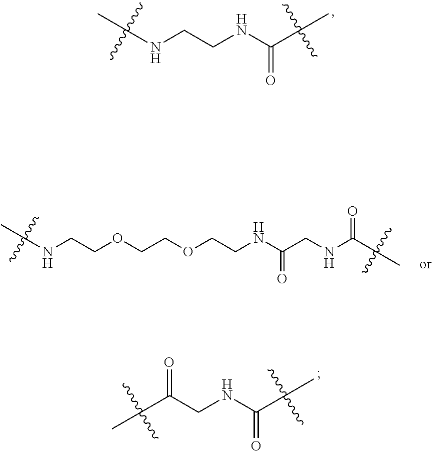 [SMILES: *NCCNC(*)=O ; *NCCOCCOCCNC(=O)CNC(*)=O ; *C(=O)CNC(*)=O]

AC is an anticancer drug.

In some embodiments, L₃ is selected from

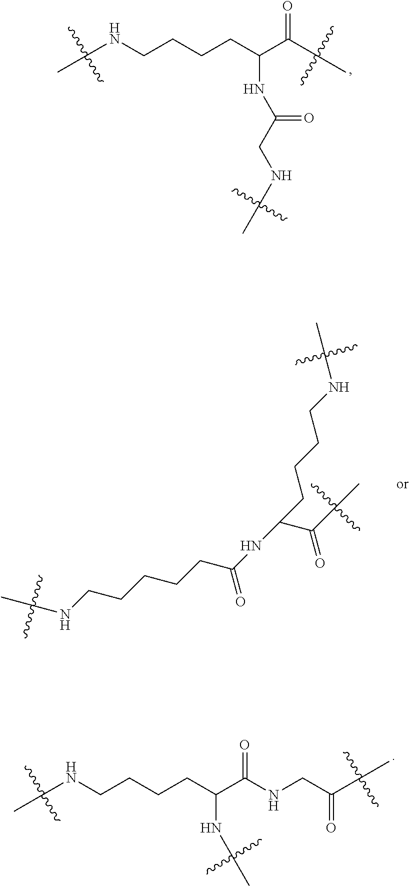 [SMILES: *NCCCCC(NC(=O)CN*)C(*)=O ; *NCCCCCC(=O)NC(CCCCN*)C(*)=O ; *NCCCCC(N*)C(=O)NCC(*)=O]

In some embodiments, L₃ is selected from

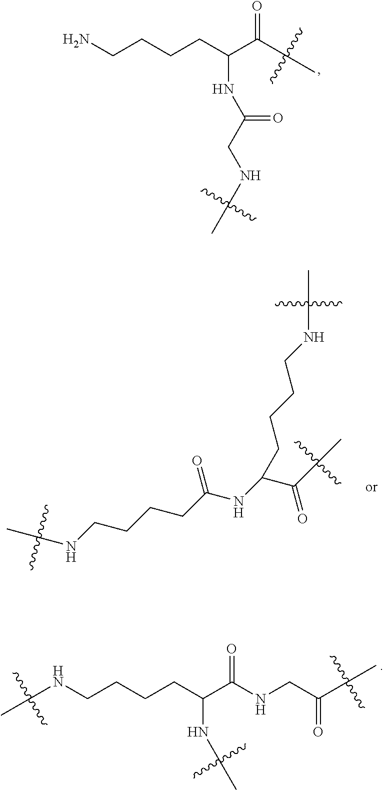 [SMILES: *NCC(=O)NC(CCCCN)C(*)=O ; *NCCCCC(=O)NC(CCCCN*)C(*)=O ; *NCCCCC(N*)C(=O)NCC(*)=O]

In some embodiments, j₂ is 4.

In some embodiments, Z₂, Z₁ and Z₀ are each independently selected from

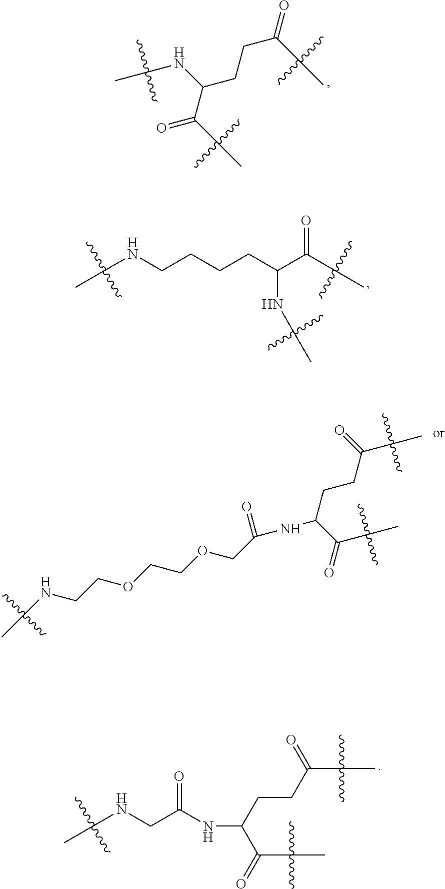 [SMILES: *NC(CCC(*)=O)C(*)=O ; *NCCCCC(N*)C(*)=O ; *NCCOCCOCC(=O)NC(CCC(*)=O)C(*)=O ; *NCC(=O)NC(CCC(*)=O)C(*)=O]

In some embodiments, Z₂ is selected from

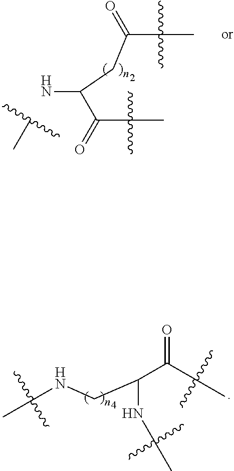

In some embodiments, Z₂ is selected from

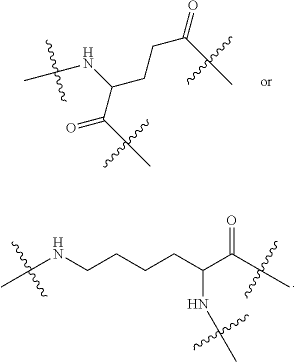 [SMILES: *NC(CCC(*)=O)C(*)=O ; *NCCCCC(N*)C(*)=O]

In some embodiments, Z₁ is selected from

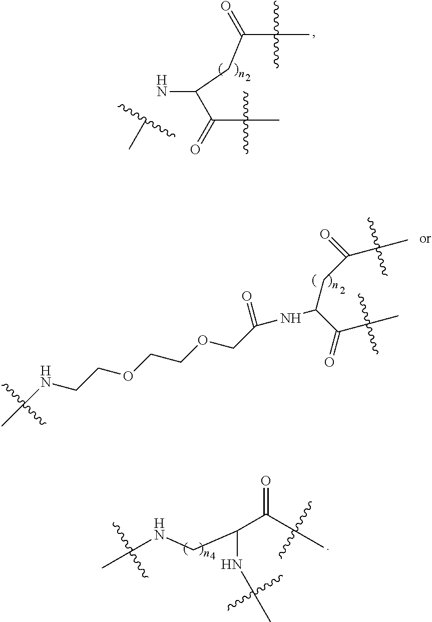

In some embodiments, Z₁ is selected from

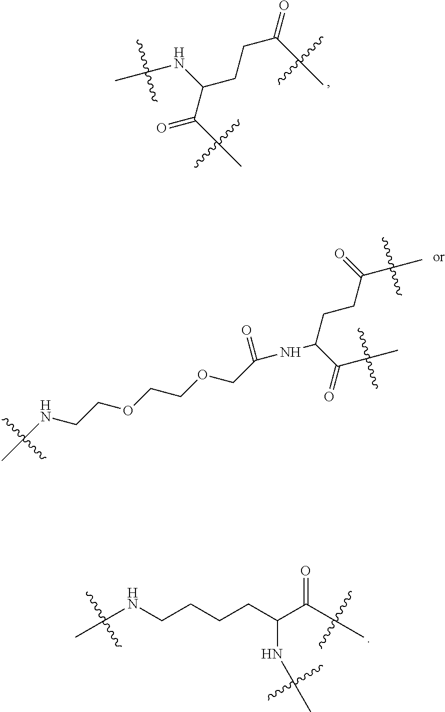 [SMILES: *NC(CCC(*)=O)C(*)=O ; *NCCOCCOCC(=O)NC(CCC(*)=O)C(*)=O ; *NCCCCC(N*)C(*)=O]

In some embodiments, Z₀ is selected from

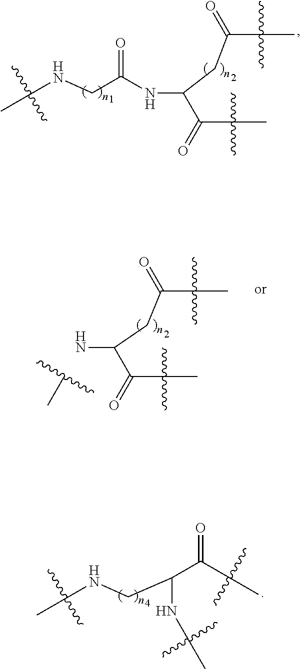

In some embodiments, Z₀ is selected from

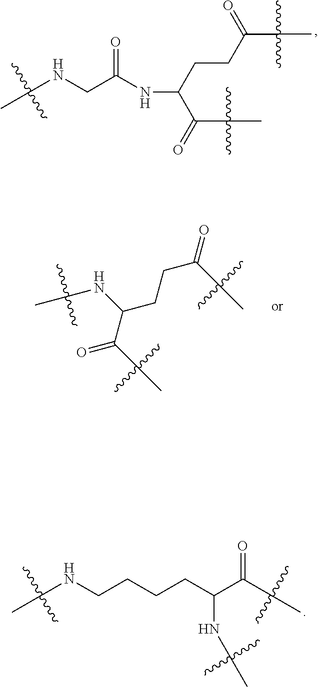 [SMILES: *NCC(=O)NC(CCC(*)=O)C(*)=O ; *NC(CCC(*)=O)C(*)=O ; *NCCCCC(N*)C(*)=O]

In some embodiments, n₁ is selected from 1, 2 or 3.

In some embodiments, n₁ is 1.

In some embodiments, n₂ is selected from 1, 2 or 3.

In some embodiments, n₂ is 2.

In some embodiments, n₄ is selected from 3, 4 or 5.

In some embodiments, n₄ is selected from 4 or 5.

In some embodiments, n₄ is 4.

In some embodiments, AC is selected from AXT, PCB, 5FU, DXM, SRM, IMQ or LNL.

In some embodiments, AC is selected from IMQ, LNL or 5FU.

According to a seventh aspect of the present disclosure, the present disclosure provides a polyethylene glycol-drug conjugate represented by Formula (VI), a stereoisomer thereof or a pharmaceutically acceptable salt thereof,

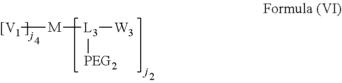

where:

### M is

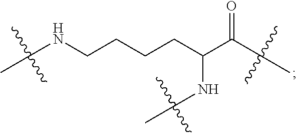 [SMILES: *NCCCCC(N*)C(*)=O]

### L₃ is

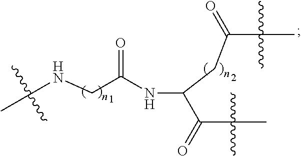

PEG₂ is a single-armed polyethylene glycol chain segment, PEG₂ is connected to L₃ through a carbonyl group, and a number-average molecular weight of PEG₂ is 5k to 40k, preferably 5k to 10k or 10k to 40k, and more preferably 10k;
j₂ and j₄ are each independently selected from 1, 2, 3, 4 or 5;

### W₃ is

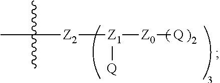

Z₂, Z₁ and Z₀ are each independently selected from

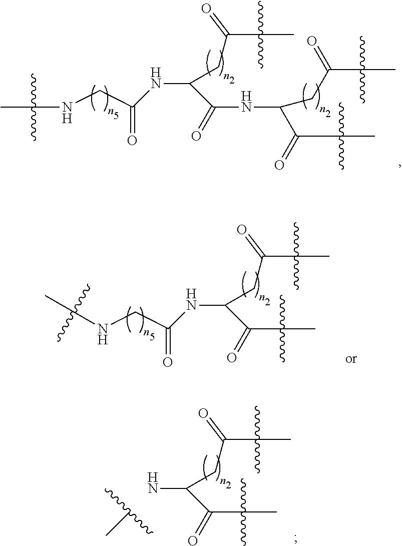

### Q is —N-AC;

### N is

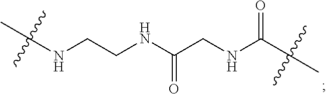 [SMILES: *NCCNC(=O)CNC(*)=O]

AC is an anticancer drug;

### V₁ is

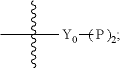

### Y₀ is

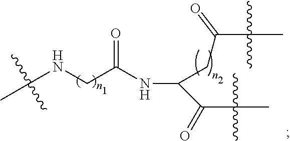

### P is -Lᵥ-T;

### Lᵥ is

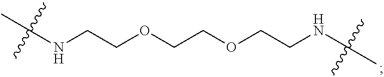 [SMILES: *NCCOCCOCCN*]

### T is FA; and

n₁, n₂ and n₅ are each independently selected from 1, 2, 3, 4, 5, 6, 7 or 8.

In some embodiments, L₃ is

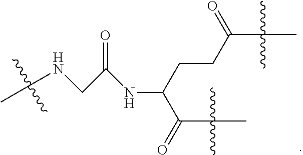 [SMILES: *NCC(=O)NC(CCC(*)=O)C(*)=O]

In some embodiments, j₂ is selected from 1, 2 or 3.

In some embodiments, j₂ is 2.

In some embodiments, j₄ is selected from 1, 2 or 3.

In some embodiments, j₄ is 1.

In some embodiments, Z₂, Z₁ and Z₀ are each independently selected from

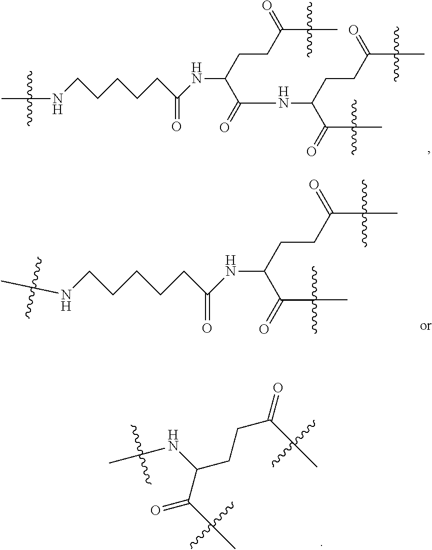

In some embodiments, Z₂ is

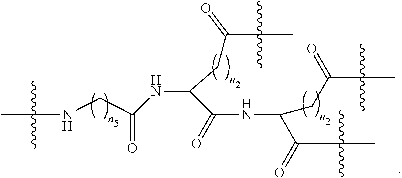

In some embodiments, Z₂ is

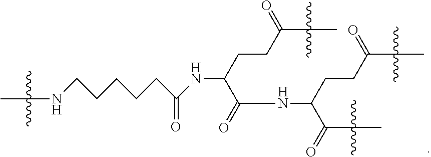 [SMILES: *NCCCCCC(=O)NC(CCC(*)=O)C(=O)NC(CCC(*)=O)C(*)=O]

In some embodiments, Z₁ is

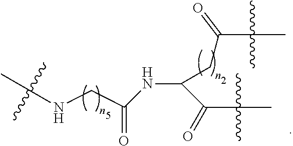

In some embodiments, Z₁ is

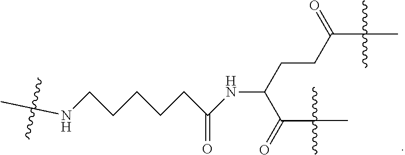 [SMILES: *NCCCCCC(=O)NC(CCC(*)=O)C(*)=O]

In some embodiments, Z₀ is

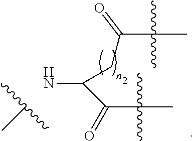

In some embodiments, Z₀ is

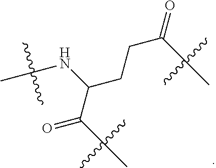 [SMILES: *NC(CCC(*)=O)C(*)=O]

In some embodiments, AC is selected from AXT, PCB, 5FU, DXM, SRM, IMQ or LNL.

In some embodiments, AC is 5FU.

In some embodiments, Y₀ is

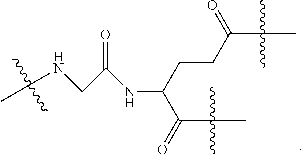 [SMILES: *NCC(=O)NC(CCC(*)=O)C(*)=O]

In some embodiments, n₁ is selected from 1, 2 or 3.

In some embodiments, n₁ is 1.

In some embodiments, n₂ is selected from 1, 2 or 3.

In some embodiments, n₂ is 2.

In some embodiments, n₅ is selected from 4, 5 or 6.

In some embodiments, n₅ is 5.

According to an eighth aspect of the present disclosure, the present disclosure provides a polyethylene glycol-drug conjugate, a stereoisomer thereof or a pharmaceutically acceptable salt thereof, where the polyethylene glycol-drug conjugate is selected from:

According to a ninth aspect of the present disclosure, the present disclosure provides an intermediate for preparing the foregoing polyethylene glycol-drug conjugate, the stereoisomer thereof or the pharmaceutically acceptable salt thereof, where the intermediate is selected from:

According to a tenth aspect of the present disclosure, the present disclosure provides a pharmaceutical composition including the foregoing polyethylene glycol-drug conjugate, the stereoisomer thereof or the pharmaceutically acceptable salt thereof; and optionally, the composition further includes one or more pharmaceutically acceptable auxiliary materials such as a carrier and/or an excipient. The carrier and/or excipient includes but is not limited to: an ion exchanger, aluminum oxide, aluminum stearate, lecithin, serum protein such as human serum albumin, buffer substances such as phosphate, glycerol, sorbic acid, potassium sorbate, partial glyceride mixture of saturated vegetable fatty acids, water, salts or electrolytes, such as protamine sulfate, disodium hydrogen phosphate, potassium hydrogen phosphate, sodium chloride, zinc salts, colloidal silicon oxide, magnesium trisilicate, polyvinylpyrrolidone, cellulose material, polyethylene glycol, sodium carboxymethylcellulose, polyacrylate, beeswax, polyethylene-polyoxypropylene block polymer and lanolin.

The pharmaceutical composition can be prepared into any pharmaceutically acceptable dosage form. The pharmaceutical composition may also be administered to an individual in need of such treatment in any suitable mode of administration, such as oral, parenteral, rectal or pulmonary administration. When used for oral administration, the pharmaceutical composition can be prepared into a conventional solid preparation, for example, a tablet, a capsule, a pill or granule, or can also be prepared into an oral liquid preparation such as an oral liquid formulation, an oral suspension or syrup. When the pharmaceutical composition is prepared into an oral preparation, an appropriate filler, a binder, a disintegrant, a lubricant or the like can be added. When used for parenteral administration, the pharmaceutical composition can be prepared into an injection, including an injection liquid, sterile powder for injection and a concentrated solution for injection. When making injections, conventional methods in the existing pharmaceutical field can be used. During preparation of the injections, no additives may be added, or appropriate additives may be added according to the properties of the drugs. When used for rectal administration, the pharmaceutical composition can be prepared into a suppository or the like. When used for transpulmonary administration, the pharmaceutical composition can be prepared into an inhalant or a spray. Preferably, the pharmaceutical composition in the present disclosure can be prepared into an injection such as an injection solution. Optionally, normal saline is used as a carrier of the injection.

According to an eleventh aspect of the present disclosure, the present disclosure provides use of the polyethylene glycol-drug conjugate, the stereoisomer thereof or the pharmaceutically acceptable salt thereof in preparation of a drug for treatment and/or prevention of a disease (for example, cancer), where the disease is a disease to be treated by an active ingredient in the polyethylene glycol-drug conjugate.

According to another aspect of the present disclosure, the present application provides a method for treatment and/or prevention of a disease (for example, cancer), including administering a therapeutically effective dose of the polyethylene glycol-drug conjugate, the stereoisomer thereof or the pharmaceutically acceptable salt thereof to a subject in need thereof. Preferably, the disease is a disease to be treated by an active ingredient in the polyethylene glycol-drug conjugate. A dosage regimen can be adjusted to provide the best desired response. For example, a single injection may be administered, several divided doses may be administered over time, or the dosage may be proportionally reduced or increased as the exigencies of the therapeutic situation indicate. It should be noted that the dosage may vary depending on the type and severity of the condition to be alleviated, and may include single or multiple doses. It should be further understood that, for any particular individual, a specific dosage regimen should be adjusted over time based on the individual needs and the professional discretion of the personnel administering the composition or supervising administration of the composition.

According to another aspect of the present disclosure, the present application provides a polyethylene glycol-drug conjugate, a stereoisomer thereof or a pharmaceutically acceptable salt thereof, for use in the treatment and/or prevention of a disease (for example, cancer). Preferably, the disease is a disease to be treated by an active ingredient in the polyethylene glycol-drug conjugate.

In some embodiments, the disease is an ophthalmic disease. In some embodiments, the cancer is eye cancer.

In some embodiments, the disease is a disease associated with choroidal neovascularization (CNV).

In some embodiments, the disease is selected from the group consisting of choroidal neovascularization (CNV), diabetic retinopathy, central exudative retinochoroiditis, macular degeneration (for example, AMD), high myopia retinopathy and the like.

In the present disclosure, cancer refers to a cell proliferative disease state, including but not limited to: colon cancer, leukemia, lymphoma, bladder cancer, bone cancer, brain tumor, medulloblastoma, glioma, breast cancer, adenoma/carcinoid, adrenocortical carcinoma, islet cell carcinoma, cervical cancer, endometrial cancer, ovarian cancer, colorectal cancer, skin cancer, esophageal cancer, eye cancer, gallbladder cancer, gastric cancer, head and neck cancer, liver cancer, melanoma, Kaposi's sarcoma, kidney cancer, oral cancer, lung cancer, nasopharyngeal cancer, neuroblastoma, ovarian cancer, pancreatic cancer, thyroid cancer, parathyroid/penile cancer, prostate cancer, urethral cancer, vaginal cancer, vulvar cancer, anal cancer, and sarcoma, and preferably, eye cancer, and including metastasis of the preceding cancer.

As used herein, the term “subject” includes humans or non-human animals. An exemplary human subject includes a human subject (referred to as a patient) suffering from a disease (for example, the disease described herein) or a normal subject. In the present disclosure, “non-human animals” include all vertebrates such as non-mammals (such as birds, amphibians or reptiles) and mammals such as non-human primates, domestic animals and/or domesticated animals (such as sheep, dogs, cats, cows and pigs).

In the present disclosure, the term “effective dose” refers to an amount of a compound that can alleviate one or more of treated symptoms to some extent when administered.

In the present disclosure, the term “treatment” means reversion, alleviation and inhibition of a disorder or condition to which such term applies, or progression of one or more symptoms of such disorder or condition, or prevention of such disorder or condition, or one or more symptoms of such disorder or condition.

The polyethylene glycol-drug conjugate in the present disclosure uses amino acids, polypeptides or the like as connecting chains, and uses dicarboxylic acids or polycarboxylic acids with amino groups (for example, natural amino acids with two carboxyl groups) or carboxylic acids with diamino groups or polyamino groups (for example, natural amino acids with two amino groups) or polycarboxylic acids as connecting bridges to couple multiple identical or different drug molecules together by forming amide bonds. The type, proportion and drug loading of the drug can be adjusted. In some embodiments, the activated PEG reacts with the amino groups on the backbone via carboxyl groups to form amide bonds. In some other embodiments, the activated PEG reacts with the carboxyl groups on the backbone via amino groups to form amide bonds. Therefore, those skilled in the art can understand that in the present disclosure, when “PEG₂ is connected to L₃ through a carbonyl group or an amino group”, the “carbonyl group or amino group” does not mean that an additional carbonyl group or amino group is connected, but means that the activated PEG residue is connected to L₃ through its terminal carbonyl group or amino group. Other similar definitions can be understood with reference to the foregoing content. In some embodiments, the molecular weight of the PEG includes terminal amino groups.

In the present disclosure, the active ingredient suitable for coupling with polyethylene glycol can be a drug molecule with at least one amino group, hydroxyl group, carboxyl group or acyl group, for example, a drug molecule with at least one amino group, hydroxyl group, carboxyl group or acyl group with anti-tumor activity, for example, AXT, PCB, 5FU, DXM, SRM, IMQ and LNL, which have the following meanings:

| Abbreviation | Name | CAS No. |
|---|---|---|
| PCB | Palbociclib | 571190-30-2 |
| AXT | Axitinib | 319460-85-0 |
| 5FU | 5-FU | 51-21-8 |
| DXM | Dexamethasone | 50-02-2 |
| SRM | Rapamycin | 53123-88-9 |
| IMQ | IMIQUIMOD | 99011-02-6 |
| LNL | Lenalidomide | 191732-72-6 |

In the present disclosure, the connection sites between the following drug molecules and other parts of the overall structure of the polyethylene glycol-drug conjugate are shown in the table below, specifically the positions indicated by “ [symbol] ”.

| Abbreviation | Connection site |
|---|---|
| PCB | 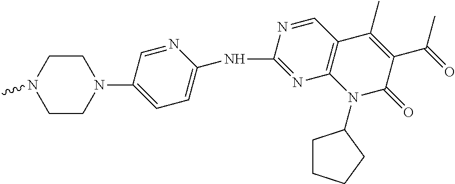 |
| AXT | 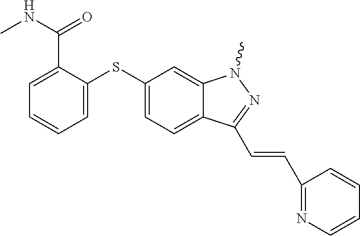 |
| 5FU | 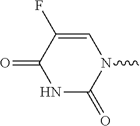 |
| DXM | 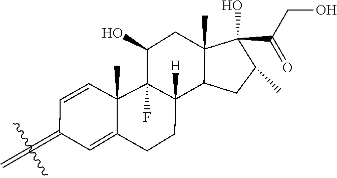 |
| SRM | 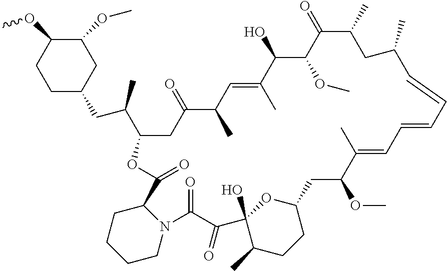 |
| IMQ | 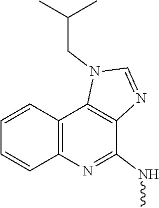 |
| LNL | 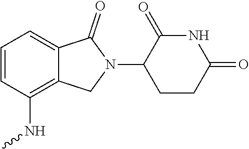 |

In addition, FA represents folic acid with a structural formula of

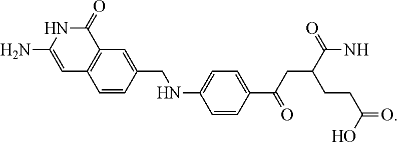 [SMILES: NC(=O)C(CCC(=O)O)CC(=O)c1ccc(NCc2ccc3cc(N)[nH]c(=O)c3c2)cc1]

The connection site between FA and another part of the overall structure of the polyethylene glycol-drug conjugate is

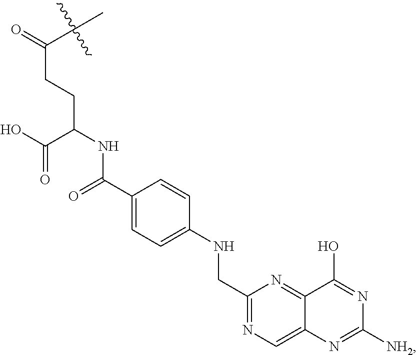 [SMILES: *C(=O)CCC(NC(=O)c1ccc(NCc2ncc3nc(N)nc(O)c3n2)cc1)C(=O)O]

i.e., the position indicated by “ [symbol] ”.

The structural formula of PPT-iRGD is

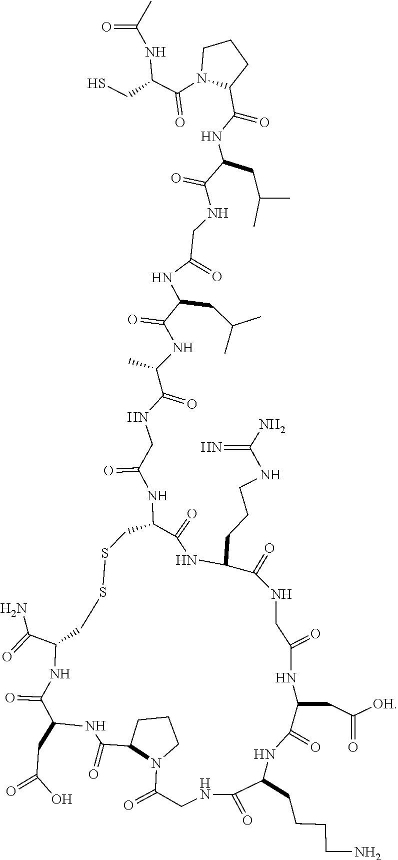

The connection site between PPT-iRGD and another part of the overall structure of the polyethylene glycol-drug conjugate is a terminal thiol group

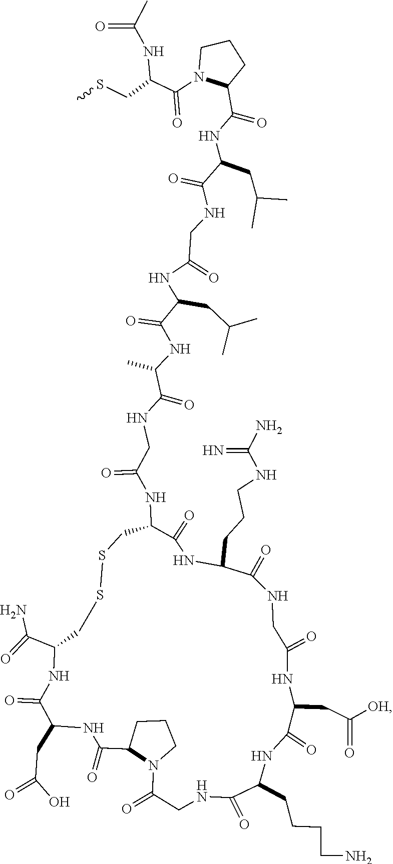

i.e., the position indicated by “ [symbol] ”.

As used herein, “PEG” is the abbreviation of polyethylene glycol and refers to homopolymers with the repeating unit of —CH₂CH₂O—, including single-arm polyethylene glycol, multi-arm polyethylene glycol and their derivatives, such as derivatives with reactive functional groups such as amino or carboxyl groups at the terminal group. In the structural formula according to the present disclosure, the letter n in the subscript of the repeating unit of polyethylene glycol represents the degree of polymerization of polyethylene glycol.

As used herein, unless expressly stated otherwise, the expressions “each . . . independently is” and “ . . . and . . . are each independently” used throughout the description are interchangeable and should be understood in a broad sense, which can mean that specific options represented by the same symbols in different groups do not affect each other, or can mean that specific options represented by the same symbols in the same group do not affect each other.

As used herein, the “pharmaceutically acceptable salts” of the compound in the present disclosure include acid addition salts and base addition salts of the compounds, for example, hydrochloride, hexafluorophosphate or meglumine salt and the like. The “pharmaceutically acceptable salts” include but are not limited to formate, acetate, propionate, benzoate, maleate, fumarate, succinate, tartrate, citrate, ascorbate, α-ketoglutarate, α-glycerophosphate, alkyl sulfonate or aryl sulfonate; and preferably, the alkyl sulfonate is methyl sulfonate or acetyl sulfonate; and the aryl sulfonate is benzenesulfonate or p-toluenesulfonate. Suitable inorganic salts may also be formed including, but not limited to, hydrochloride, hydrobromide, hydroiodate, nitrate, bicarbonate, carbonate, sulfate, phosphate and the like.

As used herein, the tilde “ [symbol] ” in a structural formula refers to a position at which another group is bonded to the structure represented by the structural formula.

### BRIEF DESCRIPTION OF DRAWINGS

FIG. 1 shows changes in a left eye spot and fundus angiography examination results of mouse No. 5 before and after modeling and administration.

FIG. 2 shows changes in a right eye spot and fundus angiography examination results of mouse No. 6 before and after modeling and administration.

FIG. 3 shows changes in a right eye spot and fundus angiography examination results of mouse No. 7 before and after modeling and administration.

FIG. 4 shows changes in a left eye spot and fundus angiography examination results of mouse No. 7 before and after modeling and administration.

### DETAILED DESCRIPTION

The embodiments of the present disclosure will be described in detail below with reference to examples, but persons skilled in the art will understand that the following examples are only used to illustrate the present disclosure and should not be construed as limitation on the scope of the present disclosure. For an example for which a detailed condition is not specified, the example is conducted under a regular condition or a condition recommended by a manufacturer. Any used reagent or instrument for which a manufacturer is not specified is a regular commercially available product.

Meanings of the abbreviations in the examples are as follows:
G Glycine residue
L Leucine residue
F Phenylalanine residue
Asp Aspartic acid residue
E Glutamic acid residue
Glu Glutamic acid residue
DMF N,N-dimethylformamide
TFA Trifluoroacetic acid
t-Bu Tert-butyl
Bn Benzyl
Boc Tert-butoxycarbonyl
Fmoc Fluorenylmethyloxycarbonyl
HOBT 1-hydroxybenzotriazole
Ts p-toluenesulfonyl
HBTU O-benzotriazole-tetramethyluronium hexafluorophosphate
LPT Lapatinib
DIEA N,N-diisopropylethylamine
SB7 SB-743921
EA Ethyl acetate
PCB Palbociclib
TMP 2,4,6-Trimethylpyridine
NPB Niraparib
PyAOP (3H-1,2,3-triazolo[4,5-b]pyridin-3-oxyl)tris-1-pyrrolidinylphosphonium hexafluorophosphate
LC NH₂—CH₂CH₂O—CH₂CH₂O—CH₂—COOH or —NH—CH₂CH₂O—CH₂CH₂O—CH₂—CO—

Sources and structures of some raw materials are as follows:
M-NH₂-10K·HCl
JenKem mPEG-CH₂CH₂—NH₂HCl
M-SCM-10K

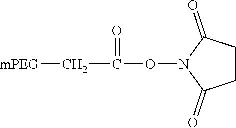

JenKem,

The present disclosure is further illustrated below with reference to specific examples.

### Example 1 Preparation of Compounds

### Synthesis of Compound 39-205

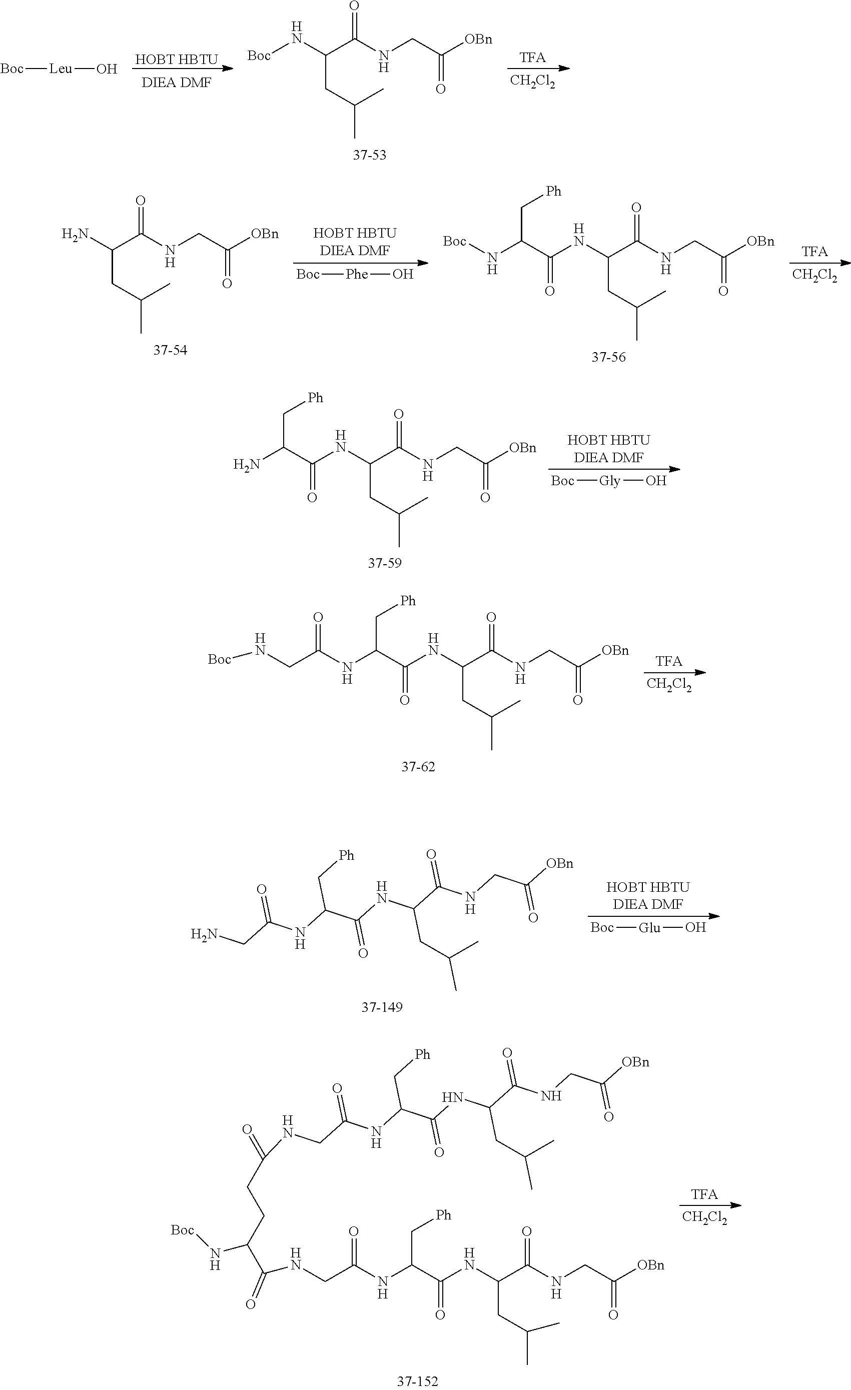

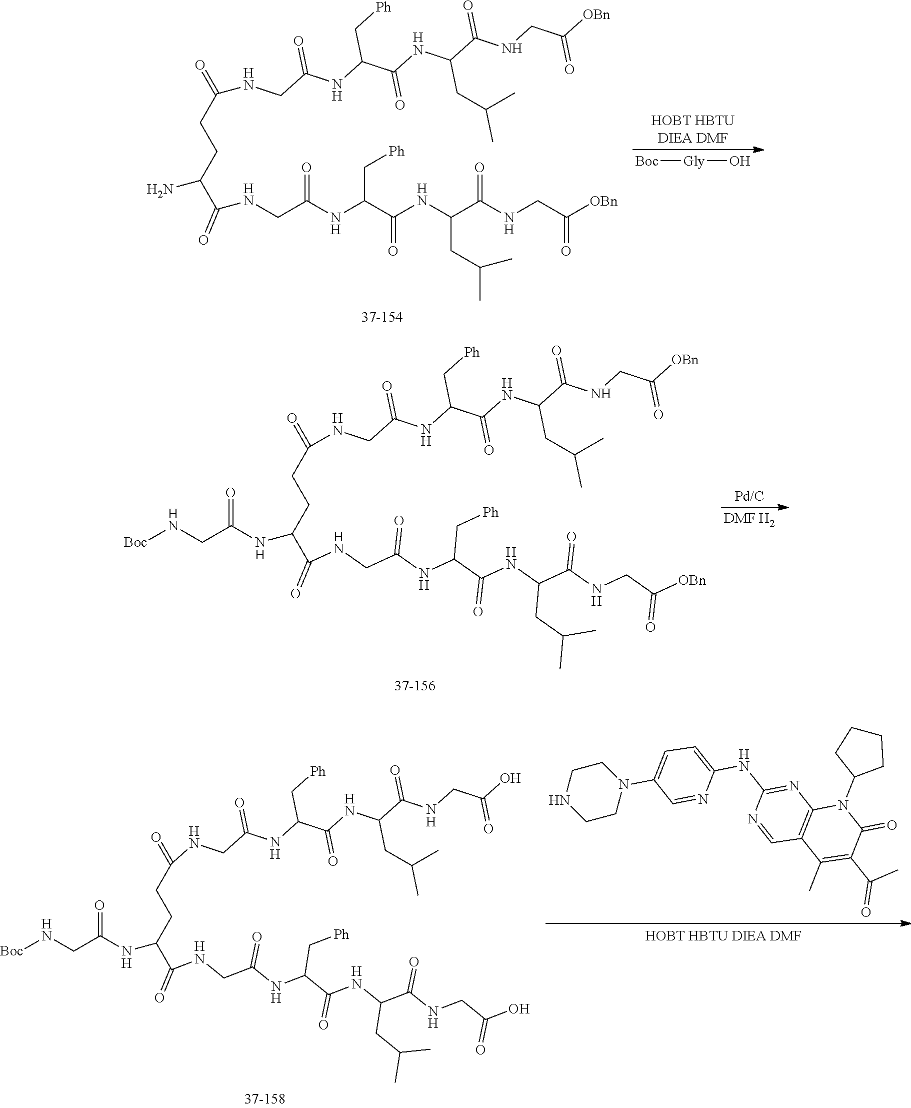

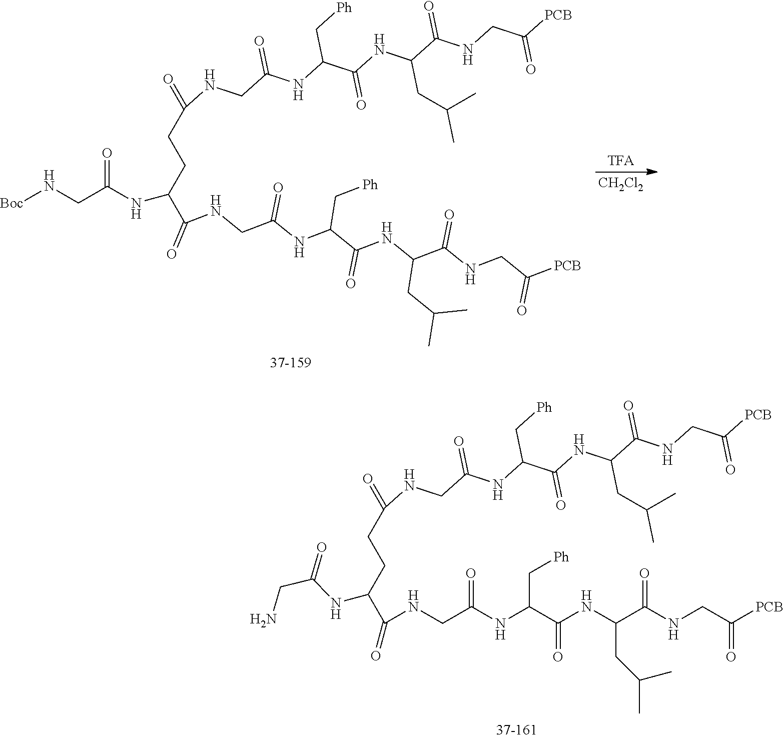

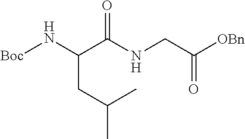 [SMILES: CC(C)CC(NC(=O)OC(C)(C)C)C(=O)NCC(=O)OCc1ccccc1]

Boc-Leu-OH·H₂O (40 g, 160.44 mmol, Innochem), Gly-OBn·TsOH (56.837 g, 168.462 mmol, Ark pharm), HBTU (66.93 g, 176.48 mmol, Aladdin), HOBT (23.85 g, 176.48 mmol, Aladdin) were added to a 1000 mL flask, DMF (250 mL) was added for dissolution, the reactant solution was kept at −5° C. and stirred for about 20 minutes before DIEA (145.85 mL, 882.4356 mmol) was slowly added dropwise, then the resultant solution was further stirred at −5° C. for 1 hour, and then stirred overnight at room temperature. After the reaction ended, the reactant solution was transferred to a 2 L separatory funnel, and added with a saturated sodium bicarbonate solution (250 mL) and ethyl acetate (300 mL) for extraction to obtain an organic phase, the aqueous phase was rinsed with ethyl acetate (200 mL×3), the organic phases were combined, rinsed with saturated brine (200 mL×2), concentrated and evaporated to dryness, and the resultant product was dry loaded for column chromatography with elution using a mixed solution of 30% to 40% EA in petroleum ether, to obtain 60.7 g of product 37-53.

37-54

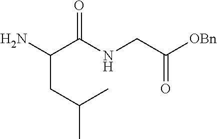 [SMILES: CC(C)CC(N)C(=O)NCC(=O)OCc1ccccc1]

The product 37-53 (60.7 g, 160.44 mmol) was added into a 1000 mL flask, dichloromethane (40 mL) and TFA (95 mL, 1283.52.9 mmol) were added, and the reactant solution was stirred overnight at room temperature. After the reaction ended, the reactant solution was transferred to a 2 L separatory funnel, a saturated sodium bicarbonate solution (350 mL) and ethyl acetate (300 mL) were added for extraction to obtain an organic phase, the aqueous phase was rinsed with ethyl acetate (1500 mL×2), and the organic phases were combined, concentrated and evaporated to dryness, to obtain 45 g of product 37-54.

37-56

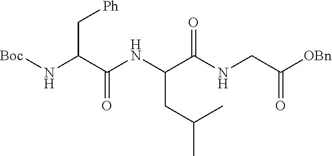 [SMILES: CC(C)CC(NC(=O)C(Cc1ccccc1)NC(=O)OC(C)(C)C)C(=O)NCC(=O)OCc1ccccc1]

The product 37-54 (45 g, 160.44 mmol), Boc-Phe-OH (40.438 g, 152.42 mmol, Aladdin), HBTU (66.93 g, 1276.48 mmol), and HOBT (23.85 g, 176.48 mmol) were added to a 1000 mL flask, DMF (250 mL) was added for dissolution, the reactant solution was kept at −5° C. and stirred for about 20 minutes before DIEA (119.85 mL, 722 mmol) was slowly added dropwise, then the resultant solution was further stirred at −5° C. for 1 hour, and then stirred overnight at room temperature. After the reaction ended, the reactant solution was transferred to a 2 L separatory funnel, a saturated sodium bicarbonate solution (350 mL) and ethyl acetate (300 mL) were added for extraction to obtain an organic phase, the aqueous phase was rinsed with ethyl acetate (200 mL×3), and the organic phases were combined, rinsed with saturated brine (250 mL×2), concentrated and evaporated to dryness, to obtain 84 g of product 37-56.

37-59

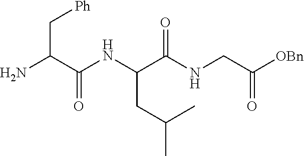 [SMILES: CC(C)CC(NC(=O)C(N)Cc1ccccc1)C(=O)NCC(=O)OCc1ccccc1]

The product 37-56 (84 g, 160.44 mmol) was added into a 1000 mL flask, dichloromethane (40 mL) and TFA (95 mL, 1283.52.9 mmol) were added, and the reactant solution was stirred overnight at room temperature. After the reaction ended, the reactant solution was transferred to a 2 L Erlenmeyer flask, a saturated sodium bicarbonate solution (350 mL) was added, a lot of bubbles were generated, solid sodium bicarbonate was slowly added until pH>7, the solid precipitated and was filtered, and the resultant filter cake was rinsed with pure water (100 mL×2) and was dried to obtain 68 g of product 37-59.

37-62

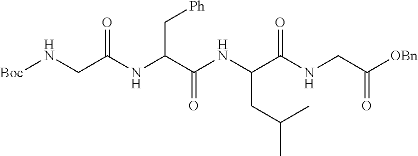 [SMILES: CC(C)CC(NC(=O)C(Cc1ccccc1)NC(=O)CNC(=O)OC(C)(C)C)C(=O)NCC(=O)OCc1ccccc1]

The product 37-59 (68.27 g, 152.42 mmol), Boc-Gly-OH (25.37 g, 144.799 mmol, Aladdin), HBTU (63.58 g, 167.66 mmol), and HOBT (63.58 g, 167.66 mmol) were added to a 1000 mL flask, DMF (250 mL) was added for dissolution, the reactant solution was kept at −5° C. and stirred for about 20 minutes before DIEA (113.3 mL, 685.89 mmol) was slowly added dropwise, then the resultant solution was further stirred at −5° C. for 1 hour, and then stirred overnight at room temperature. After the reaction ended, the reactant solution was transferred to a 2 L separatory funnel, a saturated sodium bicarbonate solution (300 mL) and ethyl acetate (350 mL) were added for extraction to obtain an organic phase, the aqueous phase was rinsed (200 mL×3), the organic phases were combined and rinsed with saturated brine (250 mL×2), the organic phase was left standing at room temperature for 1.5 hours, a solid precipitated and was filtered, and the resultant filter cake was rinsed with ethyl acetate:petroleum ether (3:7) (150 mL×5), to obtain 72.8 g of product 37-62.

37-149

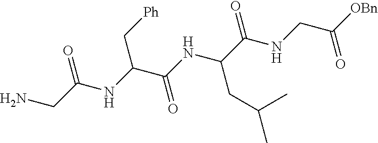 [SMILES: CC(C)CC(NC(=O)C(Cc1ccccc1)NC(=O)CN)C(=O)NCC(=O)OCc1ccccc1]

The product 37-62 (30 g, 51.4871 mmol) was added into a 250 mL flask, dichloromethane (20 mL) was added for dissolution, and trifluoroacetic acid (30.6 mL, 411.9 mmol) was added during stirring, and the reactant solution was stirred overnight at room temperature. After the reaction ended, the reactant solution was concentrated, a saturated sodium bicarbonate solution (200 mL) was added, a lot of bubbles were generated, solid sodium bicarbonate was then slowly added to adjust pH until pH>7, the solid precipitated and was filtered, and the resultant filter cake was rinsed with ionized water (150 mL×4) and was dried to obtain 24.85 g of product 37-149.

37-152

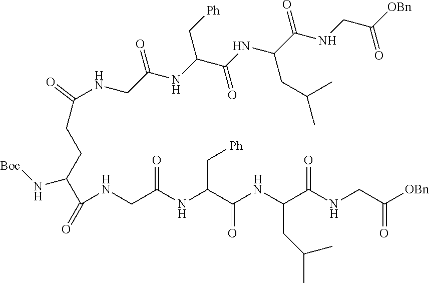 [SMILES: CC(C)CC(NC(=O)C(Cc1ccccc1)NC(=O)CNC(=O)CCC(NC(=O)OC(C)(C)C)C(=O)NCC(=O)NC(Cc1ccccc1)C(=O)NC(CC(C)C)C(=O)NCC(=O)OCc1ccccc1)C(=O)NCC(=O)OCc1ccccc1]

Boc-Glu-OH (5.7864 g, 23.4032 mmol, Ark pharm), the product 37-149 (24.85 g, 51.4871 mmol), HBTU (26.626 g, 70.2096 mmol), and HOBT (9.4874 g, 70.2096 mmol) were added to a 500 mL flask, DMF (150 mL) was added for dissolution, the reactant solution was kept at −5° C. and stirred for about 10 minutes before DIEA (34.8 mL, 210.628 mmol) was slowly added dropwise, then the resultant solution was further stirred at −5° C. for 1 hour, and then stirred overnight at room temperature. After the reaction ended, the reactant solution was transferred to a 1 L separatory funnel, added with a saturated sodium bicarbonate solution (200 mL) and ethyl acetate (300 mL) and shaken for extraction. The aqueous phase was rinsed with ethyl acetate (150 mL×1), the organic phases were combined, concentrated and evaporated to dryness, methanol (20 mL) and dichloromethane (100 mL) were added for dissolution, silica gel powder (50 g) was added, and the resultant mixture was evaporated to dryness to obtain a powder-like solid, and the powder-like solid was dry loaded for column chromatography with elution using a mixed solution of 4% to 8% methanol in dichloromethane, and the resultant product was dried in a vacuum oven to obtain 19.2 g of product 37-152, with a yield of 69.8%.

37-154

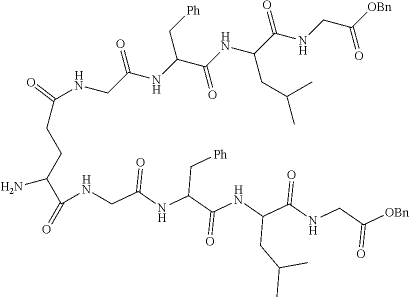 [SMILES: CC(C)CC(NC(=O)C(Cc1ccccc1)NC(=O)CNC(=O)CCC(N)C(=O)NCC(=O)NC(Cc1ccccc1)C(=O)NC(CC(C)C)C(=O)NCC(=O)OCc1ccccc1)C(=O)NCC(=O)OCc1ccccc1]

The product 37-152 (19.2 g, 16.3215 mmol) was added into a 1000 mL flask, dichloromethane (30 mL) was added, trifluoroacetic acid (9.6966 mL, 130.5722 mmol) was added, and the reactant solution was stirred overnight at room temperature. After the reaction ended, the reactant solution was concentrated to a small volume, methyl tertiary butyl ether (200 mL) was added, to obtain a solid precipitate, then suction filtration was performed, and the resultant filter cake was rinsed with methyl tertiary butyl ether (100 mL×3) and was dried to obtain 17.5 g of product 37-154.

37-156

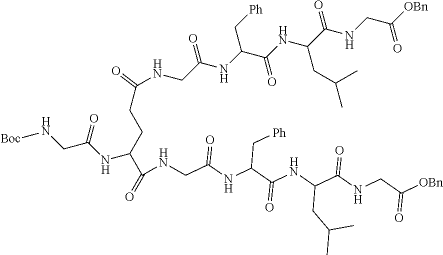 [SMILES: CC(C)CC(NC(=O)C(Cc1ccccc1)NC(=O)CNC(=O)CCC(NC(=O)CNC(=O)OC(C)(C)C)C(=O)NCC(=O)NC(Cc1ccccc1)C(=O)NC(CC(C)C)C(=O)NCC(=O)OCc1ccccc1)C(=O)NCC(=O)OCc1ccccc1]

Boc-Gly-OH (3.4310 g, 19.5858 mmol, Ark Pharm), the product 37-154 (17.5 g, 16.3215 mmol), HBTU (9.2847 g, 24.4823 mmol), and HOBT (3.3083 g, 24.4823 mmol) were added to a 1000 mL flask, DMF (150 mL) was added for dissolution, the reactant solution was kept at −5° C. and stirred for about 10 minutes before DIEA (12.14 mL, 73.4468 mmol) was slowly added dropwise, then the resultant solution was further stirred at −5° C. for 1 hour, and then stirred overnight at room temperature. After the reaction ended, ethyl acetate (200 mL), methyl tertiary butyl ether (200 mL) and n-hexane (300 mL) were added, to obtain a solid precipitate, then suction filtration was performed, and the resultant filter cake was rinsed with methyl tertiary butyl ether (100 mL×3) and was dried to obtain 19.2 g of product 37-156.

37-158

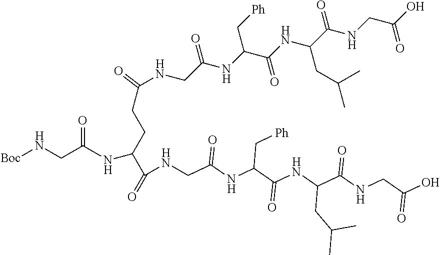 [SMILES: CC(C)CC(NC(=O)C(Cc1ccccc1)NC(=O)CNC(=O)CCC(NC(=O)CNC(=O)OC(C)(C)C)C(=O)NCC(=O)NC(Cc1ccccc1)C(=O)NC(CC(C)C)C(=O)NCC(=O)O)C(=O)NCC(=O)O]

The product 37-156 (10.499 g, 8.5125 mmol) was added into a hydrogenation reaction kettle, then 10% Pd/C catalyst (0.10 g) was added, DMF (50 mL) was further added for dissolution, hydrogen gas was injected to implement a pressure of 1.8 MPa, and the reactant solution was stirred overnight at room temperature. After the reaction ended, the reactant solution was filtered with celite, the celite was rinsed with DMF (20 mL×3), and the DMF solution was combined and used as a raw material for the next reaction.

37-159

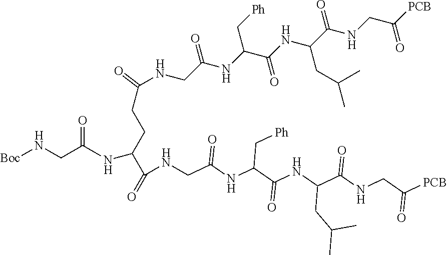

The product 37-158 (8.965 g, 0.4390 mmol), Palbociclib (8 g, 17.8763 mmol, PCB for short), HBTU (9.6848 g, 25.5375 mmol), and HOBT (3.4509 g, 25.5375 mmol) were added to a 500 mL flask, DMF (130 mL) was added for dissolution, the reactant solution was kept at −5° C. and stirred for 20 minutes before DIEA (12.6626 mL, 76.6125 mmol) was slowly added dropwise, and then the reactant solution was further stirred at −5° C. for 1 hour and then stirred overnight at room temperature. After the reaction ended, n-hexane (150 mL) and methyl tertiary butyl ether (100 mL) were added, to obtain a solid precipitate, then filtration was performed, and the resultant filter cake was rinsed with methyl tertiary butyl ether (100 mL×3) and was dried to obtain 16.3 g of product 37-159.

37-161

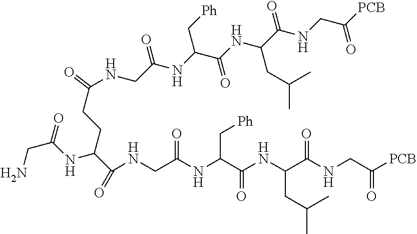

The product 37-159 (16.3 g, 8.5125 mmol) was added into a 500 mL flask, dichloromethane (30 mL) was added for dissolution, trifluoroacetic acid (18.964 mL, 255.375 mmol) was added, and the reactant solution was stirred overnight at room temperature. After the reaction ended, methyl tertiary butyl ether (250 mL) was added to obtain a solid precipitate, then filtration was performed, the resultant filter cake was rinsed with methyl tertiary butyl ether (50 mL×3), the filter cake was dissolved in dichloromethane (240 mL) and methanol (60 mL), silica gel powder (50 g) was added to obtain a mixture which was then evaporated to dryness to obtain a powder-like solid, and the powder-like solid was dry loaded for column chromatography with elution using a mixed solution of 1% ammonia and 5% to 7% methanol in dichloromethane, and the desired component was collected, concentrated and dried, to obtain 11.2 g of product 37-161 with a yield of 73%.

39-144

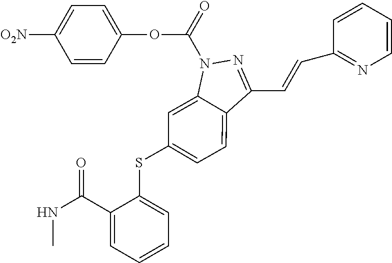 [SMILES: CNC(=O)c1ccccc1Sc1ccc2c(/C=C/c3ccccn3)nn(C(=O)Oc3ccc([N+](=O)[O-])cc3)c2c1]

AXT (0.5 g, 2.58 mmol), 4-nitrophenyl chloroformate (0.8 g, 7.74 mmol) and THE (about 100 mL) were weighed and ultrasonically dissolved to form a homogeneous phase, and the resultant solution was subjected to oil bath at 75° C., stirred during reaction and condensed to reflux. After the reaction ended, methyl tertiary butyl ether (150 mL) and n-hexane (100 mL) were added to the reactant solution, to obtain a precipitate of a solid product which was subjected to suction filtration, and was dried under vacuum to obtain the product.

33-143

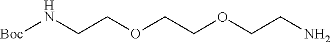 [SMILES: CC(C)(C)OC(=O)NCCOCCOCCN]

1,2-bis(2-aminoethoxy)ethane (20 mL, 136 mmol, TCI) was weighed, dichloromethane (50 mL) was added for dilution, triethylamine (37.9 mL, 272 mmol) was added, di-tert-butyl dicarbonate (29 g, 136 mmol, Accela ChemBio Co., Ltd.) was added during stirring, and the reactant solution was stirred for reaction. After the reaction ended, deionized water (200 mL) and EA (200 mL) were added for extraction, the organic phase was collected after standing and stratification, the aqueous phase was rinsed with EA (200 mL×3), and the organic phases were combined and dry loaded for column chromatography, and gradient elution was performed using 20% to 100% ethyl acetate/petroleum ether, to obtain 6.0 g of the product.

39-146

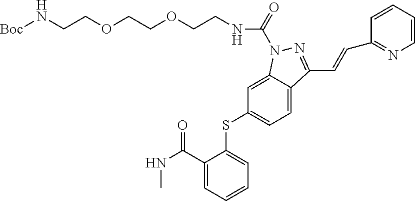 [SMILES: CNC(=O)c1ccccc1Sc1ccc2c(/C=C/c3ccccn3)nn(C(=O)NCCOCCOCCNC(=O)OC(C)(C)C)c2c1]

The product 33-143 (0.64 mL, 2.58 mmol) and triethylamine (1.07 mL, 7.74 mmol) were added to the product 39-144 (2.58 mmol), and the resultant mixture was dissolved in DMF and stirred for reaction at room temperature. After the reaction ended, deionized water (200 mL) and EA (200 mL) were added for extraction, the organic phase was collected after standing and stratification, the aqueous phase was rinsed with EA (200 mL×3), the organic phases were combined and evaporated to dryness to obtain a solid, and the solid was dried in a vacuum oven, suction filtered, and dry loaded for column chromatography.

39-147

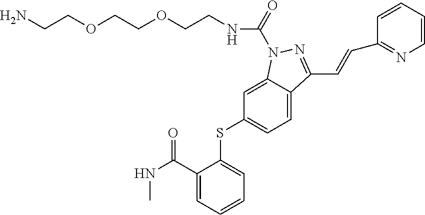 [SMILES: CNC(=O)c1ccccc1Sc1ccc2c(/C=C/c3ccccn3)nn(C(=O)NCCOCCOCCN)c2c1]

The product 39-146 (2.58 mmol) was taken, dichloromethane (10 mL) and TFA (3.8 mL, 51.6 mmol) were added, and the resultant solution was stirred for reaction at room temperature. After the reaction ended, the reactant solution was concentrated, methyl tertiary butyl ether (150 mL) and n-hexane (100 mL) were added to the reactant solution for precipitation, to obtain a precipitate of a solid product which was subjected to suction filtration, dried under vacuum, and dry loaded for column chromatography, and gradient elution was performed using 2% to 5% methanol/dichloromethane, to obtain 1.0 g of the product.

39-148

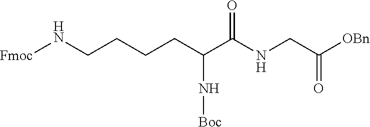

Reactants Boc-Lys(Fmoc)-OH (Aladdin, 4.0 g, 8.53 mmol), H-Gly-OBzl (2.88, 8.53 mmol), HBUT (4.85 g, 12.79 mmol), and HOBT (1.7 g, 12.79 mmol) were taken and added into a 250 mL reaction flask, DMF (40 mL) was added for dissolution, the reactant solution was left standing at −5° C. and stirred for 30 minutes, DIEA (6.3 mL, 38.38 mmol) was slowly added dropwise, and the reactant solution reacted at low temperature for 2 hours and then reacted at room temperature. After the reaction ended, the reactant solution was transferred into a 1 L separatory funnel, deionized water (200 mL) and ethyl acetate (300 mL) were added for extraction, the organic phase was separated, the aqueous phase was extracted with ethyl acetate (100 mL×3) three times, the organic phases were combined, rinsed with saturated brine (150 mL×2) twice, concentrated and evaporated to dryness, to obtain the product.

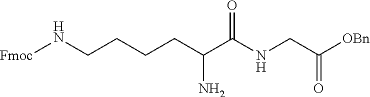

The product 39-148 (5.25 g, 8.53 mmol) was weighed, dichloromethane (5 mL) and TFA (6.3 mL, 85.3 mmol) were added, the product was ultrasonically dissolved, and the reactant solution was stirred for reaction. After the reaction ended, the reactant solution was concentrated, and methyl tertiary butyl ether (100 mL) and n-hexane (200 mL) were added for precipitation, to obtain powder.

39-152

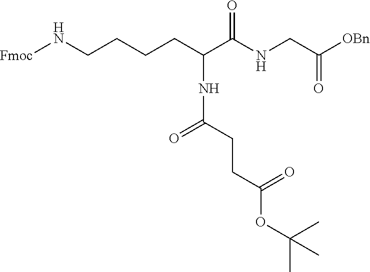

Reactants 39-149 (8.53 mmol), mono-tert-butyl succinate (1.48 g, 8.53 mmol), HBTU (4.85 g, 12.79 mmol), and HOBT (1.7 g, 12.79 mmol) were taken and added into a 250 mL reaction flask, DMF (40 mL) was added for dissolution, the reactant solution was left standing at −5° C. and stirred for 30 minutes, DIEA (6.3 mL, 38.38 mmol) was slowly added dropwise, and the reactant solution reacted at low temperature for 2 hours and then reacted at room temperature. After the reaction ended, methyl tertiary butyl ether (100 mL) and n-hexane (200 mL) were added into the reactant solution for precipitation, to obtain 6 g of powder, which was overproduction.

39-154

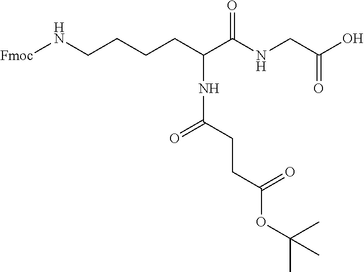

The reactant 39-152 (2.5 g, 3.72 mmol) and 10% Pd/C catalyst (0.07 g) were weighted and dissolved in DMF (30 mL), the reactant solution was put into a hydrogenation reaction kettle, the instrument was set up, hydrogen gas was injected to maintain a pressure of 18 Ps, and the reactant solution was stirred for reaction. After the reaction ended, the resultant filter cake was filtered with celite to remove Pd/C, and celite was rinsed with DMF (25 mL×4) 4 times, to obtain a product solution.

49-17

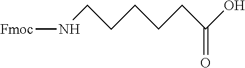

6-aminocaproic acid (4.61 g, 35.1407 mmol) was weighed and added into a 1 L flask, 150 mL of mixed solution with THF:H₂O=1:1 was added to dissolve 6-aminocaproic acid completely, the reactant solution was stirred at 0° C., then solid sodium carbonate (7.45 g, 70.2814 mmol) was added and ultrasonically dissolved, and the reactant solution was stirred at 0° C. After 30 minutes of reaction, Fmoc-CI (10 g, 38.6548 mmol) was dissolved in 30 mL of THF, and the resultant mixture was slowly added to the reactant solution dropwise, and then the reactant solution was taken out and stirred for reaction at room temperature. After the reaction ended, 50 g of citric acid was weighed and dissolved in 450 mL of deionized water, the resultant mixture was added to the reactant solution to adjust pH to 3, then the reactant solution was transferred to a 1 L separatory funnel, EA (300 mL×3) was added for extraction, and the organic phase was collected, concentrated and evaporated to dryness, and then dissolved in a solution (100 mL) of methanol/dichloromethane (1:4), silica gel powder (50 mL) was added, and the resultant mixture was evaporated to dryness to obtain a powder-like solid, and the powder-like solid was dry loaded for column chromatography with elution using a solution of 2% methanol/dichloromethane, and the desired component was collected, concentrated and dried in a vacuum oven to obtain a product (7.7 g, 86.51%).

39-80

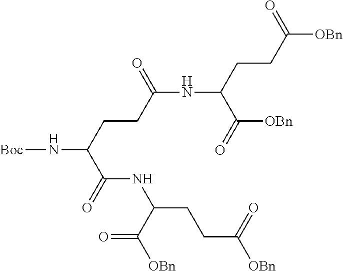 [SMILES: CC(C)(C)OC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

Boc-Glu-OH (5.0 g, 20.22 mmol), H-GLu(OBzl)-OBzl-TsOH (21.2 g, 42.46 mmol), HOBT (8 g, 60.66 mmol) and HBTU (23 g, 60.66 mmol) were weighed and added into a 250 mL reaction flask, a DMF solution (80 mL) was added for dissolution, the reactant was completely ultrasonically dissolved, the reactant solution was stirred at −5° C. for 30 minutes, DIEA (30 mL, 181 mmol) was slowly added dropwise, and the reactant solution was left standing at low temperature until the reaction ended. After the reaction ended, the reactant solution was taken out, added with deionized water (100 mL), and extracted with ethyl acetate (100 mL×3) multiple times, the organic phases were combined, rinsed with a saturated sodium chloride solution (100 mL) twice and finally concentrated and evaporated to dryness. The resultant product was dry loaded for column chromatography, and gradient elution was performed using 40% to 50% ethyl acetate/petroleum ether, and the product was collected, concentrated and evaporated to dryness.

39-84

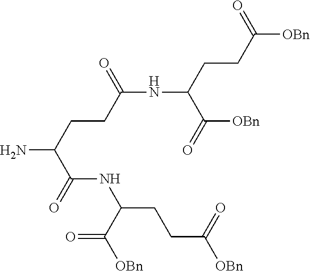 [SMILES: NC(CCC(=O)NC(CCC(=O)OCc1ccccc1)C(=O)OCc1ccccc1)C(=O)NC(CCC(=O)OCc1ccccc1)C(=O)OCc1ccccc1]

The product 39-80 (19.2 mmol) was taken, dichloromethane (5 mL) and TFA (14 mL, 192 mmol) were added, the product was completely ultrasonically dissolved, the reaction flask was sealed with a ground glass stopper, and the reactant solution was stirred for reaction at room temperature. After the reaction ended, the reactant solution was taken out, a saturated sodium bicarbonate solution (300 mL), and extracted with ethyl acetate (100 mL×3) multiple times, the organic phases were combined, rinsed with a saturated sodium chloride solution (100 mL) twice and finally concentrated and evaporated to dryness.

49-101

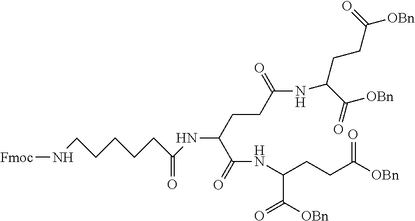

The product 39-84 (8 g, 10.4459 mmol), HBTU (5.94 g, 15.6689 mmol) and HOBT (2.12 g, 15.6689 mmol) were added to a flask containing the product 49-17 (3.69 g, 10.4459 mmol), then an appropriate amount of DMF was added for dissolution, the reactant solution was kept at −5° C., and DIEA (7.77 mL, 47.0066 mmol) was slowly added dropwise, and then the reactant solution reacted for half an hour, then taken out and stirred for reaction overnight at room temperature. After the reaction ended, a saturated NaCl solution (200 mL) was added, EA (200 mL) was added for extraction, the organic phase was collected after standing and stratification, the aqueous phase was rinsed with EA (200 mL×3), the organic phases were combined and evaporated to dryness to obtain a solid, and then the organic phases were dissolved in a solution (100 mL) of methanol/dichloromethane (1:4), silica gel powder (100 mL) was added, and the resultant mixture was evaporated to dryness to obtain a powder-like solid, and the powder-like solid was dry loaded for column chromatography with elution using a 100% dichloromethane solution, and the desired component was collected, concentrated and dried in a vacuum oven to obtain a product (9.6 g, 83.48%).

39-159

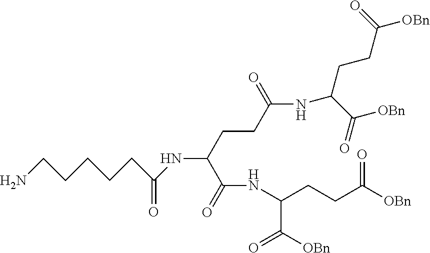 [SMILES: NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

DMF was added to a flask containing the product 49-101 (9.6 g, 8.7193 mmol), and was ultrasonically oscillated until complete dissolution, then morpholine (7.59 mL, 87.193 mmol) was added, and the resultant solution was stirred for reaction for 2 hours at room temperature. After the reaction ended, a saturated NaCl solution (200 mL) was added, EA (200 mL) was added for extraction, the organic phase was collected after standing and stratification, the aqueous phase was rinsed with EA (200 mL×3), the organic phases were combined and evaporated to dryness to obtain a solid, and then the organic phases were dissolved in a solution (100 mL) of methanol/dichloromethane (1:4), silica gel powder (100 mL) was added, and the resultant mixture was evaporated to dryness to obtain a powder-like solid, and the powder-like solid was dry loaded for column chromatography with elution using a mixed solution of 3% to 5% methanol in dichloromethane, and the desired component was collected, concentrated and dried in a vacuum oven to obtain a product (5.3 g, 69.19%).

39-158

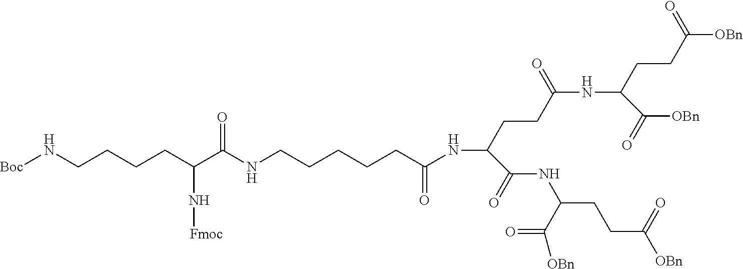

Reactants Fmoc-Lys(Boc)-OH (1.9 g, 4.08 mmol), the product 39-159 (4.08 mmol), HBUT (2.32 g, 6.12 mmol), and HOBT (0.82 g, 6.12 mmol) were taken and added into a 250 mL reaction flask, DMF (40 mL) was added for dissolution, the reactant solution was left standing at −5° C. and stirred for 30 minutes, DIEA (3.0 mL, 18.38 mmol) was slowly added dropwise, and the reactant solution reacted at low temperature for 2 hours and then reacted at room temperature. After the reaction ended, methyl tertiary butyl ether (100 mL) was added to the reactant solution, and n-hexane (200 mL) was added for precipitation, to obtain powder.

39-164

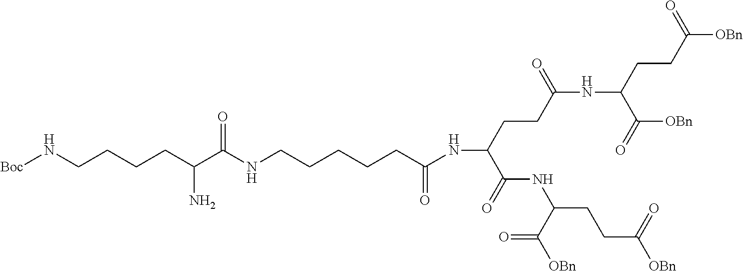 [SMILES: CC(C)(C)OC(=O)NCCCCC(N)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

The product 39-158 (4.08 mmol) was taken, DMF (40 mL) was added for dissolution, then morpholine (3.55 mL, 40.8 mmol) was added, and the resultant solution was stirred for reaction at room temperature. After the reaction ended, methyl tertiary butyl ether (100 mL) and n-hexane (200 mL) were added into the reactant solution for precipitation, to obtain 2.92 g of powder with a yield of 66.6%.

39-165

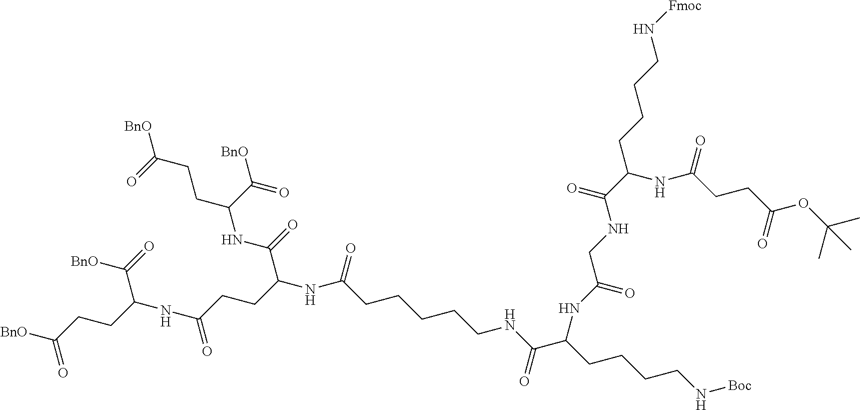

Reactants 39-154 (2.92 g, 2.63 mmol), the product 39-164 (2.63 mmol), HBTU (1.4 g, 3.95 mmol), and HOBT (0.53 g, 3.95 mmol) were taken and added into a 250 mL reaction flask, DMF (40 mL) was added for dissolution, the reactant solution was left standing at −5° C. and stirred for 30 minutes, DIEA (1.96 mL, 11.86 mmol) was slowly added dropwise, and the reactant solution reacted at low temperature for 2 hours and then reacted at room temperature. After the reaction ended, methyl tertiary butyl ether (100 mL) was added to the reactant solution, and n-hexane (200 mL) was added for precipitation, to obtain 2.6 g of powder.

39-173

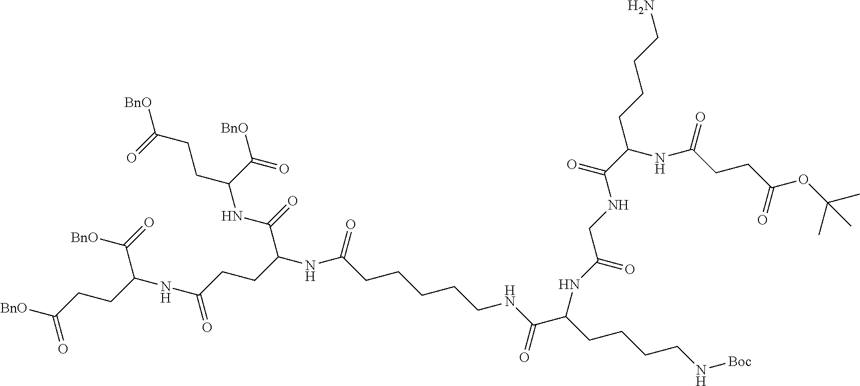 [SMILES: CC(C)(C)OC(=O)CCC(=O)NC(CCCCN)C(=O)NCC(=O)NC(CCCCNC(=O)OC(C)(C)C)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

The product 39-165 (2.6 g, 1.55 mmol) was taken, DMF (40 mL) was added for dissolution, then morpholine (1.53 mL, 15.5 mmol) was added, and the resultant solution was stirred for reaction at room temperature. After the reaction ended, methyl tertiary butyl ether (100 mL) was added, and n-hexane (200 mL) was added for precipitation, to obtain powder.

45-91

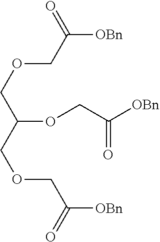 [SMILES: O=C(COCC(COCC(=O)OCc1ccccc1)OCC(=O)OCc1ccccc1)OCc1ccccc1]

Glycerol (5 g, 54.295 mmol, Innochem) was added into a 500 mL reaction flask, nitrogen was injected for protection, a solution of potassium tert-butoxide in THE (211 mL, 211.7505 mmol) was added, the reactant solution was stirred at 0° C. for 3 hours, then benzyl bromoacetate (30.964 g, 195.46 mmol) was added, and the reactant solution was stirred for 2 hours, and then reacted at room temperature. After the reaction ended, the reactant solution was first evaporated to dryness, then deionized water and ethyl acetate were added, the organic phase was separated, the aqueous phase was extracted with ethyl acetate until no product was found, the organic phases were combined, dried with anhydrous sodium sulfate powder, and subjected to suction filtration, and the filtrate was dry loaded for column chromatography. Gradient elution was performed using 1% to 2% ethyl acetate/petroleum ether, to obtain 9 g of the product, with a yield of 31%.

39-174

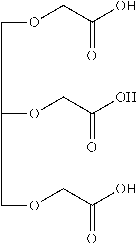 [SMILES: O=C(O)COCC(COCC(=O)O)OCC(=O)O]

The product 45-91 (0.23 g, 0.442 mmol) was added into a reaction kettle, 10% Pd/C catalyst (0.0700 g) was added, DMF (30 mL) was added for dissolution, air in the reaction kettle was pumped out with a water pump to create a vacuum, hydrogen gas was injected at a hydrogen gas pressure of 1.6 MPa, then the hydrogen gas was released, the hydrogen gas was pumped out with a water pump to create a vacuum, and then hydrogen gas was injected again, such procedure was repeated three times, and finally hydrogen gas was injected again, and the reactant solution was stirred overnight at room temperature. After the reaction ended, the reactant solution was filtered with celite, the filter cake was rinsed with DMF (20 mL×3), and the DMF solution was combined and used as a raw material for the next step.

39-176

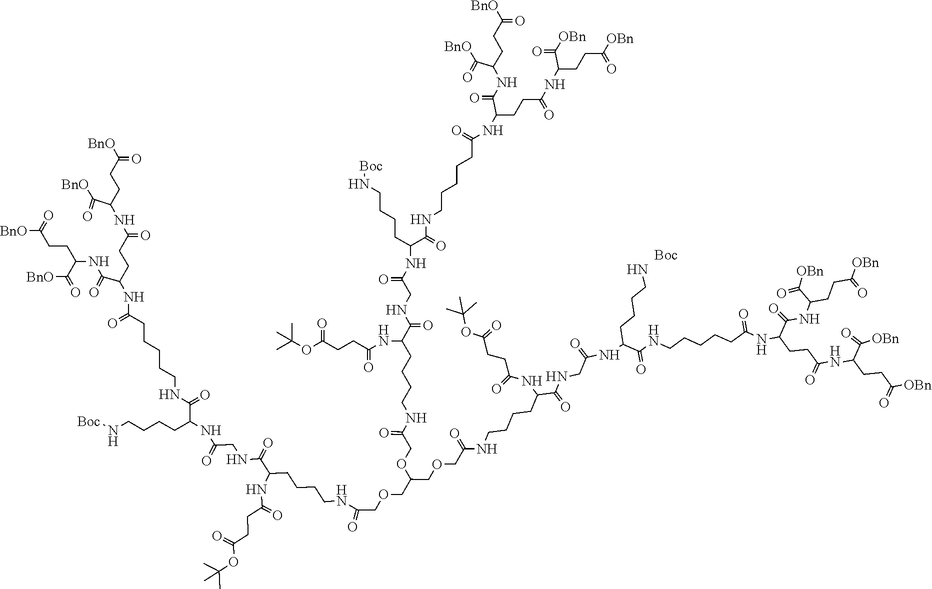

Reactants 39-174 (0.444 mmol), the product 39-173 (1.55 mmol), HBTU (0.75 g, 2.00 mmol), and HOBT (0.27 g, 2.00 mmol) were taken and added into a 250 mL reaction flask, DMF (40 mL) was added for dissolution, the reactant solution was left standing at −5° C. and stirred for 30 minutes, DIEA (0.99 mL, 6.0 mmol) was slowly added dropwise, and the reactant solution reacted at low temperature for 2 hours and then reacted at room temperature. After the reaction ended, the reactant solution was transferred into a 1 L separatory funnel, deionized water (200 mL) and ethyl acetate (300 mL) were added for extraction, the organic phase was separated, the aqueous phase was extracted with ethyl acetate (100 mL×3) three times, the organic phases were combined, rinsed with saturated brine (150 mL×2) twice, concentrated and dry loaded for column chromatography. Gradient elution was performed using 1% to 5% methanol/dichloromethane, to obtain 1 g of the product, with a yield of 50%.

39-194

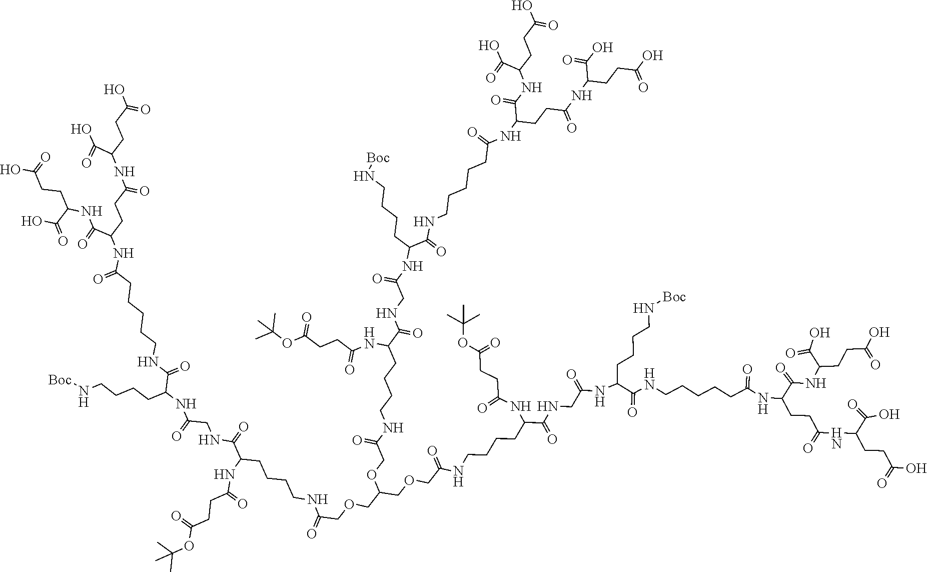 [SMILES: CC(C)(C)OC(=O)CCC(=O)NC(CCCCNC(=O)COCC(COCC(=O)NCCCCC(NC(=O)CCC(=O)OC(C)(C)C)C(=O)NCC(=O)NC(CCCCNC(=O)OC(C)(C)C)C(=O)NCCCCCC(=O)NC(CCC(=O)NC(CCC(=O)O)C(=O)O)C(=O)NC(CCC(=O)O)C(=O)O)OCC(=O)NCCCCC(NC(=O)CCC(=O)OC(C)(C)C)C(=O)NCC(=O)NC(CCCCNC(=O)OC(C)(C)C)C(=O)NCCCCCC(=O)NC(CCC(=O)NC(CCC(=O)O)C(=O)O)C(=O)NC(CCC(=O)O)C(=O)O)C(=O)NCC(=O)NC(CCCCNC(=O)OC(C)(C)C)C(=O)NCCCCCC(=O)NC(CCC(=O)NC(CCC(=O)O)C(=O)O)C(=O)NC(CCC(=O)O)C(=O)O]

The product 39-176 (0.52 g, 0.1143 mmol) was added into a reaction kettle, 10% Pd/C catalyst (0.0700 g) was added, DMF (30 mL) was added for dissolution, air in the reaction kettle was pumped out with a water pump to create a vacuum, hydrogen gas was injected at a hydrogen gas pressure of 1.6 MPa, then the hydrogen gas was released, the hydrogen gas was pumped out with a water pump to create a vacuum, and then hydrogen gas was injected again, such procedure was repeated three times, and finally hydrogen gas was injected again, and the reactant solution was stirred overnight at room temperature. After the reaction ended, the reactant solution was filtered with celite, the filter cake was rinsed with DMF (20 mL×3), and the DMF solution was combined and used as a raw material for the next step.

39-195

The product 39-194 (0.1143 mmol), the product 37-161 (2.7 g, 1.4 mmol), HOBT (0.27 g, 2.04 mmol) and HBTU (0.76 g, 2.04 mmol) were weighed and added into a 250 mL reaction flask, a DMF solution (100 mL) was added for dissolution, the reactant was completely ultrasonically dissolved, the reactant solution was stirred at −5° C. for 30 minutes, DIEA (1.0 mL, 6.14 mmol) was slowly added dropwise, and the reactant solution was left standing at low temperature until the reaction ended. After the reaction ended, methyl tertiary butyl ether (150 mL) and n-hexane (100 mL) were added to the reactant solution, to obtain a precipitate of a solid product which was subjected to suction filtration, and was dried under vacuum to obtain the product.

39-196

The product 39-195 (0.5 g, 0.019 mmol) was taken, dichloromethane (10 mL) and TEA (0.26 mL, 3.59 mmol) were added, and the resultant solution was stirred for reaction at room temperature. After the reaction ended, the reactant solution was concentrated, methyl tertiary butyl ether (150 mL) and n-hexane (100 mL) were added to the reactant solution for precipitation, to obtain a precipitate of a solid product which was subjected to suction filtration, and was dried under vacuum.

39-197

The compound 39-196 (0.019 mmol) and M-SCM-10K (0.62 g, 0.0589 mmol, JenKem) were taken and added into a 250 mL reaction kettle. The resultant mixture was dissolved in DMF (30 mL) and DIEA (1 mL) was added. The reactant solution was stirred at a low speed while protected from light. After the reaction ended, the reactant solution was concentrated, methyl tertiary butyl ether (150 mL) and n-hexane (100 mL) were added, to obtain a precipitate of a solid product which was subjected to suction filtration. The filter cake was dissolved in a mixed solvent (20% dichloromethane: 80% methanol), and dry loaded for column chromatography, and gradient elution was performed using a mixed solution of 4% to 10% methanol/dichloromethane, and the desired component was collected, concentrated and evaporated to obtain 0.4 g of the product.

39-205

The product 39-197 (0.019 mmol), the product 39-147 (0.037 g, 0.0665 mmol), HOBT (0.011 g, 0.0855 mmol) and HBTU (0.032 g, 0.0855 mmol) were weighed and added into a 250 mL reaction flask, a DMF solution (100 mL) was added for dissolution, the reactant was completely ultrasonically dissolved, the reactant solution was stirred at −5° C. for 30 minutes, DIEA (0.042 mL, 0.2565 mmol) was slowly added dropwise, and the reactant solution was left standing at low temperature until the reaction ended. After the reaction ended, methyl tertiary butyl ether (150 mL) and n-hexane (100 mL) were added to the reactant solution, to obtain a precipitate of a solid product which was subjected to suction filtration. The filter cake was dissolved in a mixed solvent (20% dichloromethane: 80% methanol), and dry loaded for column chromatography, and gradient elution was performed using a mixed solution of 4% to 10% methanol/dichloromethane, and the desired component was collected, concentrated and evaporated to obtain 0.4 g of the product.

¹H-NMR (600 MHz, DMSO-d₆) δ 10.18-10.07 (m, 24H), 8.98-8.89 (m, 24H), 8.41-8.36 (m, 4H), 8.34-8.31 (m, 3H), 8.25-8.15 (m, 47H), 8.14-8.00 (m, 82H), 7.99-7.93 (m, 26H), 7.92-7.82 (m, 60H), 7.72-7.64 (m, 6H), 7.53-7.42 (m, 30H), 7.36-7.28 (m, 12H), 7.26-7.18 (m, 107H), 7.16-7.11 (m, 31H), 7.10-7.08 (m, 3H), 7.01-6.99 (m, 3H), 5.94-5.67 (m, 27H), 4.63-4.51 (m, 27H), 4.40-4.32 (m, 27H), 4.24-4.19 (m, 18H), 4.08-3.91 (m, 57H), 3.56-3.44 (m, 2856H), 3.21-3.07 (m, 142H), 2.90-2.82 (m, 52H), 2.79-2.71 (m, 48H), 2.45-2.37 (m, 93H), 2.36-2.01 (m, 216H), 1.96-1.68 (m, 165H), 1.66-1.41 (m, 174H), 0.99-0.69 (m, 177H).

### Synthesis of Compound 80-9 (DXM-AXT=18.3)

54-66

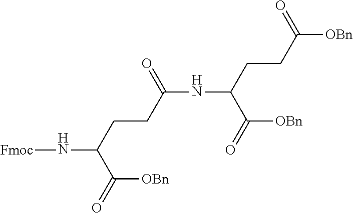

Fmoc-Glu-OBn (9.2 g, 20.0 mmoL, Ark Pharm), diphenylmethyl glutamate (10.0 g, 20.0 mmol, Ark Pharm), HBTU (11.4 g, 30.0 mmoL) and HOBT (4.1 g, 30 mmoL) were weighted and added into a 500 mL flask, 150 mL of a DMF solution was added for dissolution, the reactant solution was stirred at −5° C. for 30 minutes, DIEA (15.0 mL, 90 mmol) was slowly added dropwise, and the reactant solution reacted for 2 hours and then placed at room temperature for reaction until the end. After the reaction ended, deionized water (150 mL) and ethyl acetate (250 mL) were added for extraction to obtain an organic phase, the aqueous phase was rinsed with ethyl acetate (150 mL×2), and the organic phases were combined, rinsed with saturated brine (150 mL×2), evaporated to dryness, and dried in a vacuum oven for 4 hours to obtain the product to be used as a raw material for the next reaction.

61-116

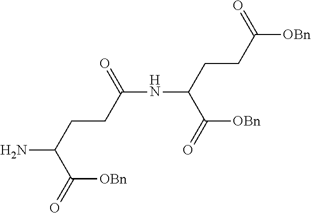 [SMILES: NC(CCC(=O)NC(CCC(=O)OCc1ccccc1)C(=O)OCc1ccccc1)C(=O)OCc1ccccc1]

The product 54-66 (13.6 g, 172841 mmol) was added into a 500 mL flask, DMF (30 mL) was added for dissolution, and then morpholine (15.1 mL, 172.841 mmol) was added, and the reactant solution was stirred for 3 hours at room temperature. After the reaction ended, the reactant solution was transferred into a 1 L separatory funnel, deionized water (150 mL) and ethyl acetate (250 mL) were added for extraction to obtain an organic phase, the aqueous phase was rinsed with ethyl acetate (150 mL×2), and the organic phases were combined, rinsed with saturated brine (150 mL×2), evaporated to dryness, and dried in a vacuum oven for 4 hours to obtain the product to be used as a raw material for the next reaction.

61-117

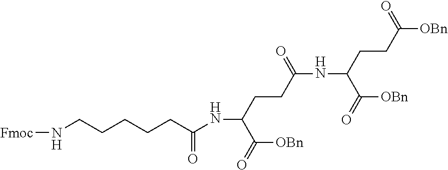

The product 61-116 (17.2841 mmoL), Fmoc-AH-OH (synthesized in a synthesis method of the product 49-17, 4.9 g, 13.8273 mmol), HBTU (7.9 g, 20.7410 mmoL) and HOBT (2.8 g, 20.7410 mmoL) were weighted and added into a 500 mL flask, DMF (150 mL) was added for dissolution, the reactant solution was stirred at −5° C. for 30 minutes, DIEA (10.3 mL, 62.2229 mmoL) was slowly added dropwise, and the reactant solution reacted for 2 hours and then placed at room temperature for reaction until the end. After the reaction ended, deionized water (150 mL) and ethyl acetate (250 mL) were added for extraction to obtain an organic phase, the aqueous phase was rinsed with ethyl acetate (150 mL×2), and the organic phases were combined, rinsed with saturated brine (150 mL×2), concentrated and evaporated to dryness, and dry loaded for column chromatography with elution using a dichloromethane solution, collection and concentration. The organic phases were evaporated to dryness and dried in a vacuum oven for 4 hours to obtain 10.3 g of the product to be used as a raw material for the next reaction, with a yield of 84.4%.

61-118

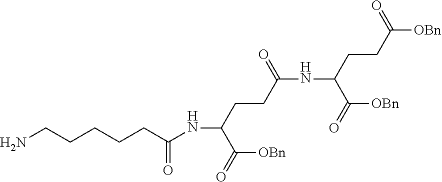 [SMILES: NCCCCCC(=O)NC(CCC(=O)NC(CCC(=O)OCc1ccccc1)C(=O)OCc1ccccc1)C(=O)OCc1ccccc1]

The product 61-117 (2.4 g, 2.7211 mmol) was added to a 250 mL round-bottomed flask, and dissolved in DMF (50 mL), and then morpholine (2.1 mL, 27.211 mmol) was added, and the reactant solution was stirred and reacted for 2 hours at the room temperature until the reaction ended. The reactant solution was transferred to a 1 L separatory funnel, saturated sodium chloride solution (100 mL) was added, the reaction flask was rinsed with ethyl acetate, the mixed phases in the separatory funnel were evenly mixed by shaking, the organic phase was separated, the aqueous phase was extracted with ethyl acetate (200 mL×3) three times, and the organic phases were combined and rinsed with saturated sodium chloride solution (100 mL) three times. The products of the organic phases were combined and suction filtered, the filtrate was concentrated, evaporated to dryness, and then dried in a vacuum box to obtain the product.

61-119

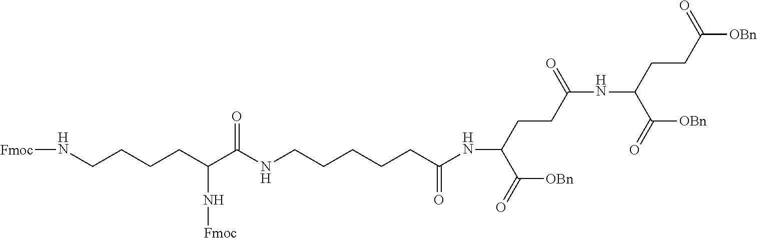

The product 61-118 (1.75 g, 2.7211 mmoL), Fmoc-Lys(Fmoc)-OH (1.3 g, 2.1767 mmol, Aladdin), HBTU (1.5 g, 4.0817 mmoL) and HOBT (0.5 g, 4.0817 mmoL) were weighted and added into a 500 mL flask, DMF (150 mL) was added for dissolution, the reactant solution was stirred at −5° C. for 30 minutes, DIEA (2.0 mL, 12.2450 mmoL) was slowly added dropwise, and the reactant solution reacted overnight. After the reaction ended, deionized water (100 mL) and ethyl acetate (150 mL) were added for extraction to obtain an organic phase, the aqueous phase was rinsed with ethyl acetate (150 mL×2), and the organic phases were combined, subjected to column chromatography with elution using 1% methanol/dichloromethane solution, and the resultant product was collected, concentrated, evaporated to dryness, and dried in a vacuum oven for 4 hours to obtain 2.0 g of the product to be used as a raw material for the next reaction, with a yield of 75.47%.

61-122

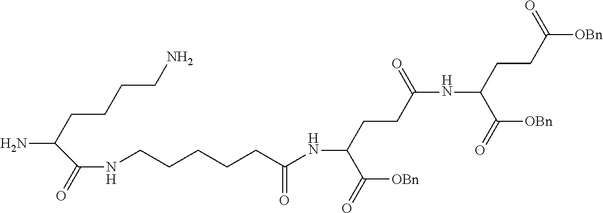 [SMILES: NCCCCC(N)C(=O)NCCCCCC(=O)NC(CCC(=O)NC(CCC(=O)OCc1ccccc1)C(=O)OCc1ccccc1)C(=O)OCc1ccccc1]

The product 61-119 (2.0 g, 1.6415 mmol) was added to a 250 mL round-bottomed flask and dissolved in DMF (50 mL), then morpholine (2.9 mL, 32.8291 mmol) was added, and the reactant solution was stirred for 3 hours for reaction at room temperature until the reaction ended. 200 mL of a mixed solution of methyl tertiary butyl ether/n-hexane (1:5) was added and shaken, the supernatant was discarded after standing, such procedure was repeated three times, then methyl tertiary butyl ether (200 mL) was added, to obtain a solid precipitate, then suction filtration was performed, and the filter cake was dried in a vacuum oven to obtain product 61-122 (1.0 g, 78.7%) to be used as a raw material for the next reaction.

61-123

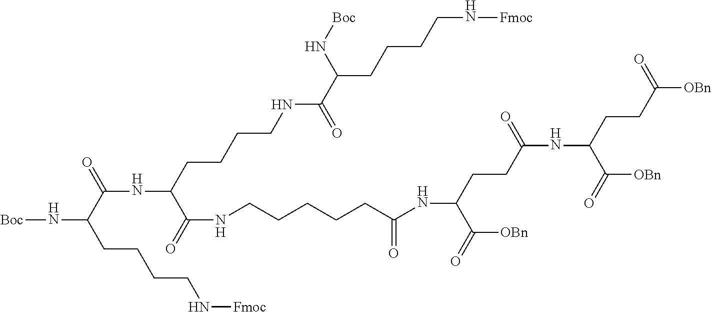

The product 61-122 (1.0 g, 1.2921 mmoL), Boc-Lys(Fmoc)-OH (1.3 g, 2.8425 mmol), HBTU (1.5 g, 3.8762 mmoL) and HOBT (0.5 g, 3.8762 mmoL) were weighed and added into a 500 mL flask, DMF (150 mL) was added for dissolution, the reactant solution was stirred at −5° C. for 30 minutes, and DIEA (1.9 mL, 11.6286 mmoL) was slowly added dropwise. After the reaction ended, a mixed solution (200 mL) of methyl tertiary butyl ether/n-hexane (1:5) was added and shaken, the supernatant was discarded after standing, such procedure was repeated three times, then methyl tertiary butyl ether (200 mL) was added, to obtain a solid precipitate, then suction filtration was performed, and the filter cake was collected, subjected to column chromatography with elution using 2.5% methanol/dichloromethane solution, and the resultant product was collected, concentrated, evaporated to dryness and dried in a vacuum oven for 4 hours to obtain product 61-123 (1.1 g, 50.93%) to be used as the raw material for the next reaction.

56-214

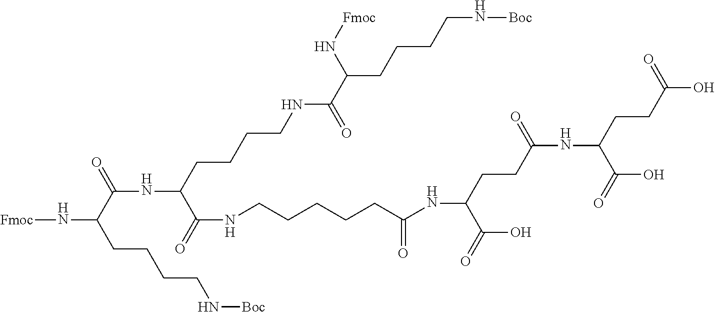

The product 61-123 (0.5 g, 0.2960 mmoL) and 10% Pd/C catalyst (10 mg) were added into a hydrogenation reaction instrument, then DMF (30 mL) was added for dissolution, the solvent submerged a stirring bar, and the hydrogenation reaction instrument was sealed. After three pumping operations and three injection operations (pumping out the air in the reaction system with a vacuum water pump for about 3 minutes—injecting hydrogen gas—pumping out hydrogen gas—injecting hydrogen gas—pumping out hydrogen gas—injecting hydrogen gas), a pressure reading on the hydrogenation reaction instrument was 18 Psi, and then the reactant solution reacted overnight at room temperature. On the next day, after it was detected that the reaction ended via a TLC plate, post-treatment was carried out, the reactant solution was taken out and evenly added dropwise to a suction filtration funnel filled with compacted celite, the reaction instrument was rinsed with DMF (20 mL×3) until the reaction instrument was clean and contained no product, to obtain reaction product 56-214.

56-217

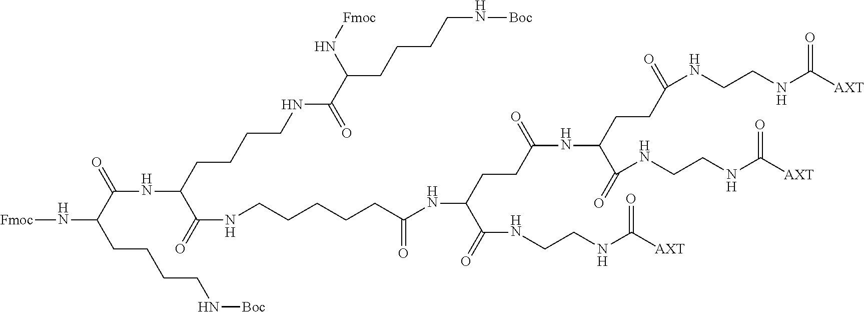

The product 56-214 (0.2960 mmoL), the product 53-215 (0.47 g, 0.9865 mmol), DMTMM (0.3 g, 1.081 mmoL) and NMM (0.15 mL, 1.3500 mmoL) were weighed and added into a 250 mL flask, DMF (30 mL) was added for dissolution, and the reactant solution was stirred for reaction at room temperature for 3 hours. After the reaction ended, a mixed solution (200 mL) of methyl tertiary butyl ether/n-hexane (1:5) was added and shaken, the supernatant was discarded after standing, such procedure was repeated three times, then methyl tertiary butyl ether (200 mL) was added, to obtain a solid precipitate, then suction filtration was performed, and the filter cake was collected, subjected to column chromatography with elution using 3% methanol/dichloromethane solution, and the resultant product was collected, concentrated, evaporated to dryness and dried in a vacuum oven for 4 hours to obtain product 56-217 (0.4 g, 66.67%) to be used as the raw material for the next reaction.

56-220

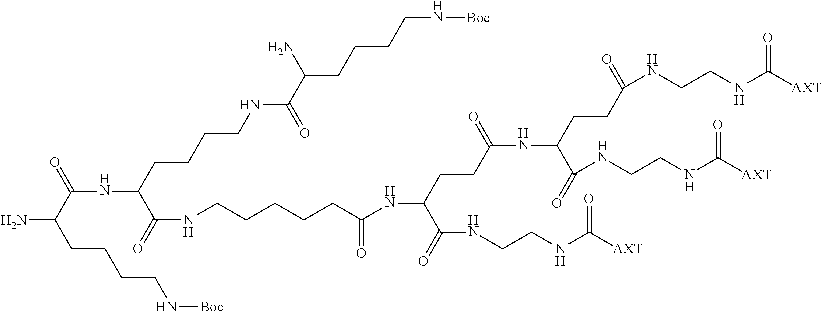

The product 56-217 (0.4 g, 0.1437 mmol) was taken, DMF (40 mL) was added for dissolution, then morpholine (0.1 mL, 1.05 mmol) was added, and the resultant solution was stirred for reaction at room temperature. After the reaction ended, methyl tertiary butyl ether (150 mL) and n-hexane (100 mL) were added to the reactant solution, to obtain a precipitate of a solid product which was subjected to suction filtration, and was dried under vacuum to obtain 0.3 g of the product, with a yield of 90.90%.

56-221

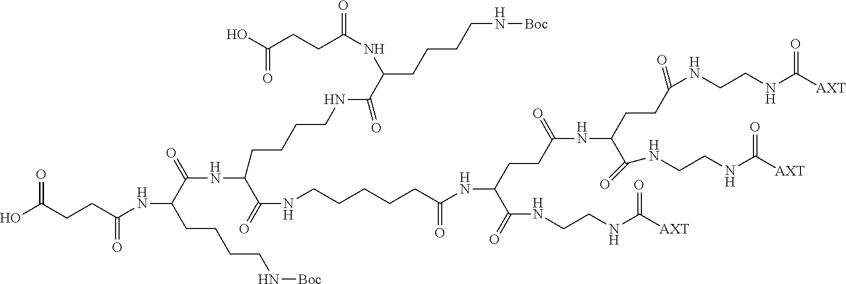

After the product 56-220 (0.3 g, 0.1283 mmol) was added to DMF for dissolution, DIEA (0.33 mL, 2 mmol) was added, and succinic anhydride (0.15 g, 1.3 mmol) was added for dissolution after 30 minutes of stirring and reaction at room temperature. After the reaction ended, methyl tertiary butyl ether (150 mL) and n-hexane (100 mL) were added to the reactant solution, to obtain a precipitate of a solid product which was subjected to suction filtration, and was dried under vacuum to obtain 0.35 g of the product, which was overproduction.

56-182

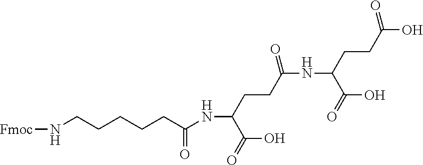

The raw material 57-170 (6 g, 7.69 mmol) was added into a 250 mL flask, dichloromethane (20 mL) was added for dissolution, then TFA (25.7 mL, 346.05 mmol) was added, and the reactant solution was stirred overnight at room temperature. After the reaction ended, dichloromethane in the reactant solution was drained, then methyl tertiary butyl ether (80 mL) was added for precipitation, to obtain a solid precipitate, then suction filtration was performed, and the resultant filter cake was rinsed with methyl tertiary butyl ether (40 mL×3), collected and dried in the vacuum oven to obtain 2.98 g of the product, with a yield of 63.40%.

56-183

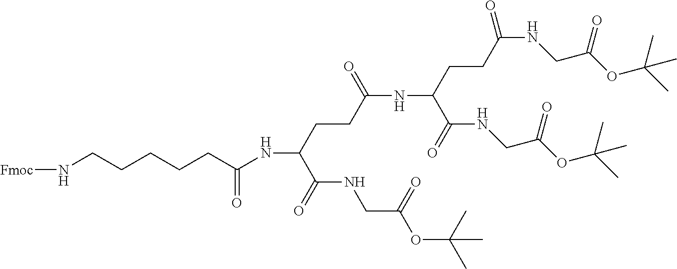

The product 56-182 (2.98 g, 4.87 mmol), glycine tert-butyl ester hydrochloride (2.7 g, 16.08 mmol, Accela), HBTU (8.3 g, 21.9 mmol) and HOBT (2.96 g, 21.9 mmol) were added to a 500 mL flask, the reactant solution was kept at −5° C. and stirred for about 20 minutes before DIEA (7.2 mL, 43.83 mmol) was slowly added dropwise. The resultant solution further reacted at −5° C. for 1 hour and then stirred overnight at room temperature. After the reaction ended, methyl tertiary butyl ether (80 mL) was added for precipitation, to obtain a solid precipitate, then suction filtration was performed, and the resultant filter cake was rinsed with methyl tertiary butyl ether (40 mL×3), collected and dried in the vacuum oven to obtain 4.9 g of the product, which was overproduction.

56-184

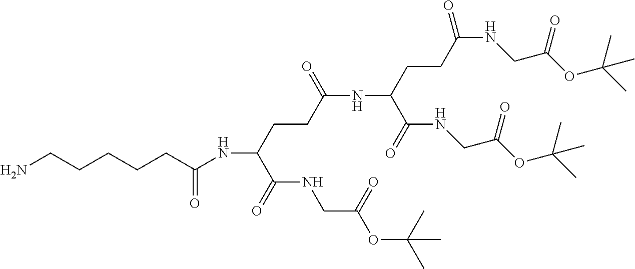 [SMILES: CC(C)(C)OC(=O)CNC(=O)CCC(NC(=O)CCC(NC(=O)CCCCCN)C(=O)NCC(=O)OC(C)(C)C)C(=O)NCC(=O)OC(C)(C)C]

The product 56-183 (4.6 g, 4.87 mmol) was taken, DMF (40 mL) was added for dissolution, then morpholine (5.4 mL, 15 mmol) was added, and the resultant solution was stirred for reaction at room temperature. After the reaction ended, methyl tertiary butyl ether (150 mL) and n-hexane (100 mL) were added to the reactant solution, to obtain a precipitate of a solid product which was subjected to suction filtration, and was dried under vacuum to obtain 3 g of the product, with a yield of 85.71%.

56-187

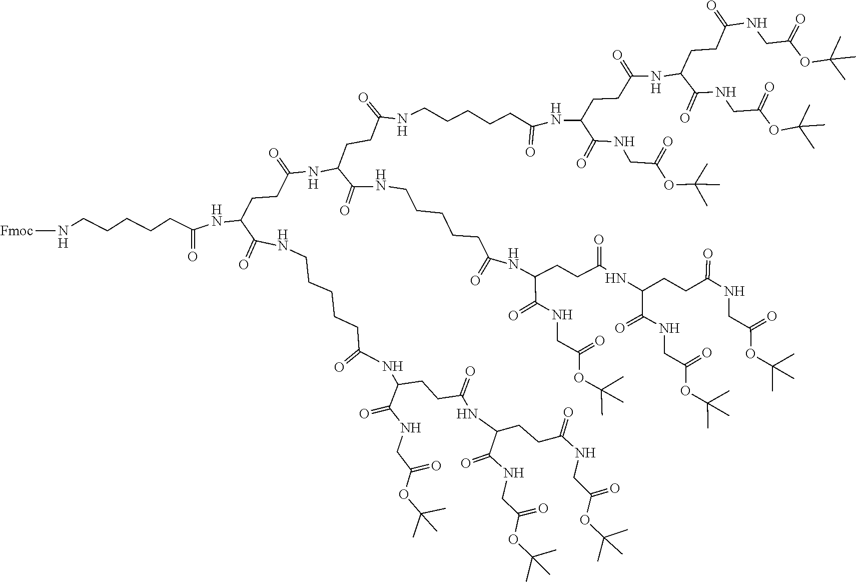

The product 56-182 (0.4 g, 0.8 mmol), the product 56-184 (2.1 g, 2.88 mmol), HBTU (1.37 g, 3.6 mmol) and HOBT (0.49 g, 3.6 mmol) were added to a 500 mL flask, the reactant solution was kept at −5° C. and stirred for about 20 minutes before DIEA (1.2 mL, 7.2 mmol) was slowly added dropwise. The resultant solution further reacted at −5° C. for 1 hour and then stirred overnight at room temperature. After the reaction ended, methyl tertiary butyl ether (80 mL) was added for precipitation, to obtain a solid precipitate, then suction filtration was performed, and the resultant filter cake was rinsed with methyl tertiary butyl ether (40 mL×3), collected and dried in the vacuum oven to obtain 1.2 g of the product, with a yield of 54.79%.

56-189

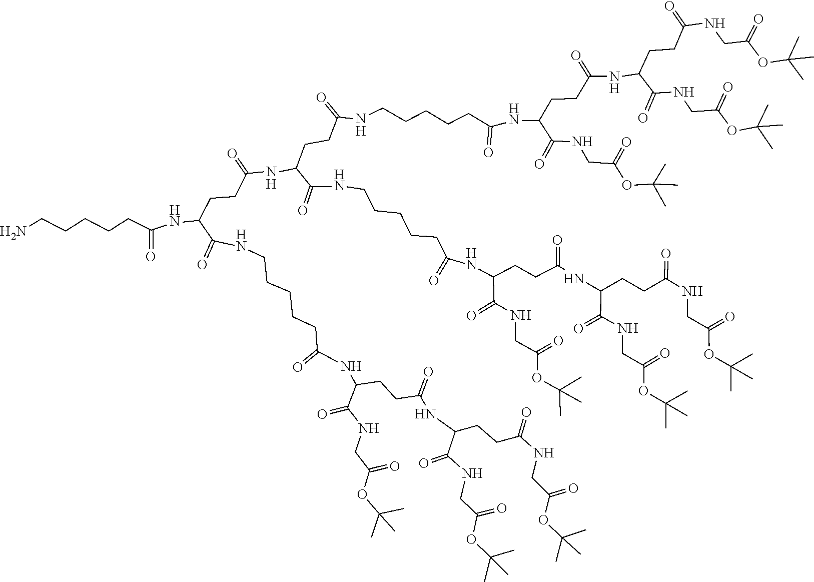 [SMILES: CC(C)(C)OC(=O)CNC(=O)CCC(NC(=O)CCC(NC(=O)CCCCCNC(=O)CCC(NC(=O)CCC(NC(=O)CCCCCN)C(=O)NCCCCCC(=O)NC(CCC(=O)NC(CCC(=O)NCC(=O)OC(C)(C)C)C(=O)NCC(=O)OC(C)(C)C)C(=O)NCC(=O)OC(C)(C)C)C(=O)NCCCCCC(=O)NC(CCC(=O)NC(CCC(=O)NCC(=O)OC(C)(C)C)C(=O)NCC(=O)OC(C)(C)C)C(=O)NCC(=O)OC(C)(C)C)C(=O)NCC(=O)OC(C)(C)C)C(=O)NCC(=O)OC(C)(C)C]

The product 56-187 (1.2 g, 0.437 mmol) was taken, DMF (40 mL) was added for dissolution, then morpholine (0.49 mL, 6.56 mmol) was added, and the resultant solution was stirred for reaction at room temperature. After the reaction ended, methyl tertiary butyl ether (150 mL) and n-hexane (100 mL) were added to the reactant solution, to obtain a precipitate of a solid product which was subjected to suction filtration, and was dried under vacuum to obtain 1.5 g of the product, which was overproduction.

56-232

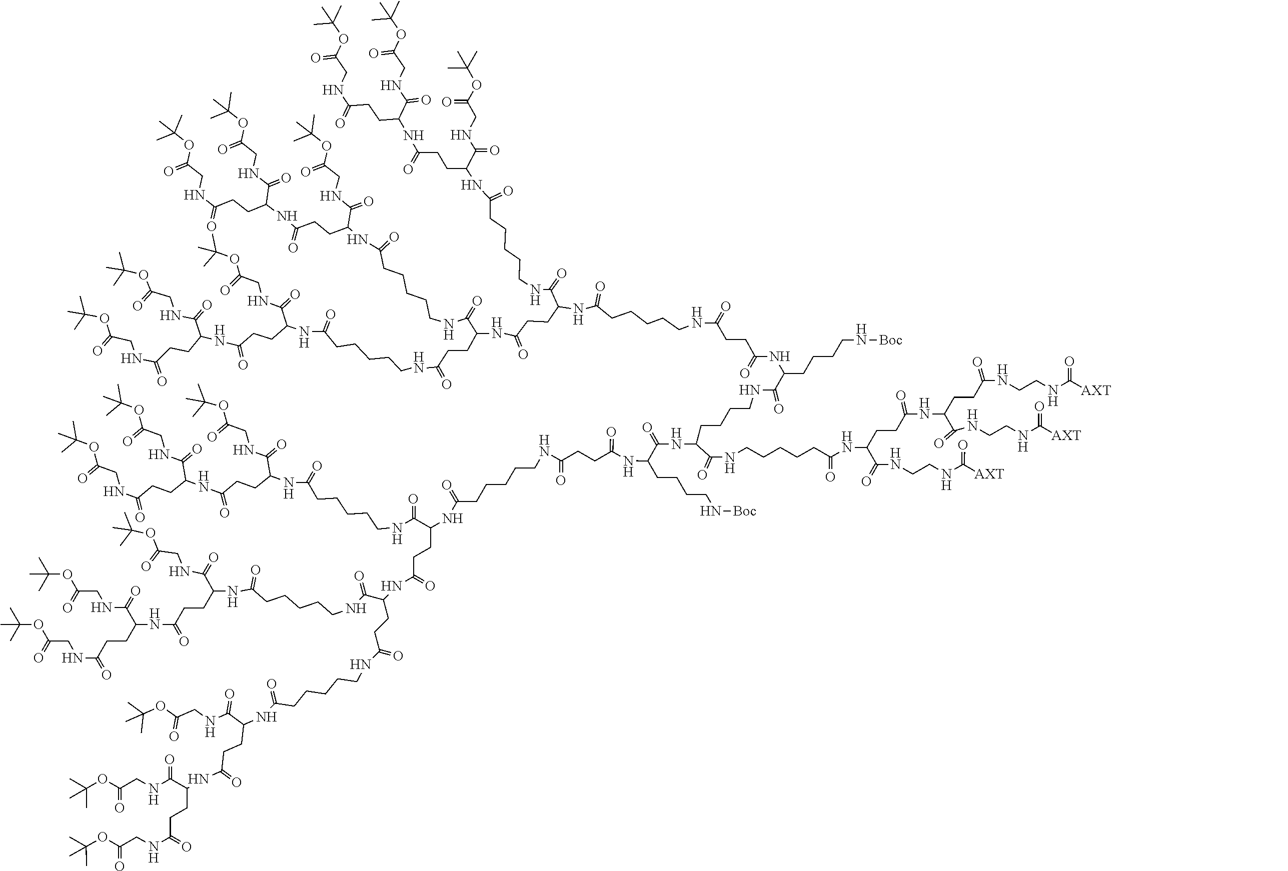

The product 56-221 (0.2 g, 0.16 mmol), the product 56-189 (1 g, 0.39 mmol), HBTU (0.18 g, 0.48 mmol) and HOBT (0.06 g, 0.48 mmol) were added to a 500 mL flask, the reactant solution was kept at −5° C. and stirred for about 20 minutes before DIEA (0.24 mL, 1.44 mmol) was slowly added dropwise. The resultant solution stirred overnight at room temperature. After the reaction ended, methyl tertiary butyl ether (80 mL) was added for precipitation, to obtain a solid precipitate, then suction filtration was performed, and the resultant filter cake was rinsed with methyl tertiary butyl ether (40 mL×3), collected and dried in the vacuum oven to obtain 1.2 g of the product.

56-233

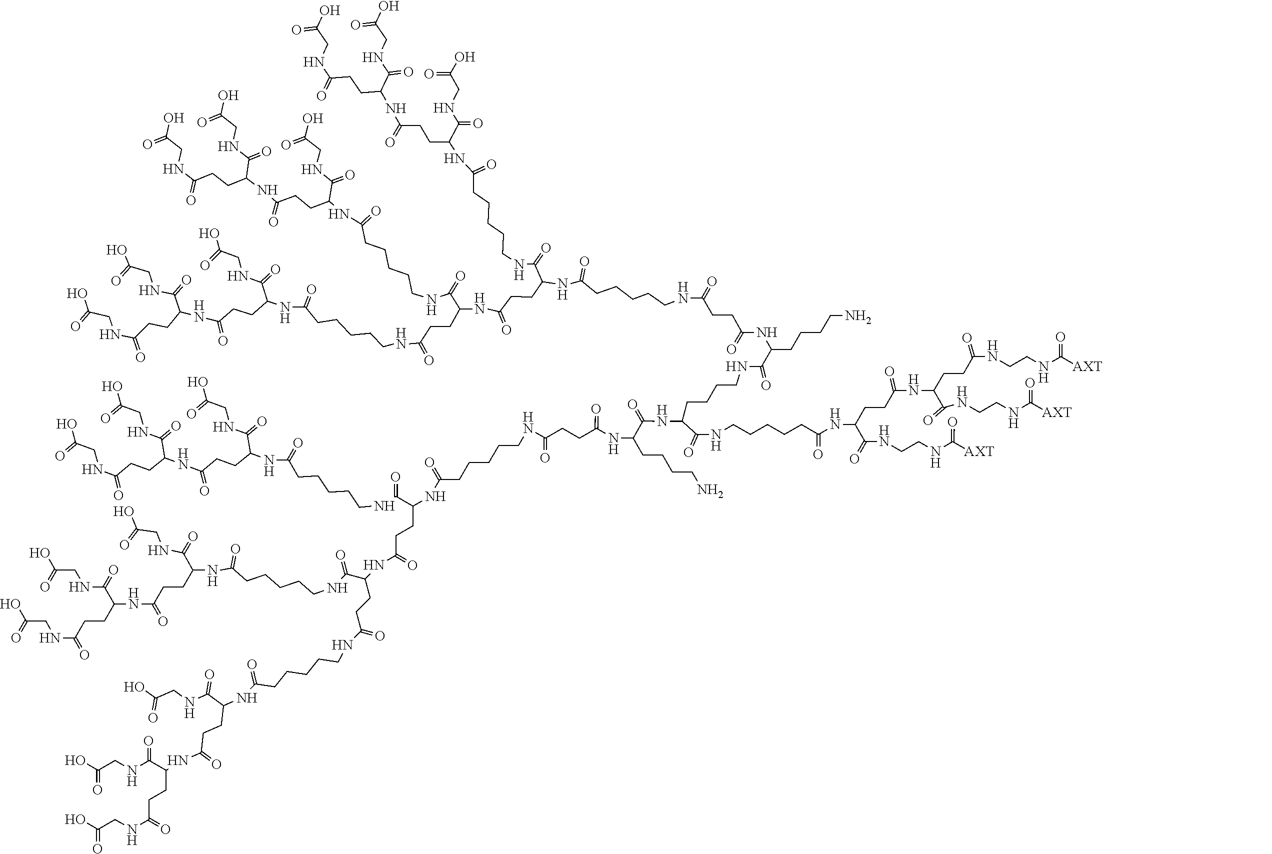

The product 56-232 (0.98 g, 0.16 mmol) was added to a 250 mL round-bottomed flask, and dissolved in dichloromethane (30 mL), and then TFA (0.14 mL, 1.6 mmol) was added, and the reactant solution was stirred for reaction at the room temperature overnight. After the reaction ended, the reactant solution was concentrated under reduced pressure and evaporated, then dichloromethane (5 mL) was added for dissolution, the reactant solution was taken out, methyl tertiary butyl ether (170 mL) was added for precipitation, then n-hexane (150 mL×3) was added for precipitation three times, and the reactant solution was drained and then dried in a vacuum box to obtain 0.5 g of the product, with a yield of 49.50%.

80-3

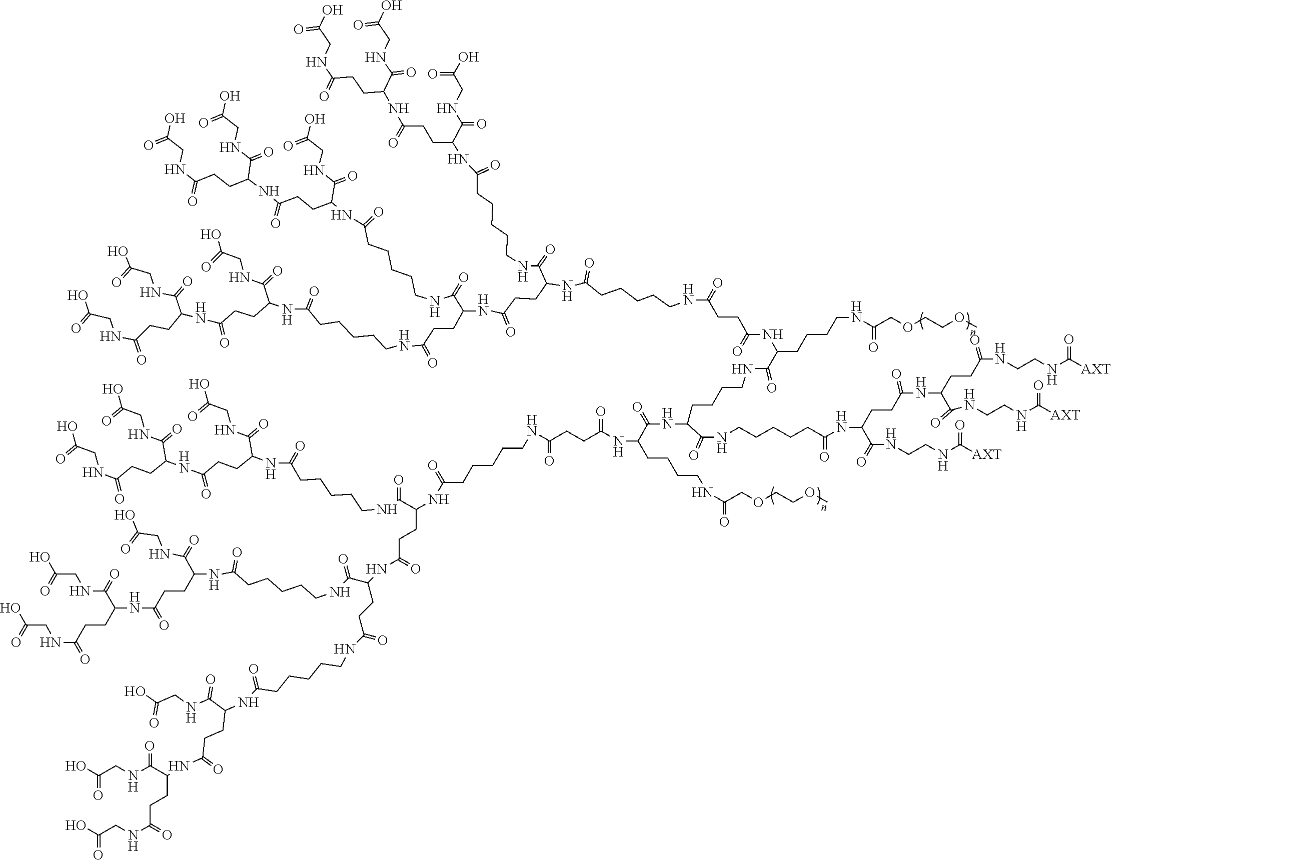

The product 56-233 (0.27 g, 0.043 mmoL) and M-SCM-10K (1 g, 0.094 mmoL) were weighed and added into a 250 mL flask, DMF (80 mL) was added for dissolution, the reactant solution was stirred at −5° C. for 30 minutes, and DIEA (0.063 mL, 0.387 mmoL) was slowly added dropwise. After reaction at −5° C. for 30 minutes, the reactant solution was taken out and reacted at room temperature. After it was detected that the reaction ended via TLC, 150 mL of a mixed solution of methyl tertiary butyl ether/n-hexane (1:5) was added and shaken, the supernatant was discarded after standing, such procedure was repeated three times, then 150 mL of methyl tertiary butyl ether was added, to obtain a solid precipitate, then suction filtration was performed, and dry loaded for column chromatography with elution using the mixed solution, and the product was collected, concentrated, evaporated to dryness and dried in a vacuum box to obtain 1.1 g of the product, with a yield of 94.01%.

80-6

The product 80-3 (0.2 g, 0.007 mmol), the product 53-213 (0.076 g, 0.15 mmol), HBTU (0.18 g, 0.48 mmol) and HOBT (0.06 g, 0.48 mmol) were added to a 500 mL flask, the reactant solution was kept at −5° ° C. and stirred for about 20 minutes before DIEA (0.24 mL, 1.44 mmol) was slowly added dropwise. The resultant solution stirred overnight at room temperature. After the reaction ended, methyl tertiary butyl ether (80 mL) was added for precipitation, to obtain a solid precipitate, then suction filtration was performed, and the resultant filter cake was rinsed with methyl tertiary butyl ether (40 mL×3), collected and dried in the vacuum oven to obtain 0.2 g of the product, with a yield of 80.00%.

80-9

The product 80-6 (0.2 g, 0.0055 mmol) was added to a 250 mL round-bottomed flask and dissolved in THE (50 mL), then TBAF (0.6 g, 0.99 mmol) was added, and the reactant solution was stirred for reaction at room temperature until the reaction ended. The reactant solution was concentrated under reduced pressure and evaporated, 150 mL of a mixed solution of methyl tertiary butyl ether/n-hexane (1:5) was added and shaken, to obtain a solid precipitate, then suction filtration was performed, the filter cake was dry loaded for column chromatography with elution using the mixed solution, and the product was collected, concentrated, evaporated to dryness and dried in a vacuum box to obtain 0.11 g of the product, with a yield of 58.51%.

¹H-NMR (600 MHz, DMSO-d₆) δ 11.5-10.78 (m, 10H), 10.73-10.35 (m, 38H), 8.49-8.23 (m, 17H), 8.19-7.96 (m, 30H), 7.95-7.81 (m, 14H), 7.55-6.95 (m, 19H), 6.81-6.58 (m, 14H), 6.55-6.40 (m, 7H), 6.35-6.18 (m, 10H), 6.05-5.91 (m, 3H), 5.28-5.21 (m, 3H), 5.19-5.08 (m, 15H), 4.98-4.91 (m, 8H), 4.81-4.75 (m, 9H), 3.98-3.87 (m, 165H), 3.51 (s, 1420H), 3.43-3.36 (m, 172H), 3.20-3.14 (m, 150H), 2.79-2.69 (m, 124H), 1.45-1.28 (m, 131H), 0.91-0.81 (m, 223H).

### Synthesis of Compound 61-104

51-43

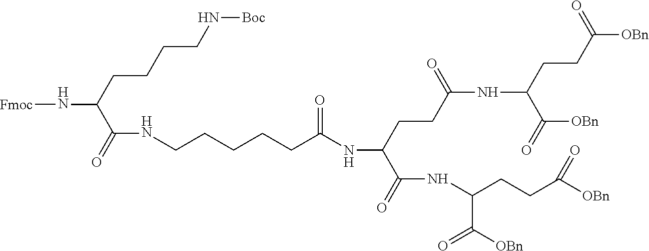

The product 39-159 (4.922 g, 5.60 mmol), Fmoc-Lys(Boc)-OH (2.624 g, 5.60 mmol), HOBT (1.135 g, 8.4 mmol) and HBTU (3.186 g, 8.4 mmol) were added into a 500 mL reaction flask, a DMF solution (100 mL) was added for dissolution, the reactant was completely ultrasonically dissolved, the reactant solution was stirred at −5° C. for 30 minutes, DIEA (4.165 mL, 25.2 mmol) was slowly added dropwise, and the reactant solution was left standing at low temperature until the reaction ended.

51-47

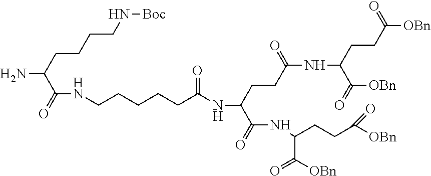 [SMILES: CC(C)(C)OC(=O)NCCCCC(N)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

DMF was added to a flask containing product 51-43 (5.1 g, 3.88 mmol), and was ultrasonically oscillated until complete dissolution, then morpholine (3.380 mL, 38.8 mmol) was added, and the resultant solution was stirred for reaction for 2 hours at room temperature. After the reaction ended, deionized water (200 mL) and EA (200 mL) were added for extraction, the organic phase was collected after standing and stratification, the aqueous phase was rinsed with EA (200 mL×3), the organic phases were combined and evaporated to dryness to obtain a solid, and the solid was dried in a vacuum oven.

51-49

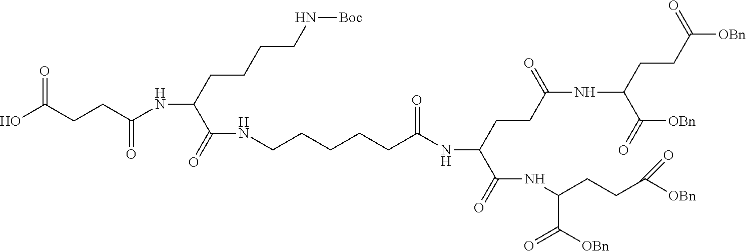 [SMILES: CC(C)(C)OC(=O)NCCCCC(NC(=O)CCC(=O)O)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

After the product 51-47 was added to and dissolved in DMF, half of a volume (1.94 mmol) of the reactant solution was taken, succinic anhydride (0.582 g, 5.82 mmol) was added for dissolution, DIEA (1.281 mL, 7.76 mmol) wad added, and the reactant solution was stirred for reaction at room temperature. After the reaction ended, methyl tertiary butyl ether (150 mL) and n-hexane (100 mL) were added to the reactant solution, to obtain a precipitate of a solid product which was subjected to suction filtration, and was dried under vacuum to obtain the product.

51-51

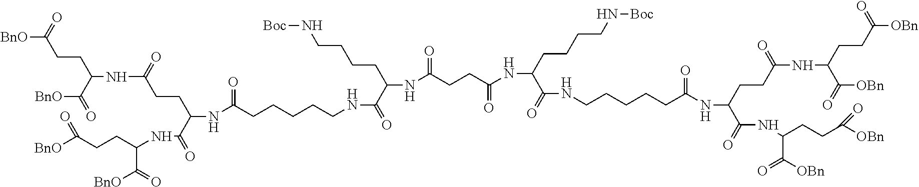 [SMILES: CC(C)(C)OC(=O)NCCCCC(NC(=O)CCC(=O)NC(CCCCNC(=O)OC(C)(C)C)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

The product 51-47 (2.148 g, 1.94 mmol), the product 51-49 (2.342, 1.94 mmol), HOBT (0.393 g, 2.91 mmol) and HBTU (1.104 g, 2.91 mmol) were weighed and added into a 250 mL reaction flask, a DMF solution (100 mL) was added for dissolution, the reactant was completely ultrasonically dissolved, the reactant solution was stirred at −5° C. for 30 minutes, DIEA (1.443 mL, 8.73 mmol) was slowly added dropwise, and the reactant solution was left standing at low temperature until the reaction ended. After the reaction ended, deionized water (200 mL) and EA (200 mL) were added for extraction, the organic phase was collected after standing and stratification, the aqueous phase was rinsed with EA (200 mL×3), the organic phases were combined and evaporated to dryness to obtain a solid, and the solid was dried in a vacuum oven, suction filtered, and dry loaded for column chromatography with gradient elution using 3% to 6% methanol/dichloromethane, and the product was collected.

61-71

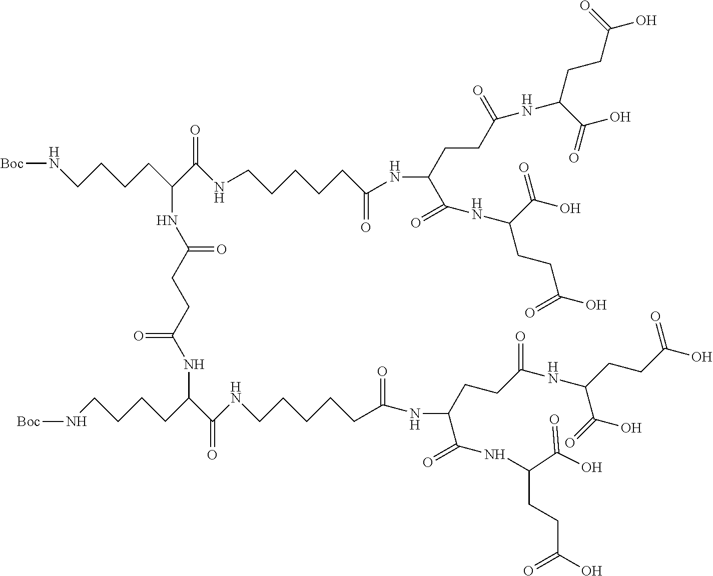 [SMILES: CC(C)(C)OC(=O)NCCCCC(NC(=O)CCC(=O)NC(CCCCNC(=O)OC(C)(C)C)C(=O)NCCCCCC(=O)NC(CCC(=O)NC(CCC(=O)O)C(=O)O)C(=O)NC(CCC(=O)O)C(=O)O)C(=O)NCCCCCC(=O)NC(CCC(=O)NC(CCC(=O)O)C(=O)O)C(=O)NC(CCC(=O)O)C(=O)O]

The raw material 51-51 (0.5 g, 0.2177 mmoL) and 10% Pd/C catalyst (50 mg) were added into a hydrogenation reaction instrument, then DMF (30 mL) was added for dissolution, the solvent submerged a stirring bar, and the hydrogenation reaction instrument was sealed. After three pumping operations and three injection operations (pumping out the air in the reaction system with a vacuum water pump for about 3 minutes—injecting hydrogen gas—pumping out hydrogen gas—injecting hydrogen gas—pumping out hydrogen gas—injecting hydrogen gas), a pressure reading on the hydrogenation reaction instrument was 18 Psi, and then the reactant solution reacted overnight at room temperature. On the next day, after it was detected that the reaction ended via a TLC plate, post-treatment was carried out, the reactant solution was taken out and evenly added dropwise to a suction filtration funnel filled with compacted celite, the reaction instrument was rinsed with DMF (20 mL×3) and DMF was added to celite dropwise until celite was clean and contained no product, to obtain reaction product to be used for the next reaction.

61-72

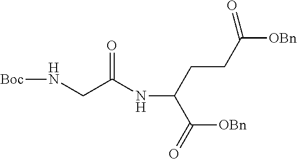 [SMILES: CC(C)(C)OC(=O)NCC(=O)NC(CCC(=O)OCc1ccccc1)C(=O)OCc1ccccc1]

Boc-Gly-OH (5.25 g, 30.02 mmol), L-glutamate dibenzyl ester p-toluenesulfonate (15.0 g, 30.02 mmol, Aladdin), HOBT (6.08 g, 45.03 mmol) and HBTU (17.07 g, 45.03 mmol) were added in a 500 mL round-bottomed flask and dissolved in DMF (150 mL), and the mixed solution was stirred at −5° C. for 30 minutes. Then DIEA (22.33 mL, 135.11 mmol) was slowly added dropwise, then the resultant solution reacted at low temperature for 2 hours, then the reaction instrument was put at room temperature and the resultant solution was stirred for reaction overnight. After the reaction ended, the reactant solution was transferred to a 1000 mL separatory funnel, a saturated sodium bicarbonate solution (200 mL), and extracted with ethyl acetate (200 mL×3) three times, organic phases were combined and then rinsed with saturated sodium chloride solution (100 mL×3) three times, and then the organic phases were dried with anhydrous sodium sulfate and subjected to suction filtration. After the filtrate was concentrated, silica gel powder (60 g) was added, the resultant mixture was dry loaded for column chromatography with elution using ethyl acetate/petroleum ether (3:7), and the product was collected, concentrated, evaporated to dryness and then dried in a vacuum box to obtain 15.2 g of the product.

61-92

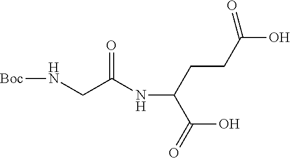 [SMILES: CC(C)(C)OC(=O)NCC(=O)NC(CCC(=O)O)C(=O)O]

The raw material 61-72 (1.28 g, 2.6582 mmoL) and 10% Pd/C catalyst (10 mg) were added into a hydrogenation reaction instrument, then DMF (30 mL) was added for dissolution, the solvent submerged a stirring bar, and the hydrogenation reaction instrument was sealed. After three pumping operations and three injection operations (pumping out the air in the reaction system with a vacuum water pump for about 3 minutes—injecting hydrogen gas—pumping out hydrogen gas—injecting hydrogen gas—pumping out hydrogen gas—injecting hydrogen gas), a pressure reading on the hydrogenation reaction instrument was 18 Psi, and then the reactant solution reacted overnight at room temperature. On the next day, after it was detected that the reaction ended via a TLC plate, post-treatment was carried out, the reactant solution was taken out and evenly added dropwise to a suction filtration funnel filled with compacted celite, the reaction instrument was rinsed with DMF (20 mL×3) and DMF was added to celite dropwise until celite was clean and contained no product, to obtain reaction product to be used for the next reaction.

61-93

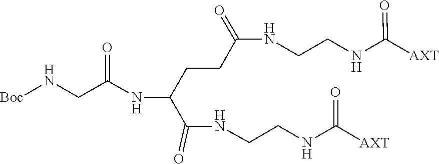

The product 61-92 (0.6 g, 1.9237 mmol), the product 53-215 (2.0 g, 4.2322 mmol), HBTU (2.2 g, 5.7711 mmol) and HOBT (0.78 g, 5.7711 mmol) were added in a 250 mL round-bottomed flask and dissolved in DMF (100 mL), and the mixed solution was stirred at −5° C. for 30 minutes. Then DIEA (2.9 mL, 17.3133 mmol) was slowly added dropwise, then the resultant solution reacted at low temperature for 2 hours, then the reaction instrument was put at room temperature and the resultant solution was stirred for reaction overnight. After it was detected that the reaction ended via a TLC plate, post-treatment was carried out, the reactant solution was taken out, methyl tertiary butyl ether (150 mL) was added for precipitation, then n-hexane (150 mL×3) was added for precipitation three times, and the reactant solution was drained and then dried in a vacuum box to obtain the product.

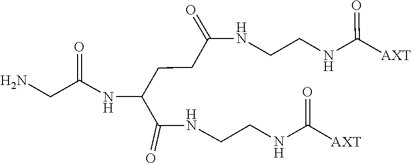

The product 61-93 (2.33 g, 1.9237 mmol) was added to a 250 mL round-bottomed flask, and dissolved in dichloromethane (30 mL), and then TFA (1.4 mL, 19.237 mmol) was added, and the reactant solution was stirred for reaction at the room temperature overnight. After the reaction ended, the reactant solution was concentrated under reduced pressure and evaporated, then dichloromethane (5 mL) was added for dissolution, then n-hexane (100 mL) was added for precipitation, the supernatant was discarded, such operation was repeated twice, the precipitate was evaporated to dryness and weighted, and subjected to column purification due to overweight, then dry loaded for column chromatography with elution using 1% NH₃·H₂O/12% methanol/dichloromethane, and the product was collected, concentrated, evaporated to dryness and then dried in a vacuum box to obtain 2.1 g of the product, with a yield of 98.13%.

61-96

The product 61-95 (1.46 g, 1.3104 mmol), the product 61-71 (0.23 g, 0.1489 mmol), HBTU (0.68 g, 1.7868 mmol) and HOBT (0.24 g, 1.7868 mmol) were added in a 250 mL round-bottomed flask and dissolved in DMF (100 mL), and the mixed solution was stirred at −5° C. for 30 minutes. Then DIEA (0.9 mL, 5.3604 mmol) was slowly added dropwise, then the resultant solution reacted at low temperature for 2 hours, then the reaction instrument was put at room temperature and the resultant solution was stirred for reaction overnight. After it was detected that the reaction ended via a TLC plate, post-treatment was carried out, the reactant solution was taken out, methyl tertiary butyl ether (150 mL) was added for precipitation, then n-hexane (150 mL×3) was added for precipitation three times, the reactant solution drained and dry loaded for column chromatography with elution using 1% NH₃·H₂O/10% methanol/dichloromethane, and the product was collected, concentrated, evaporated to dryness and then dried in a vacuum box to obtain 1.01 g of the product, with a yield of 67.33%.

61-103

The product 61-96 (1.01 g, 0.0977 mmol) was added to a 250 mL round-bottomed flask, and dissolved in dichloromethane (30 mL), and then TFA (0.14 mL, 1.954 mmol) was added, and the reactant solution was stirred for reaction at the room temperature overnight. After the reaction ended, the reactant solution was concentrated under reduced pressure and evaporated, then dichloromethane (5 mL) was added for dissolution, the reactant solution was taken out, methyl tertiary butyl ether (170 mL) was added for precipitation, then n-hexane (150 mL×3) was added for precipitation three times, and the reactant solution was drained and then dried in a vacuum box to obtain 1.27 g of the product.

61-104

The product 61-103 (1.05 g, 0.0493 mmol) and M-SCM-10K (1.0 g, 0.0986 mmol, JenKem) were added into a 250 mL round-bottomed flask and dissolved in DMF (100 mL), and the mixed solution was stirred at −5° C. for 30 minutes. Then DIEA (0.4 mL, 2.465 mmol) was slowly added dropwise, then the resultant solution reacted at low temperature for 2 hours, then the reaction instrument was put at room temperature and the resultant solution was slowly stirred for reaction. After it was detected that the reaction ended via a TLC plate, post-treatment was carried out, the reactant solution was taken out, methyl tertiary butyl ether (150 mL) was added for precipitation, then n-hexane (150 mL×3) was added for precipitation three times, the reactant solution drained and dry loaded for column chromatography with elution using 9% methanol/dichloromethane, and the product was collected, concentrated, evaporated to dryness and then dried in a vacuum box to obtain 0.9 g of the product, with a yield of 57.89%.

¹H-NMR (600 MHz, DMSO-d₆) δ 8.63-8.55 (m, 16H), 8.52-8.43 (m, 16H), 8.42-8.35 (m, 16H), 8.32-8.28 (m, 16H), 8.26-8.18 (m, 16H), 8.14-8.06 (m, 16H), 8.00-7.83 (m, 32H), 7.83-7.69 (m, 33H), 7.69-7.59 (m, 18H), 7.51-7.44 (m, 16H), 7.31-7.27 (m, 48H), 7.18-7.15 (m, 16H), 7.13-7.07 (m, 16H), 7.06-7.02 (m, 16H), 5.78-5.74 (m, 3H), 4.28-4.17 (m, 22H), 3.91-3.79 (m, 30H), 3.71-3.65 (m, 34H), 3.52-3.49 (m, 1876H), 3.25-3.23 (m, 17H), 2.91-2.87 (m, 4H), 2.81-2.64 (m, 58H), 2.38-2.28 (m, 7H), 2.26-2.02 (m, 26H), 2.00-1.64 (m, 22H), 1.13-1.03 (m, 9H)

### Synthesis of Compound 51-151

61-62

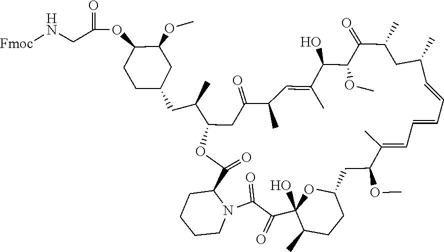

Rapamycin (SRM for short, 5 g, 5.4964 mmol), Fmoc-Gly-OH (1.8 g, 6.0164 mmol) and DMAP (0.13 g, 1.0934 mmol) were weighed and added to a 500 mL flask, then an appropriate amount of dichloromethane was added for dissolution, the reactant solution was kept at 0° C. and stirred for 30 minutes, DCC (3.4 g, 16.4083 mmol) was added, and the reactant solution reacted for half an hour, then taken out and stirred for reaction overnight at room temperature. After the reaction ended, a saturated NaCl solution (100 mL) was added, EA (100 mL) was added for extraction, the organic phase was collected after standing and stratification, the aqueous phase was rinsed with EA (100 mL×3), and the organic phases were combined and evaporated to dryness, silica gel powder (50 mL) was added, and the resultant mixture was evaporated to dryness to obtain a powder-like solid, and the powder-like solid was dry loaded for column chromatography with elution using 30% to 50% ethyl acetate in petroleum ether, collection, concentration and then drying in an oven to obtain product 61-62 (5.4 g, 69%) to be used as the raw material for the next reaction.

61-64

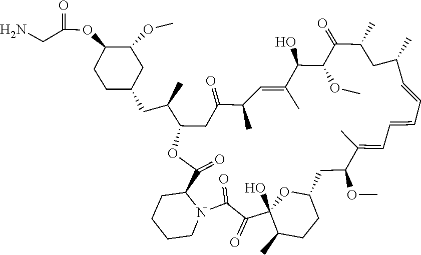

DMF was added to a flask containing the product 61-62 (5.4 g, 4.5247 mmol), and was ultrasonically oscillated until complete dissolution, then morpholine (3.9 mL, 45.247 mmol) was added, the resultant solution was stirred for reaction at room temperature, and a reaction procedure was monitored via the TLC plate. After the reaction ended, a saturated NaCl solution (100 mL) was added, EA (100 mL) was added for extraction, the organic phase was collected after standing and stratification, the aqueous phase was rinsed with EA (100 mL×3), and the organic phases were combined and evaporated to dryness, silica gel powder (50 mL) was added, and the resultant mixture was evaporated to dryness to obtain a powder-like solid, and the powder-like solid was dry loaded for column chromatography with elution using 1.5% to 10% methanol in dichloromethane, collection, concentration and then drying in an oven to obtain product 61-64 (1.8 g, 41%) to be used as the raw material for the next reaction.

61-66

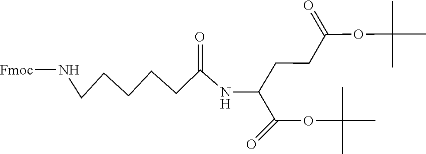

Fmoc-AH-OH (synthesized in a synthesis method of the product 49-17, 5.0 g, 14.1479 mmol), glutamic acid di-tert-butyl ester (4.6 g, 15.5627 mmol, Aladdin), HBTU (8.0 g, 21.2218 mmol) and HOBT (2.9 g, 21.2218 mmol) were weighted and added into a 500 mL flask, then an appropriate amount of DMF was added for dissolution, the reactant solution was kept at −5° C., and DIEA (10.5 mL, 63.6654 mmol) was slowly added dropwise, and then the reactant solution reacted for half an hour, then taken out and stirred for reaction overnight at room temperature. After the reaction ended, a saturated NaCl solution (100 mL) was added, EA (100 mL) was added for extraction, the organic phase was collected after standing and stratification, the aqueous phase was rinsed with EA (100 mL×3), and the organic phases were combined and concentrated to obtain a yellow-brown oily product (11.1 g, overproduction), to be used as the raw material for the next reaction.

61-67

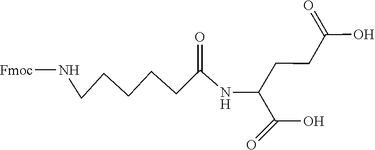

CH₂Cl₂ was added to a flask containing the product 61-66 (8.4 g, 14.1479 mmol), and was ultrasonically oscillated until complete dissolution, then TFA (16.0 mL, 141.479 mmol) was added, and the resultant solution was stirred for reaction overnight at room temperature. After the reaction ended, CH₂Cl₂ in the reactant solution was evaporated, then n-hexane (200 mL) and methyl tertiary butyl ether (40 mL) were added, the supernatant was discarded, such operation was repeated three times, then methyl tertiary butyl ether (200 mL) and a small amount of n-hexane (40 mL) were added for oscillation, to obtain a solid precipitate, then suction filtration was performed, and the resultant filter cake was rinsed with methyl tertiary butyl ether (100 mL×3) and was dried in a vacuum oven to obtain product 61-67 (6.3 g, 92.6%). The product was used as a raw material for the next reaction.

61-70

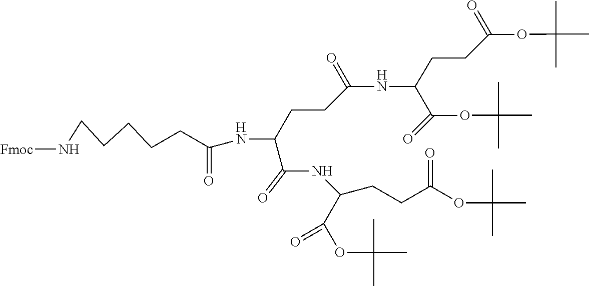

The product 61-67 (6.3 g, 13.0562 mmol), glutamic acid di-tert-butyl ester (8.5 g, 28.7236 mmol), HBTU (14.9 g, 39.1686 mmol) and HOBT (5.3 g, 39.1686 mmol) were weighted and added into a 500 mL flask, then an appropriate amount of DMF was added for dissolution, the reactant solution was kept at −5° C., and DIEA (19.4 mL, 117.5058 mmol) was slowly added dropwise, and then the reactant solution reacted for half an hour, then taken out and stirred for reaction overnight at room temperature. After the reaction ended, n-hexane (200 mL) and methyl tertiary butyl ether (40 mL) were added, the supernatant was discarded, such operation was repeated three times, then methyl tertiary butyl ether (200 mL) and a small amount of n-hexane (40 mL) were added for oscillation, to obtain a solid precipitate, then suction filtration was performed, and the resultant filter cake was rinsed with methyl tertiary butyl ether (200 mL×3) and subjected to elution with a mixed solution of 5% to 10% methanol in dichloromethane, collection, concentration, and drying in a vacuum oven to obtain product 61-70 (6.0 g, 47.6%). The product was used as a raw material for the next reaction.

51-91

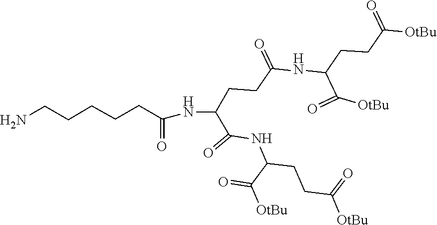

The product 61-70 (4.0 g, 4.14 mmol) was taken, DMF (40 mL) was added for dissolution, then morpholine (7.2 mL, 82.8 mmol) was added, and the resultant solution was stirred for reaction at room temperature. After the After the reaction ended, the reactant solution was taken out, added with deionized water (100 mL), and extracted with ethyl acetate (100 mL×3) multiple times, the organic phases were combined, rinsed with a saturated sodium chloride solution (100 mL) twice and evaporated to dryness.

51-92

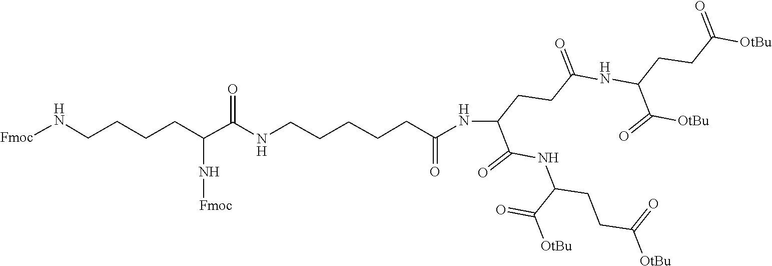

The Fmoc-Lys(Fmoc)-OH (2.44 g, 4.14 mmol), the product 51-91 (4.14 mmol), HOBT (0.83 g, 6.21 mmol) and HBTU (2.35 g, 6.21 mmol) were weighted and added into a 250 mL reaction flask, a DMF solution (80 mL) was added for dissolution, the reactant was completely ultrasonically dissolved, the reactant solution was stirred at −5° C. for 30 minutes, DIEA (4.49 mL, 27.2 mmol) was slowly added dropwise, and the reactant solution was taken out after 1 hour, and stirred for reaction at room temperature. After the reaction ended, methyl tertiary butyl ether (150 mL) and n-hexane (100 mL) were added to the reactant solution, to obtain a precipitate of a solid product which was subjected to suction filtration. The resultant filter cake was dry loaded for column chromatography, and gradient elution was performed using 3% methanol/dichloromethane, and the resultant product was collected, concentrated and evaporated to obtain the product.

51-94

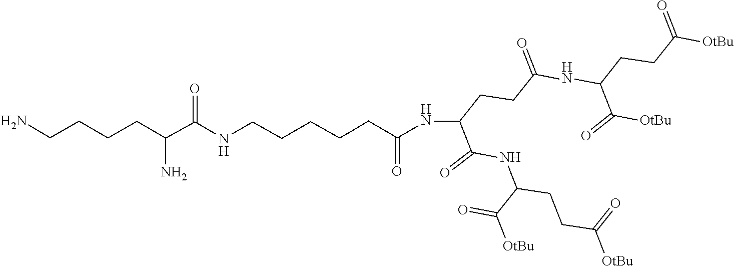 [SMILES: CC(C)(C)OC(=O)CCC(NC(=O)CCC(NC(=O)CCCCCNC(=O)C(N)CCCCN)C(=O)NC(CCC(=O)OC(C)(C)C)C(=O)OC(C)(C)C)C(=O)OC(C)(C)C]

The product 51-92 (0.11 g, 0.084 mmol) was taken, DMF (40 mL) was added for dissolution, then piperidine (0.166 mL, 1.68 mmol) was added, and the resultant solution was stirred for reaction at room temperature. After the After the reaction ended, the reactant solution was taken out, added with deionized water (100 mL), and extracted with ethyl acetate (100 mL×3) multiple times, the organic phases were combined, rinsed with a saturated sodium chloride solution (100 mL) twice and evaporated to dryness.

51-109

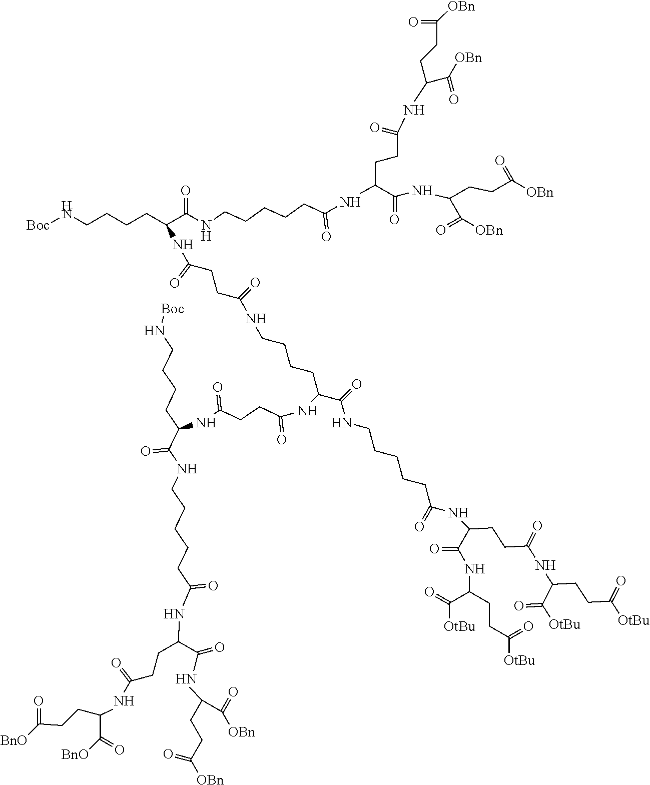

Reactants 51-49 (0.7, 0.5786 mmol), the product 51-94 (0.263 mmol), HBTU (0.299 g, 0.789 mmol), and HOBT (0.106 g, 0.789 mmol) were taken and added into a 250 mL reaction flask, DMF (40 mL) was added for dissolution, the reactant solution was left standing at −5° C. and stirred for 30 minutes, DIEA (0.39 mL, 2.2 mmol) was slowly added dropwise, and the reactant solution reacted at low temperature for 2 hours and then reacted at room temperature. After the reaction ended, methyl tertiary butyl ether (150 mL) and n-hexane (100 mL) were added to the reactant solution for precipitation, to obtain a precipitate of a solid product which was subjected to suction filtration, and was dried under vacuum to obtain 0.39 g of the product.

51-115

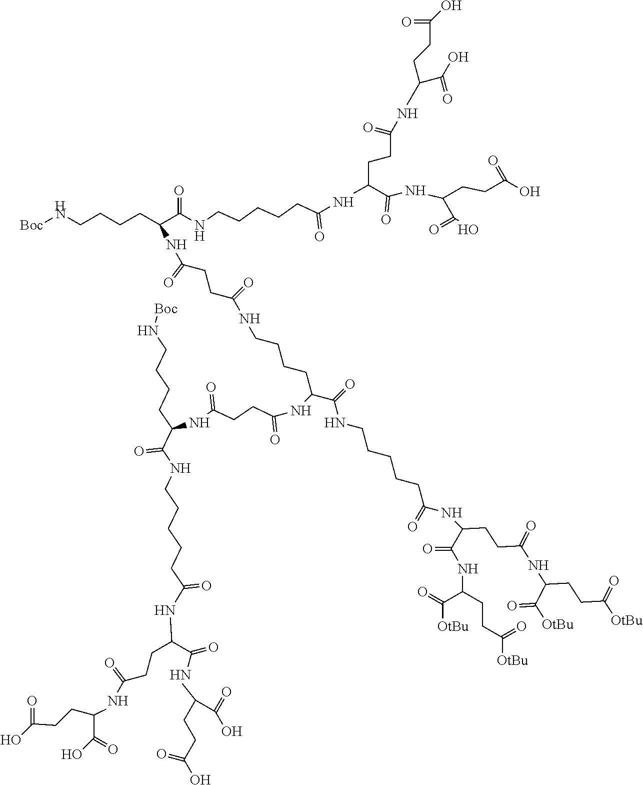

The product 51-109 (0.39 g, 0.12 mmol) was added into a reaction kettle, 10% Pd/C catalyst (0.0700 g) was added, DMF (30 mL) was added for dissolution, air in the reaction kettle was pumped out with a water pump to create a vacuum, hydrogen gas was injected at a hydrogen gas pressure of 0.16 MPa, then the hydrogen gas was released, the hydrogen gas was pumped out with a water pump to create a vacuum, and then hydrogen gas was injected again, such procedure was repeated three times, and finally hydrogen gas was injected again, and the reactant solution was stirred overnight at room temperature. After the reaction ended, the reactant solution was filtered with celite, the filter cake was rinsed with DMF (20 mL×3), and the DMF solution was combined and used as a raw material for the next step.

51-117

Reactants 51-115 (0.12 mmol), the product 61-95 (1.29, 1.05 mmol), HBTU (0.54 g, 1.44 mmol), and HOBT (0.19 g, 1.44 mmol) were taken and added into a 250 mL reaction flask, DMF (40 mL) was added for dissolution, the reactant solution was left standing at −5° C. and stirred for 30 minutes, DIEA (0.71 mL, 4.32 mmol) was slowly added dropwise, and the reactant solution reacted at low temperature for 2 hours and then reacted at room temperature. After the reaction ended, methyl tertiary butyl ether (150 mL) and n-hexane (100 mL) were added to the reactant solution for precipitation, to obtain a precipitate of a solid product which was subjected to suction filtration, and was dried under vacuum.

51-140

The product 51-117 (2.58 mmol) was taken, dichloromethane (10 mL) and TEA (3.8 mL, 51.6 mmol) were added, and the resultant solution was stirred for reaction at room temperature. After the reaction ended, the reactant solution was concentrated, methyl tertiary butyl ether (150 mL) and n-hexane (100 mL) were added to the reactant solution for precipitation, to obtain a precipitate of a solid product which was subjected to suction filtration, dried under vacuum, and dry loaded for column chromatography, and gradient elution was performed using 2% to 5% methanol/dichloromethane, to obtain 1.0 g of the product.

51-142

The compound 51-140 (0.07 mmol) and M-SCM-10K (1.5 g, 0.14 mmol, JenKem) were taken and added into a 250 mL reaction kettle. The resultant mixture was dissolved in DMF (30 mL) and DIEA (3.0 mL, 18.2 mmol) was added. The reactant solution was stirred at a low speed while protected from light. After the reaction ended, the reactant solution was concentrated, methyl tertiary butyl ether (150 mL) and n-hexane (100 mL) were added, to obtain a precipitate of a solid product which was subjected to suction filtration, and was dried under vacuum to obtain the product.

51-151

The product 51-142 (0.035 mmol), the product 61-64 (0.19 g, 0.197 mmol), HOBT (0.035 g, 0.262 mmol) and HBTU (0.099 g, 0.262 mmol) were weighted and added into a 250 mL reaction flask, a DMF solution (80 mL) was added for dissolution, the reactant was completely ultrasonically dissolved, the reactant solution was stirred at −5° C. for 30 minutes, DIEA (0.13 mL, 0.787 mmol) was slowly added dropwise, and the reactant solution was taken out after 1 hour, and stirred for reaction at room temperature. After the reaction ended, methyl tertiary butyl ether (150 mL) and n-hexane (100 mL) were added to the reactant solution, to obtain a precipitate of a solid product which was subjected to suction filtration. The resultant filter cake was dry loaded for column chromatography, and gradient elution was performed using a mixed solution of 4% to 10% methanol/dichloromethane, and the desired component was collected, concentrated and evaporated to obtain 0.6 g of the product.

¹H-NMR (600 MHz, DMSO-d₆) δ 8.66-8.55 (m, 21H), 8.54-8.42 (m, 21H), 8.42-8.35 (m, 21H), 8.33-8.27 (m, 24H), 8.26-8.17 (m, 26H), 8.16-8.02 (m, 32H), 8.01-7.84 (m, 39H), 7.84-7.69 (m, 46H), 7.69-7.55 (m, 25H), 7.53-7.41 (m, 24H), 7.39-7.23 (m, 84H), 7.23-7.19 (m, 4H), 7.16-7.05 (m, 24H), 6.99-6.90 (m, 4H), 5.91-5.61 (m, 23H), 4.30-4.13 (m, 23H), 4.12-3.96 (m, 10H), 3.88-3.80 (m, 14H), 3.79-3.72 (m, 20H), 3.71-3.63 (m, 31H), 3.58-3.41 (m, 1898), 3.25-3.20 (m, 55), 3.19-3.14 (m, 21H), 3.13-3.08 (m, 18H), 3.07-3.99 (m, 17H), 2.98-2.91 (m, 11H), 2.90-2.86 (m, 6H) 2.79-2.61 (m, 78H), 2.35-2.29 (m, 15H), 2.14-2.03 (m, 42H), 2.01-1.97 (m, 4H), 1.96-1.83 (m, 20H) 1.82-1.60 (m, 29H), 1.55-1.29 (m, 46H), 1.27-1.12 (m, 21H), 1.10-0.99 (m, 9H), 0.91-0.76 (m, 4H).

### Synthesis of Compound 68-114

53-211

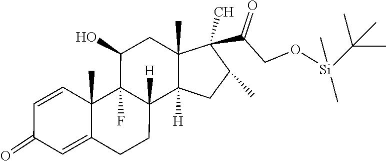

Reactants TBDMS-Cl (2.1 g, 14 mmol), DXM (5 g, 12.7 mmol) and imidazole (1.7 g, 25.5 mmol) were taken and added into the reaction flask, DMF was added for ultrasonic dissolution, nitrogen gas was injected for protection, the reactant solution was stirred at low temperature of 0° C., and the reaction was monitored via TLC. After the reaction ended, the reactant solution was extracted with ethyl acetate and saturated sodium bicarbonate, evaporated, and extracted with ethyl acetate and saturated deionized water to obtain 5.6 g of the product, with a yield of 78.6%.

53-213

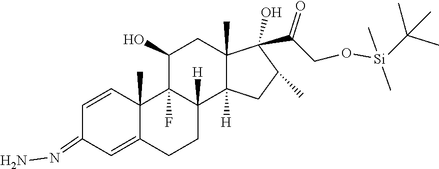

Reactants 53-211 (5.6 g, 11 mmol), hydrazine hydrate (3.3 mml, 66 mmol) and acetic acid (0.12 g, 2.2 mmol) were taken and added into the reaction flask, methanol was added for ultrasonic dissolution, the reactant solution was stirred at room temperature, and the reaction was monitored via TLC. After the reaction ended, the reactant solution was concentrated with a rotary evaporator, extracted with ethyl acetate and saturated deionized water, concentrated, and dried in an oven to obtain 4.5 g of the product, with a yield of 80.6%.

53-212

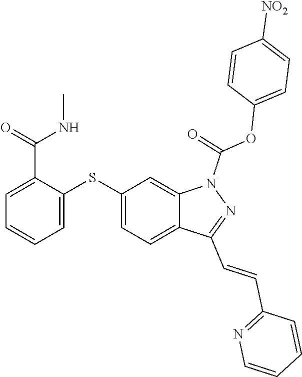 [SMILES: CNC(=O)c1ccccc1Sc1ccc2c(/C=C/c3ccccn3)nn(C(=O)Oc3ccc([N+](=O)[O-])cc3)c2c1]

Reactants 4-nitrophenyl chloroformate (2.1 g, 10.2 mmol), AXT (2 g, 5.1 mmol) and imidazole (1.7 g, 25.5 mmol) were taken and added into a reaction flask, THE was added for ultrasonic dissolution, a reactant was stirred at 70° C., and the reaction was monitored via TLC. After the reaction ended, methyl tertiary butyl ether/n-hexane was added for precipitation, and suction filtration was performed with a sand core funnel, and the filter cake was collected. The previous step was repeated twice, and the filter cake was transferred to a flask for drying, to obtain 3.8 g of the product, which was overproduction.

53-214

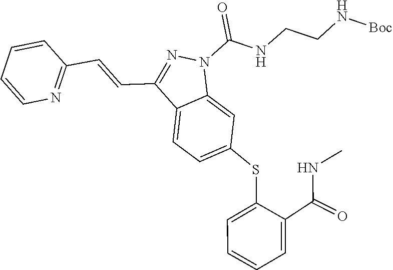 [SMILES: CNC(=O)c1ccccc1Sc1ccc2c(/C=C/c3ccccn3)nn(C(=O)NCCNC(=O)OC(C)(C)C)c2c1]

Reactants 53-212 (2.7 g, 5 mmol) and Boc-ethylenediamine (0.8 mml, 5 mmol) were taken, triethylamine (2.0, 14.6 mmol) and DMF were added for dissolution, the reactant solution was added into the reaction flask, the reactant solution was stirred at room temperature, and the reaction was monitored via TLC. After the reaction ended, the reactant solution was concentrated with a rotary evaporator, extracted with ethyl acetate and saturated deionized water repeatedly twice, concentrated, and dried in an oven to obtain 2.8 g of the product, with a yield of 96.6%.

53-215

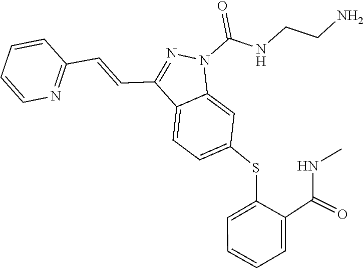 [SMILES: CNC(=O)c1ccccc1Sc1ccc2c(/C=C/c3ccccn3)nn(C(=O)NCCN)c2c1]

Reactant 53-214 (2.8 g, 4.9 mmol) was taken, 15 mL of dichloromethane was added for dissolution, then TFA (7.3 mL 98 mmol) was added, the reactant solution was stirred for reaction at room temperature, and the reaction was monitored via TLC. After the reaction ended, the reactant solution was concentrated with a rotary evaporator, methyl tertiary butyl ether/n-hexane was added for precipitation, and suction filtration was performed with a sand core funnel, and the filter cake was collected. The previous step was repeated twice, and the filter cake was transferred to a flask for drying. The resultant solid was dissolved in a mixed solvent, silica gel powder was added, and the resultant mixture was evaporated to dryness and was subjected to column chromatography using an eluent of 2% methanol/dichloromethane. 2.1 g of the product was obtained, with a yield of 91.3%.

48-154

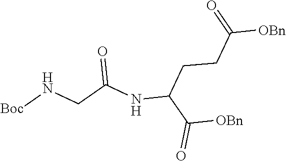 [SMILES: CC(C)(C)OC(=O)NCC(=O)NC(CCC(=O)OCc1ccccc1)C(=O)OCc1ccccc1]

Reactant glutamate dibenzyl ester p-toluenesulfonate (5 g, 10 mmol), Boc-glycine (1.7 g, 10 mmol), HOBT (2 g, 15 mmol) and HBTU (5.7 g, 15 mmol) were added into the reaction flask, DMF was added for ultrasonic dissolution, the reactant solution was stirred at low temperature of 0° C., and DIEA (7.4 mml, 45 mmol) was added dropwise. After dropwise addition, the reactant solution was taken out and further stirred for reaction for 1 hour at room temperature, and the reaction was monitored via TLC. After the reaction ended, deionized water and ethyl acetate were added for extraction, and the organic phase was collected; and the aqueous phase was further extracted twice, the organic phases were combined and evaporated to dryness, silica gel powder was added, and the resultant mixture was subjected to column chromatography using an eluent of 35% ethyl acetate/petroleum ether. 4.6 g of the product was obtained, with a yield of 93.9%.

51-194

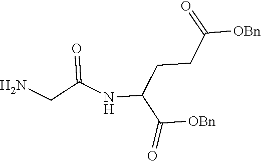 [SMILES: NCC(=O)NC(CCC(=O)OCc1ccccc1)C(=O)OCc1ccccc1]

Reactant 48-154 (2.6 g, 5.3 mmol) was taken, 20 mL of dichloromethane was added for dissolution, then TFA (8 mL 106 mmol) was added, the reactant solution was stirred for reaction at room temperature, and the reaction was monitored via TLC. After the reaction ended, the reactant solution was evaporated to dryness with a rotary evaporator, ethyl acetate and saturated sodium bicarbonate solution were added for extraction, the organic phase was collected, the aqueous phase was repeatedly extracted twice, and the organic phases were combined and evaporated to dryness, to obtain 2.1 g of product, which was overproduction.

51-197

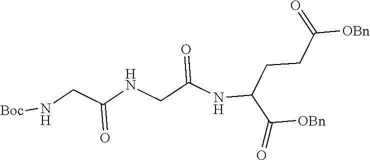 [SMILES: CC(C)(C)OC(=O)NCC(=O)NCC(=O)NC(CCC(=O)OCc1ccccc1)C(=O)OCc1ccccc1]

Reactant 51-194 (5.3 mmol), Boc-glycine (0.93 g, 5.3 mmol, Innochem), HOBT (0.34 g, 7.6 mmol) and HBTU (1 g, 7.6 mmol) were added into the reaction flask, DMF was added for ultrasonic dissolution, the reactant solution was stirred at low temperature of 0° C., and DIEA (44 mml, 264.07 mmol) was added dropwise. After dropwise addition, the reactant solution was taken out and further stirred for reaction for 1 hour at room temperature, and the reaction was monitored via TLC. After the reaction ended, deionized water and ethyl acetate were added for extraction, and the organic phase was collected; and the aqueous phase was further extracted twice, the organic phases were combined and evaporated to dryness, silica gel powder was added, and the resultant mixture was subjected to column chromatography using an eluent of 35% ethyl acetate/petroleum ether. 1.6 g of the product was obtained, with a yield of 55.9%.

51-199

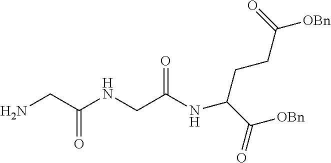 [SMILES: NCC(=O)NCC(=O)NC(CCC(=O)OCc1ccccc1)C(=O)OCc1ccccc1]

Reactant 51-197 (1.6 g, 3.0 mmol) was taken, 10 mL of dichloromethane was added for dissolution, then TFA (2.2 mL, 30 mmol) was added, the reactant solution was stirred for reaction at room temperature, and the reaction was monitored via TLC. After the reaction ended, the reactant solution was evaporated to dryness with a rotary evaporator, ethyl acetate and saturated sodium bicarbonate solution were added for extraction, the organic phase was collected, the aqueous phase was repeatedly extracted twice, and the organic phases were combined and evaporated to dryness, to obtain 1.6 g of product, which was overproduction.

51-200

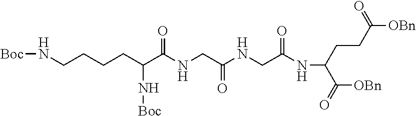 [SMILES: CC(C)(C)OC(=O)NCCCCC(NC(=O)OC(C)(C)C)C(=O)NCC(=O)NCC(=O)NC(CCC(=O)OCc1ccccc1)C(=O)OCc1ccccc1]

Reactant 51-199 (3.0 mmol), Boc-Lys-(Boc)-OH (1.6 g, 3.0 mmol, China Pharmaceutical Research & Development Center Co., Ltd.), HOBT (0.6 g, 4.5 mmol) and HBTU (1.7 g, 4.5 mmol) were added into the reaction flask, DMF was added for ultrasonic dissolution, the reactant solution was stirred at low temperature of 0° C., and DIEA (2.2 mml, 13.5 mmol) was added dropwise. After dropwise addition, the reactant solution was taken out and further stirred for reaction for 1 hour at room temperature, and the reaction was monitored via TLC. After the reaction ended, deionized water and ethyl acetate were added for extraction, and the organic phase was collected; and the aqueous phase was further extracted twice, the organic phases were combined and evaporated to dryness, silica gel powder was added, and the resultant mixture was subjected to column chromatography using an eluent of 2% methanol/dichloromethane. 2.0 g of the product was obtained, with a yield of 86.7%.

51-206

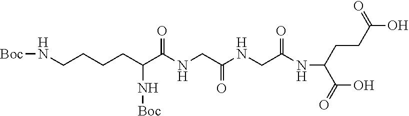 [SMILES: CC(C)(C)OC(=O)NCCCCC(NC(=O)OC(C)(C)C)C(=O)NCC(=O)NCC(=O)NC(CCC(=O)O)C(=O)O]

The product 51-200 (2.0 g, 2.6 mmol) was weighed and added into a hydrogenation mini-reactor, 30 mL of DMF was added for dissolution, then 30 mg of 10% Pd/c (a raw material was washed down a reactor wall with a rubber tip dropper) was added, a magnetic stirrer was added, and the reactor was sealed. An intake valve and an extraction valve were opened, the reactor was vacuumed, the extraction valve was closed, H₂ was injected, the reactor was deflated again, and such procedure was repeated 3 times. Then H₂ was injected to maintain air pressure in the hydrogenation kettle at 2 MPa, the reactant solution was stirred overnight, and after the reaction ended on the plate, the reactant solution was filtered with celite, and the filtrate was obtained for later use.

51-207

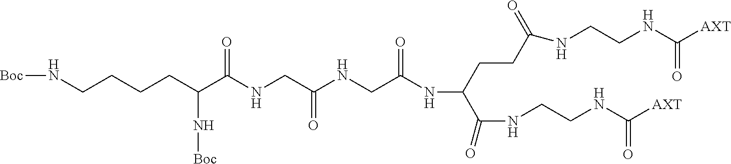

Reactants 51-206 (0.9 mmol) and 53-215 (1.1 g, 2.0 mmol), HOBT (0.4 g, 1.5 mmol) and HBTU (1.0 g, 1.5 mmol) were added into the reaction flask, DMF was added for ultrasonic dissolution, the reactant solution was stirred at low temperature of 0° C., and DIEA (2.4 mml, 4.2 mmol) was added dropwise. After dropwise addition, the reactant solution was taken out and further stirred for reaction for 1 hour at room temperature, and the reaction was monitored via TLC. After the reaction ended, saturated sodium bicarbonate solution and ethyl acetate were added for extraction, and the organic phase was collected; and the aqueous phase was further extracted twice, a product precipitated and was suction filtered, to obtain 0.8 g of the product, with a yield of 61.5%.

51-215

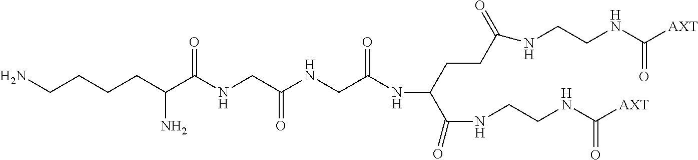

Reactant 51-207 (45 mg, 0.04 mmol) was taken, 10 mL of dichloromethane was added for dissolution, then TFA (0.3 mL, 4 mmol) was added, the reactant solution was stirred for reaction at room temperature, and the reaction was monitored via TLC. After the reaction ended, the reactant solution was evaporated to dryness with a rotary evaporator, ethyl acetate and saturated sodium bicarbonate solution were added for extraction, and the resultant mixture was evaporated to dryness. Dichloromethane was added for dissolution, methyl tertiary butyl ether and n-hexane were added for precipitation, and suction filtration was performed with a sand core funnel, and the filter cake was collected. After the filter cake was dried via suction, the resultant product was dissolved in dichloromethane, methyl tertiary butyl ether and n-hexane were added for precipitation, and suction filtration was performed with a sand core funnel, and the filter cake was collected. The previous step was repeated twice, and the filter cake was transferred to a flask for drying, to obtain 0.5 g of product, which was overproduction.

51-209

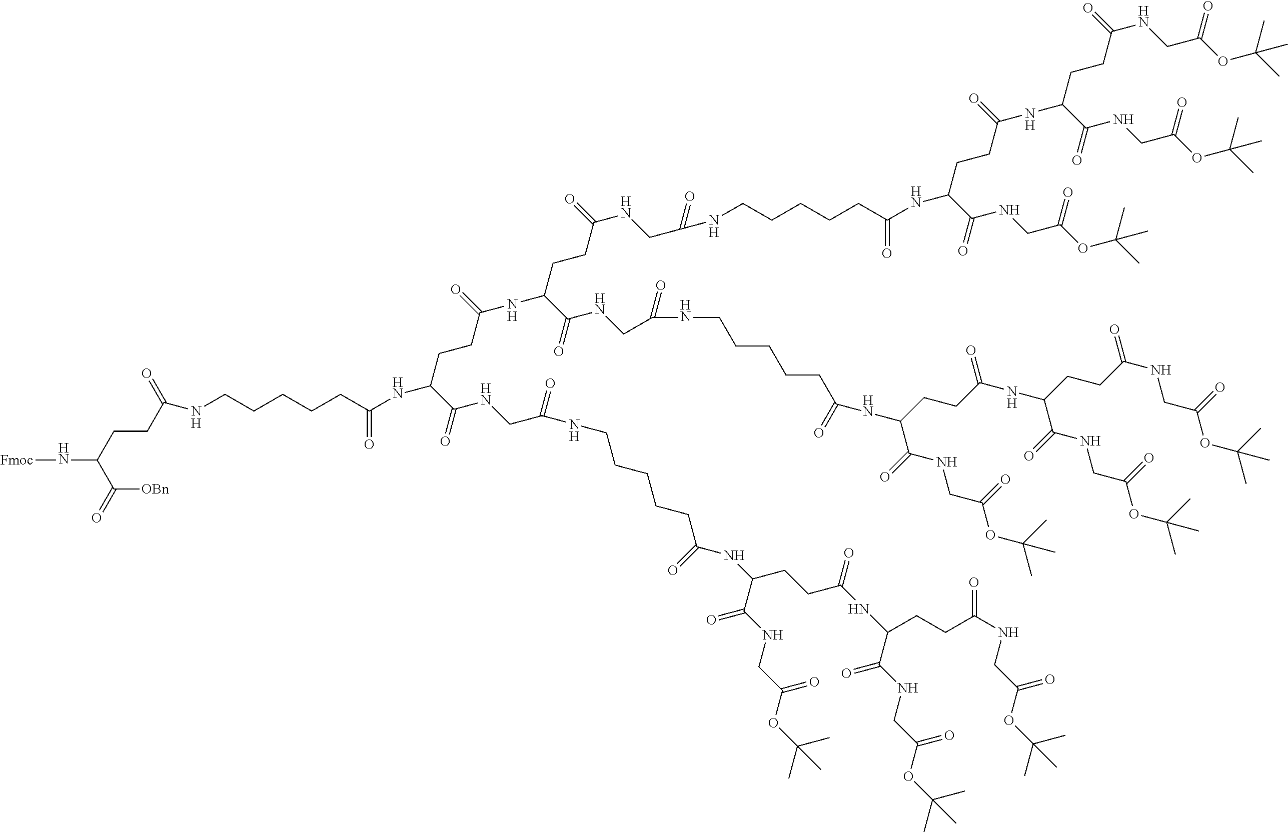

Reactants 56-189 (0.2 g, 0.9 mmol) and Fmoc-Glu-OH (0.036 g, 0.9 mmol, Innochem), HOBT (16 mg, 0.12 mmol) and HBTU (45 mg, 0.12 mmol) were added into the reaction flask, DMF was added for ultrasonic dissolution, the reactant solution was stirred at low temperature of 0° C., and DIEA (0.06 mml, 0.36 mmol) was added dropwise. After dropwise addition, the reactant solution was taken out and further stirred for reaction for 1 hour at room temperature, and the reaction was monitored via TLC. After the reaction ended, saturated sodium bicarbonate solution and ethyl acetate were added for extraction, and the organic phase was collected, and the aqueous phase was further extracted twice, and the resultant mixture was evaporated to dryness, to obtain 0.25 g of the product.

51-210

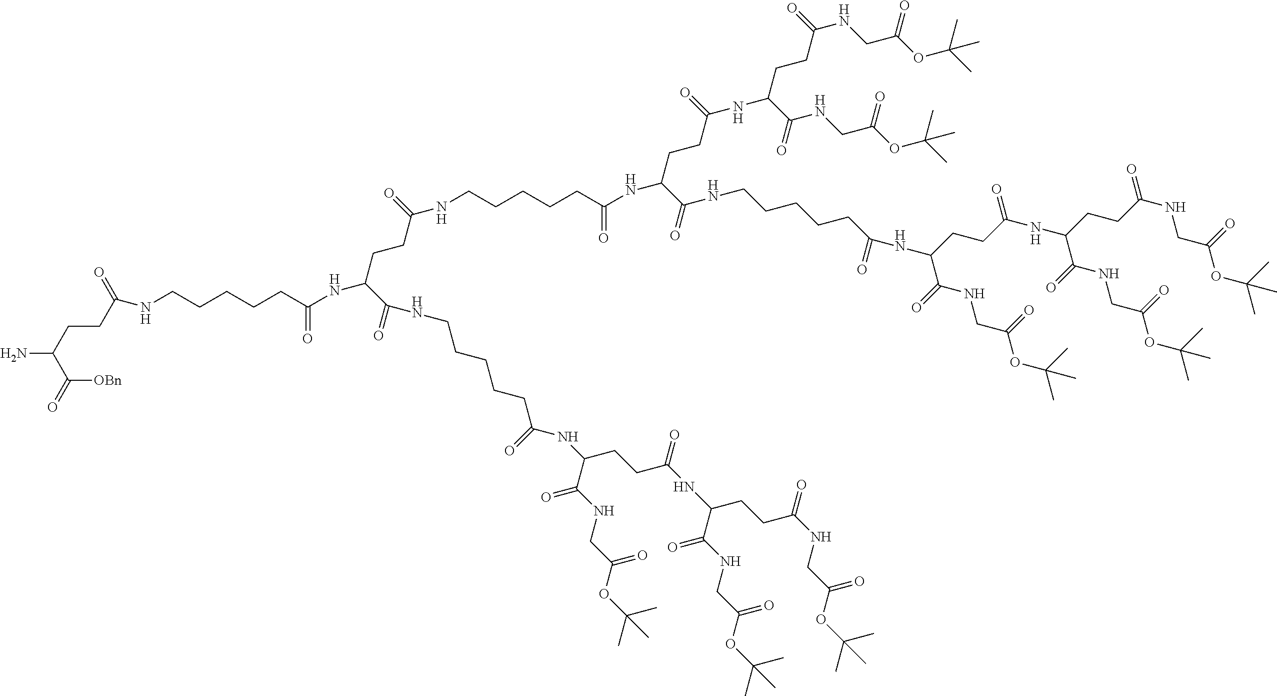 [SMILES: CC(C)(C)OC(=O)CNC(=O)CCC(NC(=O)CCC(NC(=O)CCCCCNC(=O)CCC(NC(=O)CCCCCNC(=O)CCC(N)C(=O)OCc1ccccc1)C(=O)NCCCCCC(=O)NC(CCC(=O)NC(CCC(=O)NCC(=O)OC(C)(C)C)C(=O)NCC(=O)OC(C)(C)C)C(=O)NCC(=O)OC(C)(C)C)C(=O)NCCCCCC(=O)NC(CCC(=O)NC(CCC(=O)NCC(=O)OC(C)(C)C)C(=O)NCC(=O)OC(C)(C)C)C(=O)NCC(=O)OC(C)(C)C)C(=O)NCC(=O)OC(C)(C)C]

Reactant 51-209 (0.25 g, 0.08 mmol) was taken, 20 mL of DMF was added for dissolution, then morpholine (0.2 mL, 2 mmol) was added, the reactant solution was stirred for reaction at room temperature, and the reaction was monitored via TLC. After the reaction ended, methyl tertiary butyl ether and n-hexane were added for precipitation, and suction filtration was performed to obtain a solid. 0.2 g of product was obtained.

68-96

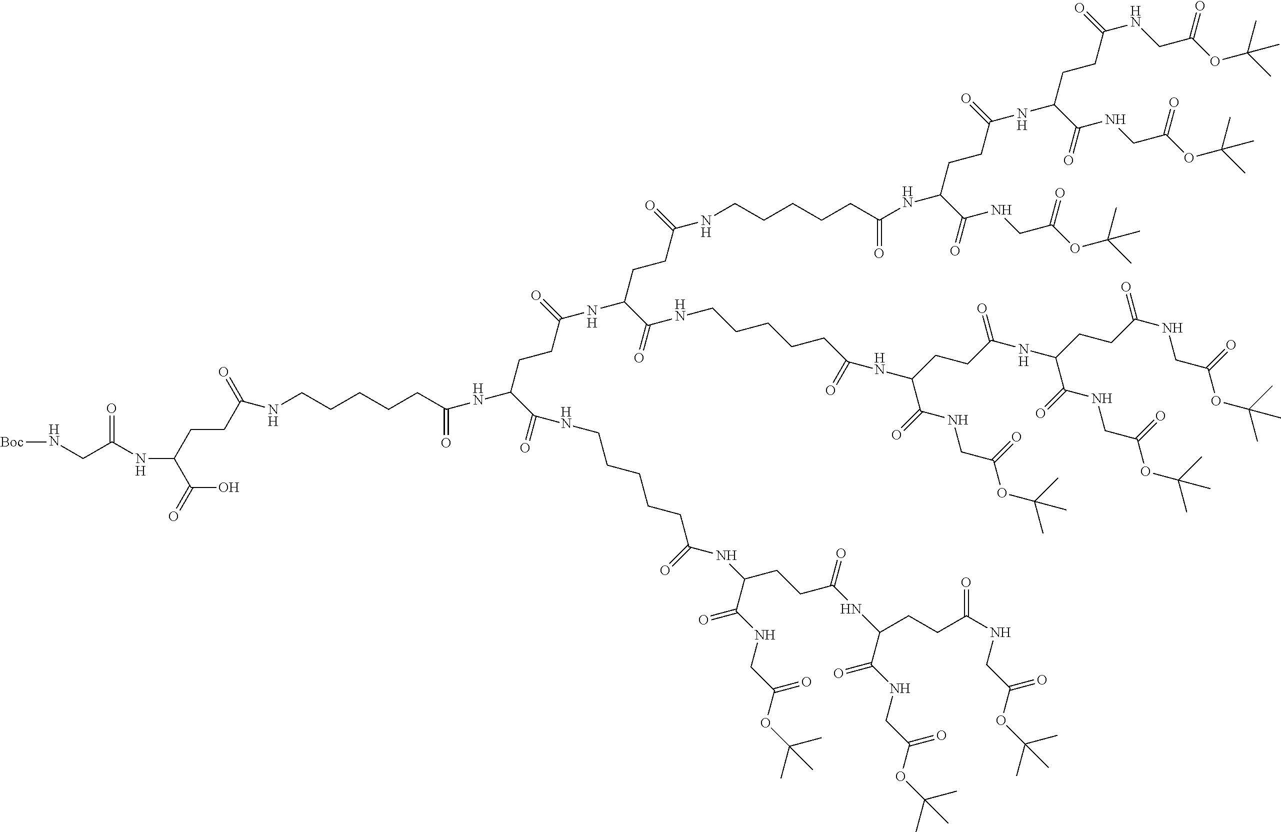 [SMILES: CC(C)(C)OC(=O)CNC(=O)CCC(NC(=O)CCC(NC(=O)CCCCCNC(=O)CCC(NC(=O)CCC(NC(=O)CCCCCNC(=O)CCC(NC(=O)CNC(=O)OC(C)(C)C)C(=O)O)C(=O)NCCCCCC(=O)NC(CCC(=O)NC(CCC(=O)NCC(=O)OC(C)(C)C)C(=O)NCC(=O)OC(C)(C)C)C(=O)NCC(=O)OC(C)(C)C)C(=O)NCCCCCC(=O)NC(CCC(=O)NC(CCC(=O)NCC(=O)OC(C)(C)C)C(=O)NCC(=O)OC(C)(C)C)C(=O)NCC(=O)OC(C)(C)C)C(=O)NCC(=O)OC(C)(C)C)C(=O)NCC(=O)OC(C)(C)C]

Reactants 51-210 (0.41 g, 0.15 mmol) and Boc-Gly-OH (35 mg, 0.20 mmol, Innochem), HOBT (31 mg, 0.23 mmol) and HBTU (88 mg, 0.23 mmol) were added into the reaction flask, DMF was added for ultrasonic dissolution, the reactant solution was stirred at low temperature of 000, and DIEA (0.11 mml, 0.70 mmol) was added dropwise. After dropwise addition, the reactant solution was taken out and further stirred for reaction for 1 hour at room temperature, and the reaction was monitored via TLC. After the reaction ended, methyl tertiary butyl ether and n-hexane were added for precipitation, and suction filtration was performed to obtain a solid. 0.46 g of product was obtained, which was overproduction.

68-98

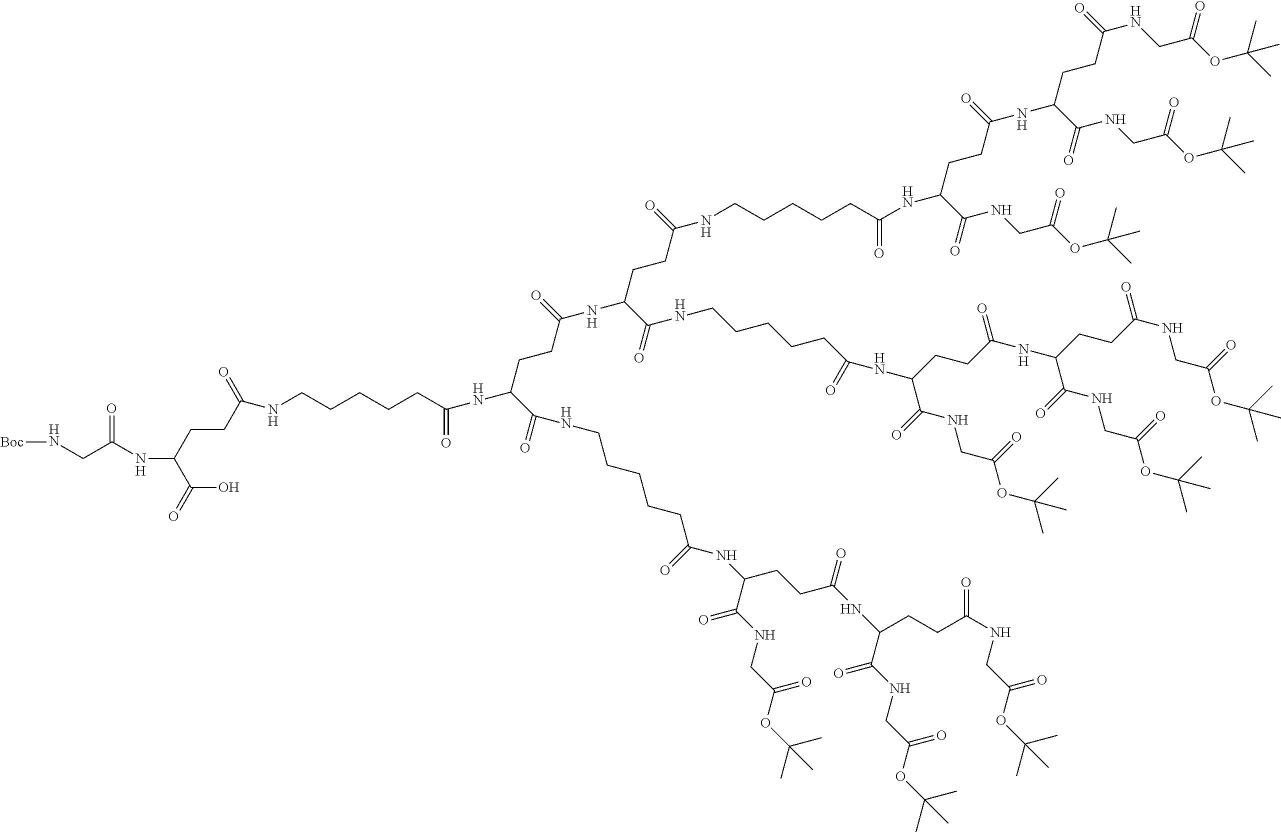 [SMILES: CC(C)(C)OC(=O)CNC(=O)CCC(NC(=O)CCC(NC(=O)CCCCCNC(=O)CCC(NC(=O)CCC(NC(=O)CCCCCNC(=O)CCC(NC(=O)CNC(=O)OC(C)(C)C)C(=O)O)C(=O)NCCCCCC(=O)NC(CCC(=O)NC(CCC(=O)NCC(=O)OC(C)(C)C)C(=O)NCC(=O)OC(C)(C)C)C(=O)NCC(=O)OC(C)(C)C)C(=O)NCCCCCC(=O)NC(CCC(=O)NC(CCC(=O)NCC(=O)OC(C)(C)C)C(=O)NCC(=O)OC(C)(C)C)C(=O)NCC(=O)OC(C)(C)C)C(=O)NCC(=O)OC(C)(C)C)C(=O)NCC(=O)OC(C)(C)C]

The product 68-96 (0.15 mmol) was weighed and added into a hydrogenation mini-reactor, NMP (30 mL) was added for dissolution, then 10% Pd/C catalyst (20 mg) (a raw material was washed down a reactor wall with a rubber tip dropper) was added, a magnetic stirrer was added, and the reactor was sealed. An intake valve and an extraction valve were opened, the reactor was vacuumed, the extraction valve was closed, H₂ was injected, the reactor was deflated again, and such procedure was repeated 3 times. Then H₂ was injected to maintain air pressure in the hydrogenation kettle at 2 MPa, the reactant solution was stirred overnight, and after the reaction ended on the plate, the reactant solution was filtered with celite, and the filtrate was obtained for later use.

68-102

Reactants 68-98 (0.15 mmol) and 51-215 (0.0703 mmol), HOBT (20 mg, 0.15 mmol) and HBTU (58 mg, 0.15 mmol) were added into the reaction flask, DMF was added for ultrasonic dissolution, the reactant solution was stirred at low temperature of 000, and DIEA (0.06 mml, 0.36 mmol) was added dropwise. After dropwise addition, the reactant solution was taken out and further stirred for reaction for 1 hour at room temperature, and the reaction was monitored via TLC. After the reaction ended, methyl tertiary butyl ether and n-hexane were added for precipitation, and suction filtration was performed to obtain a solid. 0.37 g of product was obtained, with a yield of 77.08%.

68-106

Reactant 68-102 (0.37 g, 0.05 mmol) was taken, 10 mL of dichloromethane was added for dissolution, then TEA (1.8 mL, 24 mmol) was added, the reactant solution was stirred for reaction at room temperature, and the reaction was monitored via TLC. After the reaction ended, the reactant solution was evaporated to dryness with a rotary evaporator, ethyl acetate and saturated sodium bicarbonate solution were added for extraction, and the resultant mixture was evaporated to dryness. Dichloromethane was added for dissolution, methyl tertiary butyl ether and n-hexane were added for precipitation, and suction filtration was performed with a sand core funnel, and the filter cake was collected. After the filter cake was dried via suction, the resultant product was dissolved in dichloromethane, methyl tertiary butyl ether and n-hexane were added for precipitation, and suction filtration was performed with a sand core funnel, and the filter cake was collected. The previous step was repeated twice, and the filter cake was transferred to a flask for drying, to obtain 0.25 g of product, with a yield of 83.33%.

68-107

Reactant 68-106 (0.25 g, 0.04 mmol) and M-SCM-10K (1.01 g, 0.10 mmol, JenKem) were taken and dissolved in dichloromethane (15 mL), then DIEA (0.2 mL, mmol) was added, the reactant solution was stirred for reaction at room temperature while protected from light, and the reaction was monitored via TLC. After the reaction ended, methyl tertiary butyl ether/n-hexane was added for precipitation, and suction filtration was performed with a sand core funnel, and the filter cake was collected. The previous step was repeated twice, and the filter cake was transferred to a flask for drying, to obtain 0.68 g of product, with a yield of 58.62%.

68-111

Reactants 68-107 (0.2 g, 0.0076 mmol) and 53-213 (0.14 g, 0.2736 mmol), DCC (0.03 g, 0.1642 mmol) and DMAP (5 mg, 0.0410 mmol) were added into the reaction flask, DMF was added for ultrasonic dissolution, the reactant solution was stirred for reaction for 5 hours at room temperature, and the reaction was monitored via TLC. After the reaction ended, methyl tertiary butyl ether and n-hexane were added for precipitation, and suction filtration was performed to obtain a solid. 0.16 g of the product was obtained, with a yield of 59.70%.

68-114

Reactant 68-111 (0.16 g, 0.0045 mmol) and TBAF (0.13 g, 0.4074 mmol) were taken, THE was added for dissolution, then five drops of concentrated hydrochloric acid were added, the reactant solution was stirred for reaction overnight at room temperature, and the reaction was monitored via TLC. After the reaction ended, the reactant solution was concentrated with a rotary evaporator, methyl tertiary butyl ether/n-hexane was added for precipitation, and suction filtration was performed with a sand core funnel, and the filter cake was collected. Then the filter cake was dissolved in a mixed solvent of methanol/dichloromethane (1:5), silica gel powder (20 mL) was added to obtain a mixture which was then evaporated to dryness to obtain a powder solid, and the powder solid was dry loaded for column chromatography with elution using a mixed solution of 6% to 10% methanol in dichloromethane, and the desired component was collected, concentrated and evaporated to dryness, to obtain 0.10 g of the product, with a yield of 71.42%.

¹H-NMR (600 MHz, DMSO-d₆) δ 8.50-7.71 (m, 102H), 7.31-6.93 (m, 15H), 6.73-6.33 (m, 17H), 6.22-5.68 (m, 20H), 4.24-4.06 (m, 14H), 3.66-3.60 (m, 22H), 3.51 (s, 2817H), 3.42-3.37 (m, 28H), 3.22-3.12 (m, 149H), 2.65-2.61 (m, 10H), 2.42-2.37 (m, 12H), 2.20-2.05 (m, 40H), 1.64-1.52 (m, 148H), 1.47-1.40 (m, 24H), 1.36-1.27 (m, 158H), 0.99-0.90 (m, 220H), 0.79-0.63 (m, 11H).

### Synthesis of Compound 48-124

36-228

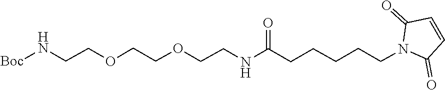 [SMILES: CC(C)(C)OC(=O)NCCOCCOCCNC(=O)CCCCCN1C(=O)C=CC1=O]

6-maleimidocaproic acid (3 g, 14.2032 mmol, Energy Chemical), the product 37-160 (3.9 g, 15.6235 mmol), HBTU (8 g, 21.3048 mmol), and HOBT (2.8 g, 21.3048 mmol) were added to a 250 mL flask, 30 mL of DMF was added for dissolution, the reactant solution was kept at −5° C. and stirred for reaction for 30 minutes before DIEA (10.5 mL, 63.9144 mmol) was slowly added dropwise, then the resultant solution further reacted for 1 hour, and then stirred and reaction overnight at room temperature. After the reaction ended, the reactant solution was transferred to a 1 L separatory funnel, saturated brine (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 10.6 g of product 36-228.

36-229

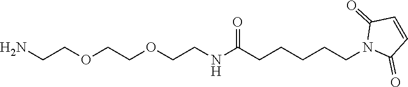 [SMILES: NCCOCCOCCNC(=O)CCCCCN1C(=O)C=CC1=O]

The product 36-228 (10.6 g, 14.2032 mmol) was added into a 500 mL flask, dichloromethane (20 mL) was added, then TFA (10.5 mL, 142.032 mmol) was added, and the reactant solution was stirred overnight at room temperature. After the reaction ended, the reactant solution was concentrated to 10 mL, methyl tertiary butyl ether (200 mL) was added, to obtain a powder precipitate, then suction filtration was performed, and the filter cake was rinsed with methyl tertiary butyl ether (50 mL×3). Then the filter cake was dissolved in a solution (200 mL) (20% methanol/80% dichloromethane), silica gel powder (60 mL) was added, and the resultant mixture was evaporated to dryness to obtain a powder solid, and the powder solid was dry loaded for column chromatography with elution using a mixed solution of 4% to 40% methanol in dichloromethane, and the desired component was collected, concentrated and evaporated to dryness, to obtain 1.5 g of product 36-229, with a yield of 30.9%.

36-239

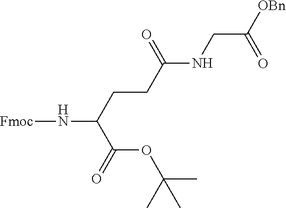

Fmoc-Glu-OtBu (4.2 g, 9.9182 mmol, Accela), H-Gly-OBzl-HCl (2 g, 9.9182 mmol, Bide Pharmatech Co., Ltd.), HBTU (5.6 g, 14.8773 mmol), and HOBT (2 g, 14.8773 mmol) were added to a 250 mL flask, 30 mL of DMF was added for dissolution, the reactant solution was kept at −5° C. and stirred for reaction for 30 minutes before DIEA (9 mL, 54.5499 mmol) was slowly added dropwise, then the resultant solution further reacted for 1 hour, and then stirred and reaction overnight at room temperature. After the reaction ended, the reactant solution was taken out, added with deionized water (100 mL), and extracted with ethyl acetate (100 mL×3) multiple times, the organic phases were combined, rinsed with a saturated sodium chloride solution (100 mL) twice and finally concentrated and evaporated to dryness, to obtain 7.8 g of product 36-239.

36-240

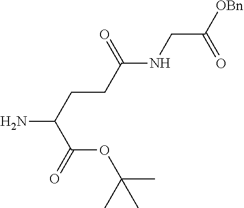 [SMILES: CC(C)(C)OC(=O)C(N)CCC(=O)NCC(=O)OCc1ccccc1]

The product 36-239 (7.8 g, 9.9182 mmol) was added into a 250 mL flask, DMF was added for dissolution, and then morpholine (8.6 mL, 99.182 mmol) was added, and the reactant solution was stirred overnight at room temperature. After the reaction ended, the reactant solution was transferred to a 1 L separatory funnel, a saturated sodium bicarbonate solution (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 5.4 g of product 36-240.

36-241

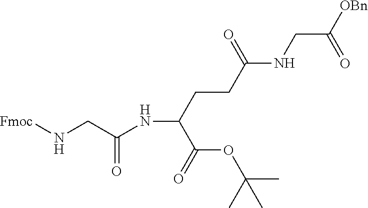

The product 36-240 (5.4 g, 9.9182 mmol), Fmoc-Gly-OH (2.6 g, 8.9264 mmol), HBTU (5.6 g, 14.8773 mmol) and HOBT (2 g, 14.8773 mmol) were added to a 250 mL flask, 30 mL of DMF was added for dissolution, the reactant solution was kept at −5° C. and stirred for reaction for 30 minutes before DIEA (7.3 mL, 44.6319 mmol) was slowly added dropwise, then the resultant solution further reacted for 1 hour, and then stirred and reaction overnight at room temperature. After the reaction ended, the reactant solution was transferred to a 1 L separatory funnel, saturated brine (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 4.86 g of product 36-241, with a yield of 78.3%.

36-244

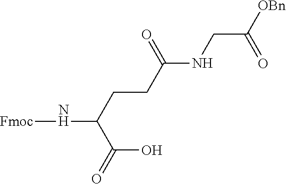

The product 36-241 (4.86 g, 9.9182 mmol) was added into a 500 mL flask, dichloromethane (20 mL) was added, then TFA (7.36 mL, 99.182 mmol) was added, and the reactant solution was stirred overnight at room temperature. After the reaction ended, the reactant solution was concentrated to 10 mL, methyl tertiary butyl ether (200 mL) was added, to obtain a powder precipitate which was suction filtered, and dried to obtain 3.45 g of product 36-244, with a yield of 86.6%.

48-63

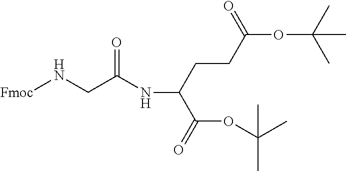

The Fmoc-Gly-OH (10.74 g, 36.32566 mmol), L-glutamic acid di-tert-butyl ester hydrochloride (10.8 g, 36.3256 mmol, Innochem), HBTU (15.2 g, 39.9580 mmol) and HOBT (5.4 g, 39.9580 mmol) were weighted, an appropriate amount of DMF was added for dissolution, the reactant solution was kept at −5° C., and DIEA (10.27 mL, 62.1522 mmol) was slowly added dropwise, and then the reactant solution reacted for half an hour, then taken out and stirred for reaction overnight at room temperature. After the reaction ended, the reactant solution was transferred to a 1 L separatory funnel, saturated brine (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness. Then the organic phases were dissolved in dichloromethane (200 mL), silica gel powder (60 mL) was added to obtain a mixture which was then evaporated to dryness to obtain a powder solid, and the powder solid was dry loaded for column chromatography with elution using a mixed solution of 20% to 40% ethyl acetate in petroleum ether, and the desired component was collected, concentrated and evaporated to dryness, to obtain 19.4 g of product 48-63, with a yield of 99.5%.

48-64

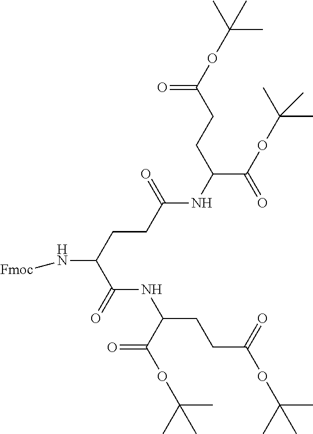

L-glutamic acid di-tert-butyl ester hydrochloride (3.24 g, 10.9364 mmol, Innochem), Fmoc-Glu-OH (2.00, 5.4141 mmol, Aladdin), HOBT (2.2 g, 16.2423 mmol) and HBTU (6.16 g, 16.2423 mmol) were weighed and added into a 250 mL reaction flask, a DMF solution (40 mL) was added for dissolution, the reactant was completely ultrasonically dissolved, the reactant solution was stirred at −5° C. for 30 minutes, DIEA (9.9 mL, 59.5551 mmol) was slowly added dropwise, and the reactant solution was left standing at low temperature until the reaction ended. After the reaction ended, the reactant solution was transferred to a 1 L separatory funnel, saturated sodium chloride (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness. Then the organic phases were dissolved in dichloromethane (200 mL), silica gel powder (40 mL) was added to obtain a mixture which was then evaporated to dryness to obtain a powder solid, and the powder solid was dry loaded for column chromatography with elution using a mixed solution of 30% to 40% ethyl acetate in petroleum ether, and the desired component was collected, concentrated and evaporated to dryness, to obtain 4.2 g of product 48-64, with a yield of 90%.

48-65

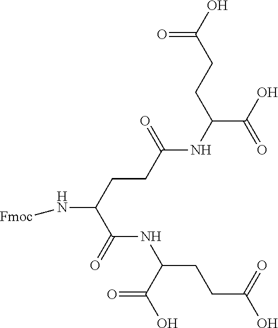

The product 48-64 (3.3 g, 3.8731 mmol) was added into a 500 mL flask, dichloromethane (10 mL) was added, then TFA (5.1 mL, 38.7310 mmol) was added, and the reactant solution was stirred overnight at room temperature. After the reaction ended, the reactant solution was concentrated to 10 mL, methyl tertiary butyl ether (200 mL) was added, to obtain a powder precipitate which was suction filtered, to obtain 3.6 g of product 48-65.

48-66

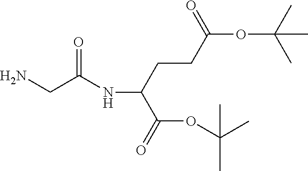 [SMILES: CC(C)(C)OC(=O)CCC(NC(=O)CN)C(=O)OC(C)(C)C]

DMF was added to a flask containing the product 48-63 (9.5 g, 36.0173 mmol), and was ultrasonically oscillated until complete dissolution, then morpholine (31.4 mL, 360.1730 mmol) was added, and the resultant solution was stirred for reaction for 2 hours at room temperature. After the reaction ended, the reactant solution was transferred to a 1 L separatory funnel, a saturated sodium bicarbonate solution (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 6.8 g of product 48-66.

48-67

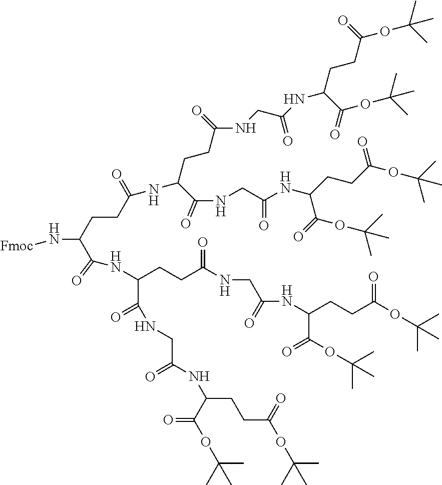

The product 48-66 (5.5 g, 17.5270 mmol), the product 48-65 (2.2 mmol, 3.5054 mmol), HBTU (7.9 g, 21.0324 mmol) and HOBT (2.8 g, 21.0324 mmol) were weighed and added into a 250 mL reaction flask, a DMF solution (80 mL) was added for dissolution, the reactant was completely ultrasonically dissolved, the reactant solution was stirred at −5° C. for 30 minutes, DIEA (12.8 mL, 77.1188 mmol) was slowly added dropwise, and the reactant solution was left standing at low temperature until the reaction ended. After the reaction ended, the reactant solution was transferred to a 1 L separatory funnel, saturated brine (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 11 g of product 48-67.

48-69

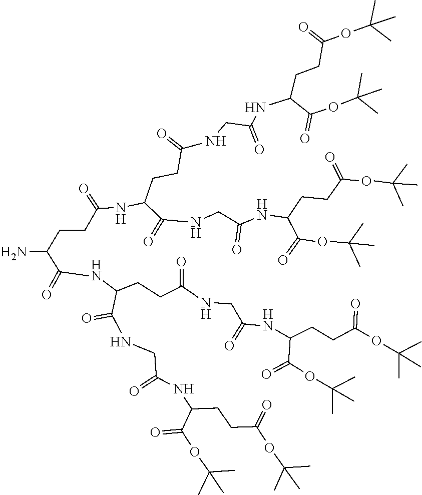 [SMILES: CC(C)(C)OC(=O)CCC(NC(=O)CNC(=O)CCC(NC(=O)CCC(N)C(=O)NC(CCC(=O)NCC(=O)NC(CCC(=O)OC(C)(C)C)C(=O)OC(C)(C)C)C(=O)NCC(=O)NC(CCC(=O)OC(C)(C)C)C(=O)OC(C)(C)C)C(=O)NCC(=O)NC(CCC(=O)OC(C)(C)C)C(=O)OC(C)(C)C)C(=O)OC(C)(C)C]

The product 48-67 (11 g, 6.0402 mmol) was taken, DMF (40 mL) was added for dissolution, then morpholine (5.3 mL, 60.4020 mmol) was added, and the resultant solution was stirred for reaction at room temperature. After the reaction ended, the reactant solution was transferred to a 1 L separatory funnel, saturated brine (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness. Then the organic phases were dissolved in dichloromethane (200 mL), silica gel powder (60 mL) was added to obtain a mixture which was then evaporated to dryness to obtain a powder solid, and the powder solid was dry loaded for column chromatography with elution using a mixed solution of 0.5% ammonia/4% to 7% methanol in dichloromethane, and the desired component was collected, concentrated and evaporated to dryness, to obtain 3.9 g of product 48-69, with a yield of 70%.

48-70

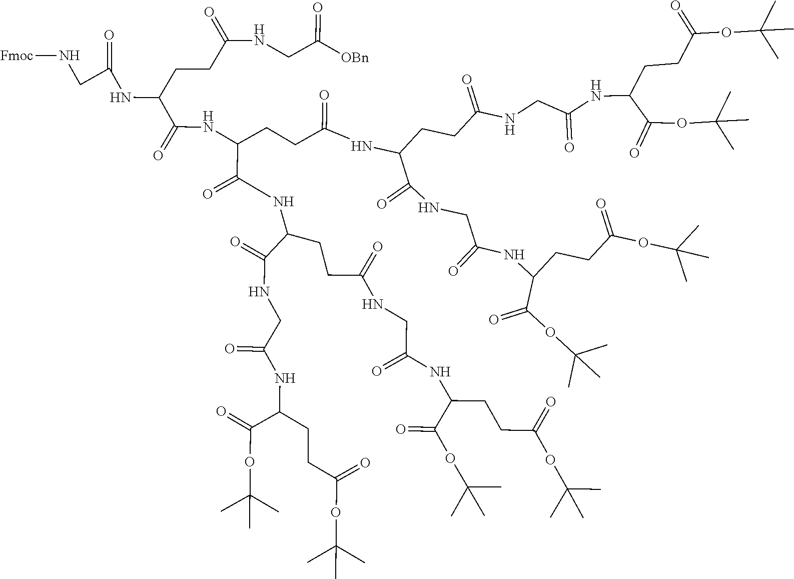

The products 36-244 (1.31 g, 2.4392 mmol) and 48-69 (3.9 g, 2.4392 mmol), HBTU (1.4 g, 3.6588 mmol) and HOBT (0.5 g, 3.6588 mmol) were added to a 250 mL flask, 30 mL of DMF was added for dissolution, the reactant solution was kept at −5° C. and stirred for reaction for 30 minutes before DIEA (2.2 mL, 13.4157 mmol) was slowly added dropwise, then the resultant solution further reacted for 1 hour, and then stirred and reaction overnight at room temperature. After the reaction ended, the reactant solution was transferred to a 1 L separatory funnel, saturated brine (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 6 g of product 48-70.

48-85

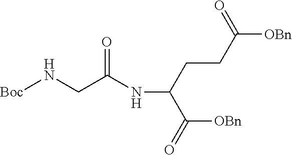 [SMILES: CC(C)(C)OC(=O)NCC(=O)NC(CCC(=O)OCc1ccccc1)C(=O)OCc1ccccc1]

Boc-Gly-OH (3 g, 17.1252 mmol, Ark Pharm), H-Glu(OBzl)-OBzl-TsOH (8.5 g, 17.1252 mmol, Ark Pharm), HOBT (3.4 g, 25.1878 mmol) and HBTU (9.7 g, 25.6878 mmol) were weighed and added into a 250 mL reaction flask, a DMF solution (80 mL) was added for dissolution, the reactant was completely ultrasonically dissolved, the reactant solution was stirred at −5° C. for 30 minutes, DIEA (12.7 mL, 77.0634 mmol) was slowly added dropwise, and the reactant solution was left standing at low temperature until the reaction ended. After the reaction ended, the reactant solution was transferred to a 1 L separatory funnel, saturated brine (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness. Then the organic phases were dissolved in dichloromethane (200 mL), silica gel powder (60 mL) was added to obtain a mixture which was then evaporated to dryness to obtain a powder solid, and the powder solid was dry loaded for column chromatography with elution using a mixed solution of 30% to 40% ethyl acetate in petroleum ether, and the desired component was collected, concentrated and evaporated to dryness, to obtain 8.5 g of product 48-85.

48-86

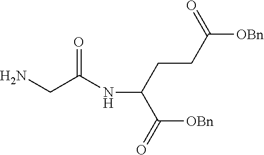 [SMILES: NCC(=O)NC(CCC(=O)OCc1ccccc1)C(=O)OCc1ccccc1]

The product 48-85 (8.5 mmol, 17.1296 mmol) was taken, dichloromethane (5 mL) and TFA (12.7 mL, 171.2960 mmol) were added, the product was completely ultrasonically dissolved, the reaction flask was sealed with a ground glass stopper, and the reactant solution was stirred for reaction at room temperature. After the reaction ended, the reactant solution was concentrated to 10 mL, methyl tertiary butyl ether (200 mL) was added, the reactant solution formed a viscous solid, then 2 to 3 mL of dichloromethane was added for dissolution, n-hexane and methyl tertiary butyl ether were added for precipitation, the previous step was repeated twice, and the reactant solution was filtered and dried, to obtain 8.5 g of product 48-86.

48-88

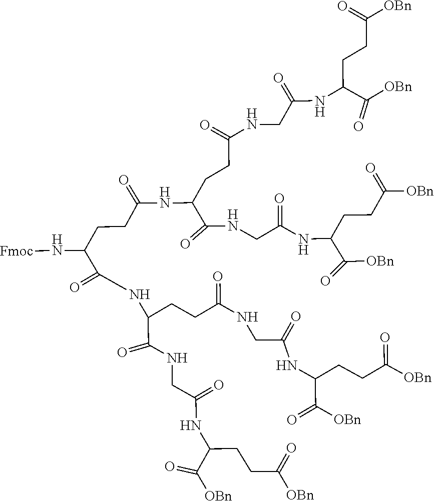

The product 48-86 (5.95 g, 15.4876 mmol), the product 48-65 (2.43, 3.8719 mmol), HOBT (3.15 g, 23.2314 mmol) and HBTU (8.8 g, 23.2314 mmol) were weighed and added into a 250 mL reaction flask, a DMF solution (80 mL) was added for dissolution, the reactant was completely ultrasonically dissolved, the reactant solution was stirred at −5° C. for 30 minutes, DIEA (16.7 mL, 100.6694 mmol) was slowly added dropwise, and the reactant solution was left standing at low temperature until the reaction ended. After the reaction ended, the reactant solution was transferred to a 1 L separatory funnel, a saturated sodium bicarbonate solution (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness. Then the organic phases were dissolved in dichloromethane (200 mL), silica gel powder (50 mL) was added to obtain a mixture which was then evaporated to dryness to obtain a powder solid, and the powder solid was dry loaded for column chromatography with elution using a mixed solution of 4.5% to 6% methanol in dichloromethane, and the desired component was collected, concentrated and evaporated to dryness, to obtain 4.4 g of product 48-88, with a yield of 54%.

48-90

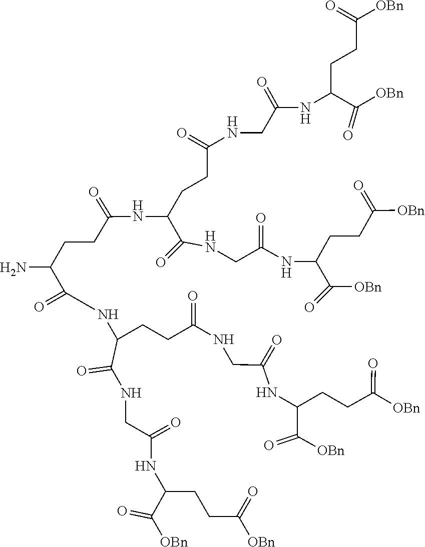 [SMILES: NC(CCC(=O)NC(CCC(=O)NCC(=O)NC(CCC(=O)OCc1ccccc1)C(=O)OCc1ccccc1)C(=O)NCC(=O)NC(CCC(=O)OCc1ccccc1)C(=O)OCc1ccccc1)C(=O)NC(CCC(=O)NCC(=O)NC(CCC(=O)OCc1ccccc1)C(=O)OCc1ccccc1)C(=O)NCC(=O)NC(CCC(=O)OCc1ccccc1)C(=O)OCc1ccccc1]

The product 48-66 (2.7 g, 1.2898 mmol) was taken, DMF (20 mL) was added for dissolution, then morpholine (1.13 mL, 12.8986 mmol) was added, and the resultant solution was stirred for reaction at room temperature. After the reaction ended, methyl tertiary butyl ether (150 mL) and n-hexane (100 mL) were added to the reactant solution for precipitation, to obtain a precipitate of a solid product which was subjected to suction filtration and was dried, to obtain 3 g of product 48-90.

48-91

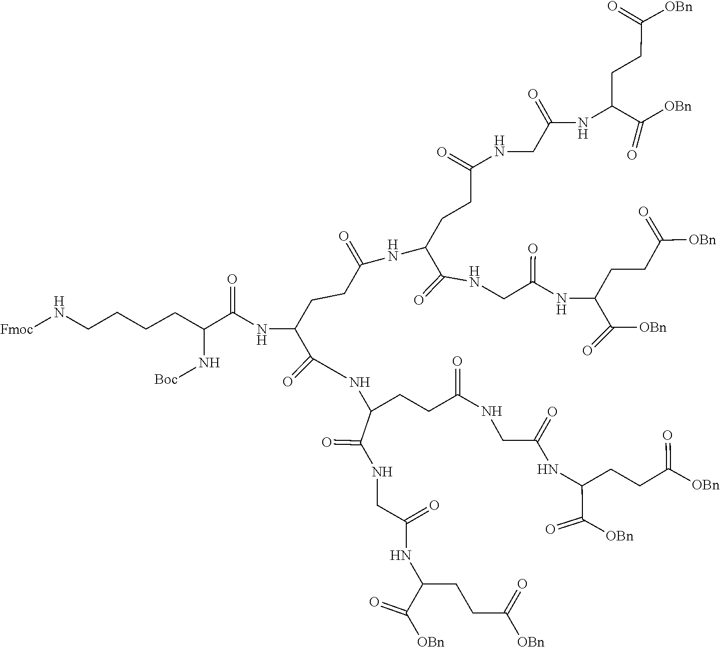

The product 48-90 (2.4 g, 1.2827 mmol), Fmoc-Lys(Boc)-OH (0.6 g, 1.2827 mmol), HOBT (0.26 g, 1.9241 mmol) and HBTU (0.73 g, 1.9241 mmol) were added into a 250 mL reaction flask, a DMF solution (40 mL) was added for dissolution, the reactant was completely ultrasonically dissolved, the reactant solution was stirred at −5° C. for 30 minutes, DIEA (1 mL, 5.7721 mmol) was slowly added dropwise, and the reactant solution was left standing at low temperature until the reaction ended. After the reaction ended, n-hexane (150 mL) and methyl tertiary butyl ether (30 mL) were added for precipitation, the supernatant was discarded, n-hexane (150 mL) and methyl tertiary butyl ether (50 mL) was further added to a lower layer of oily solution, and such operation was repeated three times, to obtain an oily substance. Methyl tertiary butyl ether (200 mL) was added, to obtain a solid precipitate, then suction filtration was performed, and the filter cake was rinsed with methyl tertiary butyl ether (50 mL×3). Then the filter cake was dissolved in a solution (200 mL) (20% methanol/80% dichloromethane), silica gel powder (60 mL) was added, and the resultant mixture was evaporated to dryness to obtain a powder solid, and the powder solid was dry loaded for column chromatography with elution using a mixed solution of 4% to 5% methanol in dichloromethane, and the desired component was collected, concentrated and evaporated to dryness, to obtain 2.3 g of product 48-91, with a yield of 79.5%.

48-93

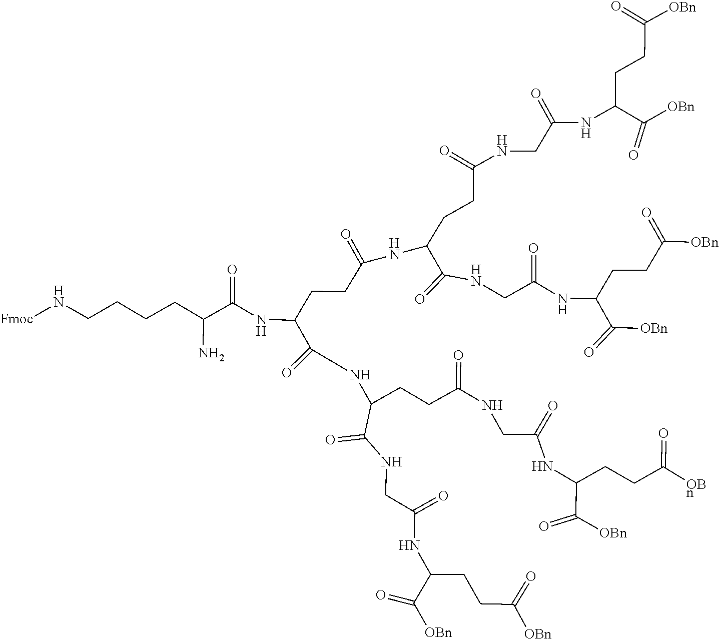

The product 48-91 (2.3 g, 0.9907 mmol) was taken, dichloromethane (5 mL) and TEA (0.75 mL, 9.9072 mmol) were added, the product was completely ultrasonically dissolved, the reaction flask was sealed with a ground glass stopper, and the reactant solution was stirred for reaction at room temperature. After the reaction ended, methyl tertiary butyl ether (150 mL) and n-hexane (100 mL) were added to the reactant solution for precipitation, to obtain a precipitate of a solid product which was subjected to suction filtration and was dried, to obtain 2.4 g of product 48-93.

48-95

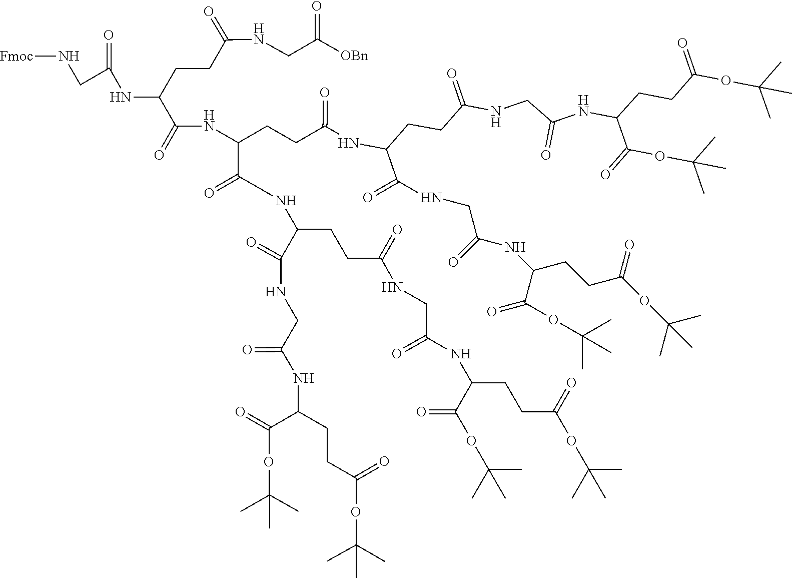

The product 48-70 (1.2 g, 0.5570 mmol) was added into a hydrogenation reaction kettle, then Pd/C (0.07 g) accounting for 10% was added, DMF (20 mL) was added, hydrogen gas was injected at a hydrogen gas pressure of 1.4 MPa, and the reactant solution was stirred overnight at room temperature. After the reaction ended, the reactant solution was filtered with celite, the filter cake was rinsed with DMF (15 mL×3), and the filtrate was added into a 250 mL round-bottomed flask and used as a raw material for the next step.

48-97

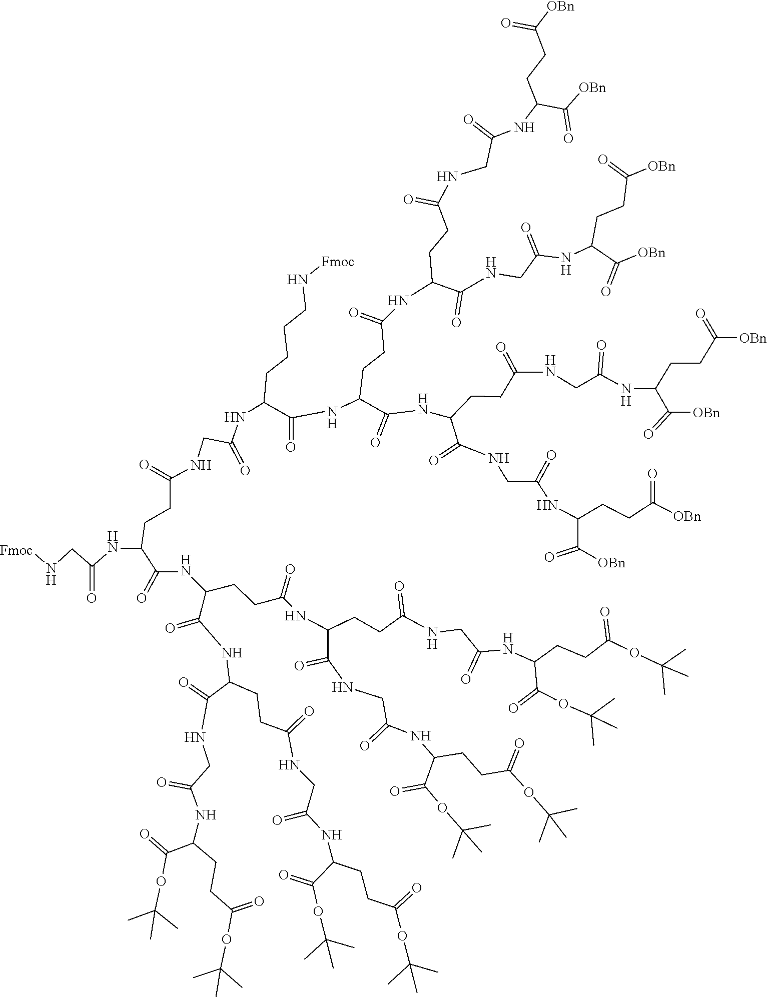

The product 48-95 (1.15 g, 0.5570 mmol), the product 48-93 (1.25 g, 0.5570 mmol), HOBT (0.12 g, 0.8355 mmol) and HBTU (0.32 g, 0.8355 mmol) were weighed and added into a 250 mL reaction flask, a DMF solution (80 mL) was added for dissolution, the reactant was completely ultrasonically dissolved, the reactant solution was stirred at −5° C. for 30 minutes, DIEA (0.5 mL, 3.0635 mmol) was slowly added dropwise, and the reactant solution was left standing at low temperature until the reaction ended. After the reaction ended, the reactant solution was transferred to a 1 L separatory funnel, saturated brine (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness. Then the organic phases were dissolved in dichloromethane (200 mL), silica gel powder (30 mL) was added to obtain a mixture which was then evaporated to dryness to obtain a powder solid, and the powder solid was dry loaded for column chromatography with elution using a mixed solution of 3% to 10% methanol in dichloromethane, and the desired component was collected, concentrated and evaporated to dryness, to obtain 1.3 g of product 48-97, with a yield of 54.8%.

48-98

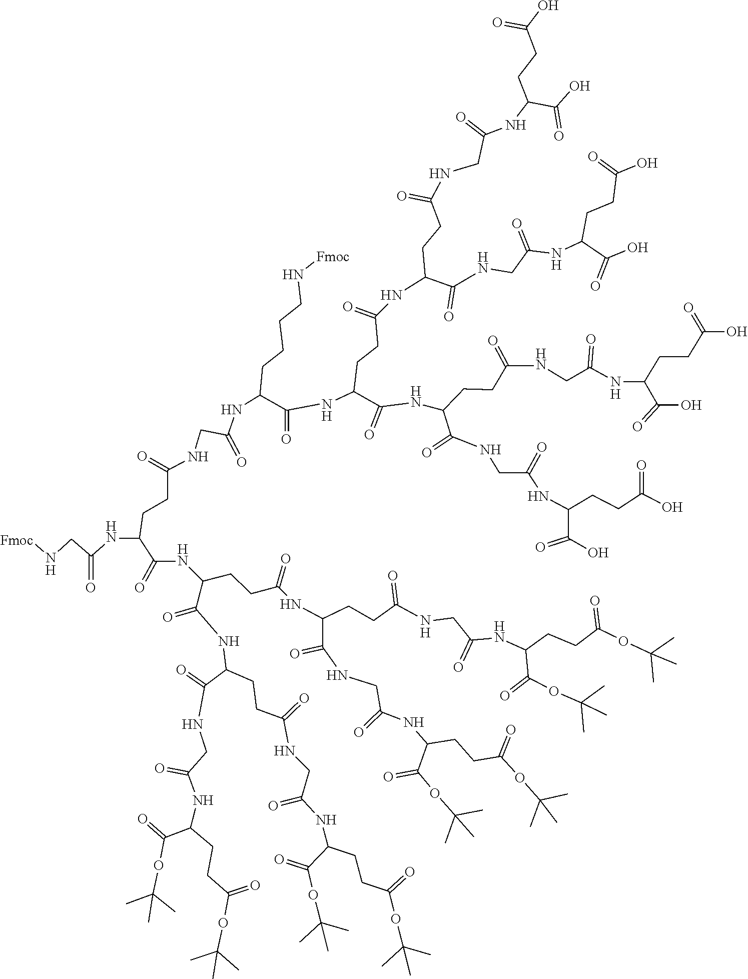

The product 48-97 (1 g, 0.2343 mmol) was added into a hydrogenation reaction kettle, then Pd/C catalyst (0.07 g) accounting for 10% was added, DMF (20 mL) was added, hydrogen gas was injected at a hydrogen gas pressure of 1.4 MPa, and the reactant solution was stirred overnight at room temperature. After the reaction ended, the reactant solution was filtered with celite, the filter cake was rinsed with DMF (15 mL×3), and the filtrate was added into a 250 mL round-bottomed flask and used as a raw material for the next step.

48-107

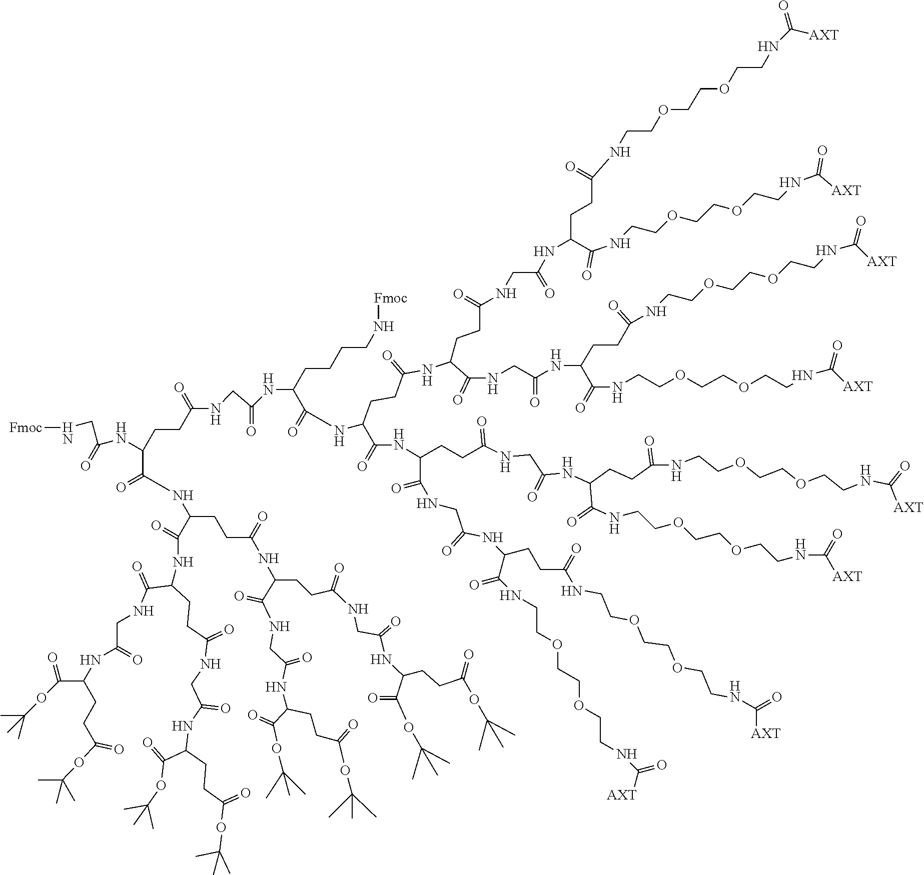

The products 48-98 (0.89 g, 0.2331 mmol) and 39-147 (1.05 g, 1.8644 mmol), HBTU (1.06 g, 2.7966 mmol) and HOBT (0.38 g, 2.7966 mmol) were added to a 250 mL flask, DMF (30 mL) was added for dissolution, the reactant solution was kept at −5° C. and stirred for reaction for 30 minutes before DIEA (1.38 mL, 8.3899 mmol) was slowly added dropwise, then the resultant solution further reacted for 1 hour, and then stirred and reaction overnight at room temperature. After the reaction ended, n-hexane (100 mL×3) was added for precipitation, a lower layer of oily solid was dissolved in a small amount of dichloromethane, methyl tertiary butyl ether was added to obtain a solid precipitate and the solid precipitate was dried to obtain 1.9 g of product 48-107.

48-108

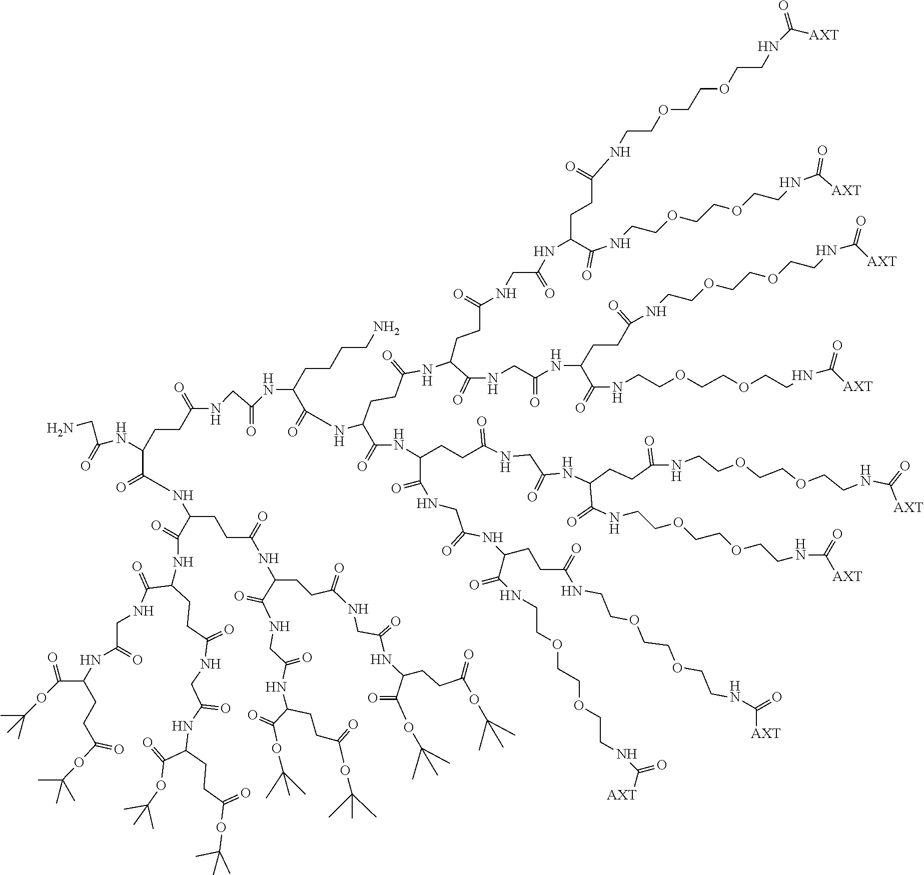

The product 48-107 (1.9 g, 0.2282 mmol) was added into a 250 mL flask, DMF was added for dissolution, and then morpholine (0.39 mL, 4.5639 mmol) was added, and the reactant solution was stirred overnight at room temperature. After the reaction ended, n-hexane (100 mL×3) was added for precipitation, a lower layer of oily solid was dissolved in a small amount of dichloromethane, methyl tertiary butyl ether was added to obtain a solid precipitate and the solid precipitate was dried to obtain 1.5 g of product 48-108, with a yield of 88%.

48-109

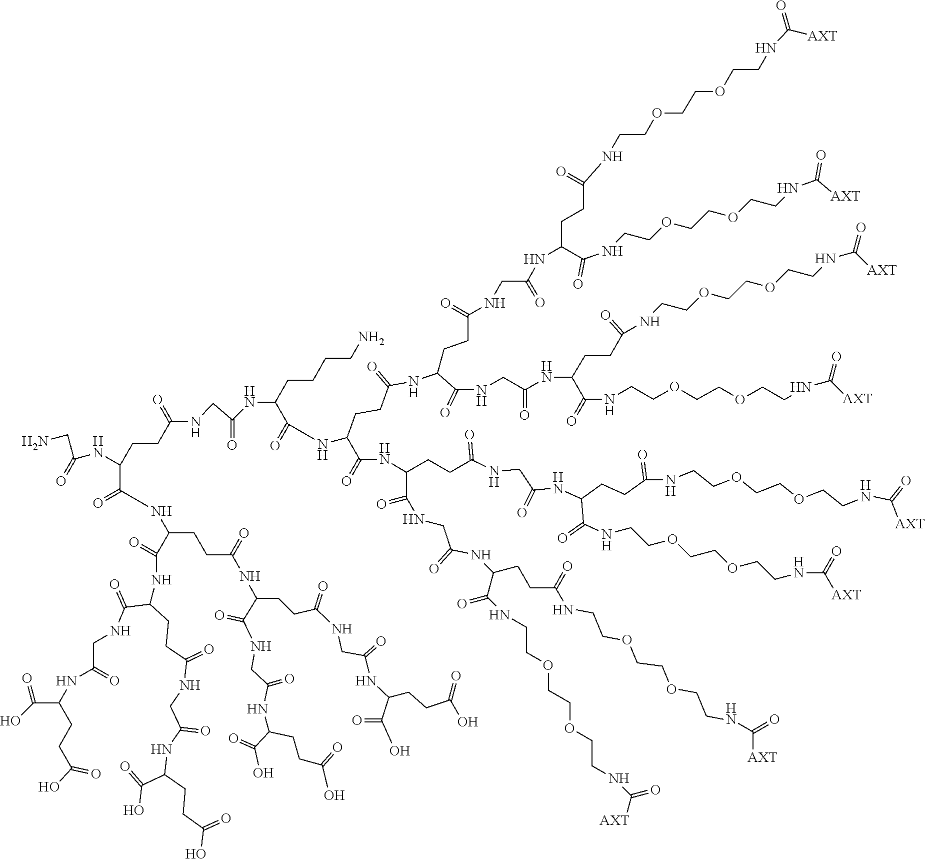

The product 48-108 (1.5 g, 0.2270 mmol) was added into a 500 mL flask, dichloromethane (20 mL) was added, then TFA (1.35 mL, 18.1635 mmol) was added, and the reactant solution was stirred overnight at room temperature. After the reaction ended, the reactant solution was concentrated to 10 mL, methyl tertiary butyl ether (200 mL) was added, to obtain a powder precipitate which was suction filtered, to obtain 1.5 g of product 48-109.

48-110

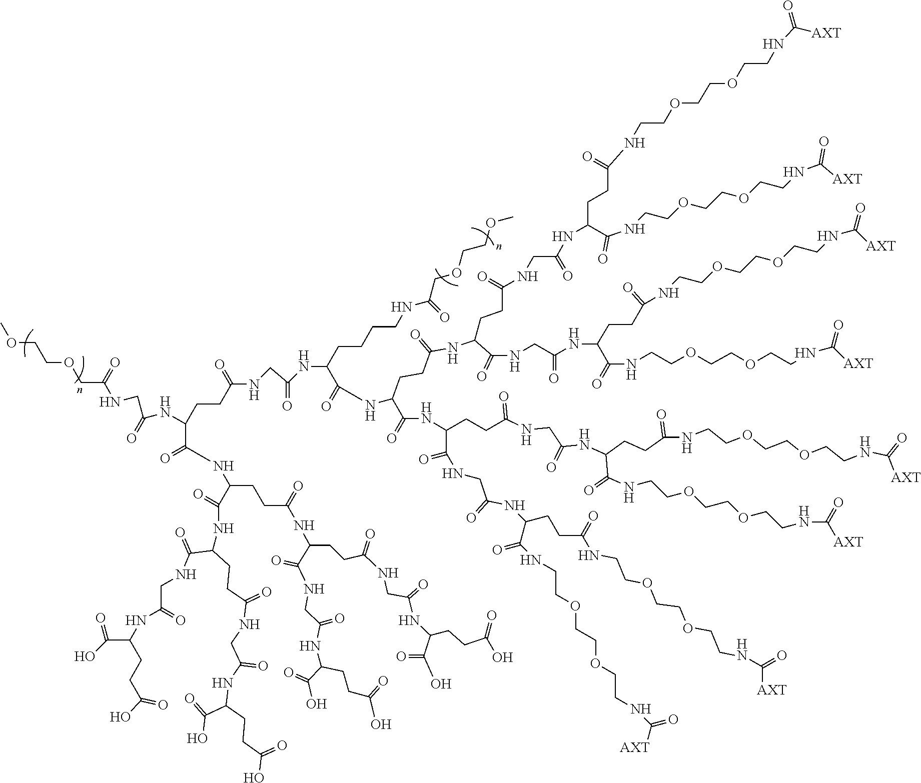

The product 48-109 (0.6 g, 0.08544 mmol) was added to a 250 mL flask, then 20 mL of DMF was added for dissolution, the reactant solution was kept at −5° C. and stirred for reaction for 30 minutes before DIEA (0.28 mL, 1.7088 mmol) was slowly added dropwise, then M-SCM-10K (2 g) was further added, and the reactant solution was stirred for reaction at a low speed for one week at room temperature. After the reaction ended, n-hexane (50 mL×3) was first added, if there is only a small amount of oily substance in the lower layer, methyl tertiary butyl ether (20 mL) was added for precipitation, to obtain a solid precipitate. Then the filter cake was dissolved in methanol/dichloromethane (1:5), 30 mL of silica gel powder was added to obtain a mixture which was then evaporated to dryness and then dry loaded for column chromatography with elution using an eluent (5% to 10% methanol/95% to 90% dichloromethane), and the filtrate was collected, concentrated and evaporated to dryness, to obtain 0.42 g of the product 48-110, with a yield of 17.5%.

48-120

The products 48-110 (0.42 g, 0.01498 mmol) and 36-229 (0.1 g, 0.2996 mmol), HBTU (0.7 g, 1.7970 mmol) and HOBT (0.2 g, 1.7970 mmol) were added to a 250 mL flask, DMF (30 mL) was added for dissolution, the reactant solution was kept at −5° C. and stirred for reaction for 30 minutes before DIEA (0.9 mL, 5.3920 mmol) was slowly added dropwise, then the resultant solution further reacted for 1 hour, and then stirred and reaction overnight at room temperature. After the reaction ended, n-hexane (50 mL×3) was first added, if there is only a small amount of oily substance in the lower layer, methyl tertiary butyl ether (20 mL) was added for precipitation, to obtain a solid precipitate. Then the filter cake was dissolved in methanol/dichloromethane (1:5), 30 mL of silica gel powder was added to obtain a mixture which was then evaporated to dryness and then dry loaded for column chromatography with elution using an eluent (5% to 10% methanol/95% to 90% dichloromethane), and the filtrate was collected, concentrated and evaporated to dryness, to obtain 0.39 g of the product 48-120, with a yield of 85%.

48-124

The product 48-120 (0.39 g, 0.01274 mmol) was added into a 500 mL flask, DMSO (30 mL) was added for dissolution, then PPT-iRGD (0.2 g, 0.1274 mmol, DGpeptides) was added, and the reactant solution was stirred at 40° C. at low speed for 2 days while protected from light. After the reaction ended, n-hexane (100 mL) and methyl tertiary butyl ether (30 mL) were added, the supernatant was discarded, n-hexane (100 mL) and methyl tertiary butyl ether (30 mL) was added to a lower layer of oily solution, and such operation was repeated three times, to obtain an oily substance. Methyl tertiary butyl ether (100 mL) was added, to obtain a solid precipitate, then filtration was performed, and the filter cake was rinsed with methyl tertiary butyl ether (30 mL×3). Then the filter cake was dissolved in a methanol (40 mL) and dichloromethane (160 mL), silica gel powder (15 g) was added, and the resultant mixture was evaporated to dryness to obtain a powder-like solid, and the powder-like solid was dry loaded for column chromatography, and gradient elution was performed using a mixed solution of 5% to 20% methanol in dichloromethane, and the desired component was collected, concentrated and evaporated to dryness, to obtain 0.18 g of product 48-124, with a yield of 32.7%.

¹H-NMR (600 MHz, DMSO-d₆) δ 7.95-7.93 (m, 51H), 7.83-7.77 (m, 50H), 7.53-7.49 (m, 50H), 7.27-7.24 (m, 5H), 7.14-7.13 (m, 5H), 7.0-6.98 (m, 5H), 6.90 (s, 2H), 6.68-6.67 (m, 6H), 6.22-6.20 (m, 24H), 6.33-6.31 (m, 8H), 3.51 (s, 1874H), 3.48 (s, 16H), 3.47-3.36 (m, 128H), 3.29-3.27 (m, 27H), 3.27-3.26 (m, 48H), 3.19-3.16 (m, 50H), 2.92-2.88 (m, 294H), 2.73 (s, 12H), 2.54 (s, 10H), 2.07-1.96 (m, 77H), 1.56-1.43 (m, 122H), 1.36-1.34 (m, 69H), 1.30-1.22 (m, 326H), 0.86-0.84 (m, 29H).

### Synthesis of Compound 43-233

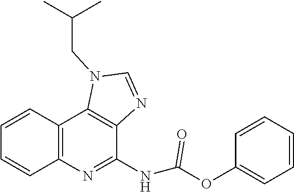 [SMILES: CC(C)Cn1cnc2c(NC(=O)Oc3ccccc3)nc3ccccc3c21]

IMQ (0.5 g, 2.0807 mmol, imiquimod) was weighed and added into a 100 mL round-bottomed flask, then CH₂Cl₂ (20 mL) was added for dissolution, triethylamine (1.17 mL, 8.3228 mmol) was added, the flask was kept at 0° C. and the reactant solution was stirred for 30 minutes, then phenyl chloroformate (0.5 mL, 4.1614 mmol) was slowly added dropwise, and after dropwise addition, the reactant solution was stirred overnight. After the reaction ended, n-hexane (100 mL) and methyl tertiary butyl ether (400 mL) were added for precipitation, such procedure was repeated three times, and the resultant product was filtered, to obtain 0.8 g of the product.

45-229

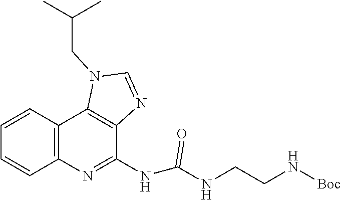 [SMILES: CC(C)Cn1cnc2c(NC(=O)NCCNC(=O)OC(C)(C)C)nc3ccccc3c21]

The product 45-227 (0.3 g, 0.8324 mmol) and Boc-ethylenediamine (0.13 g, 0.8324 mmol) were weighed and added into a 500 mL round-bottomed flask, then DMF (30 mL) was added for dissolution, DIEA (0.13 mL, 4.1620 mmol) was added, the reaction flask was subjected to oil bath at 100° C., and the reactant solution was stirred overnight. After the reaction, the resultant product was rinsed with pure water, extracted with EA three times, and the organic phase was evaporated and dried. The resultant product was dry loaded for column chromatography with elution using 2% to 3% methanol in dichloromethane, and the desired component was collected and concentrated, to obtain 0.2 g of the product, with a yield of 57%.

45-230

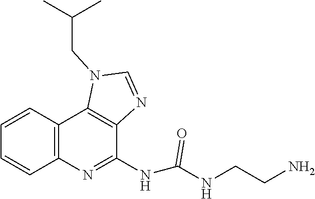 [SMILES: CC(C)Cn1cnc2c(NC(=O)NCCN)nc3ccccc3c21]

The product 45-229 (0.2 g, 0.4689 mmol) was added to a 500 mL round-bottomed flask, and dissolved in dichloromethane (10 mL), and then TFA (0.7 mL, 9.3780 mmol) was added, and the reactant solution was stirred for reaction at the room temperature overnight. After the reaction ended, the reactant solution was concentrated under reduced pressure, n-hexane (100 mL) and ethyl acetate (400 mL) were added for precipitation, such procedure was repeated three times, and the resultant product was filtered and dried, to obtain 0.2 g of the product. MALDI-TOF MS: [M+H⁺] 327.19.

25-71

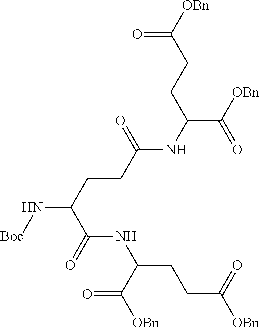 [SMILES: CC(C)(C)OC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

The Boc-Gly-OH (15.0 g, 60.6673 mmol, Ark Pharm), HBTU (69.0225 g, 182.0022 mmol, Aladdin), HOBT (24.5921 g, 182.0022 mmol, Innochem) and H-Glu(OBn)₂·TsOH (63.6473 g, 127.4014 mmol, Ark Pharm) were added to a 1000 mL round-bottomed flask, then DMF (300 mL) was added for dissolution, then the reaction flask was kept at −5° C. and the reactant solution was stirred for about 30 minutes before DIEA (90.2 mL, 546.0066 mmol) was slowly added dropwise, then the reaction flask was still kept at −5° C., and the reactant solution was stirred for reaction for 1 hour and finally was stirred overnight at room temperature. After the reaction ended, the reactant solution was transferred to a 2 L separatory funnel, a saturated sodium bicarbonate solution (400 mL) and ethyl acetate (300 mL) were added, and the mixture was shaken, left standing and extracted, to separate the aqueous phase and the organic phase. Next, a saturated sodium chloride solution (300 mL) was added to the organic phase, and the mixture was shaken, left standing and extracted, to separate the aqueous phase and the organic phase. Then the saturated sodium chloride solution (300 mL) was further added to the organic phase, and the mixture was shaken, left standing and extracted, to separate the aqueous phase and the organic phase. Finally, the organic phase was concentrated, evaporated and dried in an oven. 67.9 g of the product 25-71 was obtained.

25-73

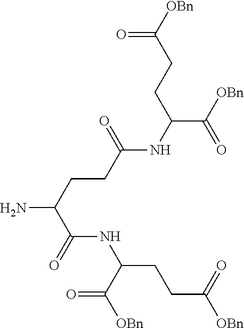 [SMILES: NC(CCC(=O)NC(CCC(=O)OCc1ccccc1)C(=O)OCc1ccccc1)C(=O)NC(CCC(=O)OCc1ccccc1)C(=O)OCc1ccccc1]

The product 25-71 (52.5355 g, 60.6673 mmol) was added into a 500 mL round-bottomed flask, dichloromethane (10 mL) was added for dissolution, and then trifluoroacetic acid (TFA, 67.6 mL, 910.0101 mmol) was added during stirring, and finally the reactant solution was stirred overnight at room temperature. After the reaction ended, the reactant solution was first concentrated under reduced pressure and evaporated to dryness to remove dichloromethane. The reactant solution was transferred to a 2 L separatory funnel, a saturated sodium bicarbonate solution (400 mL) and ethyl acetate (300 mL) were added, and the mixture was shaken, left standing and extracted, to separate the aqueous phase and the organic phase. Next, a saturated sodium bicarbonate solution (300 mL) was added to the organic phase, and the mixture was shaken, left standing and extracted, to separate the aqueous phase and the organic phase. Then deionized water (300 mL) was further added to the organic phase, and the mixture was shaken, left standing and extracted, to separate the aqueous phase and the organic phase. Finally, the organic phase was concentrated and evaporated to dryness. The organic phase was dissolved in a mixed solvent (100 mL) of 20% methanol/dichloromethane, 150 mL of silica gel powder was added to obtain a mixture which was then evaporated to dryness and was dry loaded for column chromatography. Elution was performed with an eluent (60% to 100% ethyl acetate/40% to 0% petroleum ether and 1% to 4% methanol/99% to 96% ethyl acetate), and a liquid was collected, concentrated, evaporated and dried. 44.4 g of the product 25-73 was obtained, with a yield of 95.56%.

43-162

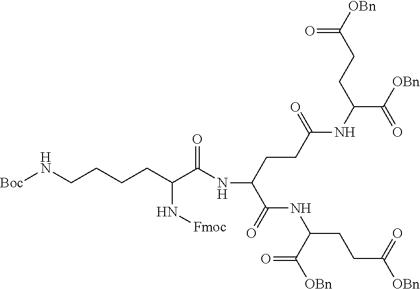

The Fmoc-Lys(Boc)-OH (3.5 g, 7.57 mmol, Ark Pharm), HBTU (4.3 g, 11.36 mmol, Aladdin), HOBT (1.5 g, 11.36 mmol, Innochem) and the product 25-73 (5.8 g, 7.57 mmol) were added to a 500 mL round-bottomed flask, then DMF (60 mL) was added for dissolution, then the reaction flask was kept at −5° C. and the reactant solution was stirred for about 30 minutes before DIEA (5.6 mL, 34.065 mmol) was slowly added dropwise, then the reaction flask was still kept at −5° C., and the reactant solution was stirred for reaction for 1 hour and finally was stirred overnight at room temperature. After the reaction ended, the reactant solution was transferred to a 1 L beaker, pure water (200 mL) was added, the reactant solution was filtered, then the filter cake was dissolved in DMF (100 mL), pure water (200 mL) was added, and the mixture was filtered. Such procedure was repeated twice, and the filter cake was dried in the oven. 8 g of the product 43-162 was obtained. The yield was 80%.

43-174

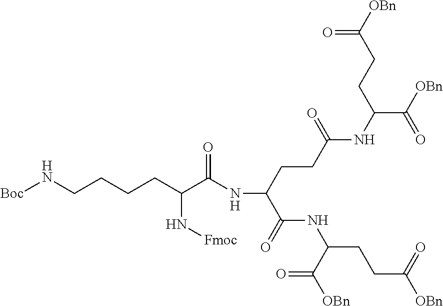

DMF (30 mL) was added to a flask containing the product 43-162 (2.8 g, 2.09 mmol), and was ultrasonically oscillated until complete dissolution, then morpholine (3.6 mL, 41.8 mmol) was added, and the resultant solution was stirred for reaction for 2 hours at room temperature. After the reaction ended, pure water (100 mL) was added, then ethyl acetate (200 mL) was added for extraction, and the organic phase was collected after standing and stratification, and then the aqueous phase was rinsed with ethyl acetate (200 mL×3), and the organic phases were collected, combined, evaporated and concentrated to about 50 mL, and then taken out. The organic phases were rinsed with the saturated sodium chloride solution (100 mL×3), collected, combined, concentrated, evaporated and dried in a vacuum oven, to obtain 3 g of product 43-174.

43-204

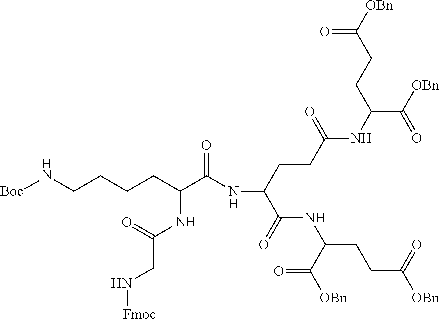

The Fmoc-Gly-OH (0.62 g, 2.09 mmol, Ark Pharm), HBTU (1.2 g, 3.135 mmol), HOBT (0.4 g, 3.135 mmol) and the product 43-174 (2 g, 2.09 mmol) were added to a 500 mL round-bottomed flask, then DMF (60 mL) was added for dissolution, then the reaction flask was kept at −5° C. and the reactant solution was stirred for about 30 minutes before DIEA (1.5 mL, 9.405 mmol) was slowly added dropwise, then the reaction flask was still kept at −5° C., and the reactant solution was stirred for reaction for 1 hour and finally was stirred overnight at room temperature. After the reaction ended, the reactant solution was transferred to a 1 L beaker, pure water (200 mL) was added, the reactant solution was filtered, then the filter cake was dissolved in DMF (100 mL), pure water (200 mL) was added, and the mixture was filtered. Then such procedure was repeated twice. The solid was dissolved in a mixed solvent (100 mL) of 20% methanol/dichloromethane. 50 mL of silica gel powder was added to obtain a mixture which was then evaporated to dryness and was dry loaded for column chromatography. Elution was performed with an eluent (2% to 4% methanol/98% to 96% dichloromethane), and a liquid was collected, concentrated, evaporated and dried. 2.1 g of the product 43-204 was obtained, with a yield of 71%.

43-209

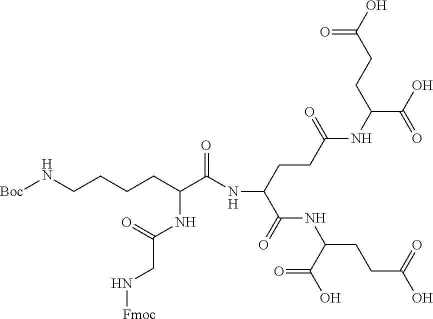

The product 43-204 (1.57 g, 1.23 mmol) was added into a hydrogenation reaction kettle, then 10% Pd/C (0.15 g) was added, DMF (40 mL) was added, hydrogen gas was injected at a hydrogen gas pressure of 1.4 MPa, and the reactant solution was stirred overnight at room temperature. After the reaction ended, the reactant solution was filtered with celite, the filter cake was rinsed with DMF (15 mL×3), and the filtrate was added into a 250 mL round-bottomed flask, to obtain product 43-209 to be used as a raw material for the next step.

43-211

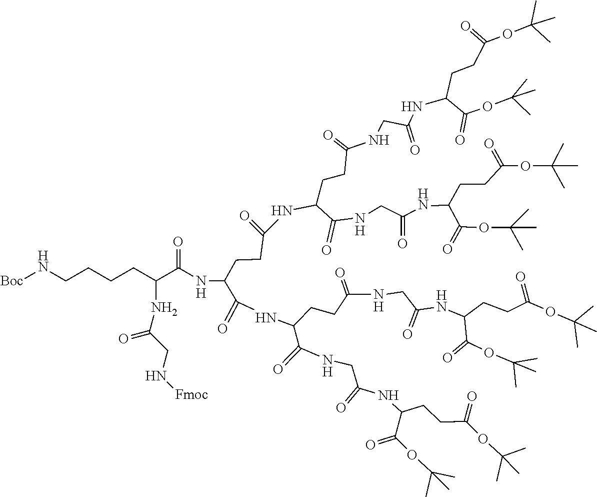

The product 43-209 (1.1 g, 1.23 mmol), HBTU (2.8 g, 7.38 mmol), HOBT (1 g, 7.38 mmol) and the product 48-66 (1.8 g, 5.90 mmol) were added to a 500 mL round-bottomed flask, then DMF (60 mL) was added for dissolution, then the reaction flask was kept at −5° C. and the reactant solution was stirred for about 30 minutes before DIEA (3.6 mL, 22.14 mmol) was slowly added dropwise, then the reaction flask was still kept at −5° C., and the reactant solution was stirred for reaction for 1 hour and finally was stirred overnight at room temperature. After the reaction ended, the reactant solution was transferred to a 1 L separatory funnel, pure water (300 mL) and ethyl acetate (200 mL) were added, and the mixture was shaken, left standing and extracted, to separate the aqueous phase and the organic phase. Next, ethyl acetate (100 mL) was added to the aqueous phase, and the mixture was shaken, left standing and extracted, to separate the aqueous phase and the organic phase. Then saturated brine (300 mL) was further added to the organic phase, and the mixture was shaken, left standing and extracted, to separate the aqueous phase and the organic phase. Finally, the organic phase was concentrated and evaporated to dryness. The organic phase was dissolved in a mixed solvent (100 mL) of 20% methanol/dichloromethane. 50 mL of silica gel powder was added to obtain a mixture which was then evaporated to dryness and was dry loaded for column chromatography. Elution was performed with an eluent (5% to 8% methanol/95% to 92% dichloromethane), and a liquid was collected, concentrated, evaporated and dried. 1.8 g of the product 43-211 was obtained, with a yield of 72%.

043-214

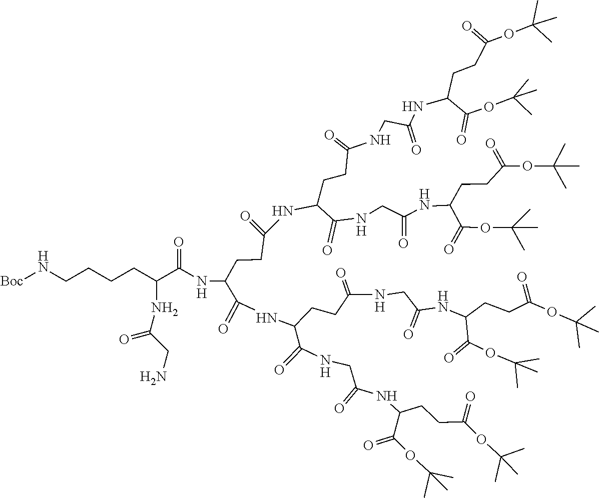 [SMILES: CC(C)(C)OC(=O)CCC(NC(=O)CNC(=O)CCC(NC(=O)CCC(NC(=O)C(CCCCNC(=O)OC(C)(C)C)NC(=O)CN)C(=O)NC(CCC(=O)NCC(=O)NC(CCC(=O)OC(C)(C)C)C(=O)OC(C)(C)C)C(=O)NCC(=O)NC(CCC(=O)OC(C)(C)C)C(=O)OC(C)(C)C)C(=O)NCC(=O)NC(CCC(=O)OC(C)(C)C)C(=O)OC(C)(C)C)C(=O)OC(C)(C)C]

DMF (30 mL) was added to a flask containing the product 43-211 (1.8 g, 0.85 mmol), and was ultrasonically oscillated until complete dissolution, then morpholine (1.5 mL, 17.09 mmol) was added, and the resultant solution was stirred for reaction for 2 hours at room temperature. After the reaction ended, the reactant solution was transferred to a 1 L separatory funnel, pure water (300 mL) and ethyl acetate (200 mL) were added, and the mixture was shaken, left standing and extracted, to separate the aqueous phase and the organic phase. Next, ethyl acetate (100 mL) was added to the aqueous phase, and the mixture was shaken, left standing and extracted, to separate the aqueous phase and the organic phase. Then saturated brine (300 mL) was further added to the organic phase, and the mixture was shaken, left standing and extracted, to separate the aqueous phase and the organic phase. Finally, the organic phase was concentrated and evaporated to dryness. The organic phase was dissolved in a mixed solvent (100 mL) of 20% methanol/dichloromethane. 50 mL of silica gel powder was added to obtain a mixture which was then evaporated to dryness and was dry loaded for column chromatography. Elution was performed with an eluent (5% to 6% methanol/95% to 94% dichloromethane), and a liquid was collected, concentrated, evaporated and dried. 1.07 g of the product 43-214 was obtained, with a yield of 62%.

21-221

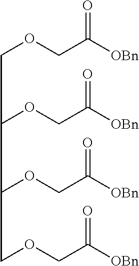 [SMILES: O=C(COCC(OCC(=O)OCc1ccccc1)C(COCC(=O)OCc1ccccc1)OCC(=O)OCc1ccccc1)OCc1ccccc1]

Erythritol (4.5 g, 36.849 mmol) was added into a 500 mL two-neck reaction flask, tetrahydrofuran (60 mL) was added, and the resultant mixture was uniformly mixed via ultrasonication and stirred at 0° C. Nitrogen was injected for protection, potassium tert-butoxide (200 mL, 184.245 mmol) was added, the reactant solution was stirred at 0° C. for 2 hours, then benzyl bromoacetate (29.187 mL, 184.245 mmol) was added, and the reactant solution was further stirred for 3 hours, and then reacted at room temperature. After the reaction ended, pure water and ethyl acetate were added for extraction of the reactant solution, the organic phase was concentrated and dry loaded for column chromatography. Gradient elution was performed using 1% to 2% ethyl acetate/petroleum ether, to obtain 5 g of the product, with a yield of 20%.

38-120

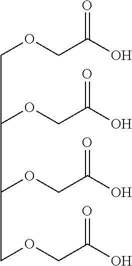 [SMILES: O=C(O)COCC(OCC(=O)O)C(COCC(=O)O)OCC(=O)O]

The product 21-221 (0.64 g, 0.9793 mmol) and 10% Pd/C (100 mg) were added into a hydrogenation reaction kettle, DMF (30 mL) was slowly added, the resultant mixture was slowly stirred and dissolved, hydrogen gas (20 psi) was injected, and the reactant solution was stirred overnight at room temperature. Next day, the reactant solution was subjected to suction filtration with a sand core funnel filled with celite to remove Pd/C and obtain a solution of the product in DMF to be used directly in the next reaction.

43-228

The product 43-214 (1.07 g, 0.57 mmol), HBTU (0.27 g, 0.714 mmol), HOBT (0.1 g, 0.714 mmol) and the product 38-120 (0.04 g, 0.1190 mmol) were added to a 500 mL round-bottomed flask, then DMF (60 mL) was added for dissolution, then the reaction flask was kept at −5° C. and the reactant solution was stirred for about 30 minutes before DIEA (0.35 mL, 2.142 mmol) was slowly added dropwise, then the reaction flask was still kept at −5° C., and the reactant solution was stirred for reaction for 1 hour and finally was stirred overnight at room temperature. After the reaction ended, methyl tertiary butyl ether (100 mL) was added, and n-hexane (200 mL) was added for precipitation, to obtain powder. The powder was dissolved in a mixed solvent (100 mL) of 20% methanol/dichloromethane. 50 mL of silica gel powder was added to obtain a mixture which was then evaporated to dryness and was dry loaded for column chromatography. Elution was performed with an eluent (3% to 10% methanol/97% to 90% dichloromethane), and a liquid was collected, concentrated, evaporated and dried. 0.2 g of product 43-228 was obtained, with a yield of 22%.

48-231

The product 43-228 (0.16 g, 0.0204 mmol) was added into a 250 mL flask, THE (20 mL) was added, then TEA (0.9 mL, 12.278 mmol) was added, and the reactant solution was subjected to oil bath (5000) and stirred overnight at room temperature. After the reaction ended, the reactant solution was concentrated to a small volume, methyl tertiary butyl ether (100 mL) was added, to obtain a powder-like solid precipitate, then suction filtration was performed, and the resultant filter cake was rinsed with methyl tertiary butyl ether (50 mL×3) and dried. Then toluene (20 mL) and dichloromethane (20 mL) were added, the resultant mixture was uniformly mixed via ultrasonication and evaporated to dryness, such procedure was repeated twice, and the resultant product was dried, to obtain 0.2 g of product 43-231.

43-232

Reactant 43-231 (0.2 g, 0.0204 mmol) were added into a 250 mL flask, DMF (20 mL) was added for dissolution, then the reactant solution was subjected to a bath at constant low temperature (−5° C.) and stirred for 30 minutes, and DIEA (0.34 mL, 2.04 mmol) was added dropwise. M-SCM-10K (1.02 g, 0.09792 mmol, JenKem) was added for dissolution, and then the reactant solution reacted for one week at room temperature while protected from light. The reactant solution was stirred at a low speed until the reaction ended, then n-hexane (120 mL) and methyl tertiary butyl ether (40 mL) were added, to obtain a solid precipitate, then filtration was performed, and the resultant filter cake was dried in a vacuum oven, to obtain 0.96 g of product 43-232.

43-233

The product 43-232 (0.96 g, 0.0204 mmol), HBTU 90.37 g, 0.9792 mmol) and HOBT (0.13 g, 0.9792 mmol) were added to a flask containing the product 45-230 (0.266 g, 0.816 mmol), then an appropriate amount of DMF was added for dissolution, the reactant solution was kept at −5° C., and DIEA (0.48 mL, 2.9376 mmol) was slowly added dropwise, and then the reactant solution reacted for half an hour, then taken out and stirred for reaction overnight at room temperature. After the reaction ended, methyl tertiary butyl ether (100 mL) was added, and n-hexane (200 mL) was added for precipitation, to obtain powder. The powder was dissolved in a mixed solvent (100 mL) of 20% methanol/dichloromethane. 50 mL of silica gel powder was added to obtain a mixture which was then evaporated to dryness and was dry loaded for column chromatography. Elution was performed with an eluent (5% to 10% methanol/95% to 90% dichloromethane), and a liquid was collected, concentrated, evaporated and dried. 0.7 g of the product 43-233 was obtained, with a yield of 63%.

¹H-NMR (400 MHz, DMSO-d₆) 8.45-8.35 (m, 56H), 8.42-8.33 (m, 24H), 8.24-8.17 (m, 28H), 7.95-7.91 (m, 102H), 7.80-7.76 (m, 55H), 7.51-7.49 (m, 36H), 7.30-7.21 (m, 34H), 5.75-5.73 (m, 69H), 4.50-4.49 (m, 3H), 4.48-4.47 (m, 4H), 4.36-4.35 (m, 3H), 4.33-4.32 (m, 7H), 4.14-4.12 (m, 17H), 3.81-3.76 (m, 6H), 3.69-3.69 (m, 11H), 3.68-3.67 (m, 44H), 3.65-3.45 (m, 3810H), 2.91-2.89 (m, 41H), 2.86-2.80 (m, 53H), 2.73-2.69 (m, 31H), 2.67-2.63 (m, 22H), 2.33-2.32 (m, 13H), 2.08-1.99 (m, 48H), 1.27-1.25 (m, 192H), 1.20-1.19 (m, 8H), 1.18-1.17 (m, 2H), 1.15-1.14 (m, 8H), 1.07-1.05 (m, 13H), 1.04-0.94 (m, 12H), 0.93-0.92 (m, 9H).

### Synthesis of Compound 37-228

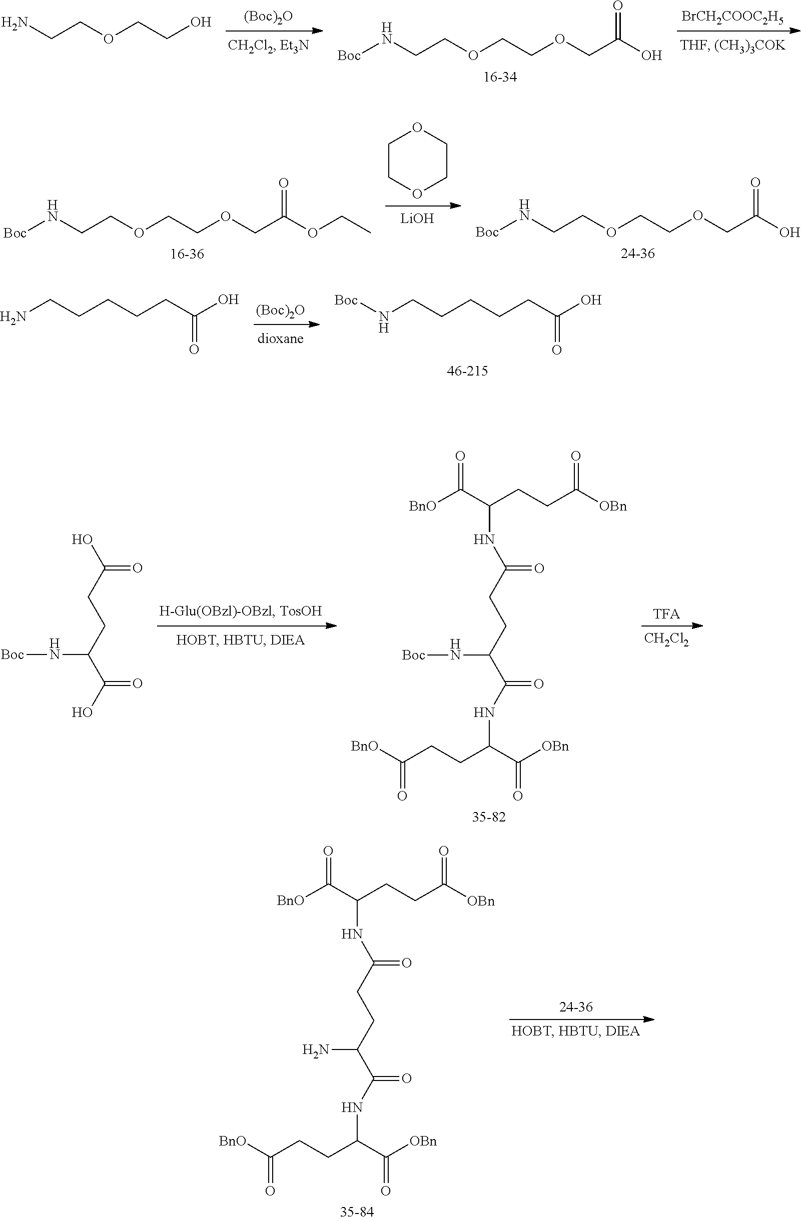

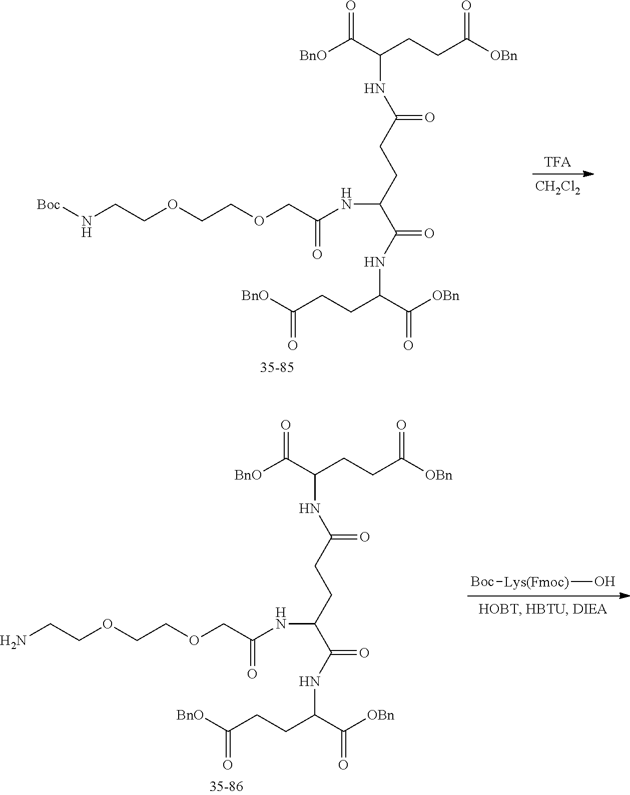

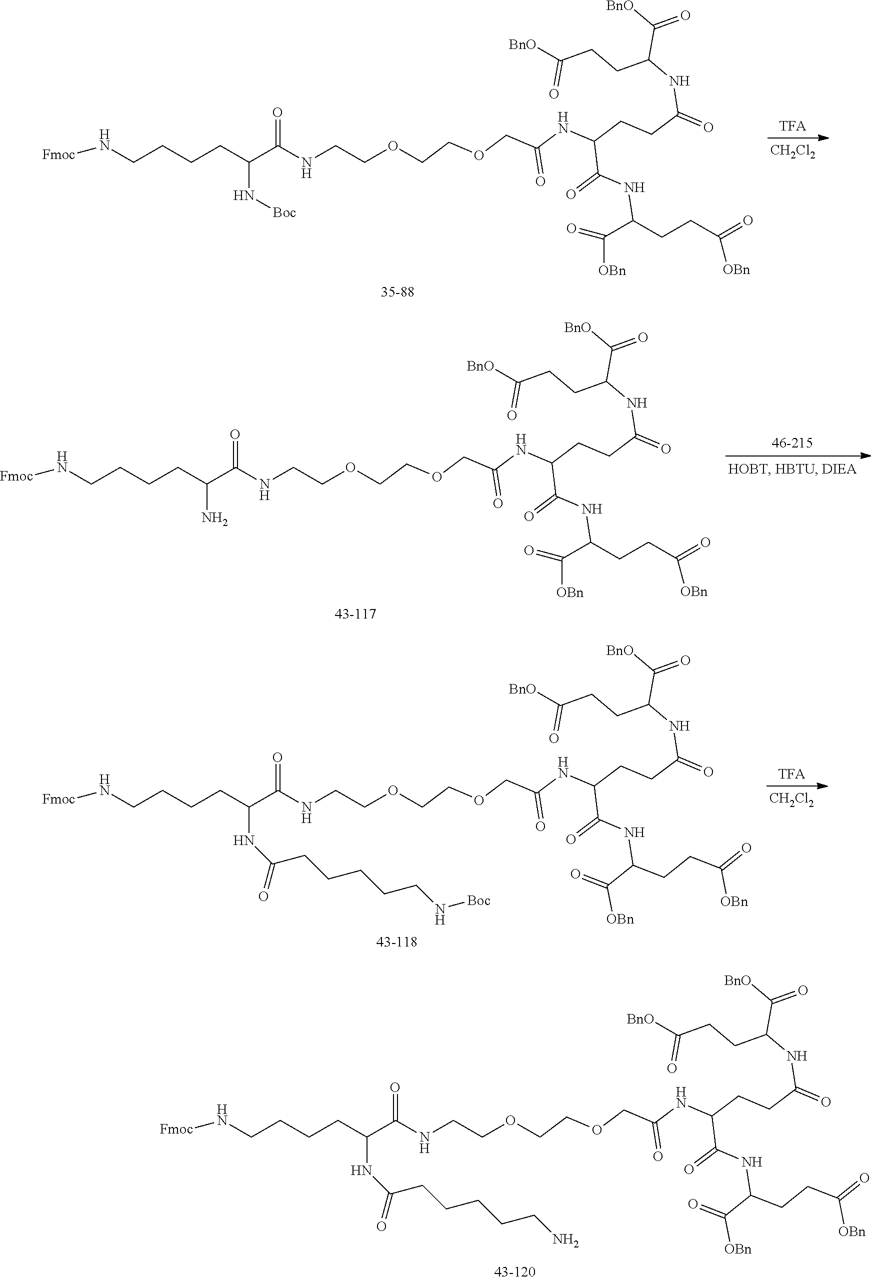

16-34

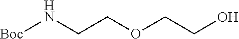 [SMILES: CC(C)(C)OC(=O)NCCOCCO]

2-(-2 aminoethoxy)ethanol (18.87 g, 190.22 mmol, Admas) was put into a 500 mL round-bottomed flask, dichloromethane (100 mL) was added for dilution, then triethylamine (38.4972 mL, 380.4452 mmol) was added, (Boc)₂O (49.8261 g, 228.2671 mmol, damas-beta) was slowly added during stirring, and the reactant solution was stirred for reaction at room temperature. After the reaction ended, the reactant solution was evaporated to dryness, then sodium bicarbonate powder was added, the resultant mixture was diluted with dichloromethane, silica gel powder was added, and the resultant mixture was evaporated to dryness and then dry loaded for column chromatography. Elution was performed using 50% ethyl acetate/petroleum ether, to obtain 27.3 g of the product, with a yield of 70%.

16-36

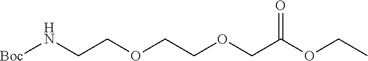 [SMILES: CCOC(=O)COCCOCCNC(=O)OC(C)(C)C]

The product 16-34 (27.3 g, 132.8144 mmol) was added into a 500 mL reaction flask, nitrogen was injected for protection, a solution of potassium tert-butoxide in THE was added, the reactant solution was kept at 0° C., then ethyl bromoacetate (17.6265 mL, 159.3773 mmol, Damas-Beta) was added, and the reactant solution was stirred for three hours, and then reacted at room temperature. After the reaction ended, the reactant solution was first evaporated to dryness, then deionized water and ethyl acetate were added, the organic phase was separated, the aqueous phase was extracted with ethyl acetate until no product was found, the organic phases were combined, dried with anhydrous sodium sulfate powder, and subjected to suction filtration, and the filtrate was dry loaded for column chromatography. Gradient elution was performed using 30% to 100% ethyl acetate/petroleum ether, to obtain 20 g of the product, with a yield of 52%.

24-36

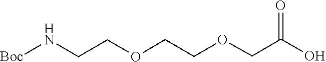 [SMILES: CC(C)(C)OC(=O)NCCOCCOCC(=O)O]

The product 16-36 (17.9 g, 61.4402 mmol) was weighted and added into a 250 mL reaction flask, 1,4-dioxane was added, lithium hydroxide (3.2386 g, 135.1685 mmol) was added during stirring, and after 30 minutes, deionized water was added until the reactant solution was clear. After the reaction ended, the reactant solution was extracted (100 mL×3) with methyl tertiary butyl ether/n-hexane (1:1) 3 times. The aqueous phase was adjusted with concentrated hydrochloric acid until the pH was equal to 1, and extracted with ethyl acetate (300 mL×3) three times, the ethyl acetate phases were combined, rinsed with saturated sodium chloride solution (100 mL×3) three times, concentrated and dry loaded for column chromatography with elution using 40% to 100% ethyl acetate/petroleum ether to obtain 10.1 g of the product, with a yield of 62%.

35-82

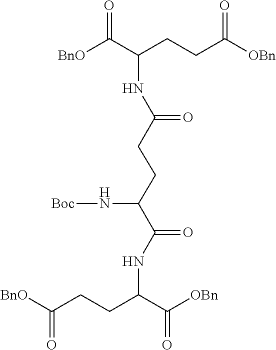 [SMILES: CC(C)(C)OC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

Boc-Glu-OH (20.00 g, 80.89 mmol, Ark Pharm), H-Glu(OBzl)-OBzl·TsOH (84.86 g, 169.87 mmol, Ark Pharm), HBTU (92.02 g, 242.66 mmol) and HOBT (32.80 g, 242.66 mmol) were added into a 500 mL flask, DMF (200 mL) was added for dissolution, the reactant was stirred at −5° C. for 30 minutes. Then DIEA (120.32 mL, 728.00 mmol) was slowly added dropwise, and the reactant solution was further stirred at −5° C. for 30 minutes, and then stirred for reaction overnight at room temperature. After the reaction ended, the reactant solution was taken out, added with deionized water (200 mL), and extracted with ethyl acetate (100 mL×3), the organic phases were combined, rinsed with a saturated sodium chloride solution (200 mL) twice and concentrated, evaporated to dryness and dried in a vacuum oven, to obtain a crude product.

35-84

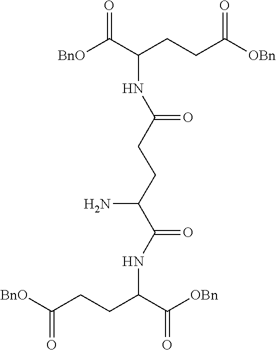 [SMILES: NC(CCC(=O)NC(CCC(=O)OCc1ccccc1)C(=O)OCc1ccccc1)C(=O)NC(CCC(=O)OCc1ccccc1)C(=O)OCc1ccccc1]

The product 35-82 (70 g, 80.89 mmol) was added into a 1000 mL round-bottomed flask, dichloromethane (50 mL) was added for dissolution, and trifluoroacetic acid (300.00 mL, 4041.5 mmol) was added, and the reactant solution reacted at room temperature. After the reaction ended, the reactant solution was taken out, added with deionized water (200 mL), and extracted with ethyl acetate (100 mL×3), the organic phases were combined, rinsed with a saturated sodium chloride solution (200 mL×2), and concentrated, silica gel powder was added and the resultant mixture was evaporated to obtain powder, and the powder was subjected to column chromatography. Gradient elution was performed (1% ammonia+5% CH₃OH, the rest was CH₂Cl₂), and the desired component was collected and concentrated, to obtain 61.95 g of product 35-84.

35-85

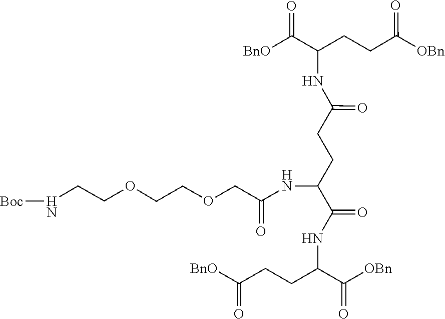 [SMILES: CC(C)(C)OC(=O)NCCOCCOCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

The product 24-36 (15.0236 g, 57.0608 mmol), HBTU (32.4596 g, 85.5912 mmol), HOBT (11.5651 g, 85.5912 mmol) and the product 35-84 (43.7 g, 57.0608 mmol) were added to a 500 mL round-bottomed flask, then DMF (150 mL) was added for dissolution, then the reaction flask was kept at −5° C. and the reactant solution was stirred for about 30 minutes before DIEA (61.5 mL, 342.3648 mmol) was slowly added dropwise, then the reaction flask was still kept at −5° C., the reactant solution was stirred for reaction for 2 hours, and the reaction flask was transferred for stirring the reactant solution overnight at room temperature. After the reaction ended, the reactant solution was first transferred to a 2 L separatory funnel, a saturated sodium bicarbonate solution (400 mL) and ethyl acetate (300 mL) were added, and the mixture was shaken, left standing and extracted, to separate the aqueous phase and the organic phase. Next, a saturated sodium chloride solution (300 mL) was added to the organic phase, and the mixture was shaken, left standing and extracted, to separate the aqueous phase and the organic phase. Then deionized water (300 mL) was further added to the organic phase, and the mixture was shaken, left standing and extracted, to separate the aqueous phase and the organic phase. Finally, the organic phases were concentrated and evaporated to dryness. The organic phase was dissolved in a mixed solvent (100 mL) of 20% methanol/dichloromethane, 150 mL of silica gel powder was added to obtain a mixture which was then evaporated to dryness and was dry loaded for column chromatography. Elution was performed with an eluent (1% ammonia/1% to 2% methanol/98% to 97% dichloromethane), and a liquid was collected, concentrated and evaporated. 42.1 g of product 35-85 was obtained, with a yield of 72.97%.

35-86

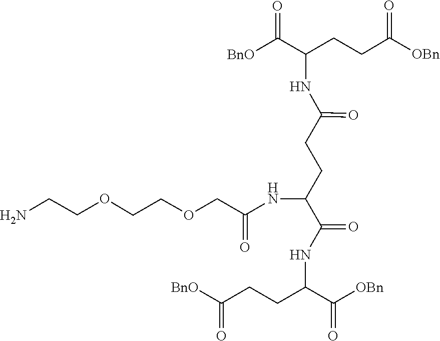 [SMILES: NCCOCCOCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1]

The product 35-85 (23.4 g, 23.14 mmol) was added into a 500 mL round-bottomed flask, dichloromethane (30 mL) was added for dissolution, and then trifluoroacetic acid (85.93 mL, 1157.13 mmol) was added during stirring, and finally the reactant solution was stirred overnight at room temperature. After the reaction ended, the reactant solution was concentrated, ethyl acetate (300 mL) and a saturated sodium bicarbonate solution (300 mL) were added, a lot of bubbles were generated, solid sodium bicarbonate was further added until pH was greater than 7, then extraction was performed, the aqueous phase was rinsed with ethyl acetate (200 mL×1), and the organic phases were combined and evaporated to dryness, to obtain 18.2 g of product 35-86, with a yield of 86%.

35-88

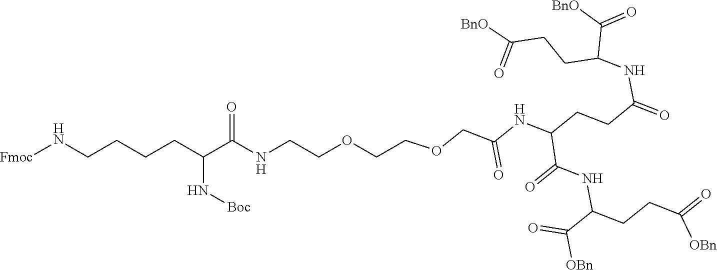

The product 35-86 (18.2 g, 19.98 mmol), HBTU (11.36 g, 29.97 mmol), HOBT (4.05 g, 29.97 mmol) and Boc-Lys(Fmoc)-OH (8.5 g, 18.16 mmol, Aladdin) were added to a 500 mL round-bottomed flask, then DMF (100 mL) was added for dissolution, then the reactant solution was stirred at 0° C. for about 30 minutes before DIEA (14.86 mL, 89.90 mmol) was slowly added dropwise, and then the reactant solution was further stirred for reaction at 0° C. overnight. After the reaction ended, the reactant solution was first transferred to a 1 L separatory funnel, added with a saturated sodium bicarbonate solution (300 mL) and ethyl acetate (200 mL) and shaken for extraction to obtain an organic phase, the aqueous phase was rinsed with ethyl acetate (200 mL×1), the organic phases were combined and concentrated to a small volume, silica gel powder was added, and the resultant mixture was evaporated to dryness and then dry loaded for column chromatography with elution using a mixed solution of 80% to 100% ethyl acetate in petroleum ether, to obtain 19.6 g of product 35-88, with a yield of 79%.

43-117

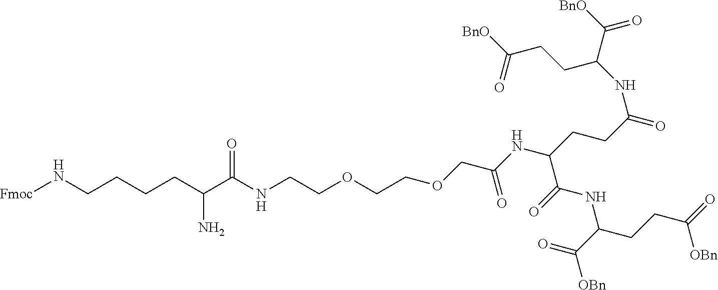

The product 35-88 (7.0 g, 5.1413 mmol) was added into a 250 mL flask, dichloromethane (20 mL) was added for dissolution, and trifluoroacetic acid (5.7270 mL, 77.1191 mmol) was added during stirring, and the reactant solution was stirred overnight at room temperature. After the reaction ended, the reactant solution was concentrated and transferred to a 1 L separatory funnel, added with a saturated sodium bicarbonate solution (300 mL) and ethyl acetate (200 mL) and shaken for extraction to obtain an organic phase, the aqueous phase was rinsed with ethyl acetate (150 mL×2), and the organic phases were combined, rinsed with saturated brine (200 mL×2), concentrated and evaporated to dryness, to obtain 6.4853 g of product 43-117, with a yield of 99%.

46-215

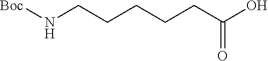 [SMILES: CC(C)(C)OC(=O)NCCCCCC(=O)O]

6-aminocaproic acid (15.00 g, 114.35 mmol) was put into a 1 L flask, 114 mL of 1 mol/L sodium hydroxide solution was added and stirred at 0° C., then dioxane (100 mL) was added, and after 5 minutes, Boc₂O (49.92 g, 228.71 mmol) was dissolved in dioxane (100 mL) and the mixture was added to the flask and stirred for reaction. After the reaction ended, the reactant solution was evaporated to dryness under reduced pressure and transferred to a 1 L separatory funnel, added with deionized water (300 mL) and ethyl acetate (200 mL) and shaken for extraction to obtain an organic phase, the aqueous phase was rinsed with ethyl acetate (200 mL×2), and the organic phases were combined and concentrated to a small volume, silica gel powder was added, and the resultant mixture was evaporated to dryness and then dry loaded for column chromatography with elution using a mixed solution of 0% to 5% methanol in dichloromethane, to obtain 22.6 g of product 46-215, with a yield of 85%.

43-118

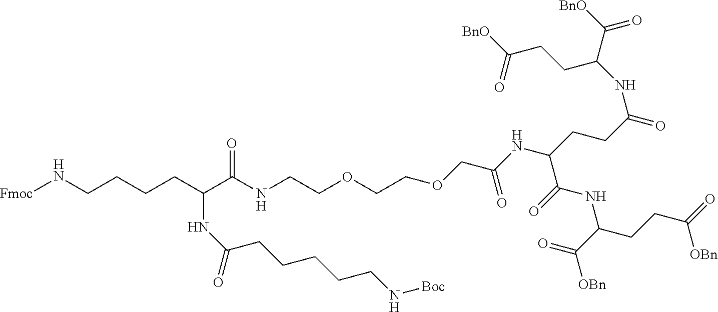

The product 43-117 (9.08 g, 7.20 mmol), HBTU (4.10 g, 10.80 mmol), HOBT (1.40 g, 10.80 mmol) and the product 46-215 (1.60 g, 7.20 mmol) were added to a 500 mL round-bottomed flask, then DMF (100 mL) was added for dissolution, then the reactant solution was stirred at 0° C. for about 30 minutes before DIEA (5.30 mL, 32.40 mmol) was slowly added dropwise, and then the reactant solution was further stirred for reaction at 0° C. overnight. After the reaction ended, the reactant solution was first transferred to a 1 L separatory funnel, added with a saturated sodium chloride solution (300 mL) and ethyl acetate (200 mL) and shaken for extraction to obtain an organic phase, the aqueous phase was rinsed with ethyl acetate (200 mL×1), and the organic phases were combined, concentrated to a small volume and evaporated to dryness.

43-120

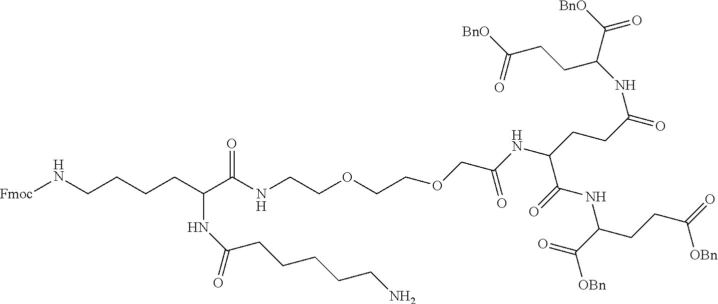

The product 43-118 (10.60 g, 7.20 mmol) was added into a 500 mL round-bottomed flask, dichloromethane (30 mL) was added for dissolution, and then trifluoroacetic acid (8.00 mL, 108.00 mmol) was added during stirring, and finally the reactant solution was stirred overnight at room temperature. After the reaction ended, the reactant solution was concentrated, ethyl acetate (300 mL) and a saturated brine solution (300 mL) were added, then extraction was performed, the aqueous phase was further extracted with ethyl acetate (200 mL×1), and the organic phases were combined and evaporated to dryness, to obtain crude product 43-120.

56-21

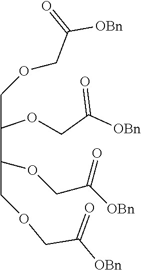 [SMILES: O=C(COCC(OCC(=O)OCc1ccccc1)C(COCC(=O)OCc1ccccc1)OCC(=O)OCc1ccccc1)OCc1ccccc1]

Erythritol (5.00 g, 40.90 mmol) was weighed and added into a three-neck flask, THE (60 mL) was added, and erythritol was dissolved ultrasonically, N₂ was injected for protection at 0° C., potassium tert-butoxide (245.40 mL, 245.40 mmol) was added, the reactant solution was stirred for 3 hours, and benzyl bromoacetate (32.40 mL, 204.70 mmol) was slowly added, then the resultant solution was stirred for reaction overnight at room temperature. After the reaction completed, the reactant solution was transferred to a 1 L separatory funnel, added with deionized water (300 mL) and ethyl acetate (200 mL) and shaken for extraction to obtain an organic phase, the aqueous phase was rinsed with ethyl acetate (200 mL×1), and the organic phases were combined and concentrated to a small volume, silica gel powder was added, and the resultant mixture was evaporated to dryness and then dry loaded for column chromatography. Gradient elution was performed using 1% to 2% ethyl acetate/petroleum ether, to obtain 6 g of product 56-21, with a yield of 21%.

43-121

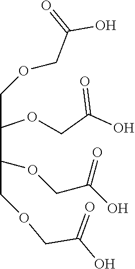 [SMILES: O=C(O)COCC(OCC(=O)O)C(COCC(=O)O)OCC(=O)O]

The product 56-21 (0.59 g, 0.83 mmol) was added into a hydrogenation reaction kettle, then 10% Pd/C catalyst (0.10 g) was added, DMF (20 mL) was further added for dissolution, hydrogen gas was injected to implement a pressure P of 1.8 MPa, and the reactant solution was stirred overnight at room temperature. After the reaction ended, the reactant solution was filtered with celite, the celite was rinsed with DMF (20 mL×3), and the DMF solution was combined and used as a raw material for the next reaction.

43-122

The product 43-120 (5.00 g, 3.60 mmol), HBTU (0.47 g, 1.24 mmol), HOBT (0.17 g, 1.24 mmol) and the product 43-121 (0.29 g, 0.83 mmol) were added to a 500 mL round-bottomed flask, then DMF (100 mL) was added for dissolution, then the reactant solution was stirred at 000 for about 30 minutes before DIEA (0.60 mL, 3.72 mmol) was slowly added dropwise, and then the reactant solution was further stirred for reaction at 0° C. overnight. After the reaction ended, the reactant solution was first transferred to a 1 L separatory funnel, added with a saturated sodium chloride solution (300 mL) and ethyl acetate (200 mL) and shaken for extraction to obtain an organic phase, the aqueous phase was rinsed with ethyl acetate (200 mL×1), and the organic phases were combined and concentrated to a small volume, silica gel powder was added, and the resultant mixture was evaporated to dryness and then dry loaded for column chromatography with elution using a mixed solution of 2% methanol and 97.5% dichloromethane, to obtain 1.3 g of product 43-122.

37-207

The product 43-122 (0.50 g, 0.0873 mmol) was added into a hydrogenation reaction kettle, then 10% Pd/C catalyst (0.03 g) was added, DMF (20 mL) was further added for dissolution, hydrogen gas was injected to implement a pressure of 1.8 MPa, and the reactant solution was stirred overnight at room temperature. After the reaction ended, the reactant solution was filtered with celite, the celite was rinsed with DMF (20 mL×3), and the DMF solution was combined and used as a raw material for the next reaction.

37-160

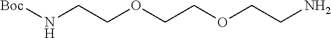 [SMILES: CC(C)(C)OC(=O)NCCOCCOCCN]

1,2-bis(2-aminoethoxy)ethane (50 g, 337.2 mmol, TCI) was added into a 1 L flask, triethylamine (37.6 mL, 270 mmol) was added, the reactant solution was stirred for 10 minutes at −5° C., (Boc)₂O was dissolved in dichloromethane, and then the resultant mixture was added into the flask dropwise, then the resultant mixture was further stirred for 10 minutes. After the reaction ended, silica gel powder (60 g) was added to obtain a mixture which was then evaporated to dryness to obtain a powder-like solid, and the powder-like solid was dry loaded for column chromatography with elution using a mixed solution of 1% ammonia and 4% methanol in dichloromethane, to obtain 28.7 g of product 37-160, with a yield of 34%.

37-208

The product 37-207 (0.0874 mmol), HBTU (0.80 g, 2.10 mmol), HOBT (0.29 g, 2.10 mmol) and the product 37-160 (0.43 g, 1.75 mmol) were added to a 500 mL round-bottomed flask, then DMF (100 mL) was added for dissolution, then the reactant solution was stirred at 000 for about 30 minutes before DIEA (1.04 mL, 6.29 mmol) was slowly added dropwise, and then the reactant solution was further stirred for reaction at 0° C. overnight. After the reaction ended, the reactant solution was first transferred to a 1 L separatory funnel, added with a saturated sodium chloride solution (300 mL) and ethyl acetate (200 mL) and shaken for extraction to obtain an organic phase, the aqueous phase was rinsed with ethyl acetate (200 mL×1), and the organic phases were combined, concentrated and evaporated to dryness, to obtain a crude product of 37-208.

37-211

The product 37-208 (0.0874 mmol) was added into a 250 mL flask, DMF (40 mL) was added for dissolution, and then morpholine (3.80 mL, 43.70 mmol) was added, and the reactant solution was stirred overnight at room temperature. After the reaction ended, the reactant solution was transferred to a 1 L separatory funnel, pure water (200 mL) and ethyl acetate (200 mL) were added for extraction to obtain an organic phase, the aqueous phase was rinsed with ethyl acetate (200 mL×1), and the organic phases were combined and concentrated, silica gel powder was added, and the resultant mixture was evaporated to dryness and subjected to column chromatography. 0.26 g of product 37-211 was obtained.

The product 37-211 (0.26 g 0.0368 mmol) was added into a 250 mL flask, DMF (40 mL) was added for dissolution, M-SCM-10K (1.715 g, 0.1619 mmol, JenKem) was added, and the reactant solution was stirred for one week at a low speed at room temperature while protected from light. After the reaction ended, methyl tertiary butyl ether (200 mL) and n-hexane (70 mL) were further added, to obtain a solid precipitate, then suction filtration was performed, and the filter cake was rinsed with methyl tertiary butyl ether (40 mL×3). Then the filter cake was dissolved in a solvent (100 mL) of methanol/dichloromethane (1:4), silica gel powder (10 mL) was added to obtain a mixture which was then evaporated to dryness to obtain a powder-like solid, and the powder-like solid was dry loaded for column chromatography with elution using a mixed solution of 1% ammonia and 5% to 7% methanol in dichloromethane, and the desired component was collected, concentrated and dried in a vacuum oven, to obtain 1.40 g of product 37-213, with a yield of 78%.

37-223

The product 37-213 (1.40 g, 0.0286 mmol) was added into a 100 mL round-bottomed flask, dichloromethane (20 m L) was added for dissolution, and then trifluoroacetic acid (4.25 mL, 57.17 mmol) was added during stirring, and finally the reactant solution was stirred overnight at room temperature. After the reaction ended, the reactant solution was evaporated to dryness under reduced pressure to remove dichloromethane, methyl tertiary butyl ether was added to obtain an oily substance, which was dried to obtain 0.8 g of crude product 37-223.

37-216

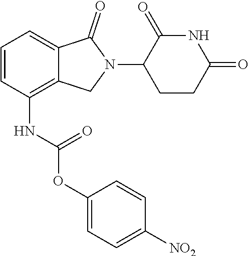 [SMILES: O=C1CCC(N2Cc3c(NC(=O)Oc4ccc([N+](=O)[O-])cc4)cccc3C2=O)C(=O)N1]

Lenalidomide (5 g, 19.2857 mmol, LNL for short) was added into a 250 mL flask and dissolved in THE (300 mL), 4-nitrophenyl chloroformate (5.83 g, 28.9285 mmol) was added under nitrogen protection, and the reactant solution was refluxed overnight at 40° C. After the reaction ended, the reactant solution was concentrated to 100 mL, then EA (200 mL) was added, to obtain a solid precipitate, then suction filtration was performed, and the filter cake was rinsed with EA (80 mL×4) and was dried to obtained 7.36 g of product 37-216, with a yield of 90%.

37-219

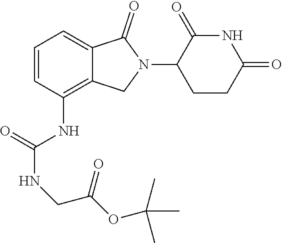 [SMILES: CC(C)(C)OC(=O)CNC(=O)Nc1cccc2c1CN(C1CCC(=O)NC1=O)C2=O]

The product 37-216 (7.36 g 17.3438 mmol) was added into a 250 mL flask, DMF (60 mL) was added for dissolution, DIEA (7.2 g, 43.36 mmol) was added, Gly-OtBu·HCl (3.2 g, 19.1 mmol, Accela) was added, and the reactant solution was stirred overnight at room temperature. After the reaction ended, n-hexane (150 mL×4) was added for precipitation, EA (100 mL) and n-hexane (200 mL) were added, to obtain a powder precipitate, then filtration was performed, and the filter cake was rinsed with EA (70 mL×3) and was dried to obtain 5.4 g of product 37-219, with a yield of 75%.

37-222

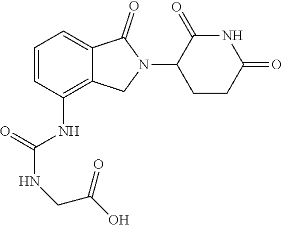 [SMILES: O=C(O)CNC(=O)Nc1cccc2c1CN(C1CCC(=O)NC1=O)C2=O]

The product 37-219 (5.4 g, 12.9674 mmol) was added into a 250 mL flask, dichloromethane (15 mL) was added for dissolution, then TFA (7.7 mL, 103.73 mmol) was added, and the reactant solution was stirred overnight at room temperature. After the reaction ended, the reactant solution was concentrated, methyl tertiary butyl ether (150 mL) was added, to obtain a powder-like solid precipitate, then suction filtration was performed, and the resultant filter cake was rinsed with methyl tertiary butyl ether (150 mL×2) and was dried to obtain 4.67 g of product 37-222, with a yield of 99%.

37-228

The products 37-223 (0.8 g, 0.0286 mmol) and 37-222 (0.206 g, 0.5717 mmol), HBTU (0.26 g, 0.686 mmol), and HOBT (0.0927 g, 0.686 mmol) were added to a 100 mL flask, DMF (25 mL) was added for dissolution, the reactant solution was kept at −5° C. and stirred for 20 minutes before DIEA (0.47 mL, 2.8587 mmol) was slowly added dropwise, and then the reactant solution was further stirred at −5° C. for 1 hour and then stirred overnight at room temperature. After the reaction ended, n-hexane (150 mL) and methyl tertiary butyl ether (100 mL) were added, to obtain an oily solid precipitate, the oily solid precipitate was dissolved in dichloromethane (30 mL) and methanol (10 mL), silica gel powder (10 g) was added to obtain a mixture which was then evaporated to dryness to obtain a powder-like solid, and the powder-like solid was dry loaded for column chromatography with elution using a mixed solution of 10% to 15% methanol in dichloromethane, and the desired component was collected, concentrated and dried, to obtain 0.15 g of product 37-228, with a yield of 10%.

¹H-NMR (600 MHz, DMSO-d₆) δ11.38-10.57 (m, 16H), 8.67-8.61 (m, 21H), 8.61-8.58 (m, 6H), 8.41-8.36 (m, 5H), 8.17-8.12 (m, 12H), 7.96-7.95 (m, 3H), 7.85-7.80 (m, 13H), 7.78-7.72 (m, 8H), 7.66-7.60 (m, 17H), 7.42-7.41 (m, 11H), 7.24-7.18 (m, 9H), 6.67-6.60 (m, 19H), 5.22-5.09 (m, 22H), 4.84-4.74 (m, 9H), 4.62-4.47 (m, 27H), 4.41-4.32 (m, 37H), 3.98-3.88 (m, 27H), 3.79-3.72 (m, 72H), 3.70-3.65 (m, 67H), 3.54-3.43 (m, 3850H), 2.95-2.86 (m, 37), 2.76-2.70 (m, 13H), 2.69-2.57 (m, 32H), 2.35-2.20 (m, 67H), 1.51-1.43 (m, 25H), 1.41-1.31 (m, 34H), 1.29-1.11 (m, 48H), 1.10-1.00 (m, 16H).

### Synthesis of Compound 57-210

57-36

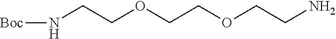 [SMILES: CC(C)(C)OC(=O)NCCOCCOCCN]

1,2-bis(2-aminoethoxy)ethane (20 mL, 136.2931 mmol, TCI) was weighed and added to a 500 mL flask, 100 mL of dichloromethane was added for dilution, Et3N (37.99 mL, 272.5862 mmol) was added, then Boc₂O (29.7 g, 136.2931 mmol) was weighed and dissolved in 100 mL of dichloromethane, and slowly added to the reactant solution dropwise at room temperature, and then reactant solution was stirred for reaction overnight at room temperature. After the reaction ended, silica gel powder was added to obtain a mixture which was then evaporated to dryness to obtain a powder-like solid, and the powder-like solid was dry loaded for column chromatography. Elution was performed using a mixed solution of 1% ammonia and 2% to 5% methanol in dichloromethane, and the desired component was collected, concentrated and dried in a vacuum oven, to obtain 10.66 g of product, with a yield of 31.47%.

57-37

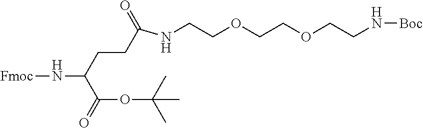

Fmoc-L-glutamic acid-5-tert-butyl ester (10.7 g, 25.1486 mmol, Innochem), HBTU (10.5 g, 27.6635 mmol) and HOBT (3.74 g, 27.6635 mmol) were added to a flask containing the product 57-36 (6.56 g, 26.4060 mmol), then an appropriate amount of DMF was added for dissolution, the reactant solution was kept at −5° C., and DIEA (9.15 mL, 55.3269 mmol) was slowly added dropwise, and then the reactant solution reacted for half an hour, then taken out and stirred for reaction overnight at room temperature. After the reaction ended, a saturated NaCl aqueous solution (200 mL) and ethyl acetate (200 mL) were added for extraction, the organic phase was collected after standing and stratification, the aqueous phase was rinsed with ethyl acetate (200 mL×3), the organic phases were combined and evaporated to dryness to obtain a solid, and the solid was collected, concentrated and dried in a vacuum oven, to obtain 20.5 g of the product, which was overproduction.

57-39

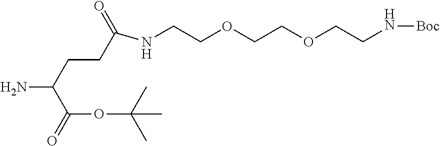 [SMILES: CC(C)(C)OC(=O)NCCOCCOCCNC(=O)CCC(N)C(=O)OC(C)(C)C]

DMF was added to a flask containing the product 57-37 (16.4919 g, 25.1486 mmol), and was ultrasonically oscillated until complete dissolution, then morpholine (21.9 mL, 251.486 mmol) was added, and the resultant solution was stirred for reaction for 2 hours at room temperature. After the reaction ended, a saturated NaCl aqueous solution (200 mL) and ethyl acetate (200 mL) were added for extraction, the organic phase was collected after standing and stratification, the aqueous phase was rinsed with ethyl acetate (200 mL×3), the organic phases were combined and evaporated to dryness to obtain a solid, and the solid was dried in a vacuum oven, to obtain 9.3 g of the product, with a yield of 85.32%.

57-54

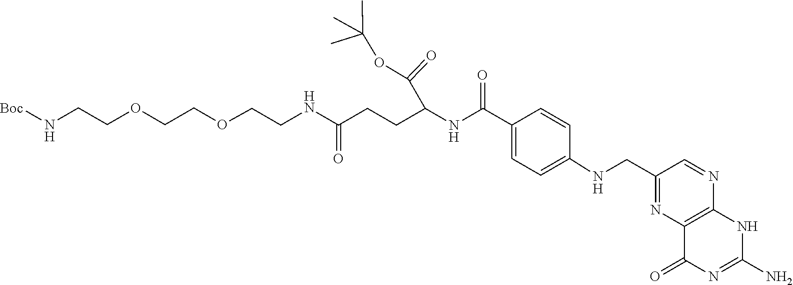 [SMILES: CC(C)(C)OC(=O)NCCOCCOCCNC(=O)CCC(NC(=O)c1ccc(NCc2cnc3[nH]c(N)nc(=O)c3n2)cc1)C(=O)OC(C)(C)C]

Pteroic acid (5.55 g, 17.7940 mmol, Innochem), HBTU (10.5 g, 27.6635 mmol) and HOBT (3.74 g, 27.6635 mmol) were added to a flask containing the product 57-39 (9.3 g, 21.3528 mmol), then an appropriate amount of DMF was added for dissolution, the reactant solution was kept at −5° C., and DIEA (9.15 mL, 55.3269 mmol) was slowly added dropwise, and then the reactant solution reacted for half an hour, then taken out and stirred for reaction overnight at room temperature. After the reaction ended, a saturated NaCl aqueous solution (200 mL) and ethyl acetate (200 mL) were added for extraction, the organic phase was collected after standing and stratification, the aqueous phase was rinsed with ethyl acetate (200 mL×3), the organic phases were combined and evaporated to dryness to obtain a solid, and the solid was collected, concentrated and dried in a vacuum oven, to obtain 7.61 g of the product.

¹H-NMR (400 MHz, DMSO-d₆) δ 11.43 (s, 1H), 8.66 (s, 1H), 8.20-8.18 (m, 1H), 7.92-7.91 (m, 1H), 7.66-7.64 (m, 2H), 7.20-7.18 (m, 1H), 6.94-6.62 (m, 7H), 4.50-4.45 (m, 2H), 4.21 (s, 1H), 3.56-3.55 (m, 2H), 3.47 (s, 4H), 3.19-3.17 (m, 2H), 3.06-3.05 (m, 2H), 2.22-2.19 (m, 2H), 2.01-1.99 (m, 1H), 1.91-1.89 (m, 1H), 1.40-1.37 (m, 18H).

57-56

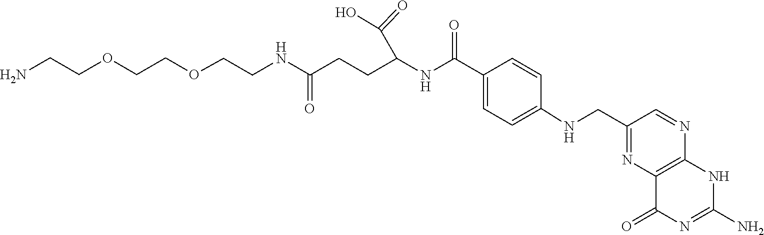 [SMILES: NCCOCCOCCNC(=O)CCC(NC(=O)c1ccc(NCc2cnc3[nH]c(N)nc(=O)c3n2)cc1)C(=O)O]

Dichloromethane was added to a flask containing the product 57-54 (7.61 g, 11.3276 mmol), and was ultrasonically oscillated until complete dissolution, then TFA (8.41 mL, 113.276 mmol) was added, and the resultant solution was stirred for reaction overnight at room temperature. After the reaction ended, n-hexane (100 mL) and methyl tertiary butyl ether (600 mL) were added for precipitation, the supernatant was discarded, then n-hexane and methyl tertiary butyl ether were added for precipitation, such procedure was repeated three times, suction filtration was performed, and a solid product was collected, to obtain 6.47 g of product, with a yield of 97.37%.

57-27

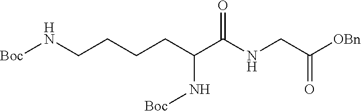 [SMILES: CC(C)(C)OC(=O)NCCCCC(NC(=O)OC(C)(C)C)C(=O)NCC(=O)OCc1ccccc1]

Boc-Lys(Boc)-OH·DCHA (3.9925 g, 7.5654 mmol, Aladdin), glycine benzyl ester hydrochloride (1.5256 g, 7.5654 mmol, Bide Pharmatech Co., Ltd.), HBTU (3.1560 g, 8.3219 mmol) and HOBT (1.1245 g, 8.3219 mmol) were weighted and added into a 250 mL flask, then an appropriate amount of DMF was added for dissolution, the reactant solution was kept at −5° C., and DIEA (2.8 mL, 16.64388 mmol) was slowly added dropwise, and then the reactant solution reacted for half an hour, then taken out and stirred for reaction overnight at room temperature. After the reaction ended, a saturated NaCl aqueous solution (200 mL) and ethyl acetate (200 mL) were added for extraction, the organic phase was collected after standing and stratification, the aqueous phase was rinsed with ethyl acetate (200 mL×3), the organic phases were combined and evaporated to dryness to obtain a solid, and the solid was collected, concentrated and dried in a vacuum oven, to obtain 3.9713 g of the product, which was overproduction.

57-31

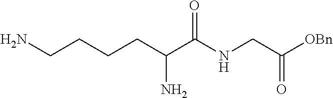 [SMILES: NCCCCC(N)C(=O)NCC(=O)OCc1ccccc1]

Dichloromethane was added to a flask containing the product 57-27 (3.7342 g, 7.5654 mmol), and was ultrasonically oscillated until complete dissolution, then TFA (5.6 mL, 75.654 mmol) was added, and the resultant solution was stirred for reaction overnight at room temperature. After the reaction ended, a saturated NaCl aqueous solution (200 mL) and ethyl acetate (160 mL) were added for extraction, the organic phase was collected after standing and stratification, the aqueous phase was rinsed with ethyl acetate (150 mL×3), the organic phases were combined and evaporated to dryness to obtain a solid, and the solid was collected, concentrated and dried in a vacuum oven, to obtain 2.4598 g of the product, which was overproduction.

57-41

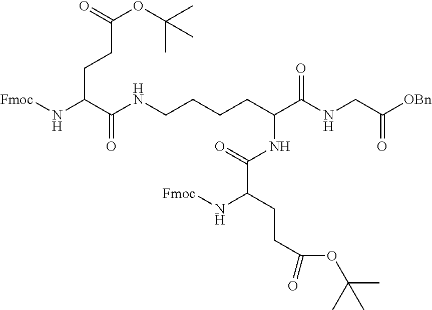

Fmoc-Glu(OtBu)-OH (7.0815 g, 16.6440 mmol, Ark Pharm), HBTU (8.6073 g, 22.6962 mmol) and HOBT (3.0667 g, 22.6962 mmol) were added to a flask containing the product 57-31 (2.2194 g, 7.5654 mmol), then an appropriate amount of DMF was added for dissolution, the reactant solution was kept at −5° C., and DIEA (15.0 mL, 90.7848 mmol) was slowly added dropwise, and then the reactant solution reacted for 1 hour, then taken out and stirred for reaction at room temperature. After the reaction ended, a saturated NaCl aqueous solution (300 mL) and ethyl acetate (200 mL) were added for extraction, the organic phase was collected after standing and stratification, the aqueous phase was rinsed with ethyl acetate (200 mL×3), the organic phases were combined and evaporated to dryness to obtain a solid, and the solid was collected, concentrated and dried in a vacuum oven, to obtain 8.6846 g of the product, which was overproduction.

57-42

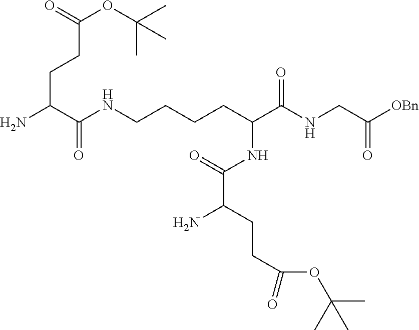 [SMILES: CC(C)(C)OC(=O)CCC(N)C(=O)NCCCCC(NC(=O)C(N)CCC(=O)OC(C)(C)C)C(=O)NCC(=O)OCc1ccccc1]

DMF was added to a flask containing the product 57-41 (8.3846 g, 7.5654 mmol), and was ultrasonically oscillated until complete dissolution, then morpholine (13.2 mL, 151.308 mmol) was added, and the resultant solution was stirred for reaction for 2 hours at room temperature. After the reaction ended, n-hexane (150 mL) and methyl tertiary butyl ether (70 mL) were added for precipitation, the supernatant was discarded, then n-hexane and methyl tertiary butyl ether were added for precipitation, and such procedure was repeated eight times, to obtain an oily solid; then methyl tertiary butyl ether (200 mL) were added for precipitation, to still obtain an oily solid; and the oily solid was evaporated to obtain a solid, and the solid was dried in a vacuum oven, to obtain 6.6653 g of the product, which was overproduction.

57-43

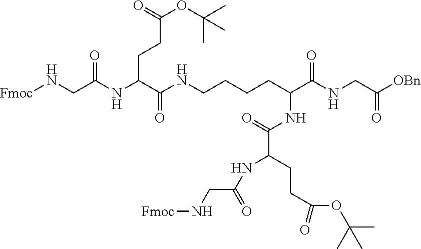

Fmoc-Gly (4.9484 g, 16.6439 mmol, Accela), HBTU (8.6073 g, 22.6962 mmol) and HOBT (3.0667 g, 22.6962 mmol) were added to a flask containing the product 57-42 (5.0219 g, 7.5654 mmol), then an appropriate amount of DMF was added for dissolution, the reactant solution was kept at −5° C., and DIEA (12.5 mL, 75.654 mmol) was slowly added dropwise, and then the reactant solution reacted for 1 hour, then taken out and stirred for reaction overnight at room temperature. After the reaction ended, a saturated NaCl aqueous solution (300 mL) and ethyl acetate (200 mL) were added for extraction, the organic phase was collected after standing and stratification, the aqueous phase was rinsed with ethyl acetate (200 mL×3), the organic phases were combined and evaporated to dryness to obtain a solid, and then the resultant solid product was dissolved in a mixed solvent of dichloromethane and methanol. The resultant product was dry loaded for column chromatography with elution using a mixed solution of 1% to 5% methanol in dichloromethane, and the desired component was collected, concentrated and dried in a vacuum oven to obtain 4.6 g of product, with a yield of 49.74%.

57-48

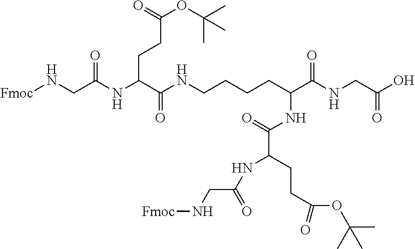

The product 57-43 (4.6 g, 3.7632 mmol) was added into a hydrogenation reaction kettle, then Pd/C catalyst (0.02 g) accounting for 10% was added, DMF (25 mL) was added, hydrogen gas was injected at a hydrogen gas pressure of 30 Psi, and the reactant solution was stirred overnight at room temperature. After the reaction ended, the reactant solution was filtered with celite, the filter cake was rinsed with DMF (15 mL×3), and the filtrate was added into a 250 mL round-bottomed flask and used as a raw material for the next step.

57-49

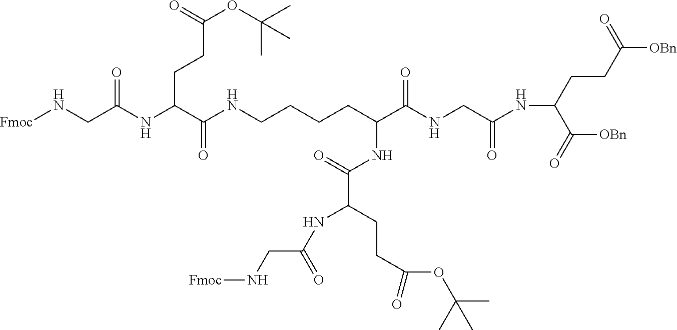

H-Glu(OBzl)-OBzl·TsOH (2.2560 g, 4.5158 mmol, Ark Pharm), HBTU (2.1407 g, 5.6448 mmol) and HOBT (0.7627 g, 5.6448 mmol) were added to a flask containing the product 57-48 (4.2609 g, 3.7632 mmol), then an appropriate amount of DMF was added for dissolution, the reactant solution was stirred at −5° C. for about 20 minutes, then DIEA (3.1 mL, 18.816 mmol) was slowly added dropwise, and the reactant solution further reacted at −5° C. for 1 hour, and then was stirred at room temperature. After the reaction ended, a saturated NaCl aqueous solution (300 mL) and ethyl acetate (200 mL) were added for extraction, the organic phase was collected after standing and stratification, the aqueous phase was rinsed with ethyl acetate (200 mL×3), the organic phases were combined and evaporated to dryness to obtain a solid, and then the resultant solid product was dissolved in a mixed solvent of dichloromethane and methanol. The resultant product was dry loaded for column chromatography with elution using a mixed solution of 1% to 4% methanol in dichloromethane, and the desired component was collected, concentrated and dried in a vacuum oven to obtain 5.1 g of product, with a yield of 94.0%.

¹H-NMR (400 MHz, DMSO-d₆) δ 8.22-8.17 (m, 2H), 8.05-8.04 (m, 1H), 8.00-7.95 (m, 1H), 7.90-7.88 (m, 4H), 7.72-7.70 (m, 3H), 7.58-7.53 (m, 1H), 7.43-7.39 (m, 4H), 7.35-7.31 (m, 11H), 7.27-7.23 (m, 3H), 7.18-7.13 (m, 5H), 5.12-5.07 (m, 3H), 4.42-4.18 (m, 9H), 3.80-3.59 (m, 7H), 3.01 (s, 2H), 2.44-2.41 (m, 2H), 2.30 (s, 5H), 2.20-2.15 (m, 4H), 2.07-2.05 (m, 1H), 1.91-1.88 (m, 3H), 1.77-1.70 (m, 3H), 1.36 (s, 18H).

57-188

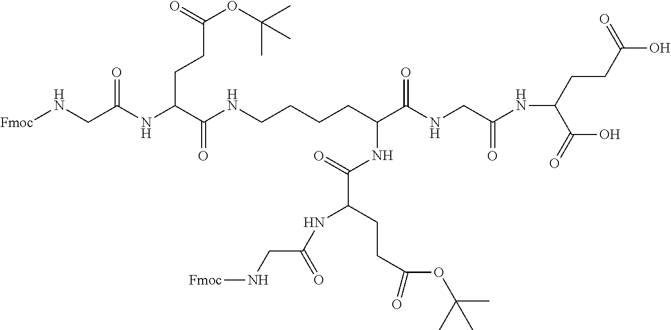

The product 57-49 (1.0 g, 0.6936 mmol) was added into a hydrogenation reaction kettle, then Pd/C (0.04 g) accounting for 10% was added, DMF (30 mL) was added, hydrogen gas was injected at a hydrogen gas pressure of 300 Psi, and the reactant solution was stirred overnight at room temperature. After the reaction ended, the reactant solution was filtered with celite, the filter cake was rinsed with DMF (15 mL×3), and the filtrate was added into a 250 mL round-bottomed flask and used as a raw material for the next step.

57-189

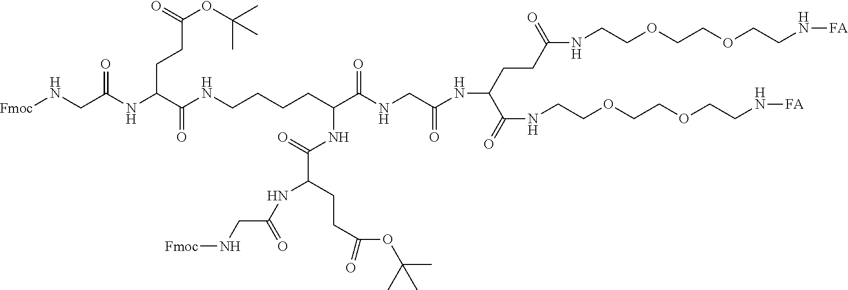

The product 57-56 (0.8722 g, 1.5259 mmol, Ark Pharm), HBTU (0.7891 g, 2.0808 mmol) and HOBT (0.2812 g, 2.0808 mmol) were added to a flask containing the product 57-188 (0.8749 g, 0.6936 mmol), then appropriate amounts of DMF and DMSO were added for dissolution, the reactant solution was stirred at −5° C. for about 20 minutes, then DIEA (1.1 mL, 6.936 mmol) was slowly added dropwise, and the reactant solution was stirred at room temperature. After the reaction ended, deionized water (200 mL) was added to obtain a solid precipitate, then suction filtration was performed to obtain a solid product, and the filter cake was rinsed with anhydrous ethanol (30 mL) three times and then rinsed with methyl tertiary butyl ether (30 mL) three times. Suction filtration was performed to obtain a solid product which was dried in the vacuum oven to obtain 1.7954 g of the product, which was overproduction.

57-190

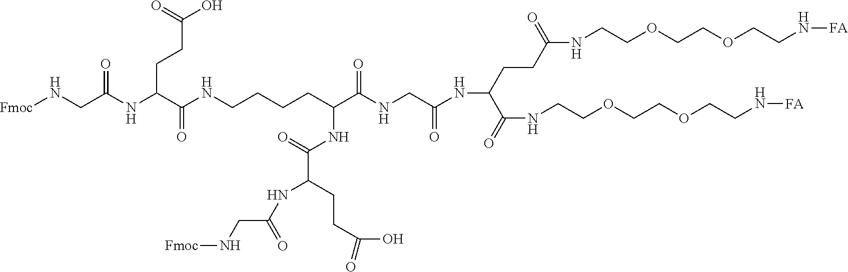

The product 57-189 (0.5 g, 0.2111 mmol) was added into a 250 mL round-bottomed flask, dichloromethane (10 mL) was added for dissolution, and TFA (0.3 mL, 4.222 mmol) was added during stirring, and finally the reaction flask was kept at room temperature and the reactant solution was stirred overnight. After the reaction ended, the reactant solution was first concentrated and evaporated to dryness to remove dichloromethane, then methyl tertiary butyl ether (100 mL) was added to the reactant solution for precipitation, to obtain a powder-like solid, filtration was performed, the filter cake was rinsed with methyl tertiary butyl ether (30 mL) three times, and finally, the filter cake was dried in the oven. 0.3957 g of the product was obtained with a yield of 83.05%.

57-191

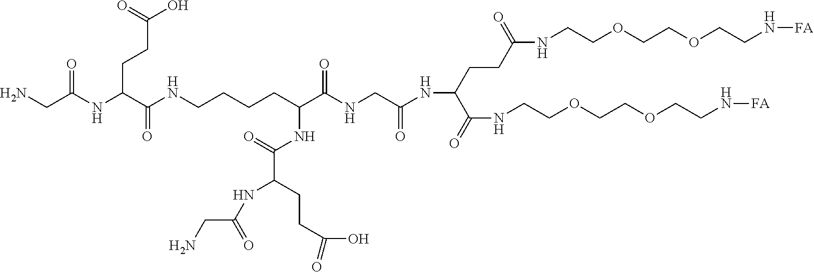

DMF was added to a flask containing the product 57-190 (0.3957 g, 0.1754 mmol), and was ultrasonically oscillated until complete dissolution, then morpholine (0.3 mL, 3.5075 mmol) was added, and the resultant solution was stirred for reaction overnight at room temperature. After the reaction ended, a solid in the reactant solution was filtered out, the filter cake was rinsed with methyl tertiary butyl ether (40 mL) four times and collected, a small amount of tetrahydrofuran was added, ultrasonication was performed, then toluene (2 mL) was added, the resultant mixture was subjected to ultrasonication and evaporated to dryness to obtain a solid, such procedure was repeated twice, and the solid was dried in a vacuum oven, to obtain 0.2847 g of the product, with a yield of 89.58%.

57-192

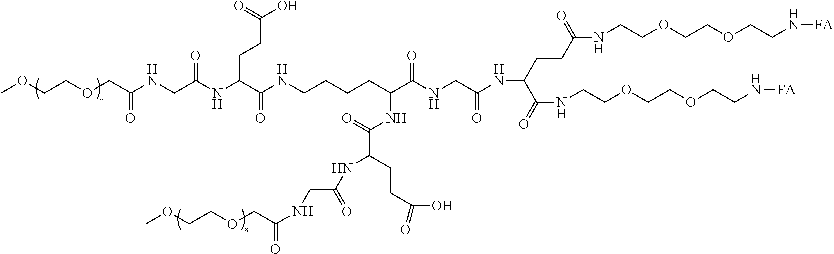

The product 57-191 (0.0789 g 0.0436 mmol) was added into a 250 mL flask, DMF (40 mL) was added for dissolution, the reactant solution was kept at −5° C. for reaction for 30 minutes before DIEA (0.2 mL, 1.09 mmol) was slowly added dropwise, then the resultant solution was further stirred at low temperature for 10 minutes before M-SCM-10K (0.1 g, 0.0436 mmol, JenKem) was added, and then the reactant solution was stirred for one week at a low speed at room temperature while protected from light. After the reaction ended, methyl tertiary butyl ether (200 mL) and n-hexane (70 mL) were added, to obtain a solid precipitate, then suction filtration was performed, and the filter cake was rinsed with methyl tertiary butyl ether (40 mL×3), and the obtained solid product was dried in the vacuum oven to obtain 0.9161 g of the product, with a yield of 93.57%.

68-65

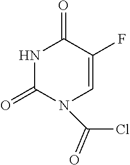 [SMILES: O=C(Cl)n1cc(F)c(=O)[nH]c1=O]

5-fluorouracil (1.0 g, 7.6877 mmol) was weighed and added into a reaction flask, dissolved in pyridine (5 mL) and stirred at 0° C. Triphosgene (1.141 g, 3.8439 mmol) was weighed and dissolved in tetrahydrofuran (4 mL), then the reactant solution was slowly added to the reaction flask dropwise, and then reactant solution was further stirred for reaction overnight at 0° C. After the reaction ended, a white solid precipitated in the reaction was filtered out to obtain a filtrate to be used as a raw material for the next reaction. 1.48 g of the product was obtained.

57-167

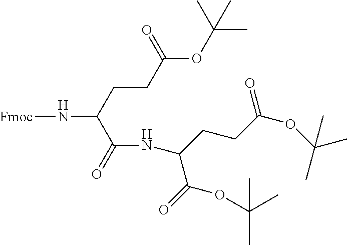

Fmoc-Glu(OtBu)-OH (7.6569 g, 17.9964 mmol, Ark Pharm), L-glutamic acid di-tert-butyl ester hydrochloride (5.3233 g, 17.9964 mmol, Innochem), HBTU (7.5074 g, 19.7960 mmol) and HOBT (2.6748 g, 19.7960 mmol) were added to a 500 mL flask, then an appropriate amount of DMF was added for dissolution, the reactant solution was kept at −5° C., and DIEA (6.5 mL, 39.5920 mmol) was slowly added dropwise, and then the reactant solution reacted for 1 hour, then taken out and stirred for reaction at room temperature. After the reaction ended, a saturated NaCl aqueous solution (250 mL) and ethyl acetate (200 mL) were added for extraction, the organic phase was collected after standing and stratification, the aqueous phase was rinsed with ethyl acetate (150 mL×3), the organic phases were combined and evaporated to dryness to obtain a solid, and the solid was collected, concentrated and dried in a vacuum oven, to obtain 12.6 g of the product, which was overproduction.

57-168

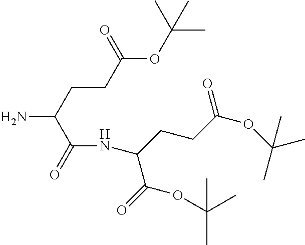 [SMILES: CC(C)(C)OC(=O)CCC(N)C(=O)NC(CCC(=O)OC(C)(C)C)C(=O)OC(C)(C)C]

DMF was added to a flask containing the product 57-167 (12.0 g, 17.9964 mmol), and was ultrasonically oscillated until complete dissolution, then morpholine (15.7 mL, 179.964 mmol) was added, and the resultant solution was stirred for reaction for 2 hours at room temperature. After the reaction ended, a saturated NaCl aqueous solution (250 mL) and ethyl acetate (200 mL) were added for extraction, the organic phase was collected after standing and stratification, the aqueous phase was rinsed with ethyl acetate (150 mL×3), the organic phases were combined and evaporated to dryness to obtain a solid, and the solid was collected, concentrated and dried in a vacuum oven, to obtain 8.7 g of the product, which was overproduction.

57-170

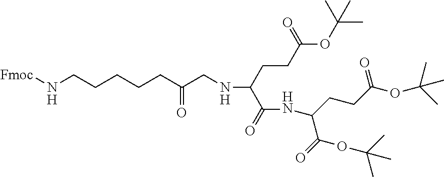

The product 49-17 (6.9961 g, 19.7960 mmol), HBTU (7.5074 g, 19.7960 mmol) and HOBT (2.6748 g, 19.7960 mmol) were added to a flask containing the product 57-168 (8.0 g, 17.9964 mmol), then an appropriate amount of DMF was added for dissolution, the reactant solution was stirred at −5° C. for about 20 minutes, then DIEA (6.5 mL, 39.5921 mmol) was slowly added dropwise, and the reactant solution further reacted at −5° C. for 1 hour, and then was stirred at room temperature. After the reaction ended, a saturated NaCl aqueous solution (250 mL) and ethyl acetate (200 mL) were added for extraction, the organic phase was collected after standing and stratification, the aqueous phase was rinsed with ethyl acetate (150 mL×3), the organic phases were combined and evaporated to dryness to obtain a solid, and then the resultant solid product was dissolved in a mixed solvent of dichloromethane and petroleum ether. The resultant product was dry loaded for column chromatography with elution using a mixed solution of 30% to 60% ethyl acetate in petroleum ether, and the desired component was collected, concentrated and dried in a vacuum oven to obtain 12.8 g of product, which was overproduction.

57-176

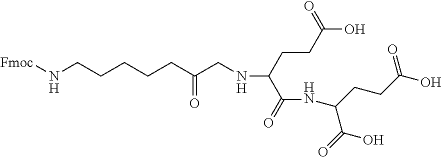

The product 57-170 (5.1 g, 6.5388 mmol) was added into a 250 mL round-bottomed flask, dichloromethane (30 mL) was added for dissolution, and TFA (14.6 mL, 196.164 mmol) was added during stirring, and finally the reaction flask was kept at room temperature and the reactant solution was stirred. After the reaction ended, the reactant solution was first concentrated and evaporated to dryness to remove dichloromethane, then methyl tertiary butyl ether (100 mL) was added to the reactant solution for precipitation, to obtain an oily solid, the supernatant was discarded, ethyl acetate (15 mL) was added, the oily solid was dissolved via ultrasonication, then n-hexane (30 mL) and methyl tertiary butyl ether (100 mL) were added for precipitation, to obtain a powder-like solid, suction filtration was performed, and the filter cake was collected, and finally, the filter cake was dried in the oven. 3.7646 g of the product was obtained with a yield of 94.13%.

57-177

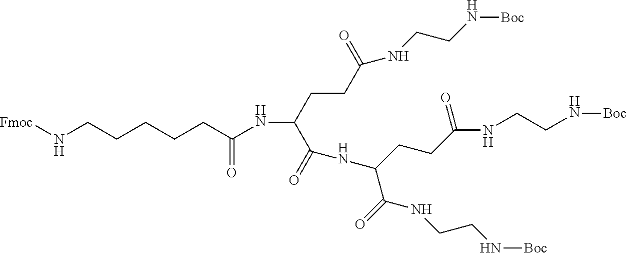

The product 57-176 (3.2152 g, 5.2567 mmol, Ark Pharm), Boc-ethylenediamine (2.7 mL, 17.3471 mmol, Aladdin), HBTU (8.9710 g, 23.6552 mmol) and HOBT (3.1963 g, 23.6552 mmol) were added to a 250 mL flask, then an appropriate amount of DMF was added for dissolution, the reactant solution was kept at −5° C., and DIEA (8.7 mL, 52.567 mmol) was slowly added dropwise, and then the reactant solution reacted for half an hour, then taken out and stirred for reaction at room temperature. After the reaction ended, n-hexane (150 mL) and methyl tertiary butyl ether (30 mL) were added for precipitation, the resultant mixture was kept at 2° C. to 8° C. for four hours, a solid precipitated, then suction filtration was performed, the filter cake was collected and dissolved in a mixed solution of methanol/dichloromethane, then n-hexane (150 mL) and methyl tertiary butyl ether (30 mL) were added for precipitation to obtain a powder-like solid, suction filtration was performed to obtain a solid product, and the solid product was dried in a vacuum oven to obtain 2.7 g of product, with a yield of 50%.

¹H-NMR (600 MHz, DMSO-d₆) δ 8.04-8.03 (m, 1H), 7.97-7.79 (m, 6H), 7.69-7.68 (m, 2H), 7.42-7.40 (m, 2H), 7.34-7.25 (m, 3H), 6.79 (s, 3H), 4.37-4.13 (m, 5H), 3.12-2.89 (m, 14H), 2.13-2.04 (m, 6H), 1.90-1.86 (m, 2H), 1.77-1.70 (m, 2H), 1.51-1.47 (m, 2H), 1.41-1.39 (m, 1H), 1.36 (s, 27H), 1.25-1.08 (m, 3H).

57-180

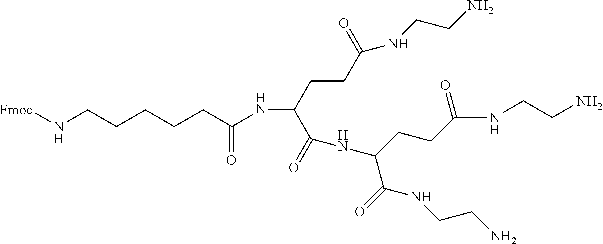

The product 57-177 (2.7 g, 2.6006 mmol) was added into a 250 mL round-bottomed flask, dichloromethane (25 mL) was added for dissolution, and TFA (5.8 mL, 78.0169 mmol) was added during stirring, and finally the reaction flask was kept at room temperature and the reactant solution was stirred. After the reaction ended, the reactant solution was first concentrated and evaporated to dryness to remove dichloromethane, then methyl tertiary butyl ether (100 mL) was added to the reactant solution for precipitation, to obtain a powder-like solid, suction filtration was performed, the filter cake was collected and dissolved in a mixed solution of methanol/dichloromethane, then methyl tertiary butyl ether (100 mL) were added for precipitation, to obtain a powder-like solid, suction filtration was performed, and the filter cake was collected, and finally, the filter cake was dried in the oven. 2.019 g of the product was obtained, which was overproduction.

57-182

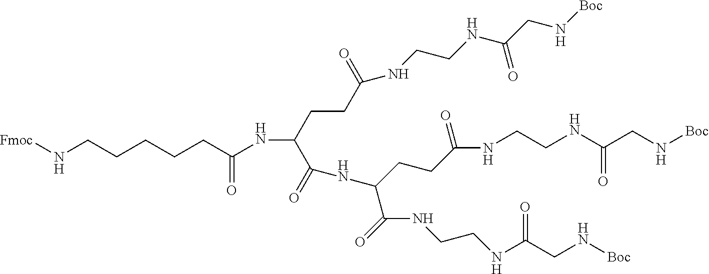

The product 57-180 (1.9190 g, 2.6006 mmol), Boc-glycine (1.5034 g, 8.5820 mmol, Aladdin), HBTU (4.4404 g, 11.7027 mmol) and HOBT (1.5821 g, 11.7027 mmol) were added to a 250 mL flask, then an appropriate amount of DMF was added for dissolution, the reactant solution was kept at −5° C., and DIEA (5.2 mL, 31.2072 mmol) was slowly added dropwise, and then the reactant solution reacted for half an hour, then taken out and stirred for reaction at room temperature. After the reaction ended, a saturated NaCl aqueous solution (250 mL) and ethyl acetate (200 mL) were added for extraction, the organic phase was collected after standing and stratification, the aqueous phase was rinsed with ethyl acetate (150 mL×3), the organic phases were combined and evaporated to dryness to obtain a solid, then methyl tertiary butyl ether (200 mL) was added for precipitation, to obtain a glue-like solid, suction filtration was performed, the filter cake was collected and dissolved in a mixed solution of methanol/dichloromethane, then methyl tertiary butyl ether (200 mL) were added for precipitation, suction filtration was performed to obtain a solid product, and the solid product was dried in a vacuum oven, to obtain 2.1 g of the product, with a yield of 65.26%. [M+Na⁺] 1231.059

57-184

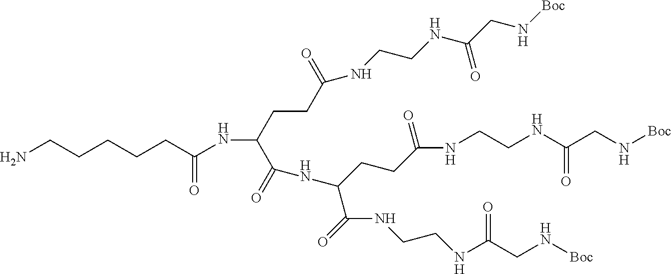 [SMILES: CC(C)(C)OC(=O)NCC(=O)NCCNC(=O)CCC(NC(=O)CCCCCN)C(=O)NC(CCC(=O)NCCNC(=O)CNC(=O)OC(C)(C)C)C(=O)NCCNC(=O)CNC(=O)OC(C)(C)C]

DMF was added to a flask containing the product 57-182 (2.1 g, 1.7364 mmol), and was ultrasonically oscillated until complete dissolution, then morpholine (1.5 mL, 17.364 mmol) was added, and the resultant solution was stirred for reaction for 2 hours at room temperature. After the reaction ended, methyl tertiary butyl ether (200 mL) was added for precipitation, to obtain a glue-like solid, suction filtration was performed, the filter cake was collected and dissolved in ethyl acetate, then methyl tertiary butyl ether (200 mL) were added for precipitation, suction filtration was performed to obtain a solid product, and the solid product was dried in a vacuum oven, to obtain 1.6 g of the product, with a yield of 94.12%.

57-186

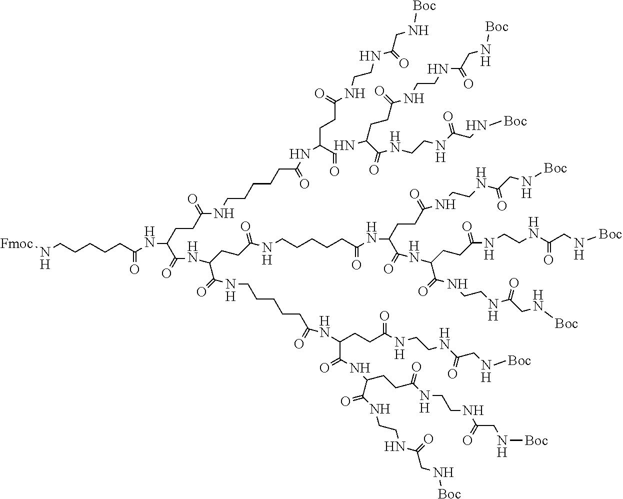

The products 57-184 (1.55 g, 1.5702 mmol) and 57-176 (0.2910 g, 0.4758 mmol), HBTU (0.8120 g, 2.1411 mmol) and HOBT (0.2893 g, 2.1411 mmol) were added to a 250 mL flask, then an appropriate amount of DMF was added for dissolution, the reactant solution was kept at −5° C., and DIEA (0.9 mL, 5.7096 mmol) was slowly added dropwise, and then the reactant solution reacted for half an hour, then taken out and stirred for reaction at room temperature. After the reaction ended, a solid precipitated, filtration was performed, the filter cake was rinsed with methyl tertiary butyl ether (30 mL), collected and dissolved in a mixed solution of methanol/dichloromethane, then methyl tertiary butyl ether (200 mL) were added for precipitation to form a glue-like solid, suction filtration was performed, and the filter cake was collected and dried in a vacuum oven, to obtain 1.3 g of the product, with a yield of 77.64%.

¹H-NMR (600 MHz, DMSO-d₆) δ 8.05-8.0 (m, 7H), 7.95-7.92 (m, 5H), 7.89-7.80 (m, 20H), 7.68-7.67 (m, 2H), 7.42-7.40 (m, 2H), 7.34-7.26 (m, 3H), 6.91-6.89 (m, 8H), 4.29-4.28 (m, 2H), 4.21-4.11 (m, 9H), 3.50-3.49 (m, 18H), 3.09-3.08 (m, 30H), 2.99-2.96 (m, 10H), 2.89 (s, 4H), 2.11-2.05 (m, 24H), 1.91, −1.86 (m, 8H), 1.76-1.72 (m, 8H), 1.47 (s, 8H), 1.37 (s, 81H), 1.11 (s, 16H).

57-193

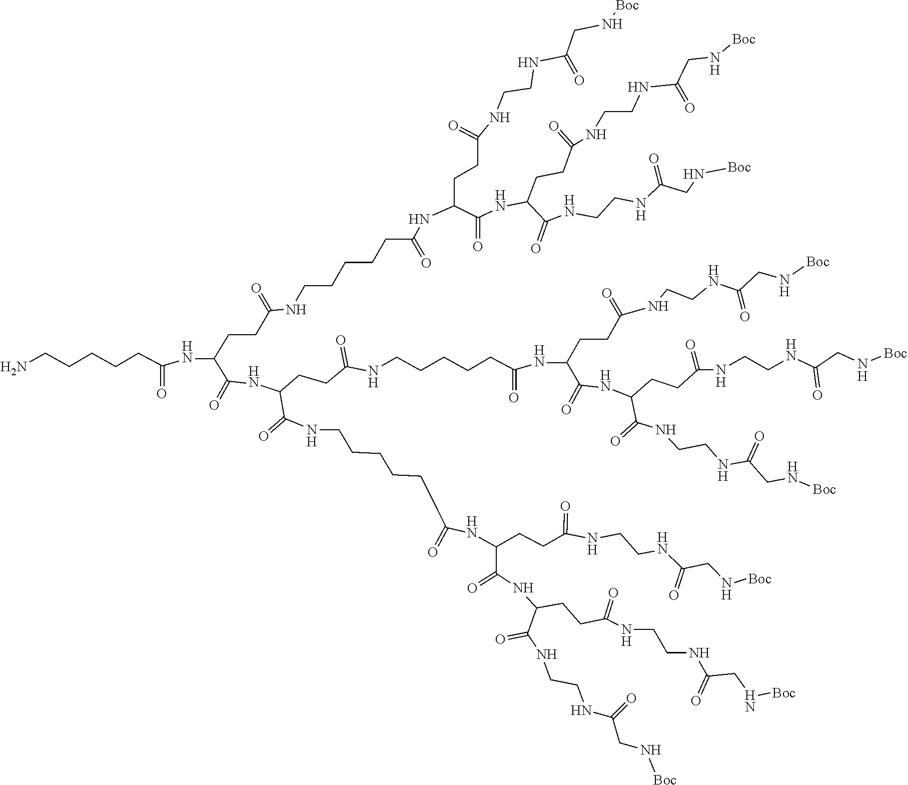 [SMILES: CC(C)(C)OC(=O)NCC(=O)NCCNC(=O)CCC(NC(=O)CCCCCNC(=O)CCC(NC(=O)CCCCCN)C(=O)NC(CCC(=O)NCCCCCC(=O)NC(CCC(=O)NCCNC(=O)CNC(=O)OC(C)(C)C)C(=O)NC(CCC(=O)NCCNC(=O)CNC(=O)OC(C)(C)C)C(=O)NCCNC(=O)CNC(=O)OC(C)(C)C)C(=O)NCCCCCC(=O)NC(CCC(=O)NCCNC(=O)CNC(=O)OC(C)(C)C)C(=O)NC(CCC(=O)NCCNC(=O)CNC(=O)OC(C)(C)C)C(=O)NCCNC(=O)CNC(=O)OC(C)(C)C)C(=O)NC(CCC(=O)NCCNC(=O)CNC(=O)OC(C)(C)C)C(=O)NCCNC(=O)CNC(=O)OC(C)(C)C]

DMF was added to a flask containing the product 57-186 (1.3 g, 0.3694 mmol), and was ultrasonically oscillated until complete dissolution, then morpholine (0.3 mL, 3.694 mmol) was added, and the reactant solution was stirred for reaction in a bath at 60° C. After the reaction ended, methyl tertiary butyl ether (200 mL) was added for precipitation, to obtain a powder-like solid, suction filtration was performed, the filter cake was rinsed with methyl tertiary butyl ether (30 mL) three times, suction filtration was performed to obtain a solid product, and the solid product was dried in a vacuum oven, to obtain 1.1 g of the product, with a yield of 90.32%. [M+Na⁺] 2419.055

57-198

The products 57-192 (0.9161 g, 0.0408 mmol) and 57-193 (0.261 g, 0.0898 mmol), HBTU (0.0464 g, 0.1224 mmol) and HOBT (0.0165 g, 0.1224 mmol) were added to a 250 mL flask, then an appropriate amount of DMF was added for dissolution, the reactant solution was kept at −5° C., and DIEA (0.1 mL, 0.6051 mmol) was slowly added dropwise, and then the reactant solution reacted for half an hour, then taken out and stirred for reaction at room temperature. After the reaction ended, n-hexane (50 mL) and methyl tertiary butyl ether (150 mL) were added for precipitation, to obtain a powder-like solid, suction filtration was performed, the filter cake was collected and dissolved in a mixed solution of methanol/dichloromethane, then n-hexane (100 mL) and methyl tertiary butyl ether (100 mL) were added for precipitation to obtain a powder-like solid, suction filtration was performed, the filter cake was rinsed with methyl tertiary butyl ether (40 mL) three times, to obtain a solid product, and the solid product was dried in a vacuum oven to obtain 0.7777 g of product, with a yield of 65.7%.

57-205

The product 57-198 (0.1 g, 0.0034 mmol) was added into a 100 mL round-bottomed flask, dichloromethane (5 mL) was added for dissolution, and TFA (1 mL, 1.224 mmol) was added during stirring, and finally the reaction flask was kept at room temperature and the reactant solution was stirred. After the reaction ended, the reactant solution was first concentrated and evaporated to dryness to remove dichloromethane, then methyl tertiary butyl ether (50 mL) was added to the reactant solution for precipitation, to obtain a powder-like solid, suction filtration was performed, the filter cake was collected and dissolved in a mixed solution of methanol/dichloromethane, then methyl tertiary butyl ether (50 mL) were added for precipitation, to obtain a powder-like solid, suction filtration was performed, and the filter cake was collected, and finally, the filter cake was dried in the oven. 0.1005 g of the product was obtained, which was overproduction.

A solution of product 57-205 (0.0925 g, 0.0034 mmol) in DMF was added to a solution of product 68-65 (0.0740 g, 0.3844 mmol) in pyridine (5 mL) and tetrahydrofuran (6 mL) and the reactant solution was stirred for reaction at room temperature. After the reaction ended, the reactant solution was first concentrated and evaporated to dryness to remove the liquid in the reactant solution, n-hexane (25 mL) and methyl tertiary butyl ether (30 mL) were added for precipitation, the supernatant was discarded, then n-hexane and methyl tertiary butyl ether were added for precipitation, such procedure was repeated three times, suction filtration was performed, and a solid product was obtained and dried in a vacuum oven to obtain 0.1395 g of the product.

¹H-NMR (600 MHz, DMSO-d₆) δ 8.10 (s, 62H), 7.27 (s, 6H), 7.18 (s, 6H), 7.10 (s, 6H), 4.13 (s, 10H), 4.02 (s, 4H), 3.94 (s, 4H), 3.51 (s, 1874H), 3.17 (s, 93H), 2.16-2.13 (m, 183H), 1.49 (s, 29H), 1.40-1.39 (m, 46H), 1.24-1.23 (m, 17H).

### Synthesis of Compound 68-67

68-12

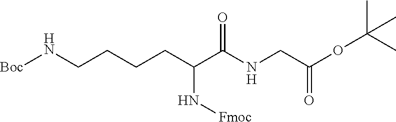

Fmoc-Lys(Boc)-OH (5 g, 10.67146 mmol, Accela), glycine tert-butyl ester hydrochloride (1.88 g, 11.2050 mmol, Accela), HBTU (6.07 g, 16.0072 mmol) and HOBT (2.16 g, 16.0072 mmol) were added to a 500 mL flask, the reactant solution was kept at −5° C. and stirred for about 20 minutes before DIEA (7.93 mL, 48.0215 mmol) was slowly added dropwise. The resultant solution further reacted at −5° C. for 1 hour and then stirred overnight at room temperature. After the reaction ended, the reactant solution was transferred to a 1 L separatory funnel, a NaCl solution (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 7.3 g of the product.

68-14

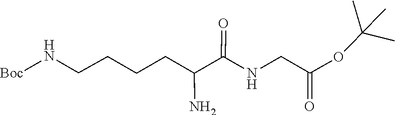 [SMILES: CC(C)(C)OC(=O)CNC(=O)C(N)CCCCNC(=O)OC(C)(C)C]

DMF was added to a flask containing the product 68-12 (10.6714 mmol), and was ultrasonically oscillated until complete dissolution, then morpholine (9.29 mL, 106.714 mmol) was added, and the resultant solution was stirred for reaction for 2 hours at room temperature. After the reaction ended, a saturated NaCl solution (200 mL) and EA (200 mL) were added for extraction, the organic phase was collected after standing and stratification, the aqueous phase was rinsed with EA (200 mL×3), the organic phases were combined and evaporated to dryness to obtain a solid, and the solid was dried in a vacuum oven, to obtain 9.8 g of the product, which was overproduction.

68-16

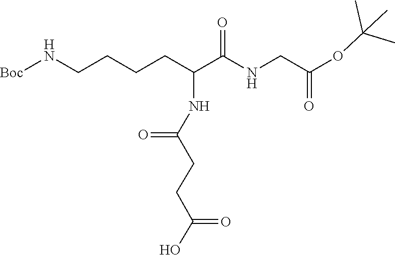 [SMILES: CC(C)(C)OC(=O)CNC(=O)C(CCCCNC(=O)OC(C)(C)C)NC(=O)CCC(=O)O]

The product 68-14 (10.6714 mmol) was added to a flask filled with succinic anhydride (3.2 g, 32.0142 mmol), then an appropriate amount of DMF was added for dissolution, the reactant solution was kept at −5° C., and DIEA (8.82 mL, 53.357 mmol) was slowly added dropwise, and then the reactant solution reacted for half an hour, then taken out and stirred for reaction overnight at room temperature. After the reaction ended, a saturated NaCl solution (200 mL) was added, EA (200 mL) was added for extraction, the organic phase was collected after standing and stratification, the aqueous phase was rinsed with EA (200 mL×3), the organic phases were combined and evaporated to dryness to obtain a solid, and then the resultant solid product was dissolved in a mixed solvent of dichloromethane and methanol. The resultant product was dry loaded for column chromatography with elution using a mixed solution of 3% to 6% methanol in dichloromethane, and the desired component was collected, concentrated and dried in a vacuum oven to obtain 2.4 g of product, with a yield of 48.97%.

68-17

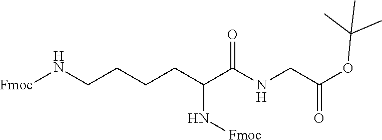

Fmoc-Lys(Fmoc)-OH (7.51 g, 10.6714 mmol, Accela), glycine tert-butyl ester hydrochloride (1.88 g, 11.2050 mmol, Accela), HBTU (6.07 g, 16.0072 mmol) and HOBT (2.16 g, 16.0072 mmol) were added to a 500 mL flask, the reactant solution was kept at −5° C. and stirred for about 20 minutes before DIEA (7.93 mL, 48.0215 mmol) was slowly added dropwise. The resultant solution further reacted at −5° C. for 1 hour and then stirred overnight at room temperature. After the reaction ended, the reactant solution was transferred to a 1 L separatory funnel, a NaCl solution (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 9.3 g of the product, which was overproduction.

68-18

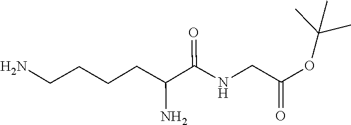 [SMILES: CC(C)(C)OC(=O)CNC(=O)C(N)CCCCN]

DMF was added to a flask containing the product 68-17 (10.6714 mmol), and was ultrasonically oscillated until complete dissolution, then morpholine (13.32 mL, 152.886 mmol) was added, and the resultant solution was stirred for reaction for 2 hours at room temperature. After the reaction ended, a saturated NaCl solution (200 mL) and EA (200 mL) were added for extraction, the organic phase was collected after standing and stratification, the aqueous phase was rinsed with EA (200 mL×3), the organic phases were combined and evaporated to dryness to obtain a solid, and the solid was dried in a vacuum oven, to obtain 8.5 g of the product, which was overproduction.

68-21

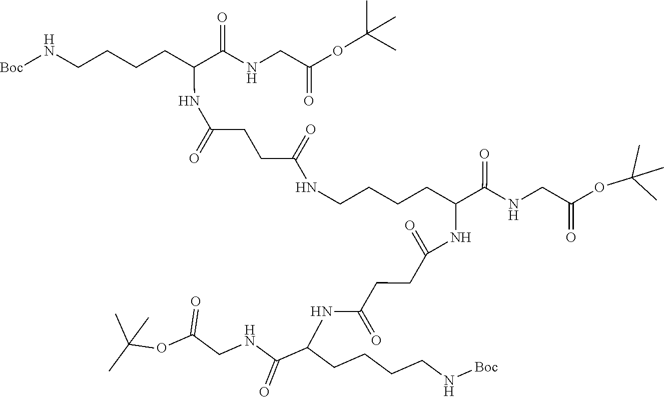 [SMILES: CC(C)(C)OC(=O)CNC(=O)C(CCCCNC(=O)OC(C)(C)C)NC(=O)CCC(=O)NCCCCC(NC(=O)CCC(=O)NC(CCCCNC(=O)OC(C)(C)C)C(=O)NCC(=O)OC(C)(C)C)C(=O)NCC(=O)OC(C)(C)C]

The product 68-16 (2.4 g, 5.2227 mmol), the product 68-18 (0.58 g, 2.3740 mmol), HBTU (2.7 g, 7.1219 mmol) and HOBT (0.96 g, 7.1219 mmol) were added to a 500 mL flask, the reactant solution was kept at −5° C. and stirred for about 20 minutes before DIEA (3.53 mL, 21.3657 mmol) was slowly added dropwise. The resultant solution further reacted at −5° C. for 1 hour and then stirred overnight at room temperature. After the reaction ended, the reactant solution was transferred to a 1 L separatory funnel, a NaCl solution (200 mL) and ethyl acetate (200 mL) were added for extraction to obtain an organic phase, the aqueous phase was rinsed with ethyl acetate (200 mL×2), the organic phases were combined and rinsed with saturated brine (200 mL×2), and the organic phases were combined and rinsed with a saturated sodium chloride solution (100 mL×2) and evaporated to dryness, to obtain 0.7 g of the product, with a yield of 25%.

¹H-NMR (600 MHz, DMSO-d₆) δ 8.25-8.18 (m, 3H), 8.07-7.98 (m, 3H), 7.85-7.78 (m, 1H), 6.78-6.72 (m, 2H), 4.25-4.13 (m, 3H), 3.76-3.61 (m, 6H), 3.02-2.96 (m, 2H), 2.92-2.83 (m, 4H), 2.41-2.27 (m, 8H), 1.65 (s, 3H), 1.53-1.43 (m, 3H), 1.42-1.19 (m, 58H); [M+Na⁺] 1164.674, [M+K⁺] 1180.652.

68-27

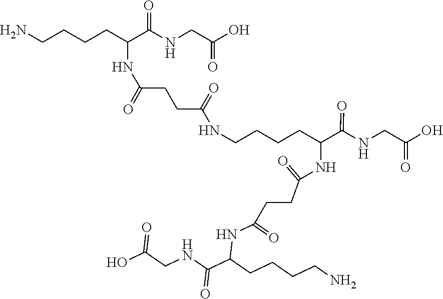 [SMILES: NCCCCC(NC(=O)CCC(=O)NCCCCC(NC(=O)CCC(=O)NC(CCCCN)C(=O)NCC(=O)O)C(=O)NCC(=O)O)C(=O)NCC(=O)O]

Dichloromethane was added to a flask containing the product 68-21 (0.2 g, 0.1750 mmol), and was ultrasonically oscillated until complete dissolution, then TFA (0.65 mL, 8.7536 mmol) was added, and the resultant solution was stirred for reaction overnight at room temperature. After the reaction ended, the reactant solution was evaporated with a rotary evaporator to obtain an oily substance, then methyl tertiary butyl ether (60 mL) was added, a powder-like solid precipitated in the reactant solution, suction filtration was performed, and the resultant filter cake was rinsed with methyl tertiary butyl ether (40 mL×3), collected and dried in the vacuum oven to obtain 0.0824 g of the product, with a yield of 60.86%.

68-32

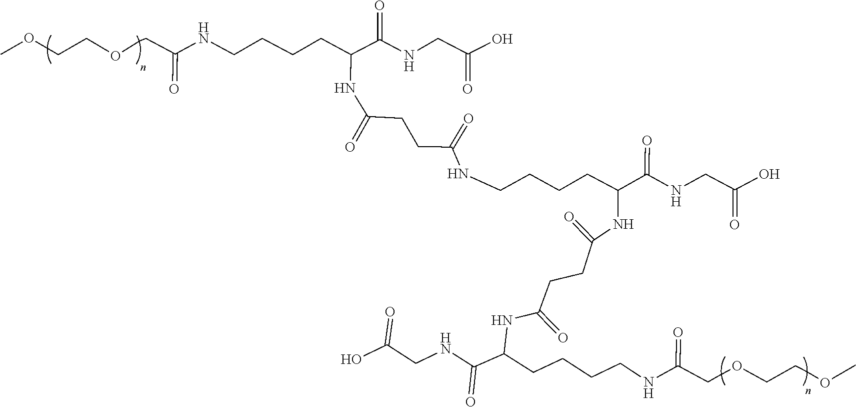

The product 68-27 (0.08 g 0.1034 mmol) was added into a 250 mL flask, DMF (20 mL) was added for dissolution, DIEA (0.38 mL, 2.3260 mmol) and a solution of M-SCM-10K (2.37 g, 0.2274 mmol, JenKem) in DMF were added dropwise slowly, and the reactant solution was stirred for reaction for one week at a low speed at room temperature while protected from light. After the reaction ended, n-hexane (25 mL) and methyl tertiary butyl ether (200 mL) were added for precipitation, the supernatant was discarded, then n-hexane and methyl tertiary butyl ether were added for precipitation, such procedure was repeated three times, suction filtration was performed, and a solid product was obtained, collected and dried in a vacuum oven to obtain 3.2 g of the product, which was overproduction.

68-48

The product 68-32 (0.6 g, 0.0280 mmol), the product 57-193 (0.3 g, 0.0924 mmol), HBTU (0.035 g, 0.0924 mmol) and HOBT (0.012 g, 0.0924 mmol) were added to a 500 mL flask, the reactant solution was kept at −5° C. and stirred for about 20 minutes before DIEA (0.03 mL, 0.1849 mmol) was slowly added dropwise. The resultant solution further reacted at −5° C. for 1 hour and then stirred overnight at room temperature. After the reaction ended, n-hexane (25 mL) and methyl tertiary butyl ether (200 mL) were added for precipitation, the supernatant was discarded, then n-hexane and methyl tertiary butyl ether were added for precipitation, such procedure was repeated three times, suction filtration was performed, to obtain a solid product, the obtained solid product was dissolved in dichloromethane and methanol, and the resultant mixture was dry loaded for column chromatography with elution using 10% to 12% methanol/dichloromethane, and the desired component was collected, concentrated and dried, to obtain 0.51 g of the product, with a yield of 58.28%.

68-60

Dichloromethane was added to a flask containing the product 68-48 (0.2 g, 0.0064 mmol), and was ultrasonically oscillated until complete dissolution, then TEA (3 mL) was added, and the resultant solution was stirred for reaction overnight at room temperature. After the reaction ended, the reactant solution was evaporated with a rotary evaporator to obtain an oily substance, then methyl tertiary butyl ether (60 mL) was added, a powder-like solid precipitated in the reactant solution, suction filtration was performed, the resultant filter cake was rinsed with methyl tertiary butyl ether (40 mL×3), the product had poor solubility, and the filter cake was collected and dried in the vacuum oven to obtain 0.28 g of the product, which was overproduction.

68-67

DMF (10 mL) was added to a flask containing the product 68-60 (0.28 g, 0.0094 mmol), and was ultrasonically oscillated until complete dissolution, a solution of product 68-65 (0.44 g, 2.3063 mmol) was added slowly dropwise at 000, and after dropwise addition, the reactant solution reacted for 2 hours, then taken out, and was stirred for reaction overnight at room temperature. After the reaction ended, the reactant solution was evaporated with a rotary evaporator to obtain an oily substance, then methyl tertiary butyl ether (60 mL) was added, a powder-like solid precipitated in the reactant solution, suction filtration was performed, the resultant filter cake was rinsed with methyl tertiary butyl ether (40 mL×3), the product had poor solubility, and the filter cake was collected and dried in the vacuum oven to obtain 0.3 g of the product.

¹H-NMR (600 MHz, DMSO-d₆) δ 8.41-7.60 (m, 132H), 7.48-7.03 (m, 23H), 6.90-6.33 (m, 6H), 4.25-4.12 (m, 54H), 3.90-3.82 (m, 57H), 3.51 (s, 4680H), 3.15-3.03 (m, 43H), 2.15-2.03 (m, 53H), 1.80-1.63 (m, 36H), 1.55-1.45 (m, 46H), 1.41-1.32 (m, 74H), 1.29-1.18 (m, 163H), 0.91-0.80 (m, 50H).

### Synthesis of Compound 41-228

41-228

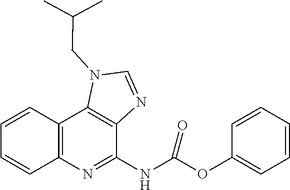 [SMILES: CC(C)Cn1cnc2c(NC(=O)Oc3ccccc3)nc3ccccc3c21]

Imiquimod (6.0 g, 24.968 mmol, Macklin) was weighed and added into a 250 mL round-bottomed flask, then CH₂Cl₂ (40 mL) was added for dissolution, triethylamine (13.5 mL, 99.87 mmol) was added, the flask was kept at 0° C. and the reactant solution was stirred for 30 minutes, then phenyl chloroformate (6.2 mL, 49.936 mmol) was slowly added dropwise, and after dropwise addition, the reactant solution was stirred overnight. After the reaction ended, n-hexane (100 mL) and methyl tertiary butyl ether (400 mL) were added for precipitation, such procedure was repeated three times, and the resultant product was filtered, to obtain 5.76 g of the product.

41-221

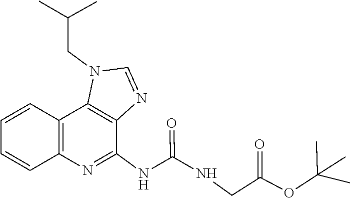 [SMILES: CC(C)Cn1cnc2c(NC(=O)NCC(=O)OC(C)(C)C)nc3ccccc3c21]

The product 41-218 (5.76 g, 15.98 mmol) and Gly-OtBu (2.7 g, 15.98 mmol, Accela) were weighed and added into a 500 mL round-bottomed flask, then DMF (30 mL) was added for dissolution, DIEA (5.27 mL, 31.96 mmol) was added, the reaction flask was subjected to oil bath at 100° C., and the reactant solution was stirred overnight. After the reaction, the resultant product was rinsed with pure water, extracted with EA three times, and the organic phase was evaporated and dried. The resultant product was dry loaded for column chromatography with elution using 2% to 3% methanol in dichloromethane, and the desired component was collected and concentrated, to obtain 4.9 g of the product.

41-224

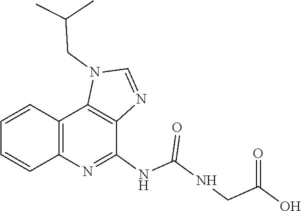 [SMILES: CC(C)Cn1cnc2c(NC(=O)NCC(=O)O)nc3ccccc3c21]

The product 41-221 (4.9 g, 12.3280 mmol) was added to a 500 mL round-bottomed flask, and dissolved in dichloromethane (30 mL), and then TFA (23 mL, 243.56 mmol) was added, and the reactant solution was stirred for reaction at the room temperature overnight. After the reaction ended, the reactant solution was concentrated under reduced pressure, n-hexane (100 mL) and ethyl acetate (400 mL) were added for precipitation, such procedure was repeated three times, and the resultant product was filtered and dried, to obtain 3.0 g of the product.

45-57

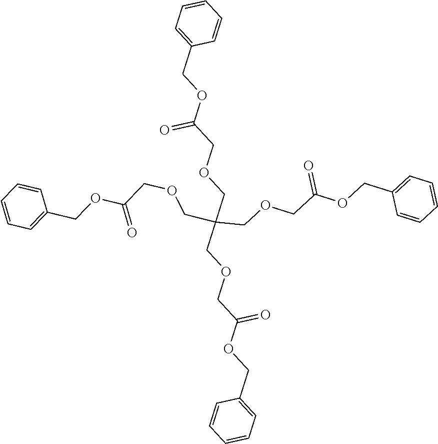 [SMILES: O=C(COCC(COCC(=O)OCc1ccccc1)(COCC(=O)OCc1ccccc1)COCC(=O)OCc1ccccc1)OCc1ccccc1]

Pentaerythritol (10 g, 73.4484 mmol) was added into a 500 mL two-neck reaction flask, tetrahydrofuran (20 mL) was added, and the resultant mixture was uniformly mixed via ultrasonication and stirred at 0° C. Nitrogen was injected for protection, potassium tert-butoxide (352 mL, 352.55 mmol) was added, the reactant solution was stirred at 37° C. for 2 hours, then benzyl bromoacetate (55 mL, 352.55 mmol) was added, and the reactant solution was further stirred for 3 hours, and then reacted at room temperature. After the reaction ended, pure water and ethyl acetate were added for extraction of the reactant solution, the organic phase was concentrated and dry loaded for column chromatography. Gradient elution was performed using 1% to 2% ethyl acetate/petroleum ether, to obtain 15 g of the product, with a yield of 28%.

41-189

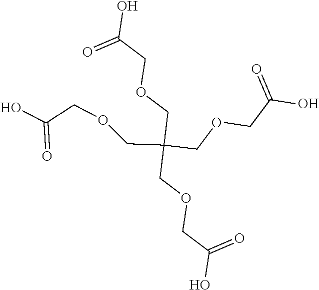 [SMILES: O=C(O)COCC(COCC(=O)O)(COCC(=O)O)COCC(=O)O]

The product 45-57 (0.35 mg, 0.4802 mmol) and 10% Pd/C catalyst (40 mg) were added into a hydrogenation reaction kettle, DMF (15 mL) was slowly added, the resultant mixture was slowly stirred and dissolved, hydrogen gas (20 psi) was injected, and the reactant solution was stirred overnight at room temperature. Next day, the reactant solution was subjected to suction filtration with a sand core funnel filled with celite to remove Pd/C and obtain a solution of the product 41-189 in DMF to be used directly in the next reaction.

45-114

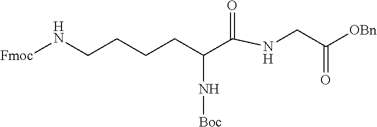

Boc-L-Lys(Fmoc)-OH (20 g, 42.6857 mmol, Aladdin), Gly-OBn (14.4 g, 42.6857 mmol, Ark Pharm), HBTU (24 g, 64.0286 mmol) and HOBT (8.65 g, 64.0286 mmol) were weighed and added into a 500 mL flask, DMF (40 mL) was added for dissolution, and the reactant solution was stirred at −5° C. for 30 minutes. Then DIEA (31.7 mL, 192.085 mmol) was slowly added dropwise. The reactant solution reacted at −5° C. overnight. After the reaction ended, pure water and ethyl acetate were added for extraction of the reactant solution. The organic phase was rinsed with saturated brine, concentrated and evaporated to dryness, to obtain 26 g of the product.

45-115

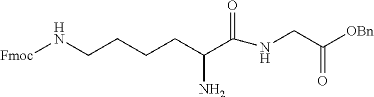

The compound 45-114 (26 g, 42.6857 mmol) was weighed, dichloromethane (20 mL) and TFA (14 mL, 190.2855 mmol) were added, the product was ultrasonically dissolved, and the reactant solution was stirred for reaction. After the reaction ended, the reactant solution was concentrated and then dissolved in ethyl acetate (100 mL), a saturated brine solution was added, the organic phase was separated, the aqueous phase was extracted with ethyl acetate (50 mL×3) three times until the aqueous phase contained no product, and the organic phases were combined, and further rinsed with saturated brine (50 mL) once, concentrated and evaporated to dryness to obtain 22 g of the product.

45-124

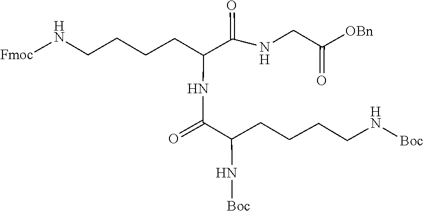

Boc-L-Lys(Boc)-OH (7.3 g, 21.334 mmol, Ark Pharm), the product 45-115 (11 g, 21.334 mmol), HBTU (12.1363 g, 32.0016 mmol) and HOBT (4.3241 g, 32.0016 mmol) were weighed and added into a 500 mL flask, DMF (80 mL) was added for dissolution, and the reactant solution was stirred at −5° C. for 30 minutes. Then DIEA (15 mL, 96.003 mmol) was slowly added dropwise. The reactant solution reacted at −5° C. overnight. After the reaction ended, pure water and ethyl acetate were added for extraction of the reactant solution. The organic phase was rinsed with saturated brine, concentrated and dry loaded for column chromatography with gradient elution using 50% to 100% ethyl acetate/petroleum ether, to obtain 15 g of the product, with a yield of 83%.

45-126

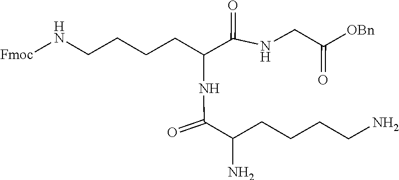

The compound 45-124 (15 g, 17.7725 mmol) was weighed, dichloromethane (30 mL) and TFA (39 mL, 533.1754 mmol) were added, the product was ultrasonically dissolved, and the reactant solution was stirred for reaction. After the reaction ended, the reactant solution was concentrated and then dissolved in ethyl acetate (100 mL), a saturated brine solution was added, the reactant solution was oscillated to separate an organic phase, the aqueous phase was extracted with ethyl acetate (150 mL×3) three times until the aqueous phase contained no product, and the organic phases were combined, and further rinsed with saturated brine (150 mL) once, concentrated and evaporated to dryness to obtain 11.44 g of the product.

45-127

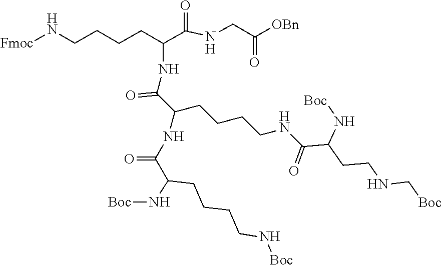

Boc-L-Lys(Boc)-OH (13.022 g, 37.5911 mmol), product 45-126 (11.4 g, 17.0868 mmol), HBTU (19.5538 g, 51.5604 mmol) and HOBT (6.9668 g, 51.5604 mmol) were weighed and added into a 500 mL flask, DMF (80 mL) was added for dissolution, and the reactant solution was stirred at −5° C. for 30 minutes. Then DIEA (25.4 mL, 153.7812 mmol) was slowly added dropwise. The reactant solution reacted at −5° C. overnight. After the reaction ended, pure water and ethyl acetate were added for extraction of the reactant solution. The organic phase was rinsed with saturated brine, concentrated and dry loaded for column chromatography with gradient elution using 1% to 55% methanol/dichloromethane, to obtain 18 g of the product, with a yield of 81%.

41-188

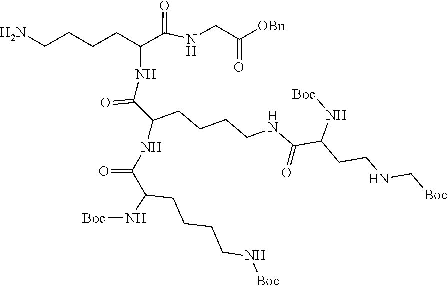 [SMILES: CC(C)(C)OC(=O)CNCCC(NC(=O)OC(C)(C)C)C(=O)NCCCCC(NC(=O)C(CCCCNC(=O)OC(C)(C)C)NC(=O)OC(C)(C)C)C(=O)NC(CCCCN)C(=O)NCC(=O)OCc1ccccc1]

The reactant 45-127 (3.0 g, 2.3067 mmol) was added to a 250 mL flask, a DMF solution (50 mL) was added, morpholine (4.0191 mL, 46.1333 mmol) was added, the reactant solution was stirred for reaction at room temperature, and after 3 hours, the reaction ended. Saturated brine (150 mL) and ethyl acetate (200 mL) were added, the organic phase was separated, the aqueous phase was extracted with ethyl acetate (150 mL×3) three times until the aqueous phase contained no product, the organic phases were combined, rinsed with saturated brine (150 mL×2) twice, and evaporated to dryness to obtain 2.48 g of the product.

41-190

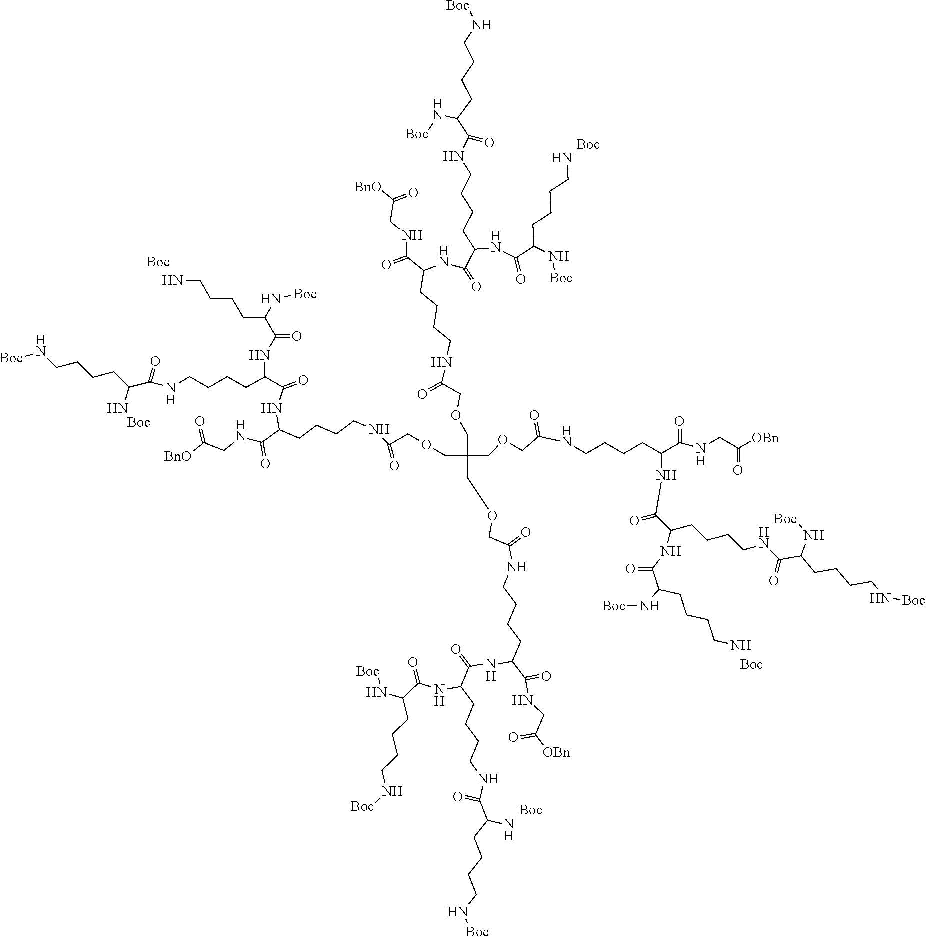

The product 41-188 (2.48 g, 2.3 mmol), product 41-189 (0.1498 g, 0.48 mmol), HBTU (1.0922 g, 2.88 mmol) and HOBT (0.3891 g, 2.88 mmol) were weighed and added into a 500 mL flask, DMF (30 mL) was added for dissolution, and the reactant solution was stirred at −5° C. for 30 minutes. Then DIEA (1.4280 mL, 8.64 mmol) was slowly added dropwise. The reactant solution reacted at −5° C. overnight. After the reaction ended, pure water and ethyl acetate were added for extraction of the reactant solution. The organic phase was rinsed with saturated brine, concentrated and dry loaded for column chromatography with gradient elution using 1% to 55% methanol/dichloromethane, to obtain 1.5 g of the product, with a yield of 68%.

41-195

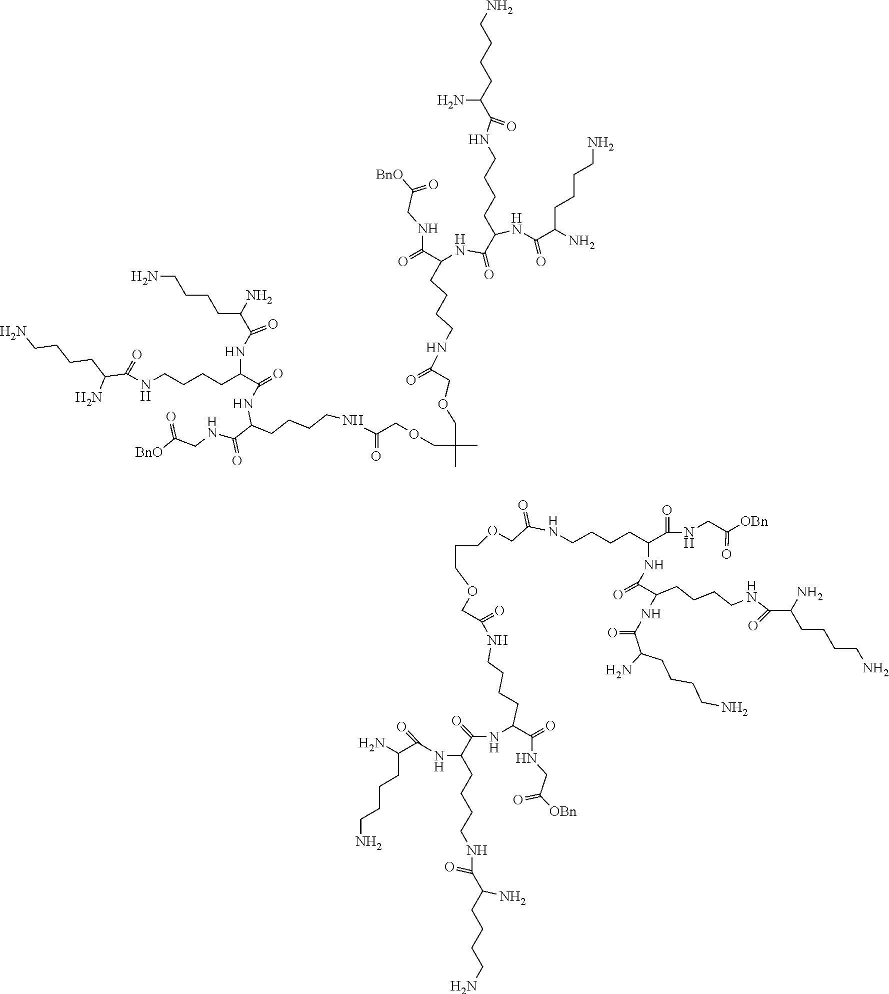 [SMILES: CC(C)(COCC(=O)NCCCCC(NC(=O)C(CCCCNC(=O)C(N)CCCCN)NC(=O)C(N)CCCCN)C(=O)NCC(=O)OCc1ccccc1)COCC(=O)NCCCCC(NC(=O)C(CCCCNC(=O)C(N)CCCCN)NC(=O)C(N)CCCCN)C(=O)NCC(=O)OCc1ccccc1 ; NCCCCC(N)C(=O)NCCCCC(NC(=O)C(N)CCCCN)C(=O)NC(CCCCNC(=O)COCCCOCC(=O)NCCCCC(NC(=O)C(CCCCNC(=O)C(N)CCCCN)NC(=O)C(N)CCCCN)C(=O)NCC(=O)OCc1ccccc1)C(=O)NCC(=O)OCc1ccccc1]

The product 41-190 (0.7 g, 0.1518 mmol) was added to a 250 mL round-bottomed flask, and dissolved in dichloromethane (20 mL), and then TFA (0.1691 mL, 2.2778 mmol) was added, and the reactant solution was stirred for reaction at the room temperature overnight. After the reaction ended, the reactant solution was concentrated under reduced pressure, n-hexane (50 mL) and methyl tertiary butyl ether (200 mL) were added for precipitation, such procedure was repeated three times, and the resultant product was filtered and dried, to obtain 0.6754 g of the product.

41-196

Boc-L-Lys(Boc)-OH (1.0517 g, 3.0358 mmol, Innochem), product 41-195 (0.6754 g, 0.1518 mmol), HBTU (1.3816 g, 3.6432 mmol) and HOBT (0.4922 g, 3.6432 mmol) were weighed and added into a 500 mL flask, DMF (80 mL) was added for dissolution, and the reactant solution was stirred at −5° C. for 30 minutes. Then DIEA (1.8 mL, 10.9296 mmol) was slowly added dropwise. The reactant solution reacted at −5° C. overnight. After the reaction ended, pure water and ethyl acetate were added for extraction of the reactant solution. The organic phase was rinsed with saturated brine, concentrated and dry loaded for column chromatography with gradient elution using 2.5% to 7% methanol/dichloromethane, to obtain 1 g of the product, with a yield of 79%.

41-206

The product 41-196 (1.0 g, 0.1202 mmol) and 10% Pd/C catalyst (300 mg) were added into a hydrogenation reaction kettle, DMF (15 mL) was slowly added, the resultant mixture was slowly stirred and dissolved, hydrogen gas (20 psi) was injected, and the reactant solution was stirred overnight at room temperature. Next day, the reactant solution was subjected to suction filtration with a sand core funnel filled with celite to remove Pd/C and obtain a solution of the product 41-206 in DMF to be used directly in the next reaction.

41-208

M-NH₂HCl-10K (2.6501 g, 0.252 mmol, JenKem), product 41-206 (0.4774 g, 0.06 mmol), HBTU (0.1365 g, 0.36 mmol) and HOBT (0.0486 g, 0.36 mmol) were weighed and added into a 500 mL flask, DMF (80 mL) was added for dissolution, and the reactant solution was stirred at −5° C. for 30 minutes. Then DIEA (0.1785 mL, 1.08 mmol) was slowly added dropwise, and the reactant solution was taken out and stirred for one week at a low speed at room temperature while protected from light. The reactant solution was stirred at a low speed until the reaction ended, then n-hexane (120 mL) and methyl tertiary butyl ether (40 mL) were added, to obtain a solid precipitate which was filtered and dry loaded for column chromatography with gradient elution using 2.5% to 7% methanol/dichloromethane, to obtain 0.5985 g of product 41-208.

41-210

The product 41-208 (0.5985 g, 0.0120 mmol) was added to a 500 mL round-bottomed flask, and dissolved in dichloromethane (30 mL), and then TFA (0.5425 mL, 5.7838 mmol) was added, and the reactant solution was stirred for reaction at the room temperature overnight. After the reaction ended, the reactant solution was concentrated under reduced pressure, n-hexane (100 mL) and methyl tertiary butyl ether (400 mL) were added for precipitation, such procedure was repeated three times, and the resultant product was filtered and dried, to obtain 0.3 g of the product.

41-228

The product 41-210 (0.3 g, 0.0064 mmol), product 41-224 (0.1763 g, 0.5165 mmol), HBTU (0.1165 g, 0.3072 mmol) and HOBT (0.0415 g, 0.3072 mmol) were weighed and added into a 500 mL flask, DMF (30 mL) was added for dissolution, and the reactant solution was stirred at −5° C. for 30 minutes. Then DIEA (0.1523 mL, 0.9216 mmol) was slowly added dropwise, and the reactant solution was taken out and stirred for one week at a low speed at room temperature while protected from light. The reactant solution was stirred at a low speed until the reaction ended, then n-hexane (120 mL) and methyl tertiary butyl ether (40 mL) were added for precipitation, to obtain 0.3 g of product 41-228.

¹H-NMR (400 MHz, DMSO-d₆) δ 8.45 (m, 1H), 8.42-8.37 (m, 3H), 8.24-8.16 (m, 4H), 7.95-7.94 (m, 169H), 7.80-7.76 (m, 25H), 7.51-7.49 (m, 17H), 7.30-7.21 (m, 45H), 5.77-5.71 (m, 9H), 4.19-4.13 (m, 71H), 3.72-3.69 (m, 12H), 3.65-3.45 (m, 3851H) 3.08-3.04 (m, 40H), 3.01-2.98 (m, 20H), 2.88-2.88 (m, 62H), 2.73-2.72 (m, 129H), 2.70-2.68 (m, 28H), 2.57-2.54 (m, 21H), 2.10-1.95 (m, 7H), 1.78-1.72 (m, 23H), 1.36-1.32 (m, 12H), 1.25-1.20 (m, 192H), 1.12-1.10 (m, 11H), 0.98-0.89 (m, 34H).

### Synthesis of Compound 56-84

56-82

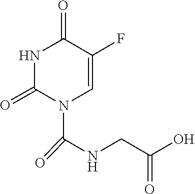 [SMILES: O=C(O)CNC(=O)n1cc(F)c(=O)[nH]c1=O]

5FU-UA-Gly-OtBu (1.0 g, 3.48 mmol, Changsha Kangpeng Pharmaceutical Co., Ltd.) was added to a 500 mL round-bottomed flask, and dissolved in dichloromethane (30 mL), and then TFA (5.95 mL, 52.22 mmol) was added, and the reactant solution was stirred for reaction at the room temperature overnight. After the reaction ended, the reactant solution was concentrated under reduced pressure, methyl tertiary butyl ether (400 mL) were added for precipitation, such procedure was repeated three times, and the resultant product was filtered and dried, to obtain 0.7 g of the product.

52-78

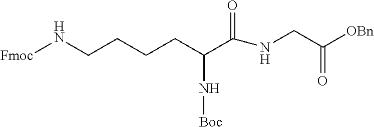

Boc-L-Lys(Fmoc)-OH (5.01 g, 10.67 mmol), Gly-OBn (2.15 g, 10.67 mmol), HBTU (6.07 g, 16.005 mmol) and HOBT (2.16 g, 16.005 mmol) were weighed and added into a 500 mL flask, DMF (40 mL) was added for dissolution, and the reactant solution was stirred at −5° C. for 30 minutes. Then DIEA (7.9 mL, 48.015 mmol) was slowly added dropwise. The reactant solution reacted at −5° C. overnight. After the reaction ended, pure water and ethyl acetate were added for extraction of the reactant solution. The organic phase was rinsed with saturated brine, concentrated and evaporated to dryness, to obtain 6.57 g of the product.

52-79

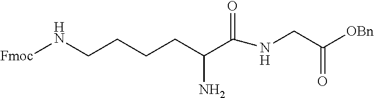

The compound 52-78 (6.57 g, 10.67 mmol) was weighed, dichloromethane (20 mL) and TFA (15.8 mL, 213.4 mmol) were added, the product was ultrasonically dissolved, and the reactant solution was stirred for reaction. After the reaction ended, the reactant solution was concentrated and then dissolved in ethyl acetate (100 mL), and rinsed with saturated brine (100 mL), the organic phase was separated, the aqueous phase was extracted with ethyl acetate (50 mL×3) three times until the aqueous phase contained no product, and the organic phases were combined, and further rinsed with saturated brine (50 mL) once, concentrated and evaporated to dryness to obtain 5.54 g of the product.

52-80

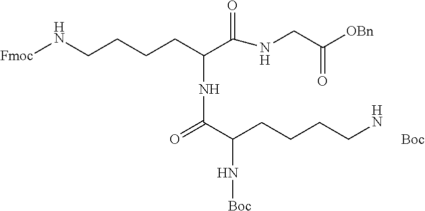

Boc-L-Lys(Boc)-OH (5.63 g, 10.76 mmol), product 52-79 (5.54 g, 10.76 mmol), HBTU (6.06 g, 16.005 mmol) and HOBT (2.16 g, 16.005 mmol) were weighed and added into a 500 mL flask, DMF (80 mL) was added for dissolution, and the reactant solution was stirred at −5° C. for 30 minutes. Then DIEA (7.9 mL, 48.015 mmol) was slowly added dropwise. The reactant solution reacted at −5° C. overnight. After the reaction ended, pure water and ethyl acetate were added for extraction of the reactant solution. The organic phase was rinsed with saturated brine, concentrated and dry loaded for column chromatography with gradient elution using 50% to 100% ethyl acetate/petroleum ether, to obtain 9.08 g of the product.

52-82

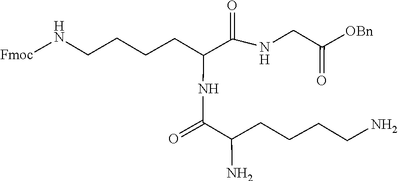

The compound 52-80 (9.08 g, 10.76 mmol) was weighed, dichloromethane (90 mL) and TFA (32 mL, 430.4 mmol) were added, the product was ultrasonically dissolved, and the reactant solution was stirred for reaction. After the reaction ended, the reactant solution was concentrated and then dissolved in ethyl acetate, then petroleum ether was added for precipitation, such procedure was repeated three times, and the resultant product was filtered and dried, to obtain 6.92 g of the product.

52-83

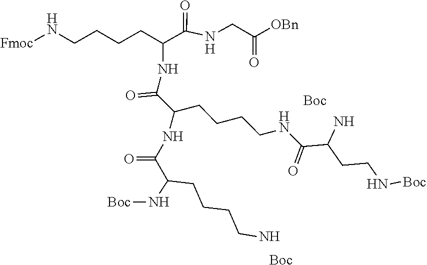

Boc-L-Lys(Boc)-OH (11.924 g, 22.596 mmol), product 52-82 (6.92 g, 10.76 mmol), HBTU (12.2418 g, 32.28 mmol) and HOBT (4.3617 g, 32.28 mmol) were weighed and added into a 500 mL flask, DMF (80 mL) was added for dissolution, and the reactant solution was stirred at −5° C. for 30 minutes. Then DIEA (16 mL, 96.84 mmol) was slowly added dropwise. The reactant solution reacted at −5° C. overnight. After the reaction ended, pure water and ethyl acetate were added for extraction of the reactant solution. The organic phase was rinsed with saturated brine, concentrated and dry loaded for column chromatography with gradient elution using 50% to 100% ethyl acetate/petroleum ether, to obtain 3.1 g of the product.

56-42

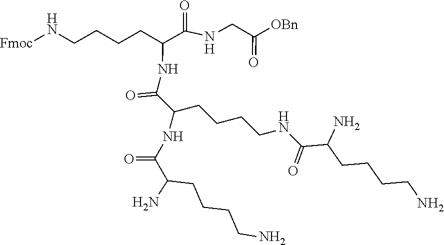

The product 52-83 (1.8 g, 1.3840 mmol) was added to a 250 mL round-bottomed flask, and dissolved in dichloromethane (20 mL), and then TFA (6.1667 mL, 83.0399 mmol) was added, and the reactant solution was stirred for reaction at the room temperature overnight. After the reaction ended, the reactant solution was concentrated under reduced pressure, n-hexane (50 mL) and methyl tertiary butyl ether (200 mL) were added for precipitation, such procedure was repeated three times, and the resultant product was filtered and dried, to obtain 1.26 g of the product.

56-43

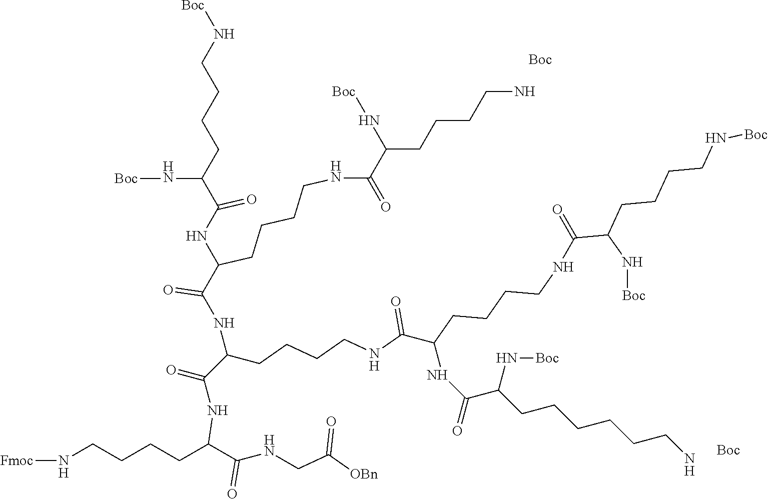

Boc-L-Lys(Boc)-OH (2.1095 g, 6.0896 mmol), product 56-42 (1.2457 g, 1.3840 mmol), HBTU (3.1492 g, 8.304 mmol) and HOBT (1.1220 g, 8.304 mmol) were weighed and added into a 500 mL flask, DMF (80 mL) was added for dissolution, and the reactant solution was stirred at −5° C. for 30 minutes. Then DIEA (4.1174 mL, 24.912 mmol) was slowly added dropwise. The reactant solution reacted at −5° C. overnight. After the reaction ended, pure water and ethyl acetate were added for extraction of the reactant solution. The organic phase was rinsed with saturated brine, concentrated and dry loaded for column chromatography with gradient elution using 2.5% to 5% methanol in dichloromethane, to obtain 1.5 g of the product.

56-47

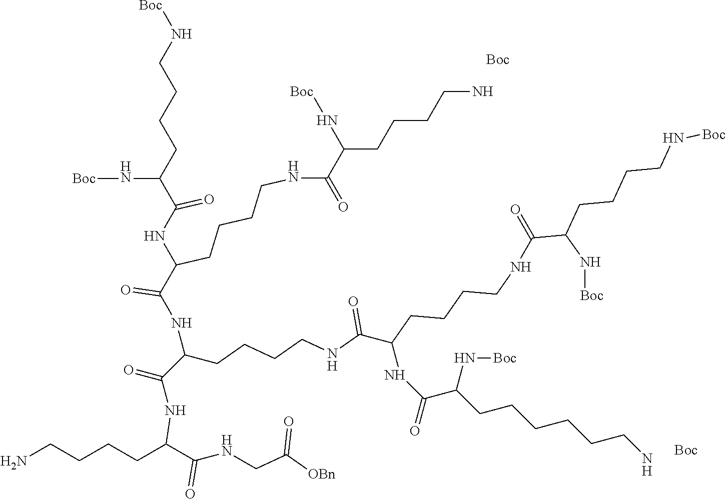 [SMILES: CC(C)(C)OC(=O)NCCCCC(NC(=O)OC(C)(C)C)C(=O)NCCCCC(NC(=O)C(CCCCCCN)NC(=O)OC(C)(C)C)C(=O)NCCCCC(NC(=O)C(CCCCNC(=O)C(CCCCN)NC(=O)OC(C)(C)C)NC(=O)C(CCCCNC(=O)OC(C)(C)C)NC(=O)OC(C)(C)C)C(=O)NC(CCCCN)C(=O)NCC(=O)OCc1ccccc1 ; "Boc" ; "Boc"]

The reactant 56-43 (1.5 g, 0.6776 mmol) was added to a 250 mL flask, a DMF solution (50 mL) was added, morpholine (0.8855 mL, 10.1638 mmol) was added, the reactant solution was stirred for reaction at room temperature, and after 3 hours, the reaction ended. Then n-hexane (50 mL) and methyl tertiary butyl ether (200 mL) were repeatedly added for precipitation three times, the reactant solution was filtered, and the organic phase was concentrated and dry loaded for column chromatography with gradient elution using 2.5% to 5% methanol in dichloromethane, to obtain 1.236 g of the product.

56-48

The product 56-47 (1.236 g, 0.6206 mmol), product 43-121 (1.3 mL, 0.1410 mmol), HBTU (0.3208 g, 0.846 mmol) and HOBT (0.1143 g, 0.846 mmol) were weighed and added into a 500 mL flask, DMF (30 mL) was added for dissolution, and the reactant solution was stirred at −5° C. for 30 minutes. Then DIEA (0.4195 mL, 2.538 mmol) was slowly added dropwise. The reactant solution reacted at −5° C. overnight. After the reaction ended, pure water and ethyl acetate were added for extraction of the reactant solution. The organic phase was rinsed with saturated brine, concentrated and dry loaded for column chromatography with gradient elution using 1% to 5% methanol in dichloromethane, to obtain 0.74 g of the product.

56-61

The product 56-48 (0.74 g, 0.0897 mmol) and 10% Pd/C catalyst (100 mg) were added into a hydrogenation reaction kettle, DMF (15 mL) was slowly added, the resultant mixture was slowly stirred and dissolved, hydrogen gas (20 psi) was injected, and the reactant solution was stirred overnight at room temperature. Next day, the reactant solution was subjected to suction filtration with a sand core funnel filled with celite to remove Pd/C and obtain a solution of the product 56-61 in DMF to be used directly in the next reaction.

56-67

The M-NH₂HCl-10K (2.1 g, 0.1988 mmol, JenKem), product 56-61 (0.3549 g, 0.045 mmol), HBTU (0.1023 g, 0.27 mmol) and HOBT (0.0365 g, 0.27 mmol) were weighed and added into a 500 mL flask, DMF (80 mL) was added for dissolution, and the reactant solution was stirred at −5° C. for 30 minutes. Then DIEA (0.1339 mL, 0.81 mmol) was slowly added dropwise, and the reactant solution was taken out and stirred for one week at a low speed at room temperature while protected from light. The reactant solution was stirred at a low speed until the reaction ended, then n-hexane (120 mL) and methyl tertiary butyl ether (40 mL) were added, to obtain a solid precipitate which was filtered and dry loaded for column chromatography with gradient elution using 2.5% to 7% methanol/dichloromethane, to obtain 0.8 g of product 56-67.

56-78

The product 56-67 (0.8 g, 0.1602 mmol) was added to a 500 mL round-bottomed flask, and dissolved in dichloromethane (30 mL), and then TFA (5.711 mL, 76.911 mmol) was added, and the reactant solution was stirred for reaction at the room temperature overnight. After the reaction ended, the reactant solution was concentrated under reduced pressure, methyl tertiary butyl ether (400 mL) were added for precipitation, such procedure was repeated three times, and the resultant product was filtered and dried, to obtain 0.652 g of the product.

56-84

The product 56-78 (0.652 g, 0.0139 mmol), product 56-82 (0.1125 g, 0.4865 mmol), HBTU (0.2530 g, 0.6672 mmol) and HOBT (0.09 g, 0.6672 mmol) were weighed and added into a 500 mL flask, DMF (30 mL) was added for dissolution, and the reactant solution was stirred at −5° C. for 30 minutes. Then DIEA (0.3308 mL, 2.0016 mmol) was slowly added dropwise, and the reactant solution was taken out and reacted for 2 hours at room temperature.

¹H-NMR (400 MHz, DMSO-d₆) δ 10.66-10.65 (m, 32H), 8.20-8.16 (m, 3H), 7.97-7.87 (m, 15H), 7.82-7.77 (m, 1H), 7.75-7.59 (m, 86H), 7.56-7.53 (m, 1H), 7.53-7.44 (m, 15H), 7.40-7.31 (m, 16H), 6.67-640 (m, 2H), 6.26-6.03 (s, 30H), 5.76-5.62 (s, 31H), 4.77-4.40 (m, 42H), 3.87-3.69 (m, 15H), 3.65-3.61 (m, 36H), 3.55-3.45 (m, 3810H), 3.13-3.00 (m, 18H), 2.92-2.81 (m, 9H), 2.78-2.70 (m, 12H), 2.68-2.65 (m, 1H), 2.64-2.59 (m, 3H), 2.07-1.73 (m, 9H), 1.63-0.93 (m, 9H).

### Example 2 Study on the Efficacy of Aflibercept Used as Positive Control and Compound 39-205 on Laser Modeling of Choroidal Neovascularization (CNV) in Mice

Experimental animals: C57BL/6J suckling mice, where the mice were all female and subjected to laser modeling three days after birth.

### Experimental Method:

A mixed solution of 20% V/V PEG300, 20% V/V anhydrous ethanol and 60% V/V normal saline was used as a solvent for compound 39-205 to prepare a 20 mg/mL solution for use in mice No. 6 and No. 7, respectively. A saline solution of aflibercept was used as a positive control for mouse No. 5.

After the mice were anesthetized, a semiconductor laser was used to perform retinal photocoagulation around optic discs under a retinoscope. An irradiation site was between blood vessels that were one optic disc diameter away from the optic disc. The generation of bubbles after photocoagulation was an indicator of breaking Bruch's membrane. The laser modeling conditions for mice No. 6 and No. 7 were 350 mw and 200 ms, and the laser modeling conditions for mouse No. 5 were 300 mw, 260 ms, and the photocoagulation time was 1 s. One week after photocoagulation, medication treatments started for the mice. The administration method was: 1 μL of each of the 20 mg/mL solution of compound 39-205 prepared above and a normal saline solution of aflibercept was injected into vitreous space.

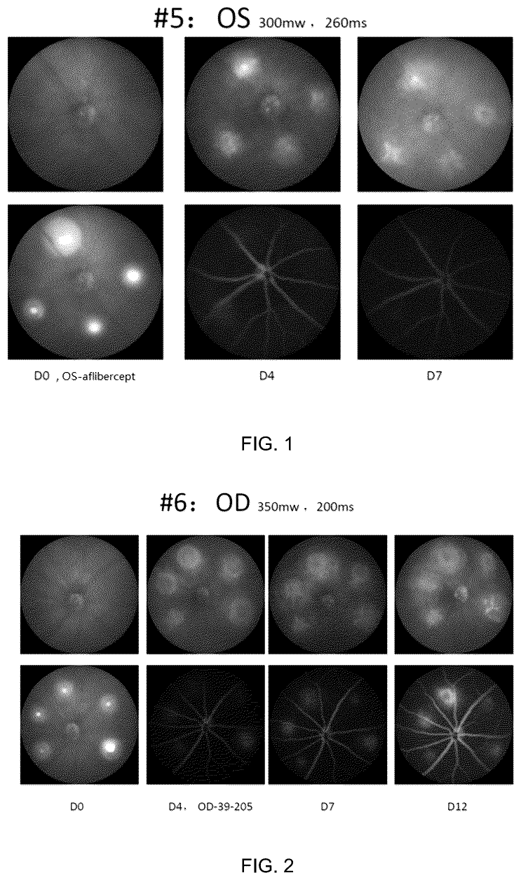

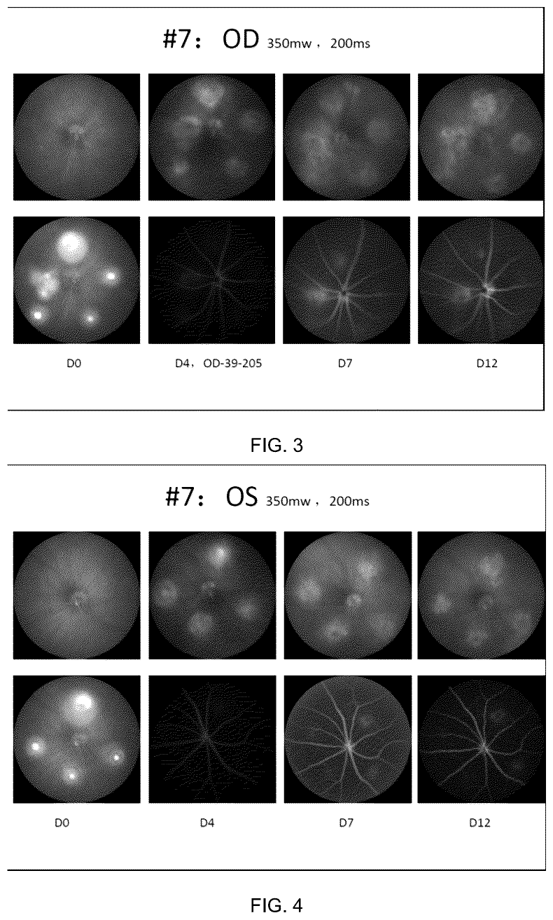

Experimental results: The experimental results are shown in FIG. 1 to FIG. 4. It can be seen from FIG. 1 that, the No. 5 laser spot did not significantly reduce, and fundus angiography was not significantly improved. It can be seen from FIG. 2 to FIG. 4 that, modeling of mice No. 6 and No. 7 was successful. The retinal blood vessels were dilated and scattered in the laser spots, there was disc-shaped fluorescence leakage within the laser spots, and the area of the laser spot could be reduced after administration.

Experimental conclusion: It can be seen from the experimental results that the compound (for example, compound 39-205) of the present application can reduce the area of the laser spot and has a therapeutic effect on choroidal neovascularization (CNV) and related ophthalmic diseases.

### Example 3 Efficacy Experiment of CT26

### 1. Name of the Experiment

Study on the efficacy of positive control (for example, 5-FU) and the compounds prepared in Example 1 on CT26 mouse transplanted tumor model.

### 2. Purpose of the Experiment

The purpose was to establish a CT26 subcutaneously transplanted tumor model in Balb/c mice, and examine the effects of all test compounds prepared in Example 1 (for example, compounds 68-67, 57-210 and 56-84) on tumor growth.

### 3. Experimental Materials

### 3.1. Main Reagents

1640 culture medium: Shanghai BasalMedia Technologies Co., LTD., catalog number: L210KJ.

FCS: Sciencell, Cat. No. 0500

Penicillin-Streptomycin Double Antibody (100×): Gibco, Cat. No. 10378-016

### Test Drug:

Control: Normal saline

Test Drugs: Control drugs and all compounds prepared in Example 1. Among them, the positive control 5-FU and the compounds 68-67, 57-210 and 56-84 from Chongqing Apgre Biotechnology Co., Ltd. were used as examples.

### 3.2. Experimental Animals

Strain: Balb/c mice, 24 SPF mice

### Supplier: Shanghai Lingchang Biotechnology Co., Ltd.

License number: SCXK (Shanghai) 2013-0018, certificate number 2013001833351
Weight: 16 g-18 g
Sex: female

### 4. Experimental Method

### 4.1. Model Establishment

(a) CT26 cells were resuscitated and amplified;
(b) After sufficient cells were amplified, the cells were collected and a cell suspension at a concentration of 2×10⁷ cells/mL was prepared by using a serum-free 1640 culture medium; and
(c) Inoculate subcutaneously on the right side of nude mice at a dose of 0.1 mL/mouse, that is, each nude mouse was inoculated with 2×10⁶ cells; and 32 mice were inoculated.

### 4.2. Grouping and Administration

4.3. The size of CT26 subcutaneous tumors was measured, and 24 tumor-bearing mice with similar tumor volumes were selected, and randomly divided into 4 groups according to the tumor volumes, with 6 mice in each group. The day of grouping was recorded as Day 1, administration started on that day and the drugs were administered every 4 days.

### 4.4. Observation and Testing

Tumor size and weight were measured every two days, and the animal status was observed and recorded.

### 4.5. Experimental Endpoint

The animals were euthanized by carbon dioxide at the endpoint. The experiment ended after the tumor-bearing mice were photographed and the tumors were removed, photographed and weighed.

### 5. Detection Indicators, Calculation and Statistical Analysis Method

### 5.1. Tumor Volume (TV)

TV=½×a×b²,

where a and b represent length and width, respectively.

### 5.2. Relative Tumor Volume (RTV, %)

$$RTV = {TV}_{1} / {TV}_{t} \times 100$$

TV₁ is the tumor volume at the time of grouping and administration (namely, d1), and TVₜ is the tumor volume at each measurement.

### 5.3. Relative Tumor Proliferation Rate T/C (%)

$$T \text{/C} ( \% ) = ( {T}_{RTV} / {C}_{RTV} ) \times 100$$

T_{RTV}: RTV of the treatment group; and C_{RTV}: RTV of the blank control group.

### 5.4. Inhibition Rate (IR) of Tumor Weight (%)

$$IR ( \% ) = ( {C}_{TW} - {T}_{TW} ) / {C}_{TW} \times 100$$

T_{TW}: tumor weight of the treatment group; C_{TW}: tumor weight of the blank control group; and TW: tumor weight.

### 5.5. Statistical Analysis

Experimental data was expressed as mean±standard deviation (mean±SD), and student's t-test was used for the weight and the tumor volume. P<0.05 indicates a significant difference, and P<0.01 indicates a highly significant difference.

Through the above experiments and results, the inventor found that the polyethylene glycol-drug conjugate of the present disclosure had a significant anti-tumor effect on subcutaneously transplanted tumors.

### Example 4 BT-474 Tumor Model Experiment in Animals

### 1. Name of the Experiment

Preliminary experiment of in vivo anti-tumor efficacy of test products (that is, all the compounds prepared in Example 1 such as compounds 48-124, 39-205, 61-104 and 51-151 and corresponding positive control compounds) on subcutaneously transplanted tumor model of human breast cancer Bt474 cells in NPG mice

### 2. Purpose of the Experiment

A subcutaneously transplanted tumor model of human breast cancer Bt474 cells in NPG mice was established to examine the anti-tumor effects of multiple test products on the subcutaneously transplanted tumors, thereby providing a basis for further pharmacodynamic experiments.

### 3. Experimental System

### 3.1. Cells and Experimental Animals

Human breast cancer BT474 cells, supplied by the Cell Resource Center of Shanghai Institute of Biological Sciences, Chinese Academy of Sciences, where culture conditions were RPM11640+10% FBS, 37° C., and 5% CO₂.

Animal Species & Strain: NPG mouse

Animal Grade: SPF

Supplier of Experimental Animals: Beijing Vitalstar Biotechnology Co., Ltd.

Age of Mice at Time of Tumor Inoculation: approximately 4 to 5 weeks.

Weight of Mice at Time of Tumor Inoculation: approximately 15 g to 18 g. Weight of animals of the same sex ranged from 80% to 120% of the average weight.

Sex and Quantity: 60 female mice were ordered, 36 animals were selected for modeling in final experiments, and the rest animals were transferred to veterinarians or euthanized.

### 3.2. Reasons for Selection

Selection Reasons for Experimental Animals: NPG mice are NPG/Vst mice independently developed by Vitalstar. Like NSG or NOG mice from aboard, NPG mice are NOD-Prkdc^{scid} Il2rg^{null} mice, and are currently internationally recognized as tool mice that have the highest degree of immunodeficiency and that are the most suitable for transplantation of human source cells.

Selection Reasons for the Quantity of Animals: The minimum quantity of animals were used in the study to meet the purpose of the study, scientific needs of the client, current scientific standards, and feasible regulatory requirements.

Use Reasons for the Animals: This strain of animals are standard animals for establishing a human source tumor model in anti-tumor experiments. A large amount of background data has been accumulated, and such animal model is found to be suitable in the same study or with similar test products. There is no other known alternative method to replace the live animal experiment.

### 3.3. Feeding

The animals were kept in independent ventilation cages (IVC), with a maximum of 6 animals of the same group in each cage. The cages were SPF animal cages. The environmental conditions were controlled to ensure room temperature of 20° C. to 26° C., relative humidity of 40% to 70%, and 12 hours of alternating light and dark.

The padding used is test padding made of wood shavings and had been sterilized via an autoclave (manufacturer: Langfang Anci District Dongfeng Paper Products Factory). Pollutants (main testing indicators: arsenic, lead, mercury, cadmium, benzene hexachloride, DDT and aflatoxin B1) were tested once a year, and a qualified institution was entrusted to conduct the testing. Microbial testing (main testing indicator: the total quantity of bacterial colonies) was conducted once a month.

### 3.4. Feed

During quarantine and domestication period, qualified rat feed (manufacturer: Beijing Keao Xieli Feed Co., Ltd.) was provided every day, and the animals had free access to the feed. Beijing Keao Xieli Feed Co., Ltd. and/or a qualified testing institution separately tested nutritional components (main testing components: crude protein, crude fat, crude fiber, water content, calcium, total phosphorus and crude ash) and physical and chemical property indicators (main testing indicators: arsenic, lead, mercury, cadmium, benzene hexachloride, DDT and aflatoxin B1) of each batch of feed. Veterinarians or their designated personnel evaluated whether contaminant content in the feed met national standards GB14924.2-2001 and GB14924.3-2010. After inoculation with estrogen tablets, the animals were fed with low-calcium feed.

### 3.5. Drinking Water

During the quarantine and domestication period and the experiment period, the animals had free access to water. Purified water (source: water filtered via a secondary reverse osmosis RO membrane by the inventor's company) was supplied in drinking water bottles. Drinking bottles and bottle stoppers were sterilized via the autoclave before use. Toxicological indicators including various pollutants (main testing indicators: arsenic, lead, mercury, cadmium, odor, taste and substances visible to the naked eye) were tested once a year by a qualified testing institution. Appearance and microbial indicators of the drinking water (main testing indicators: total bacterial count, odor, taste and substances visible to the naked eye) were tested once a month. Researchers from the Animal Experiment Department evaluated whether contaminant content in the drinking water met the national standard GB14925-2010.

### 3.6. Quarantine and Domestication

After all the animals were delivered, the veterinarian was responsible for the quarantine. The animal quarantine period was at least 2 days. Deadlines for the quarantine period and the domestication period were determined by the veterinarian. After the quarantine or domestication period ended, animals that had passed the quarantine and had similar weight were selected for the experiment.

### 3.7. Marking

Individual Marking: Before formal experimental grouping, temporary numbers from 1 to 60 were marked on the backs of the mice with a marker pen. After the grouping, the animals were marked in an ear punching method, and each animal had a unique marking number.

Cage cards: Animals in different groups were marked with cage cards in different colors. The cage cards were marked with a topic number, an animal strain, an animal group, an animal number, sex, dosage, a date of administration and IACUC number. White cage cards were used during the quarantine period.

### 4. Experimental Design

### 4.1. Model Establishment

BT474 cells were resuscitated and cell passaging and amplification was performed. When a sufficient quantity of cells were amplified, cells in a logarithmic growth phase were collected and prepared for cell inoculation.

Before cell inoculation, sustained-release estrogen tablets (17β-ESTRADIOL, Category No.: SE-121, Innovative Research of America) were implanted subcutaneously in the necks and backs of the NPG mice. The BT474 cell suspension at a concentration of 5×10⁷ to 1×10⁸ cells/mL was mixed with matrix gelatine (Matrigel Basement Membrance Matrix, BD Company) as per a volume ratio of 1:1 to obtain a cell suspension at a concentration of 2.5×10⁷ to 5×10⁷ cells/mL. 0.2 mL of the cell suspension was taken for inoculation into the right breast pad of the mouse. After the inoculation, tumor growth was observed. When the tumor volume was about 100 to 300 mm³, the animals were screened according to the tumor volume. Animals with an excessively large tumor volume and animals without tumors were not selected. Finally, 36 tumor-bearing animals were selected for the experiment.

### 4.2. Animal Grouping and Dosage

48 qualified modeling animals were randomly divided into 8 groups according to the tumor volumes, with 6 animals in each group.

### 4.3. Administration

Route of Administration: intravenous injection of negative control, positive control and compounds 48-124, 39-205, 61-104 and 51-151.

Frequency and Time of Administration: once every 4 days for 4 to 5 weeks.

Dosage: The dosage of each animal was determined based on the weight of each animal before administration.

Selection Reason for the Route of Administration: The selection reason was consistent with an intended clinical route of administration.

### 4.4. General Clinical Observations

Observation Frequency and Time: During the experiment, all the animals were generally observed twice a day.

Observation Content: mental states, behavioral activities, feed intake and the like of the animals.

### 4.5. Weight

Tested Animals: all the animals.

Testing Time: The animals were weighed after delivery, before inoculation, before grouping (that is, before the first administration) (D1), then twice a week, and before euthanasia. Animals were also weighed when they died accidentally or when they were euthanized at impending death.

### 4.6. Measurement of Tumor Diameter

Tested Animals: all the animals.

Testing Time: Long and short diameters of the tumor were measured with a vernier caliper and recorded, and the tumor volume was calculated during grouping (that is, the day of the first administration was D1), twice a week after the first administration, and before euthanasia.

The tumor volume was calculated based on the following formula:

V=½×long diameter×short diameter²

### 4.7. Tumor Volume-Based Efficacy Evaluation

A relative tumor volume (RTV) and a relative tumor proliferation rate T/C % were calculated based on the following formula:

RTV=Vₜ/V₀

Vₜ: Tumor volume obtained by measuring the tumor every day;
V₀: Initial tumor volume (before administration); and
T/C %=average RTV of the administration group/average RTV of the control group×100%.

If T/C %540% and P<0.05 after RTV of the experimental group and RTV of the model group were statistically processed, it indicated that there was an inhibitory effect on the tumor growth; or conversely, if T/C %>40%, it indicated that there was no inhibitory effect on the tumor growth.

### 4.8. Tumor Weight-Based Efficacy Evaluation

After the experiment ended, tumor nodules were removed and weighed, a tumor weight difference between the groups was obtained via comparison to further calculate a tumor inhibition rate IR_{TW}. IR_{TW}>60% was used as an effectiveness reference indicator. A calculation Formula (I)s as follows:

$${IR}_{TW} ( \% ) = ( {W}_{model} - {W}_{treatment} ) / {W}_{model} \times 100 \% .$$

### 4.9. Gross Anatomy Observation

Animals that died during the experiment and the animals that were euthanized after the observation period were grossly dissected, and the main organs were observed to see if there were any obvious abnormalities visible to the naked eye.

### 4.10. Photo Records

Photographs of euthanized animals and tumors were taken.

### 4.11. Euthanasia of Animals

According to AVMA Guidelines for the Euthanasia of Animals: 2013 Edition (the American Veterinary Medical Association, 2013), animals planned to be euthanized or dying were euthanized with excessive dosage of CO₂, and marrows were broken for confirmation.

### 4.12. Humane Endpoint

During the experiment, when the tumor load exceeded 10% of weight, the average tumor diameter exceeded 20 mm, or the tumor showed ulceration, necrosis or infection, the researcher decided whether and when to euthanize the animal according to a specific circumstance.

### 5. Data Collection and Statistical Analysis

Data Collection: Testing reference was 0.05, and the results were analyzed with reference to both statistical significance and biological significance. 2 decimal places were kept for individual data of the tumor volume and RTV. The average, T/C % and IR_{TV} % were calculated based on data before rounding, and 2 decimal places were kept for calculation results.

Statistical Analysis: In the experiment, statistical software SPSS13.0 was used to process the data, and measurement data was expressed as a standard error of the average±. A specific analysis process is as follows:

One-factor analysis of variance (ANOVA) was used for statistical analysis. If ANOVA results were of statistical significance (P≤0.05) and variances were homogeneous, the Tukey test was conducted for inter-group comparison and analyses. If the variances were not homogeneous, Dunnett's T3 test was conducted for the inter-group comparison and analyses.

Data collected when animals were euthanized out of plan, and data collected when the number (n) of samples was less than 3 was not statistically analyzed.

Through the above experiments and results, the inventor found that the polyethylene glycol-drug conjugate of the present disclosure had a significant anti-tumor effect on subcutaneously transplanted tumors (for example, subcutaneously transplanted tumor of human breast cancer BT474 cells in NPG mice).

1. A polyethylene glycol-drug conjugate represented by Formula A, a stereoisomer thereof or a pharmaceutically acceptable salt thereof, 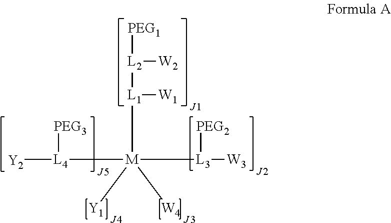
wherein:
M is selected from 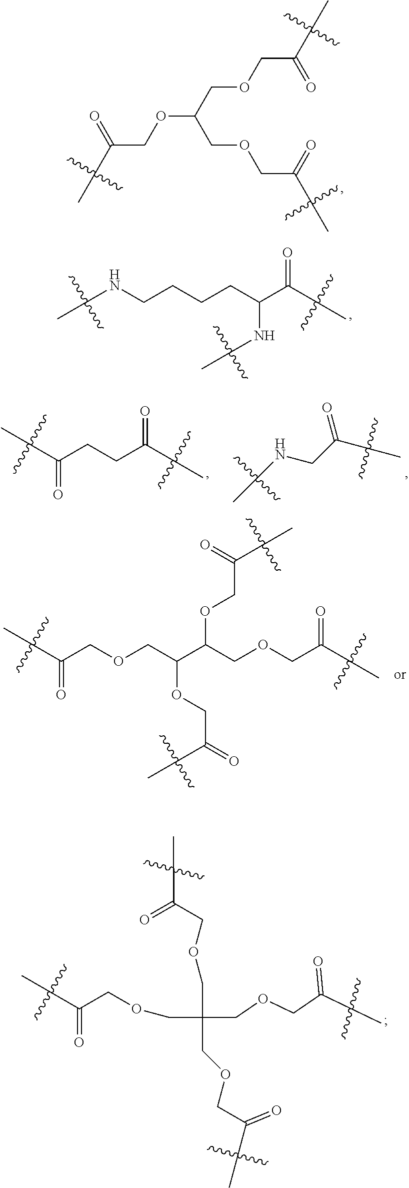 [SMILES: *C(=O)COCC(COCC(*)=O)OCC(*)=O ; *NCCCCC(N*)C(*)=O ; *C(=O)CCC(*)=O ; *NCC(*)=O ; *C(=O)COCC(OCC(*)=O)C(COCC(*)=O)OCC(*)=O ; *C(=O)COCC(COCC(*)=O)(COCC(*)=O)COCC(*)=O]
or
M is selected from 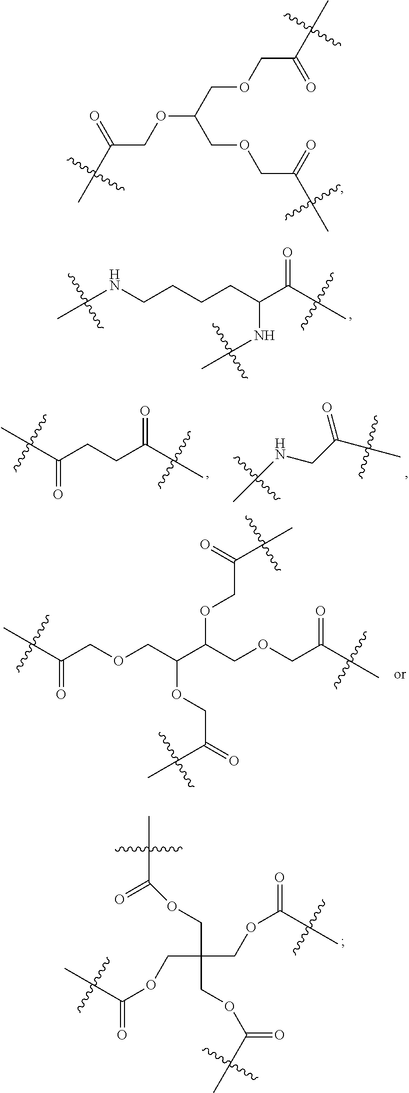 [SMILES: *C(=O)COCC(COCC(*)=O)OCC(*)=O ; *NCCCCC(N*)C(*)=O ; *C(=O)CCC(*)=O ; *NCC(*)=O ; *C(=O)COCC(OCC(*)=O)C(COCC(*)=O)OCC(*)=O ; *C(=O)OCC(COC(*)=O)(COC(*)=O)COC(*)=O]
L₁, L₂, L₃ and L₄ are each independently selected from 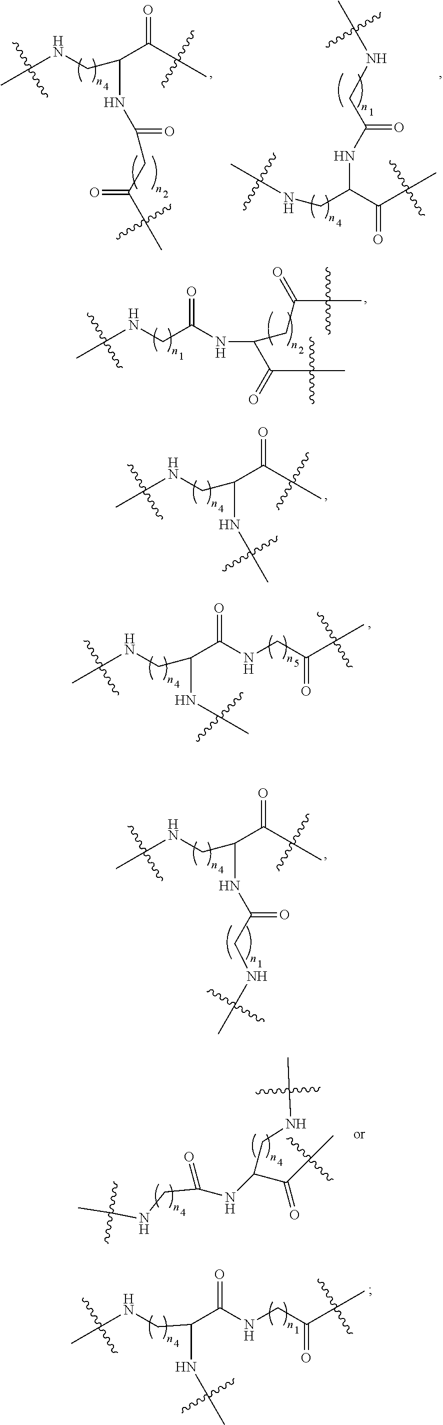
preferably, L₁, L₂, L₃ and L₄ are each independently selected from 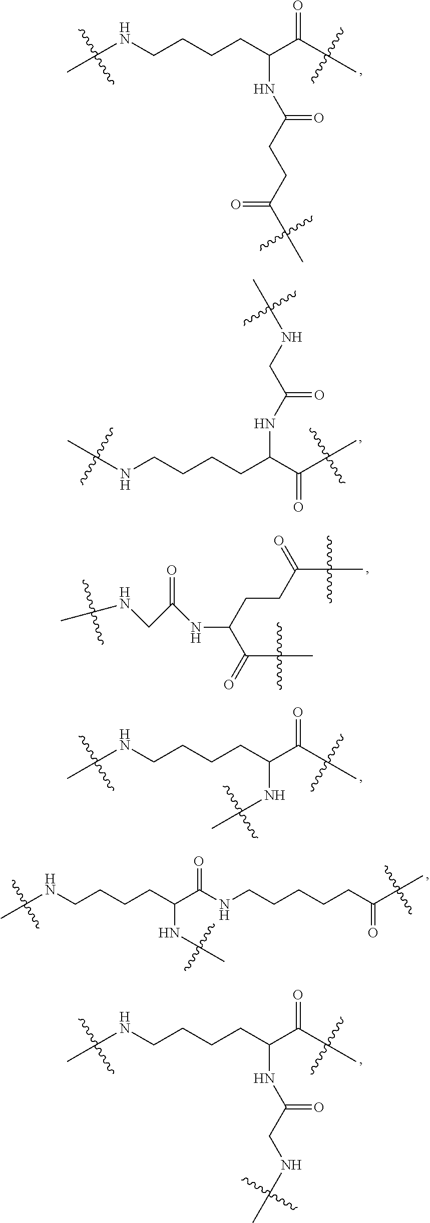 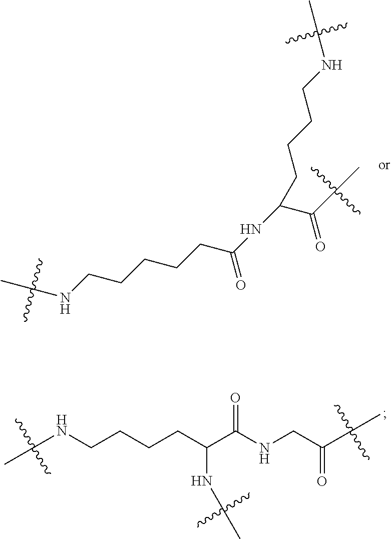 [SMILES: *NCCCCCC(=O)NC(CCCCN*)C(*)=O ; *NCCCCC(N*)C(=O)NCC(*)=O]
or
preferably, L₁, L₂, L₃ and L₄ are each independently selected from 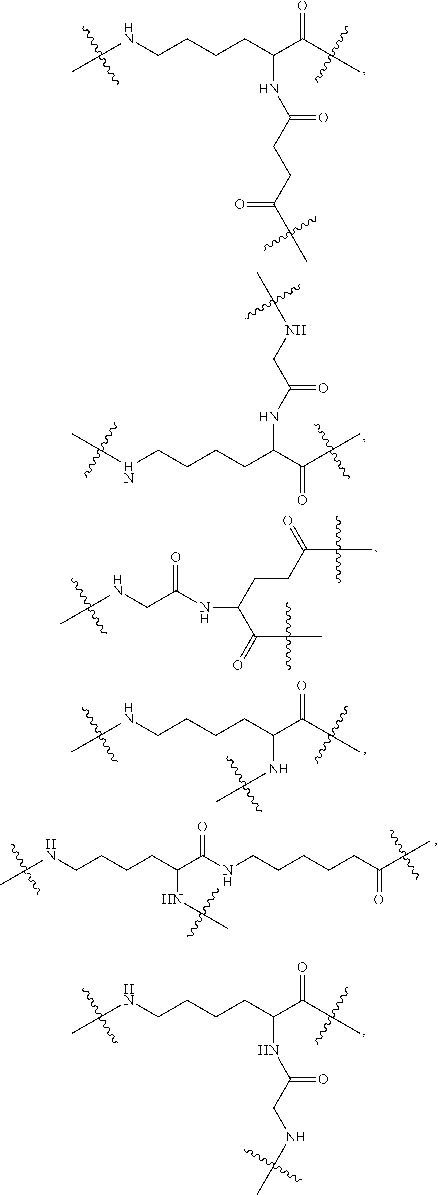 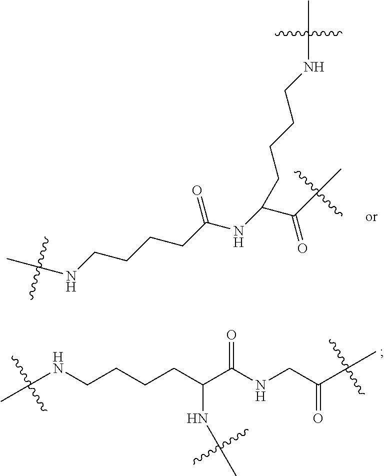 [SMILES: *NCCCCC(=O)NC(CCCCN*)C(*)=O ; *NCCCCC(N*)C(=O)NCC(*)=O]
or
preferably, L₁ is 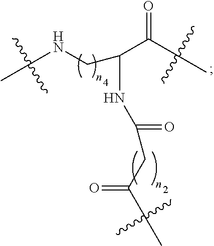
and
more preferably, L₁ is 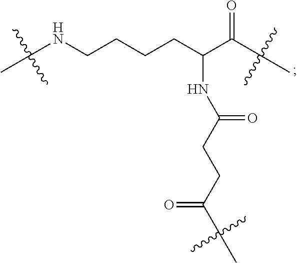 [SMILES: *NCCCCC(NC(=O)CCC(*)=O)C(*)=O]
or
preferably, L₂ is 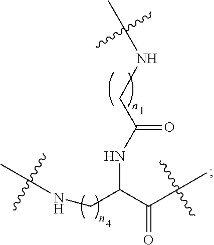
and
more preferably, L₂ is 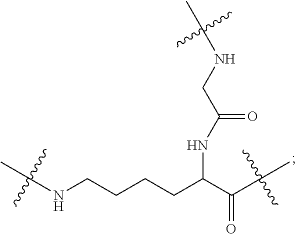 [SMILES: *NCCCCC(NC(=O)CN*)C(*)=O]
or
preferably, each L₃ is independently selected from 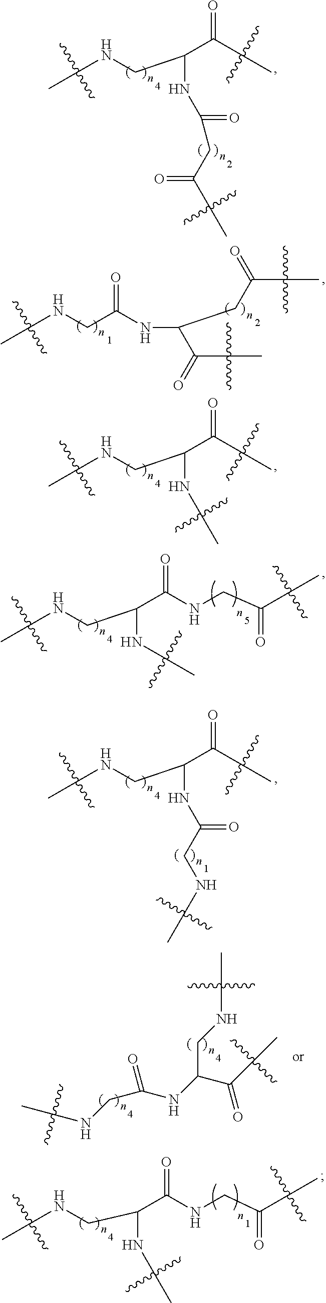
and
more preferably, each L₃ is independently selected from 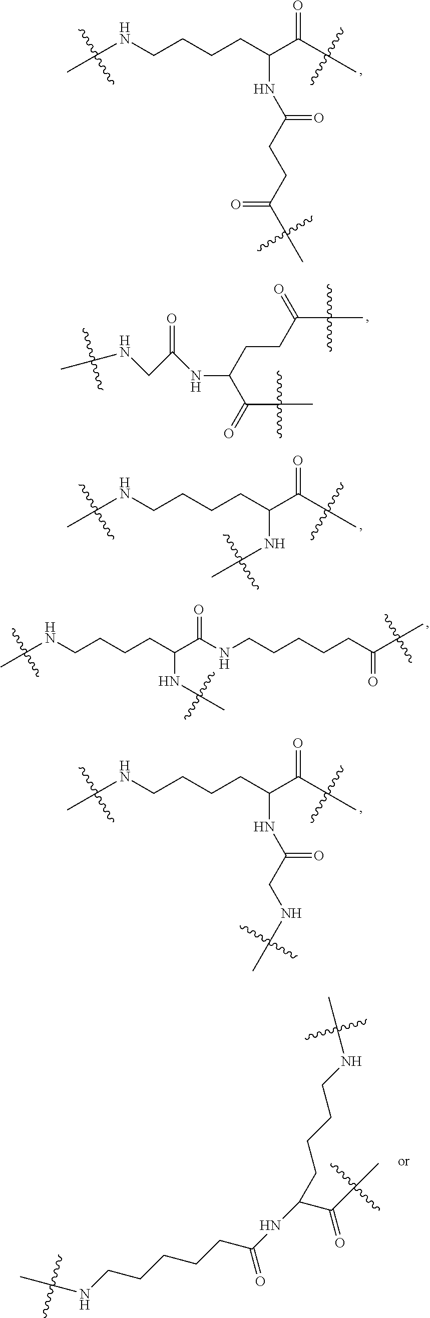 [SMILES: *NCCCCC(NC(=O)CCC(*)=O)C(*)=O ; *NCC(=O)NC(CCC(*)=O)C(*)=O ; *NCCCCC(N*)C(*)=O ; *NCCCCC(N*)C(=O)NCCCCCC(*)=O ; *NCCCCC(NC(=O)CN*)C(*)=O ; *NCCCCCC(=O)NC(CCCCN*)C(*)=O] 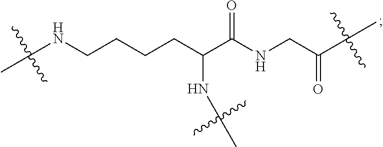 [SMILES: *NCCCCC(N*)C(=O)NCC(*)=O]
or
more preferably, each L₃ is independently selected from 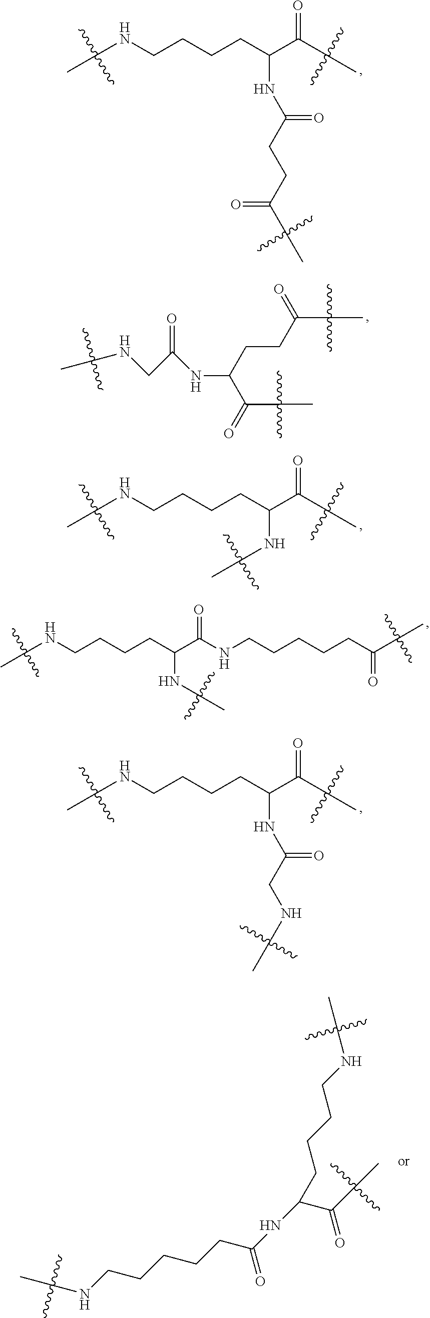 [SMILES: *NCCCCC(NC(=O)CCC(*)=O)C(*)=O ; *NCC(=O)NC(CCC(*)=O)C(*)=O ; *NCCCCC(N*)C(*)=O ; *NCCCCC(N*)C(=O)NCCCCCC(*)=O ; *NCCCCC(NC(=O)CN*)C(*)=O ; *NCCCCCC(=O)NC(CCCCN*)C(*)=O] 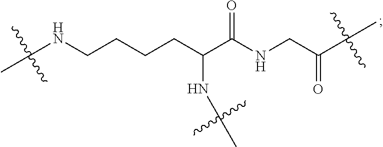 [SMILES: *NCCCCC(N*)C(=O)NCC(*)=O]
or
preferably, L₄ is 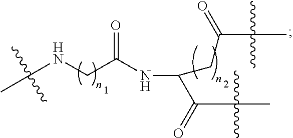
and
more preferably, L₄ is 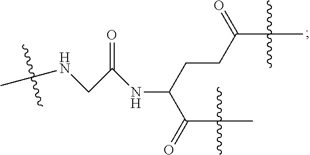 [SMILES: *NCC(=O)NC(CCC(*)=O)C(*)=O]
W₁, W₂, W₃ and W₄ are each independently selected from -Q₁, 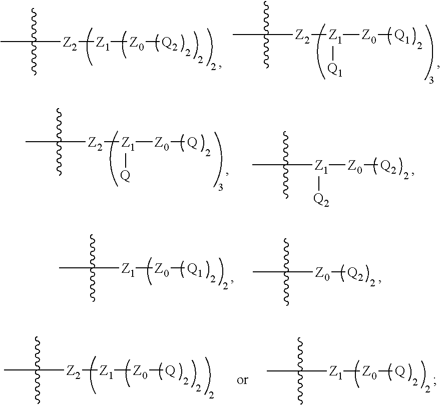
preferably, W₁ and W₂ are each independently selected from -Q₁ or 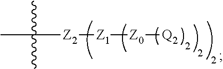
preferably, W₃ and W₄ are each independently selected from 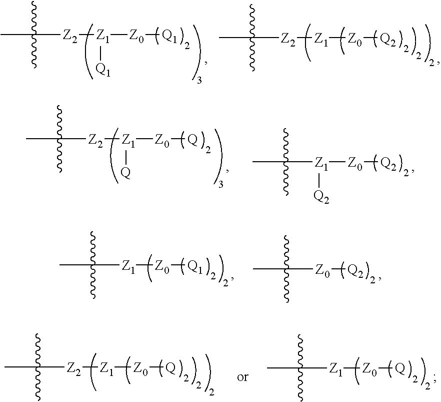
more preferably, W₁ is -Q₁;
more preferably, W₂ is 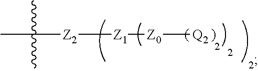
more preferably, W₃ is selected from 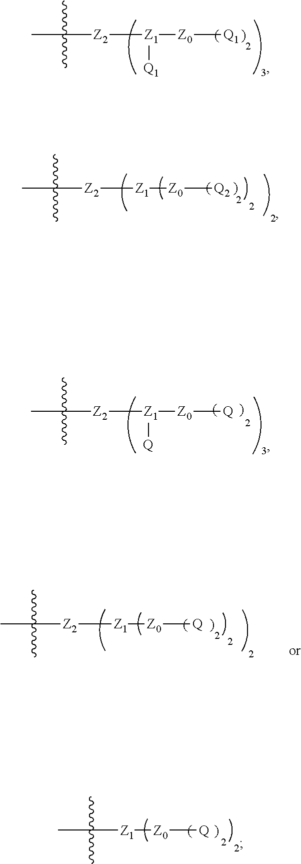
and
more preferably, W₄ is selected from 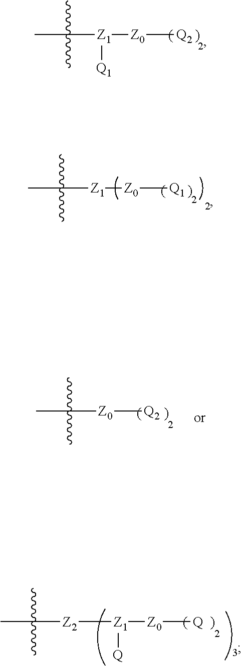
PEG₁, PEG₂ and PEG₃ are each independently a single-armed polyethylene glycol chain segment, PEG₁ is connected to L₂ through a carbonyl group, PEG₂ is connected to L₃ through a carbonyl group or an amino group, PEG₃ is connected to L₄ through a carbonyl group, and a number-average molecular weight of each of PEG₁, PEG₂ and PEG₃ is 5k to 40k, preferably 5k to 10k or 10k to 40k, and more preferably 10k;
j₁, j₂, j₃, j₄ and j₅ are each independently selected from 0, 1, 2, 3, 4 or 5, and j₁, j₂, j₃, j₄ and j₅ are not 0 at the same time;
preferably, j₁ is selected from 2, 3 or 4;
more preferably, j₁ is 3;
preferably, j₂ is selected from 1, 2, 3, 4, or 5;
more preferably, j₂ is selected from 1, 2 or 4;
preferably, j₃ is selected from 1, 2 or 3;
more preferably, j₃ is 1;
preferably, j₄ is selected from 1, 2 or 3;
more preferably, j₄ is 1;
preferably, j₅ is selected from 1, 2 or 3; and
more preferably, j₅ is 1;
Z₂, Z₁ and Z₀ are each independently selected from 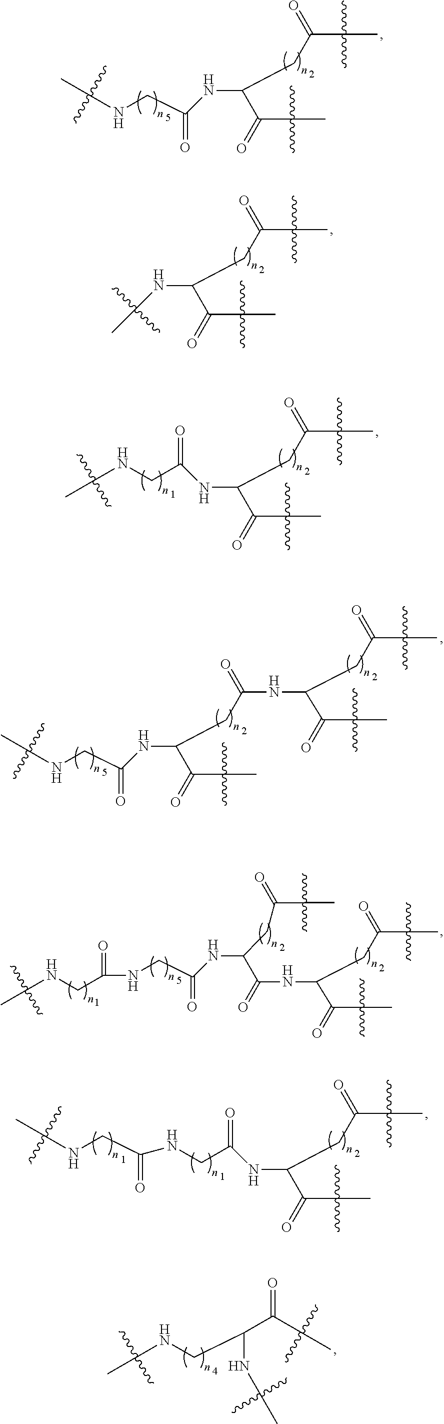 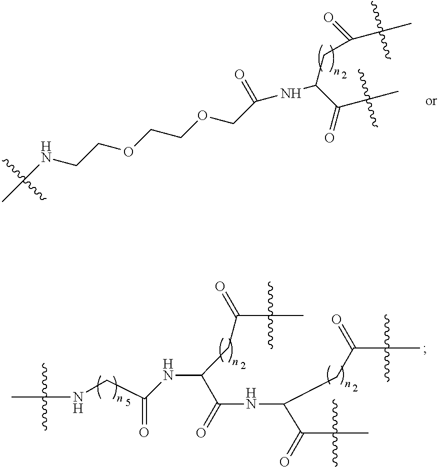
preferably, Z₂, Z₁ and Z₀ are each independently selected from 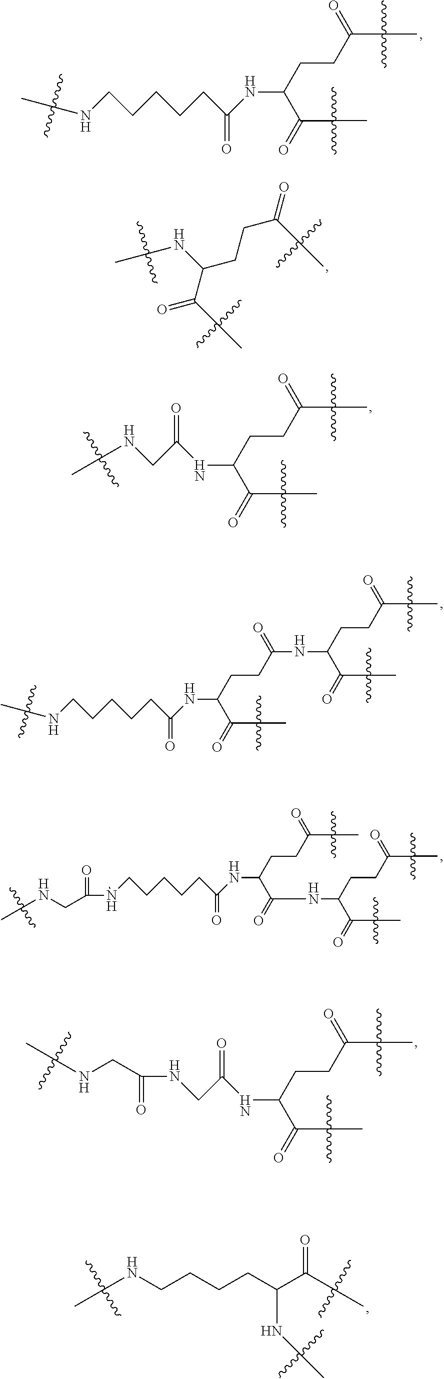 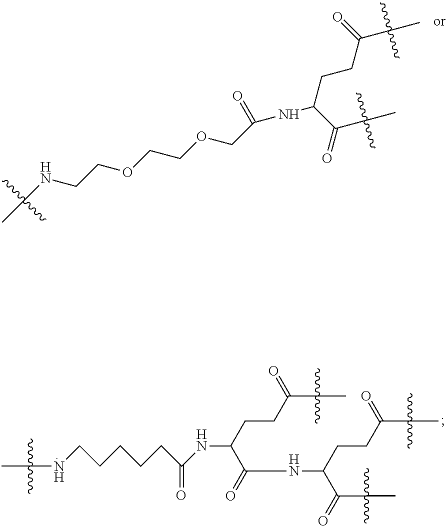 [SMILES: *NCCOCCOCC(=O)NC(CCC(*)=O)C(*)=O ; *C(=O)CCC(NC(=O)C(CCC(*)=O)NC(=O)CCCCCN(*)C)C(*)=O]
or
preferably, Z₂ is selected from 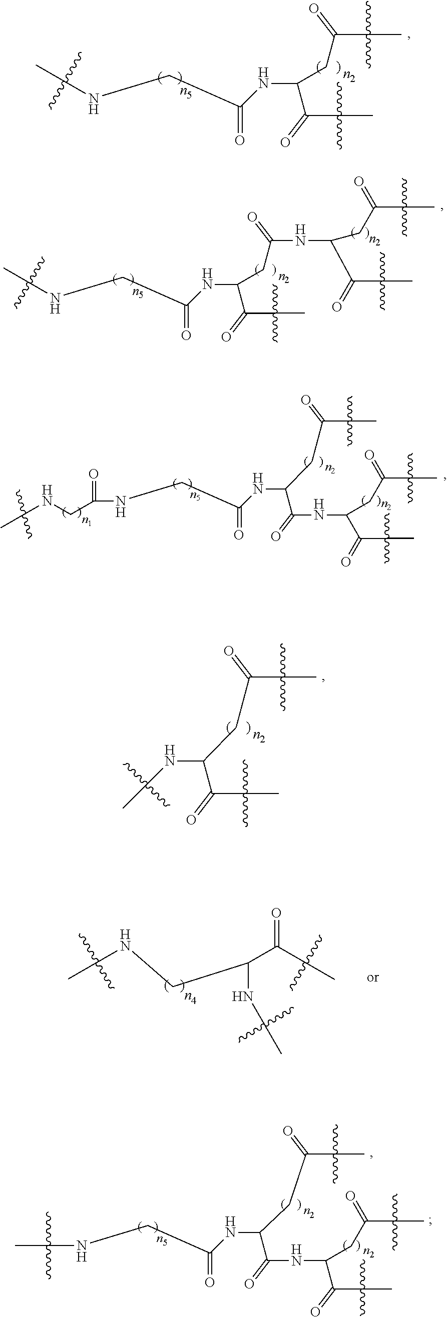
and
more preferably, Z₂ is selected from 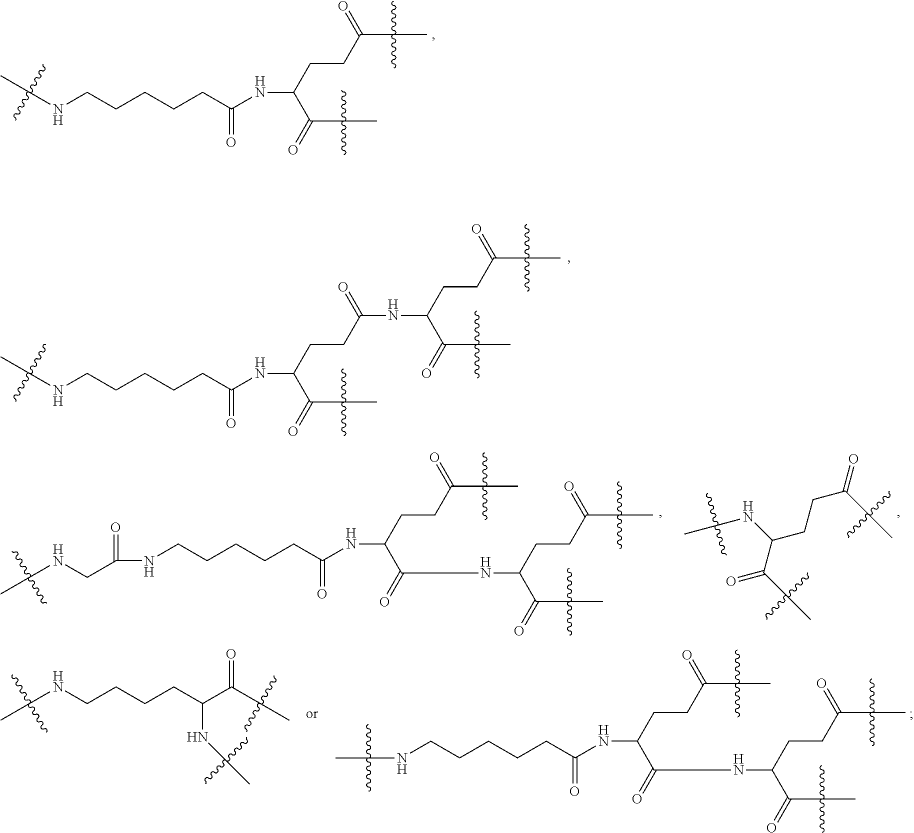 [SMILES: *NCCCCCC(=O)NC(CCC(*)=O)C(*)=O ; *NCCCCCC(=O)NC(CCC(=O)NC(CCC(*)=O)C(*)=O)C(*)=O ; *NCC(=O)NCCCCCC(=O)NC(CCC(*)=O)C(=O)NC(CCC(*)=O)C(*)=O ; *NC(CCC(*)=O)C(*)=O ; *NCCCCC(N*)C(*)=O ; *NCCCCCC(=O)NC(CCC(*)=O)C(=O)NC(CCC(*)=O)C(*)=O]
or
preferably, Z₁ is selected from 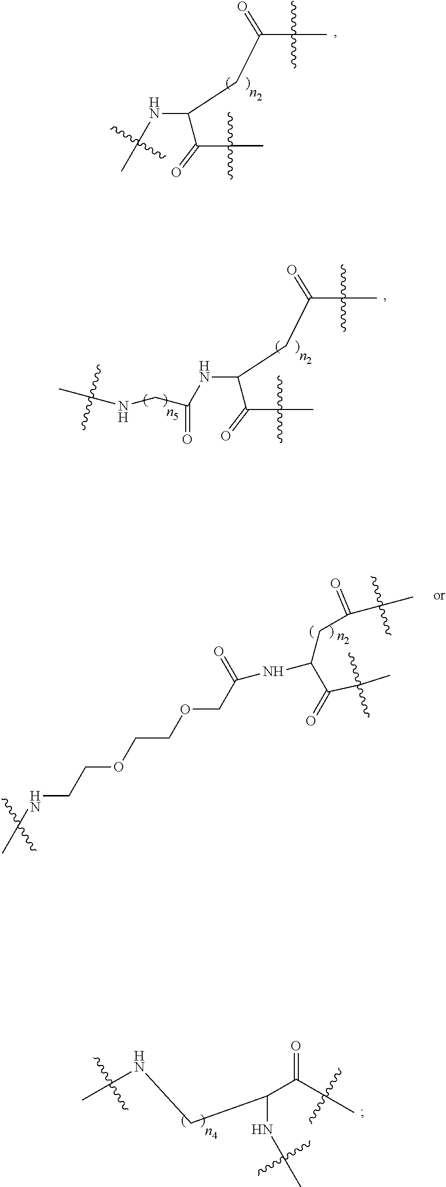
and
more preferably, Z₁ is selected from 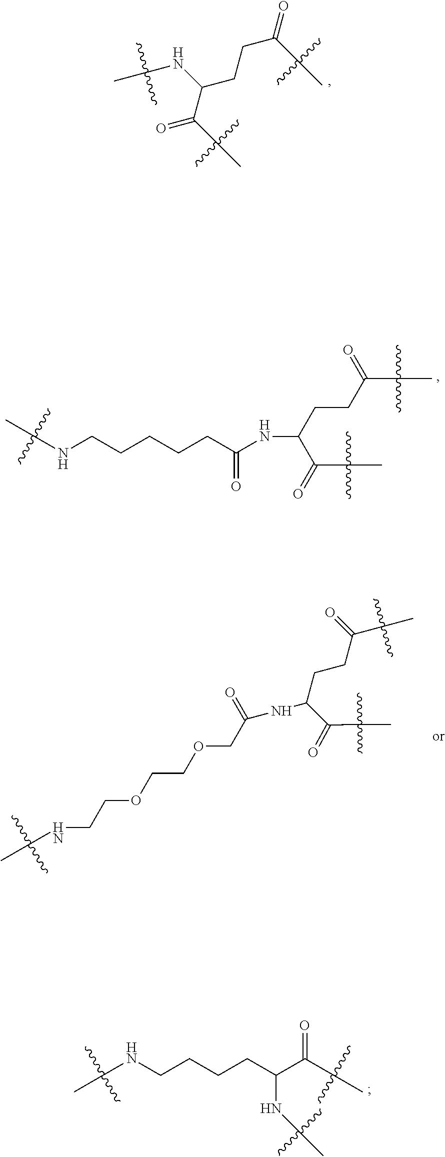 [SMILES: *NC(CCC(*)=O)C(*)=O ; *NCCCCCC(=O)NC(CCC(*)=O)C(*)=O ; *NCCOCCOCC(=O)NC(CCC(*)=O)C(*)=O ; *NCCCCC(N*)C(*)=O]
or
preferably, Z₀ is selected from 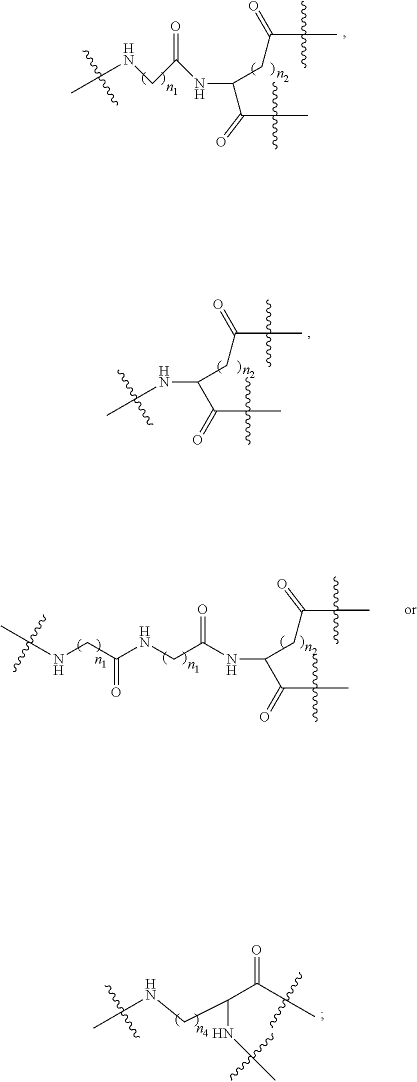
and
more preferably, Z₀ is selected from 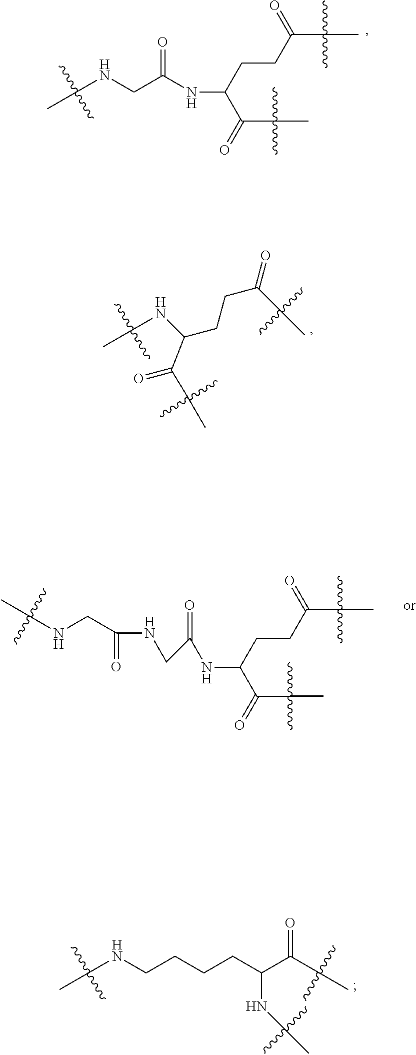 [SMILES: *NCC(=O)NC(CCC(*)=O)C(*)=O ; *NC(CCC(*)=O)C(*)=O ; *NCC(=O)NCC(=O)NC(CCC(*)=O)C(*)=O ; *NCCCCC(N*)C(*)=O]
and
Q is —N-AC;
Q₁ is —N₁-AC₁;
Q₂ is —N₂-AC₂;
N, N₁ and N₂ are each independently selected from 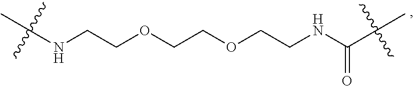 [SMILES: *NCCOCCOCCNC(*)=O]
GFLG, 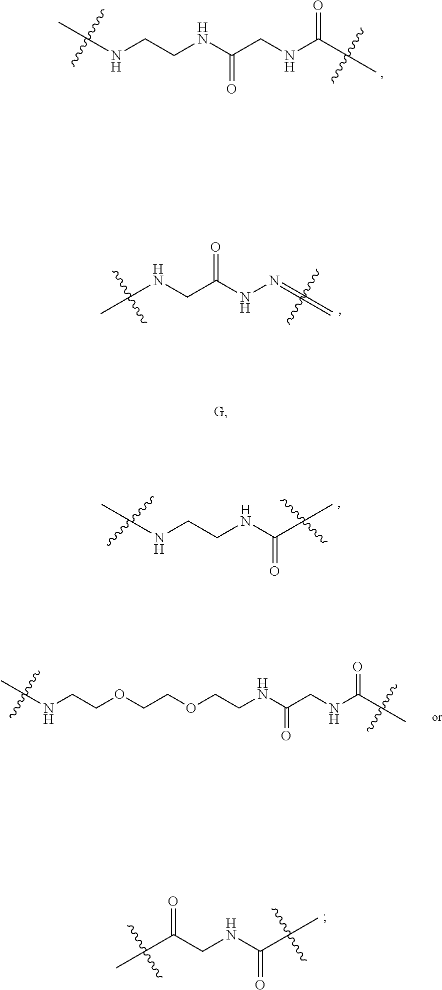 [SMILES: *NCCNC(=O)CNC(*)=O ; *NCC(=O)NN=* ; *NCCNC(*)=O ; *NCCOCCOCCNC(=O)CNC(*)=O ; *C(=O)CNC(*)=O]
preferably, N is selected from 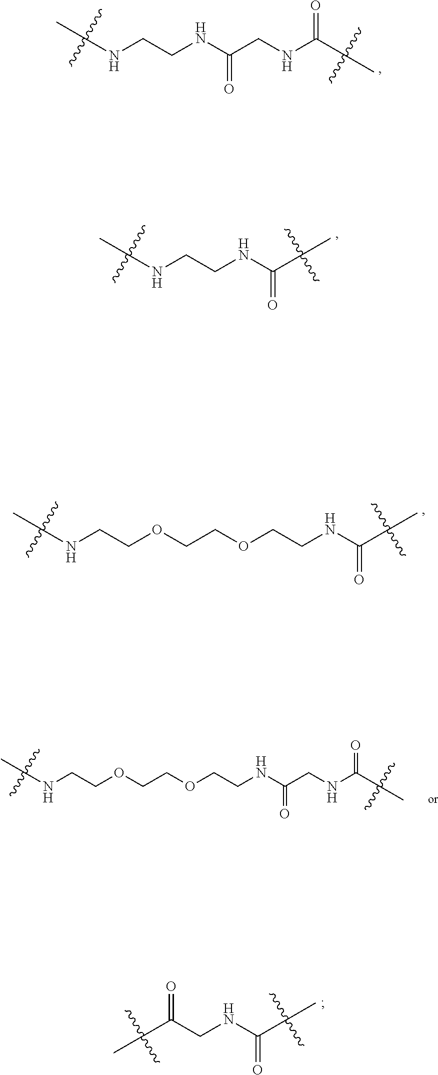 [SMILES: *NCCNC(=O)CNC(*)=O ; *NCCNC(*)=O ; *NCCOCCOCCNC(*)=O ; *NCCOCCOCCNC(=O)CNC(*)=O ; *C(=O)CNC(*)=O]
preferably, N₁ is selected from 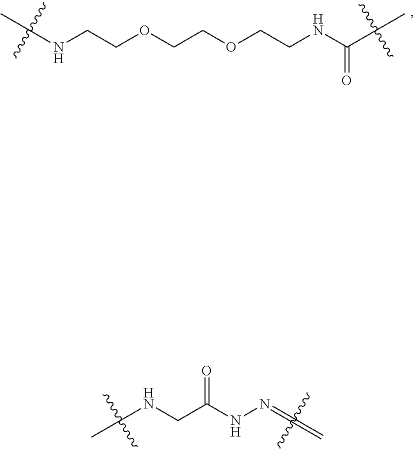 [SMILES: *NCCOCCOCCNC(*)=O ; *NCC(=O)NN=*]
or G; or
preferably, N₂ is selected from GFLG or 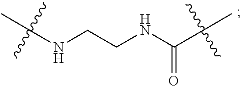 [SMILES: *NCCNC(*)=O]
AC, AC₁ and AC₂ are each independently an anticancer drug;
preferably, AC, AC₁ and AC₂ are each independently selected from AXT, PCB, 5FU, DXM, SRM, IMQ or LNL;
more preferably, AC is selected from 5FU, IMQ, LNL or AXT;
more preferably, AC₁ is selected from AXT, DXM or SRM; and
more preferably, AC₂ is selected from PCB or AXT; and
V₁ and V₂ are each independently selected from 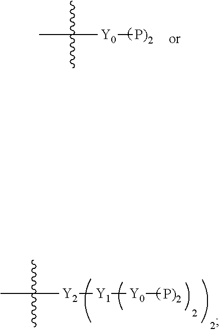
preferably, V₁ is 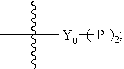
and
preferably, V₂ 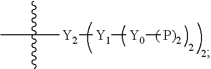
Y₂, Y₁ and Y₀ are each independently selected from 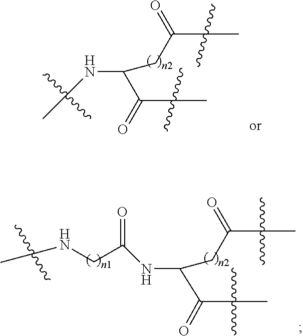
preferably, Y₂, Y₁ and Y₀ are each independently selected from 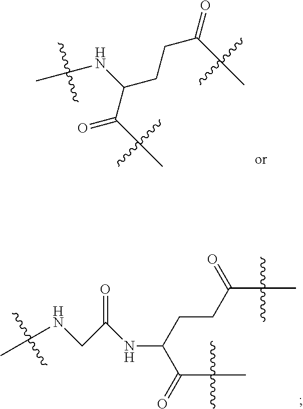 [SMILES: *NC(CCC(*)=O)C(*)=O ; *NCC(=O)NC(CCC(*)=O)C(*)=O]
or
preferably, Y₂ is 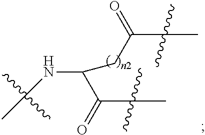
and
more preferably, Y₂ is 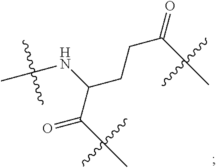 [SMILES: *NC(CCC(*)=O)C(*)=O]
or
preferably, Y₁ is 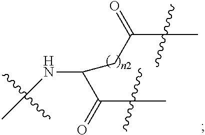
and
more preferably, Y₁ is 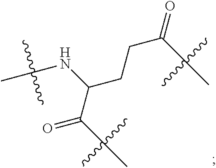 [SMILES: *NC(CCC(*)=O)C(*)=O]
or
preferably, Y₀ is 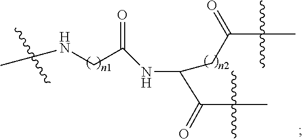
and
more preferably, Y₀ is 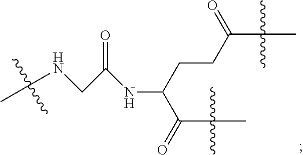 [SMILES: *NCC(=O)NC(CCC(*)=O)C(*)=O]
P is -Lᵥ-T;
Lᵥ is selected from O or H 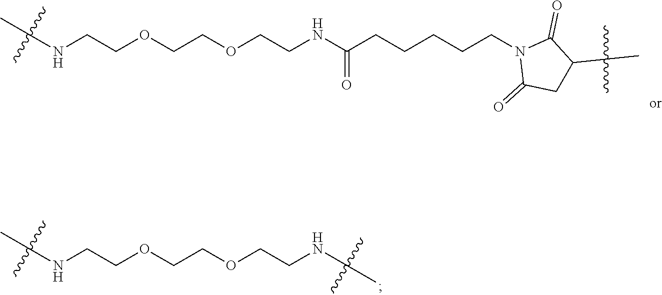 [SMILES: *NCCOCCOCCNC(=O)CCCCCN1C(=O)CC(*)C1=O ; *NCCOCCOCCN*]
T is selected from PPT-iRGD or FA; and
n₁, n₂, n₄ and n₅ are each independently selected from 1, 2, 3, 4, 5, 6, 7 or 8;
preferably, n₁ is selected from 1, 2 or 3;
more preferably, n₁ is 1;
preferably, n₂ is selected from 1, 2 or 3;
more preferably, n₂ is 2;
preferably, n₄ is selected from 3, 4 or 5;
more preferably, n₄ is selected from 4 or 5;
more preferably, n₄ is 4;
preferably, n₅ is selected from 4, 5 or 6; and
more preferably, n₅ is 5.

2. The polyethylene glycol-drug conjugate, the stereoisomer thereof or the pharmaceutically acceptable salt thereof according to claim 1, wherein the polyethylene glycol-drug conjugate has a structure represented by Formula (I), 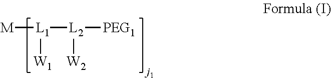
wherein:
M is 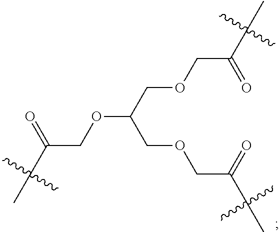 [SMILES: *C(=O)COCC(COCC(*)=O)OCC(*)=O]
L₁ is 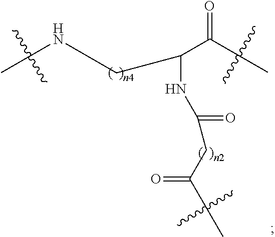
preferably, L₁ is 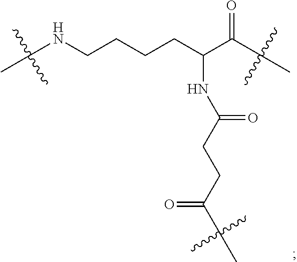 [SMILES: *NCCCCC(NC(=O)CCC(*)=O)C(*)=O]
L₂ is 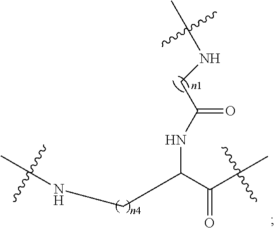
preferably, L₂ is 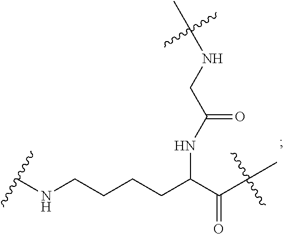 [SMILES: *NCCCCC(NC(=O)CN*)C(*)=O]
W₁ and W₂ are each independently selected from -Q₁ or 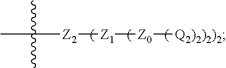
preferably, W₁ is -Q₁, and W₂ is 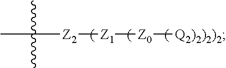
PEG₁ is a single-armed polyethylene glycol chain segment, PEG₁ is connected to L₂ through a carbonyl group, and a number-average molecular weight of PEG₁ is 5k to 40k, preferably 5k to 10k or 10k to 40k, and more preferably 10k;
j₁ is selected from 2, 3 or 4;
preferably, j₁ is 3;
Z₂, Z₁ and Z₀ are each independently selected from 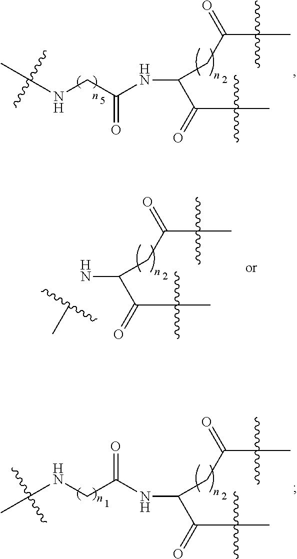
preferably, Z₂, Z₁ and Z₀ are each independently selected from 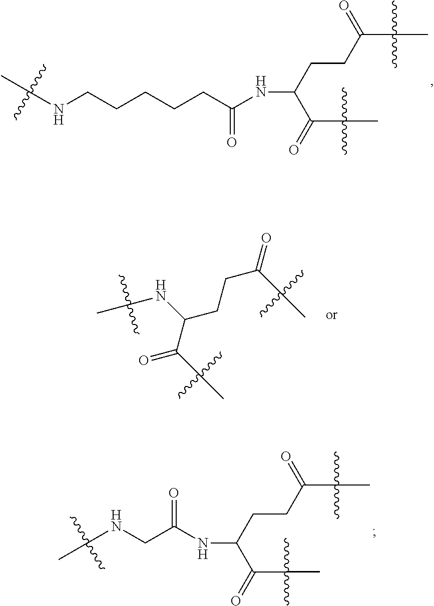 [SMILES: *NCCCCCC(=O)NC(CCC(*)=O)C(*)=O ; *NC(CCC(*)=O)C(*)=O ; *NCC(=O)NC(CCC(*)=O)C(*)=O]
or
preferably, Z₂ is 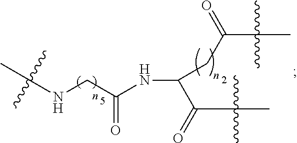
and
more preferably, Z₂ is 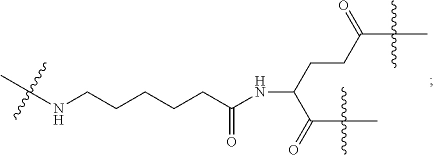 [SMILES: *NCCCCCC(=O)NC(CCC(*)=O)C(*)=O]
or
preferably, Z₁ is 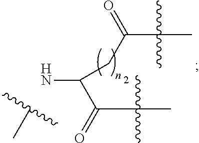
and
more preferably, Z₁ is 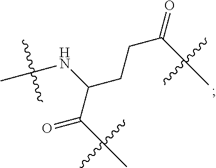 [SMILES: *NC(CCC(*)=O)C(*)=O]
or
preferably, Z₀ is 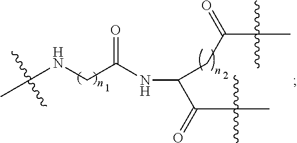
and
more preferably, Z₀ is 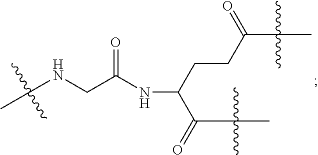 [SMILES: *NCC(=O)NC(CCC(*)=O)C(*)=O]
n₁, n₂, n₄ and n₅ are each independently selected from 1, 2, 3, 4, 5, 6, 7 or 8;
preferably, n₁ is selected from 1, 2 or 3;
more preferably, n₁ is 1;
preferably, n₂ is selected from 1, 2 or 3;
more preferably, n₂ is 2;
preferably, n₄ is selected from 3, 4 or 5;
more preferably, n₄ is 4;
preferably, n₅ is selected from 4, 5 or 6; and
more preferably, n₅ is 5;
Q₁ is —N₁-AC₁;
Q₂ is —N₂-AC₂;
N₁ and N₂ are each independently selected from 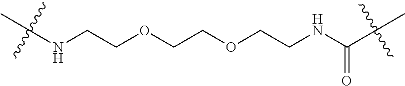 [SMILES: *NCCOCCOCCNC(*)=O]
or GFLG;
preferably, N₁ is 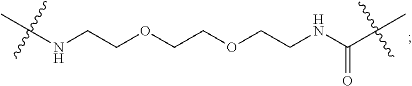 [SMILES: *NCCOCCOCCNC(*)=O]
and
preferably, N₂ is GFLG; and
AC₁ and AC₂ are each independently an anticancer drug;
preferably, AC₁ and AC₂ are each independently selected from AXT, PCB, 5FU, DXM, SRM, IMQ or LNL;
more preferably, AC₁ and AC₂ are each independently selected from AXT or PCB;
most preferably, AC₁ is AXT; and
most preferably, AC₂ is PCB.

3. The polyethylene glycol-drug conjugate, the stereoisomer thereof or the pharmaceutically acceptable salt thereof according to claim 1, wherein the polyethylene glycol-drug conjugate has a structure represented by Formula (II), 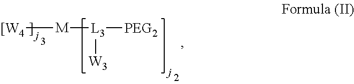
wherein:
M is 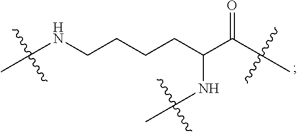 [SMILES: *NCCCCC(N*)C(*)=O]
L₃ is selected from 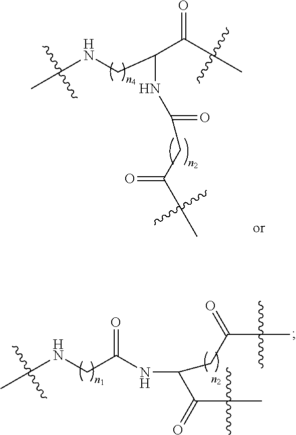
preferably, L₃ is selected from 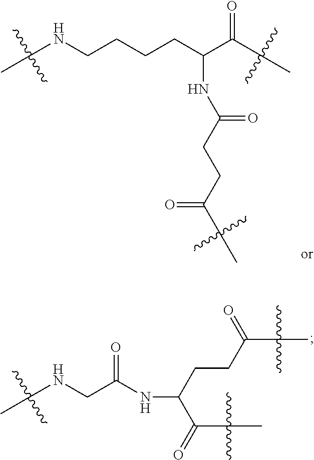
W₃ and W₄ are each independently selected from 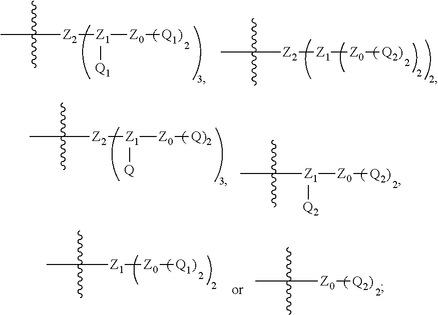
preferably, W₃ is selected from 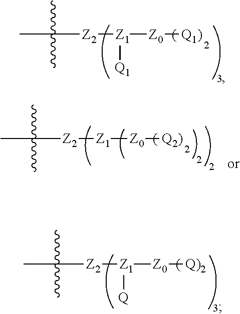
and
preferably, W₄ is selected from 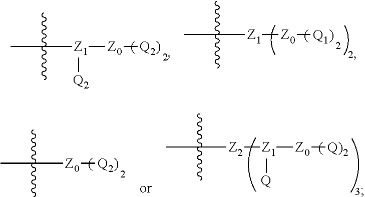
PEG₂ is a single-armed polyethylene glycol chain segment, PEG₂ is connected to L₃ through a carbonyl group, and a number-average molecular weight of PEG₂ is 5k to 40k, preferably 5k to 10k or 10k to 40k, and more preferably 10k;
j₂ and j₃ are each independently selected from 1, 2, 3 or 4;
preferably, j₂ is selected from 1, 2 or 3;
more preferably, j₂ is 2;
preferably, j₃ is selected from 1, 2 or 3; and
more preferably, j₃ is 1;
Z₂, Z₁ and Z₀ are each independently selected from 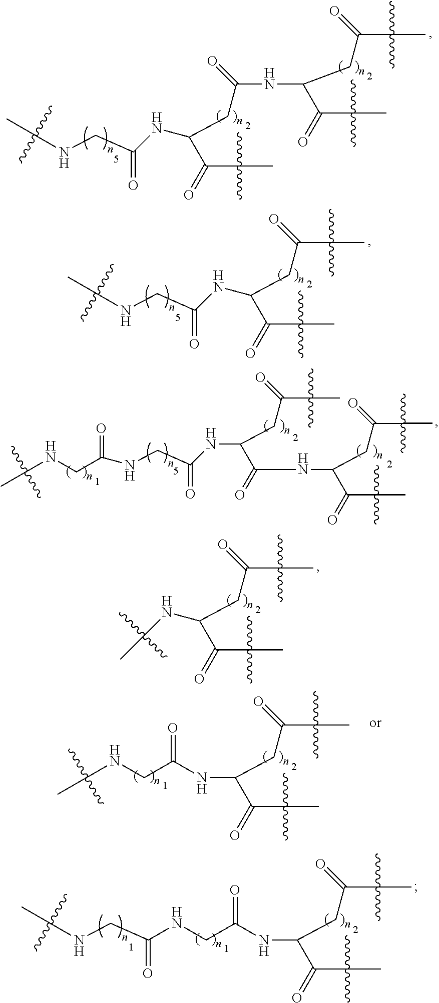
preferably, Z₂, Z₁ and Z₀ are each independently selected from 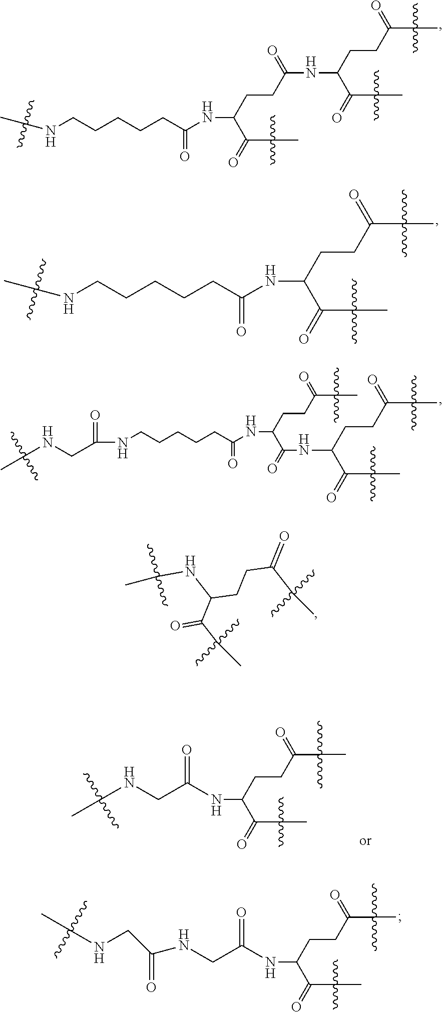
or
preferably, Z₂ is selected from 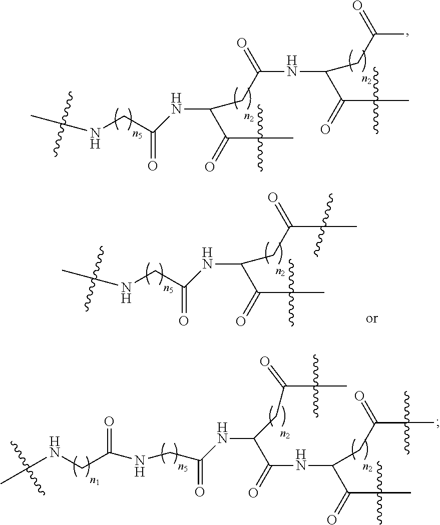
and
more preferably, Z₂ is selected from 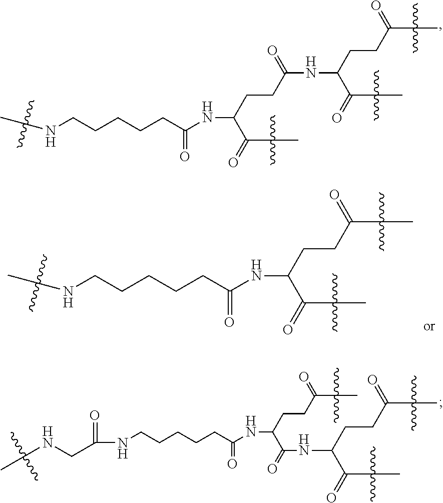
or
preferably, Z₁ is selected from 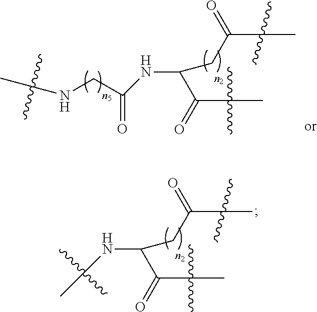
and
more preferably, Z₁ is selected from 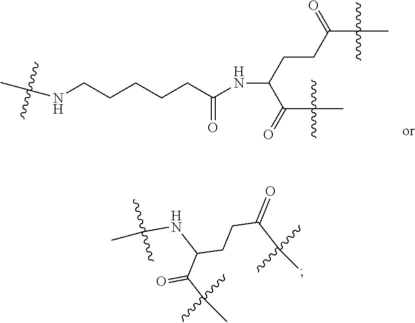
or
preferably, Z₀ is selected from 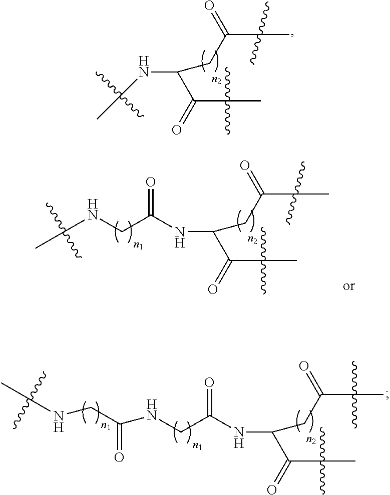
and
more preferably, Z₀ is selected from 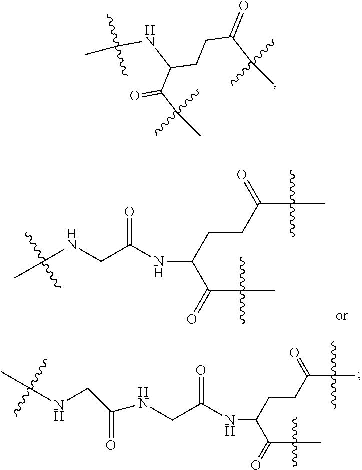
n₁, n₂, n₄ and n₅ are each independently selected from 1, 2, 3, 4, 5, 6, 7 or 8;
preferably, n₁ is selected from 1, 2 or 3;
more preferably, n₁ is 1;
preferably, n₂ is selected from 1, 2 or 3;
more preferably, n₂ is 2;
preferably, n₄ is selected from 3, 4 or 5;
more preferably, n₄ is 4;
preferably, n₅ is selected from 4, 5 or 6; and
more preferably, n₅ is 5;
Q is —N-AC;
Q₁ is —N₁-AC₁;
Q₂ is —N₂-AC₂;
N, N₁ and N₂ are each independently selected from 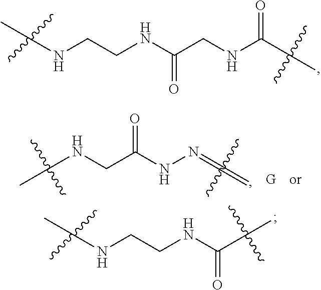
preferably, N is 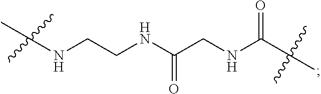
preferably, N₁ is selected from 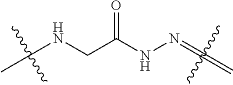 [SMILES: *NCC(=O)NN=*]
or G; and
preferably, N₂ is selected from 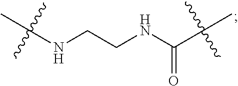 [SMILES: *NCCNC(*)=O]
and
AC, AC₁ and AC₂ are each independently an anticancer drug;
preferably, AC, AC₁ and AC₂ are each independently selected from AXT, PCB, 5FU, DXM, SRM, IMQ or LNL;
more preferably, AC, AC₁ and AC₂ are each independently selected from 5FU, DXM, SRM or AXT;
most preferably, AC is 5FU;
most preferably, AC₁ is selected from DXM or SRM; and
most preferably, AC₂ is AXT.

4. The polyethylene glycol-drug conjugate, the stereoisomer thereof or the pharmaceutically acceptable salt thereof according to claim 1, wherein the polyethylene glycol-drug conjugate has a structure represented by Formula (III), 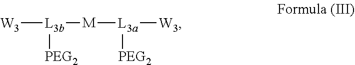
wherein:
M is 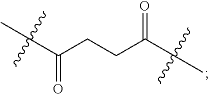 [SMILES: *C(=O)CCC(*)=O]
L₃ₐ and L_{3b} are each independently selected from 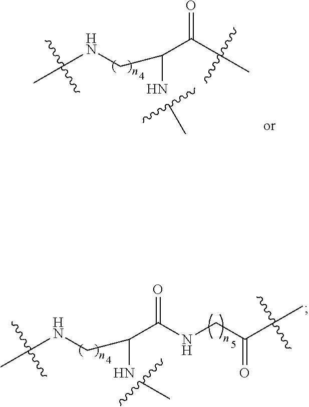
preferably, L₃ₐ and L_{3b} are each independently selected from 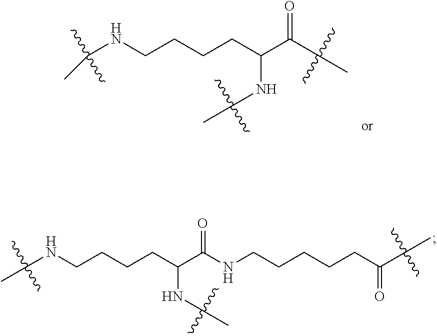 [SMILES: *NCCCCC(N*)C(*)=O ; *NCCCCC(N*)C(=O)NCCCCCC(*)=O]
or
preferably, L₃ₐ is 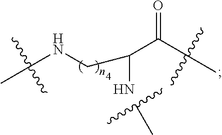
and
more preferably, L₃ₐ is 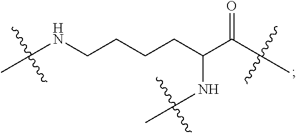 [SMILES: *NCCCCC(N*)C(*)=O]
or
preferably, L_{3b} is 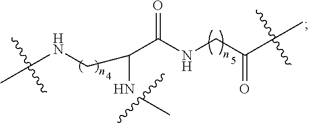
and
more preferably, L_{3b} is 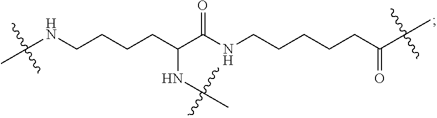 [SMILES: *NCCCCC(N*)C(=O)NCCCCCC(*)=O]
W₃ is 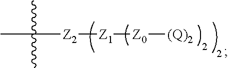
PEG₂ is a single-armed polyethylene glycol chain segment, PEG₂ is connected to L₃ₐ or L_{3b} through a carbonyl group, and a number-average molecular weight of PEG₂ is 5k to 40k, preferably 5k to 10k or 10k to 40k, and more preferably 10k;
Z₂, Z₁ and Z₀ are each independently selected from 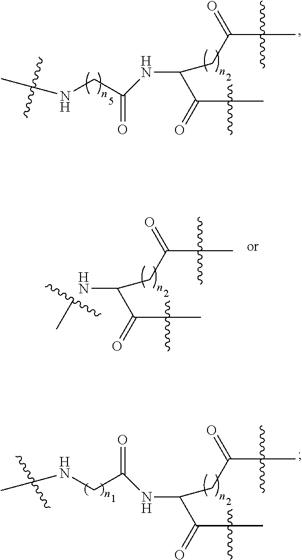
preferably, Z₂, Z₁ and Z₀ are each independently selected from 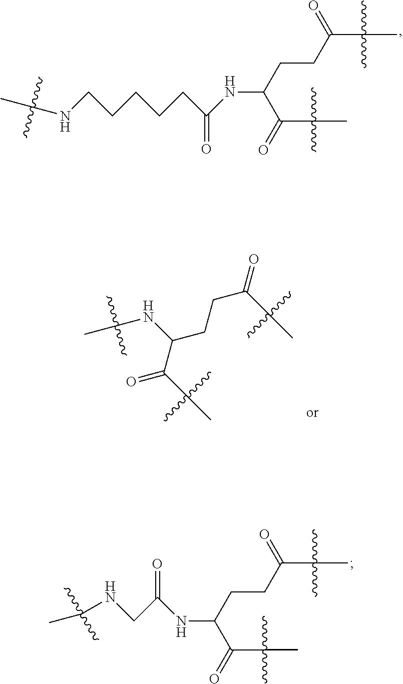 [SMILES: *NCCCCCC(=O)NC(CCC(*)=O)C(*)=O ; *NC(CCC(*)=O)C(*)=O ; *NCC(=O)NC(CCC(*)=O)C(*)=O]
or
preferably, Z₂ is 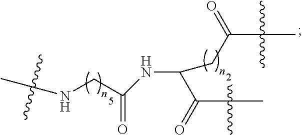
and
more preferably, Z₂ is 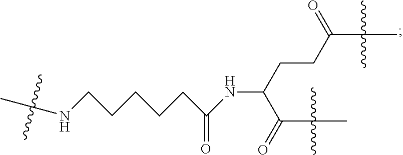 [SMILES: *NCCCCCC(=O)NC(CCC(*)=O)C(*)=O]
or
preferably, Z₁ is 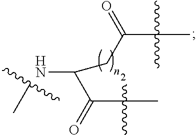
and
more preferably, Z₁ is 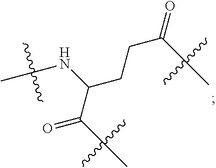 [SMILES: *NC(CCC(*)=O)C(*)=O]
or
preferably, Z₀ is 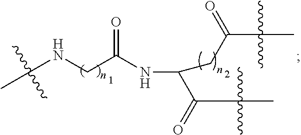
and
more preferably, Z₀ is 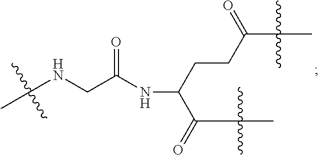 [SMILES: *NCC(=O)NC(CCC(*)=O)C(*)=O]
n₁, n₂, n₄ and n₅ are each independently selected from 1, 2, 3, 4, 5, 6, 7 or 8;
preferably, n₁ is selected from 1, 2 or 3;
more preferably, n₁ is 1;
preferably, n₂ is selected from 1, 2 or 3;
more preferably, n₂ is 2;
preferably, n₄ is selected from 3, 4 or 5;
more preferably, n₄ is 4;
preferably, n₅ is selected from 4, 5 or 6; and
more preferably, n₅ is 5;
Q is —N-AC;
N is 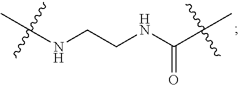 [SMILES: *NCCNC(*)=O]
and
AC is an anticancer drug;
preferably, AC is selected from AXT, PCB, 5FU, DXM, SRM, IMQ or LNL; and
more preferably, AC is AXT.

5. The polyethylene glycol-drug conjugate, the stereoisomer thereof or the pharmaceutically acceptable salt thereof according to claim 1, wherein the polyethylene glycol-drug conjugate has a structure represented by Formula (IV), 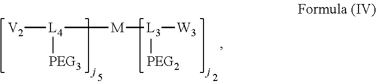
wherein:
M is 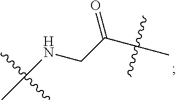 [SMILES: *NCC(*)=O]
L₃ and L₄ are each independently selected from 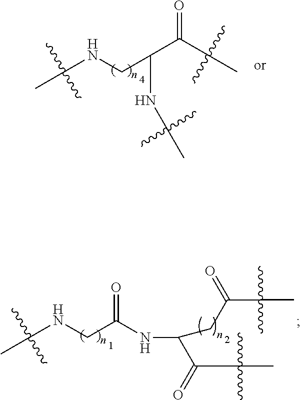
preferably, L₃ and L₄ are each independently selected from 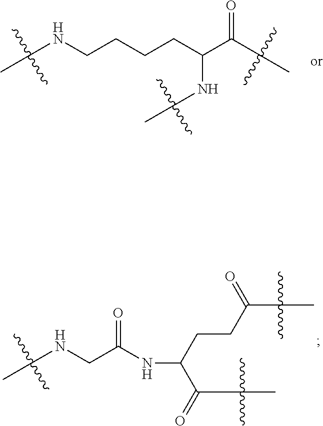 [SMILES: *NCCCCC(N*)C(*)=O ; *NCC(=O)NC(CCC(*)=O)C(*)=O]
or
preferably, L₃ is 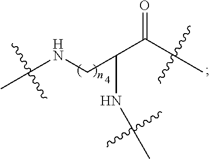
and
more preferably, L₃ is 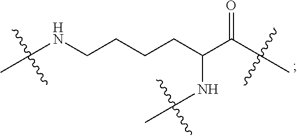 [SMILES: *NCCCCC(N*)C(*)=O]
or
preferably, L₄ is 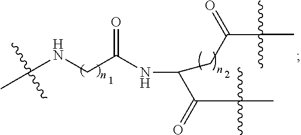
and
more preferably, L₄ is 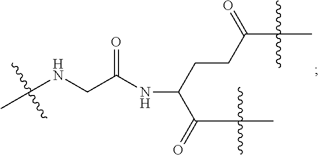 [SMILES: *NCC(=O)NC(CCC(*)=O)C(*)=O]
PEG₂ and PEG₃ are each independently a single-armed polyethylene glycol chain segment, PEG₂ is connected to L₃ through a carbonyl group, PEG₃ is connected to L₄ through a carbonyl group, and a number-average molecular weight of each of PEG₂ and PEG₃ is independently 5k to 40k, preferably 5k to 10k or 10k to 40k, and more preferably 10k;
j₂ and j₅ are each independently selected from 1, 2, 3, 4 or 5;
preferably, j₂ is selected from 1, 2 or 3;
more preferably, j₂ is 1;
preferably, j₅ is selected from 1, 2 or 3; and
more preferably, j₅ is 1;
W₃ is 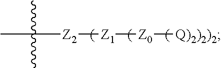
Z₂, Z₁ and Z₀ are each independently selected from 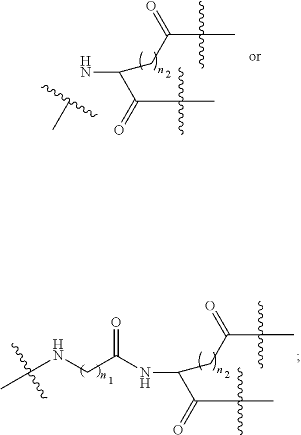
preferably, Z₂, Z₁ and Z₀ are each independently selected from 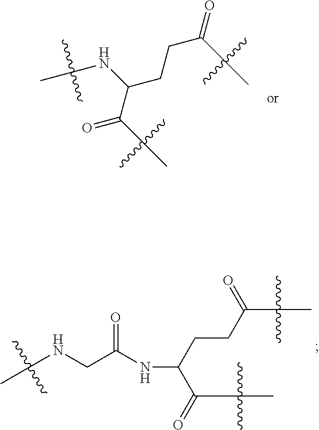 [SMILES: *NC(CCC(*)=O)C(*)=O ; *NCC(=O)NC(CCC(*)=O)C(*)=O]
or
preferably, Z₂ is 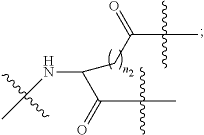
and
more preferably, Z₂ is 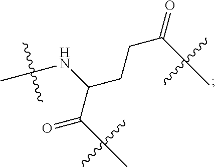 [SMILES: *NC(CCC(*)=O)C(*)=O]
or
more preferably Z₁ is 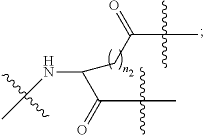
and
more preferably Z₁ is 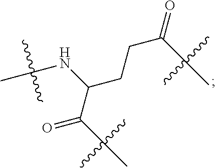 [SMILES: *NC(CCC(*)=O)C(*)=O]
or
preferably, Z₀ is 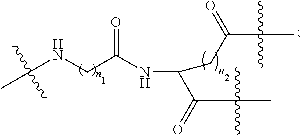
and
more preferably, Z₀ is 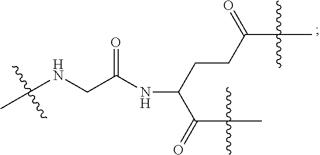 [SMILES: *NCC(=O)NC(CCC(*)=O)C(*)=O]
Q is —N-AC;
N is 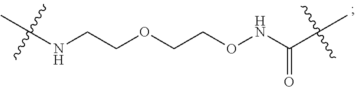 [SMILES: *NCCOCCONC(*)=O]
AC is an anticancer drug;
preferably, AC is selected from AXT, PCB, 5FU, DXM, SRM, IMQ or LNL; and
more preferably, AC is AXT;
V₂ is 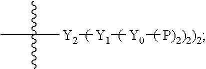
Y₂, Y₁ and Y₀ are each independently selected from 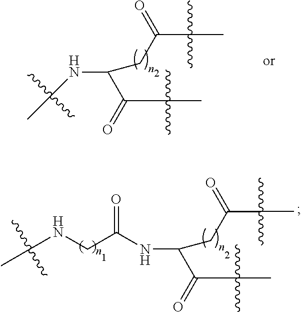
preferably, Y₂, Y₁ and Y₀ are each independently selected from 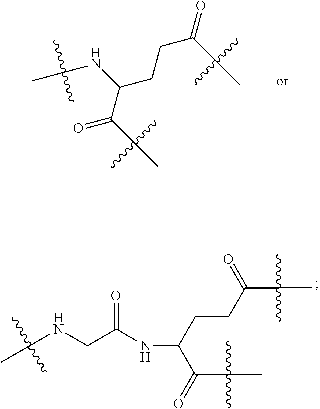 [SMILES: *NC(CCC(*)=O)C(*)=O ; *NCC(=O)NC(CCC(*)=O)C(*)=O]
or
preferably, Y₂ is 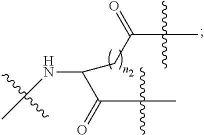
and
more preferably, Y₂ is 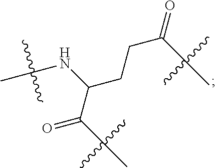 [SMILES: *NC(CCC(*)=O)C(*)=O]
or
preferably, Y₁ is 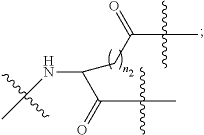
and
more preferably, Y₁ is 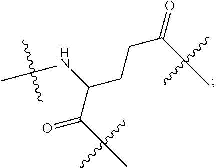 [SMILES: *NC(CCC(*)=O)C(*)=O]
or
preferably, Y₀ is 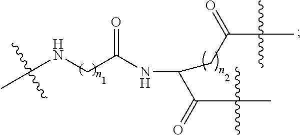
and
more preferably, Y₀ is 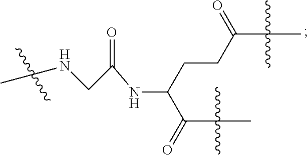 [SMILES: *NCC(=O)NC(CCC(*)=O)C(*)=O]
P is -Lᵥ-T;
Lᵥ is 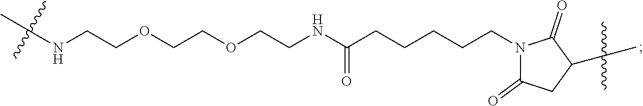 [SMILES: *NCCOCCOCCNC(=O)CCCCCN1C(=O)CC(*)C1=O]
T is PPT-iRGD; and
n₁, n₂ and n₄ are each independently selected from 1, 2, 3, 4, 5, 6, 7 or 8;
preferably, n₁ is selected from 1, 2 or 3;
more preferably, n₁ is 1;
preferably, n₂ is selected from 1, 2 or 3;
more preferably, n₂ is 2;
preferably, n₄ is selected from 3, 4 or 5; and
more preferably, n₄ is 4.

6. The polyethylene glycol-drug conjugate, the stereoisomer thereof or the pharmaceutically acceptable salt thereof according to claim 1, wherein the polyethylene glycol-drug conjugate has a structure represented by Formula (V), 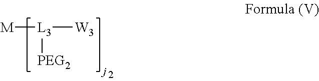
wherein:
M is selected from 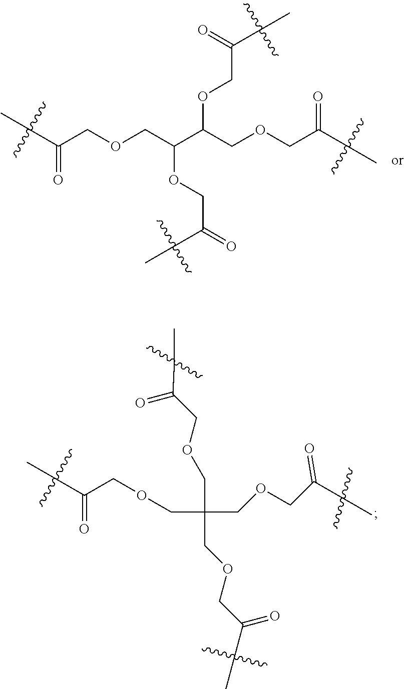 [SMILES: *C(=O)COCC(OCC(*)=O)C(COCC(*)=O)OCC(*)=O ; *C(=O)COCC(COCC(*)=O)(COCC(*)=O)COCC(*)=O]
or
M is selected from 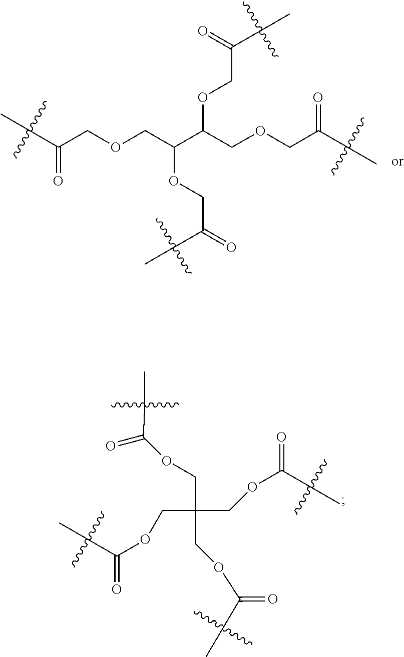 [SMILES: *C(=O)COCC(OCC(*)=O)C(COCC(*)=O)OCC(*)=O ; *C(=O)OCC(COC(*)=O)(COC(*)=O)COC(*)=O]
L₃ is selected from 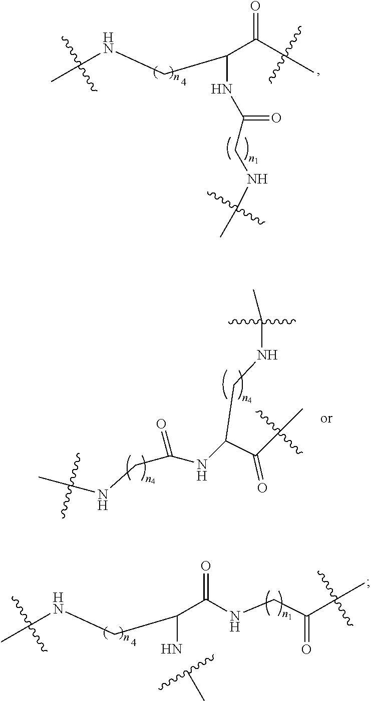
preferably, L₃ is selected from 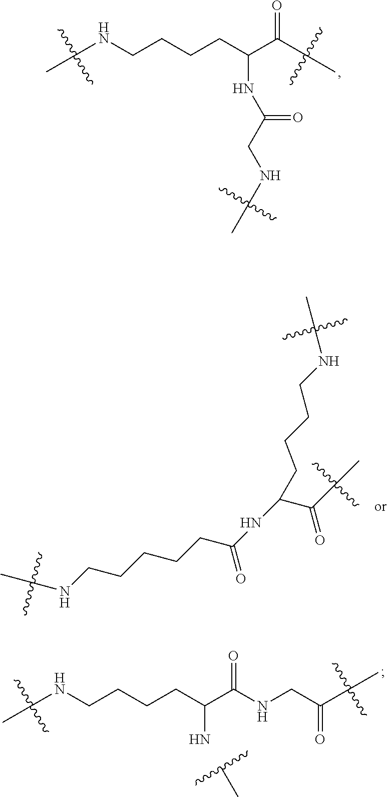
or
preferably, L₃ is selected from 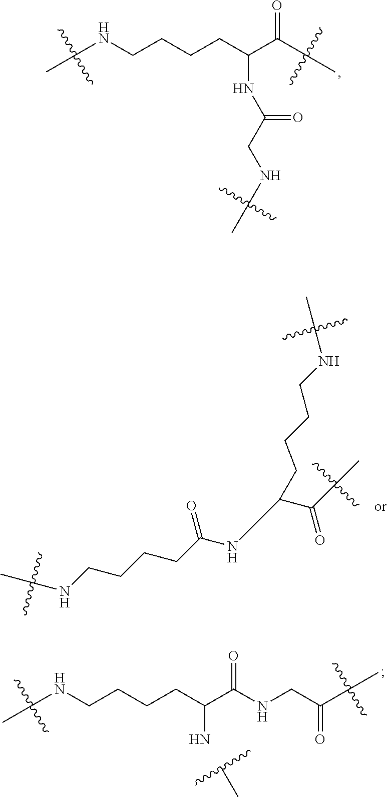
PEG₂ is a single-armed polyethylene glycol chain segment, PEG₂ is connected to L₃ through a carbonyl group or an amino group, and a number-average molecular weight of PEG₂ is 5k to 40k, preferably 5k to 10k or 10k to 40k, and more preferably 10k;
j₂ is selected from 3, 4 or 5;
preferably, j₂ is 4;
W₃ is selected from 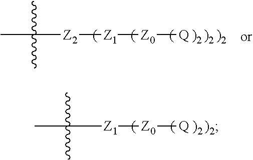
Z₂, Z₁ and Z₀ are each independently selected from 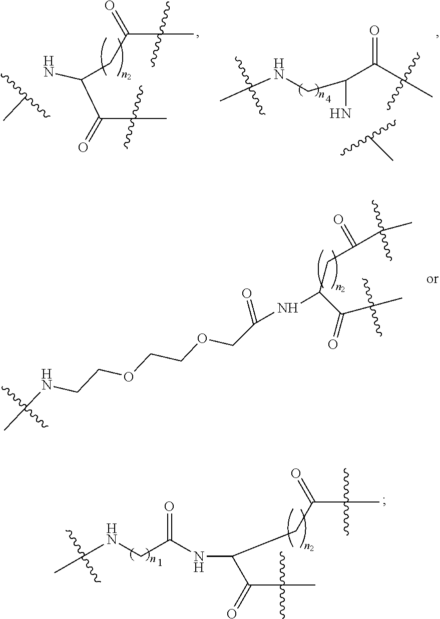
preferably, Z₂, Z₁ and Z₀ are each independently selected from 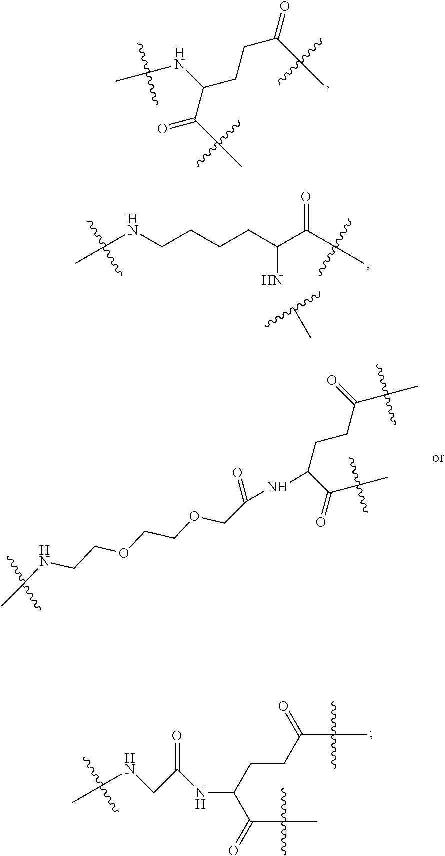
or
preferably, Z₂ is selected from 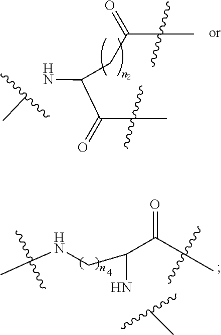
and
more preferably, Z₂ is selected from 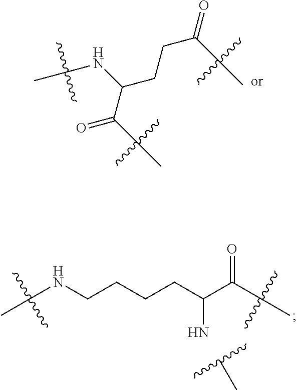
or
preferably, Z₁ is selected from 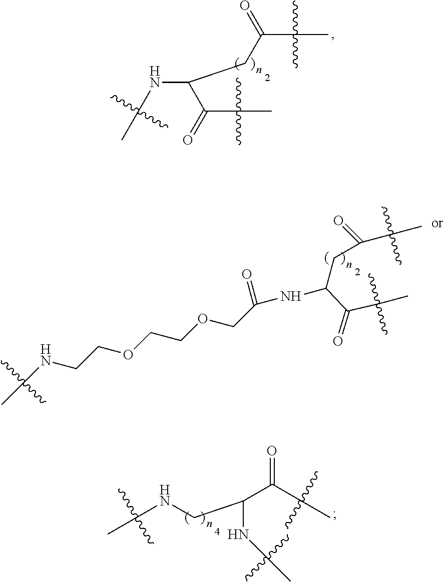
and
more preferably, Z₁ is selected from 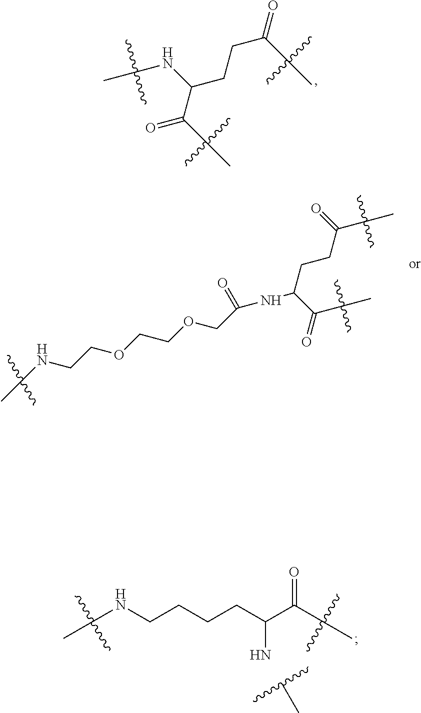
or
preferably, Z₀ is selected from 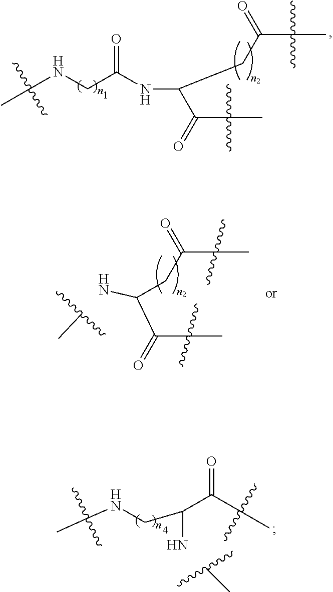
and
more preferably, Z₀ is selected from 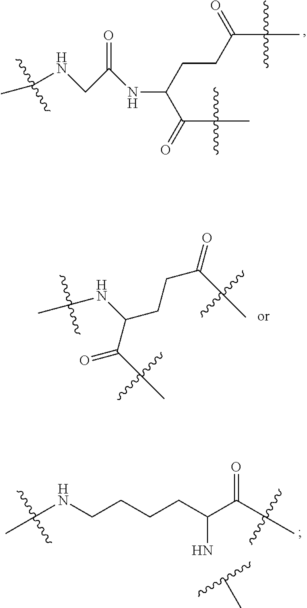
n₁, n₂ and n₄ are each independently selected from 1, 2, 3, 4, 5, 6, 7 or 8; preferably, n₁ is selected from 1, 2 or 3;
more preferably, n₁ is 1;
preferably, n₂ is selected from 1, 2 or 3;
more preferably, n₂ is 2;
preferably, n₄ is selected from 3, 4 or 5;
more preferably, n₄ is selected from 4 or 5; and
more preferably, n₄ is 4;
Q is —N-AC;
N is selected from 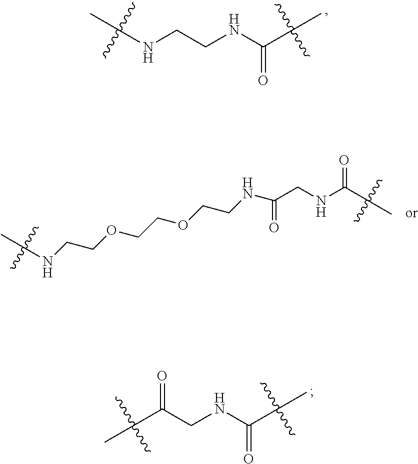 [SMILES: "," ; *NCCNC(*)=O ; *NCCOCCOCCNC(=O)CNC(*)=O ; "or" ; *C(=O)CNC(*)=O ; ";"]
and
AC is an anticancer drug;
preferably, AC is selected from AXT, PCB, 5FU, DXM, SRM, IMQ or LNL; and
more preferably, AC is selected from IMQ, LNL or 5FU.

7. The polyethylene glycol-drug conjugate, the stereoisomer thereof or the pharmaceutically acceptable salt thereof according to claim 1, wherein the polyethylene glycol-drug conjugate has a structure represented by Formula (VI), 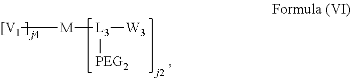
wherein:
M is 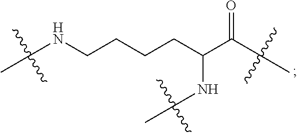 [SMILES: *NCCCCC(N*)C(*)=O ; ";"]
L₃ is 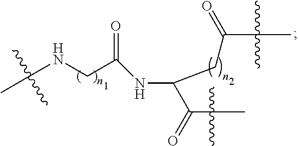
preferably, L₃ is 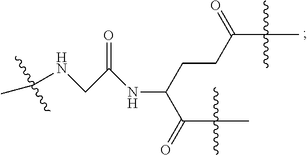 [SMILES: *NCC(=O)NC(CCC(*)=O)C(*)=O ; ";"]
PEG₂ is a single-armed polyethylene glycol chain segment, PEG₂ is connected to L₃ through a carbonyl group, and a number-average molecular weight of PEG₂ is 5k to 40k, preferably 5k to 10k or 10k to 40k, and more preferably 10k;
j₂ and j₄ are each independently selected from 1, 2, 3, 4 or 5;
preferably, j₂ is selected from 1, 2 or 3;
more preferably, j₂ is 2;
preferably, j₄ is selected from 1, 2 or 3; and
more preferably, j₄ is 1;
W₃ is 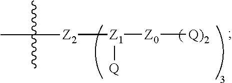
Z₂, Z₁ and Z₀ are each independently selected from 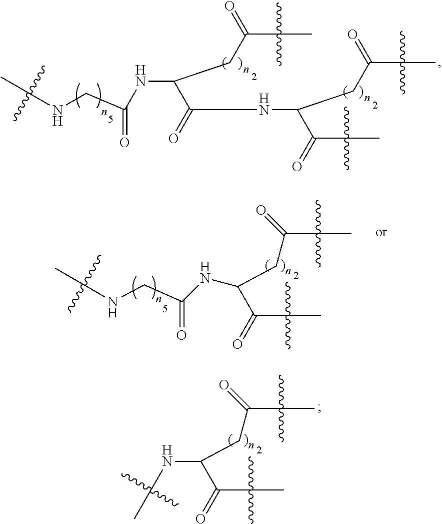
preferably, Z₂, Z₁ and Z₀ are each independently selected from 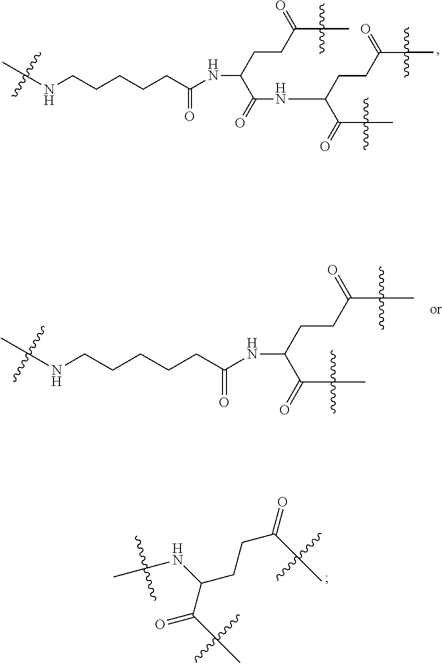 [SMILES: *NCCCCCC(=O)NC(CCC(*)=O)C(=O)NC(CCC(*)=O)C(*)=O ; *NCCCCCC(=O)NC(CCC(*)=O)C(*)=O ; *NC(CCC(*)=O)C(*)=O]
or
preferably, Z₂ is 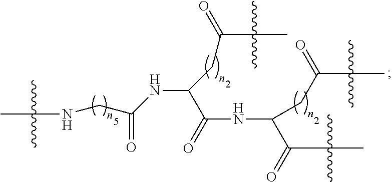
and
more preferably, Z₂ is 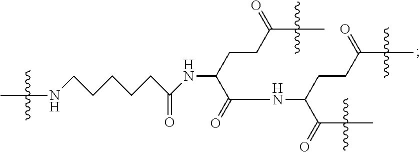 [SMILES: *NCCCCCC(=O)NC(CCC(*)=O)C(=O)NC(CCC(*)=O)C(*)=O ; ";"]
or
preferably, Z₁ is 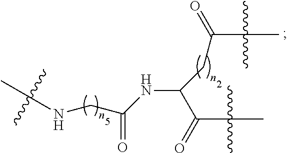
and
more preferably, Z₁ is 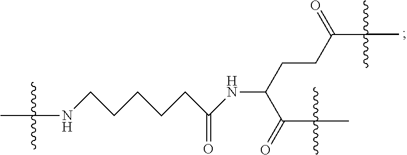 [SMILES: *NCCCCCC(=O)NC(CCC(*)=O)C(*)=O ; ";"]
or
preferably, Z₀ is 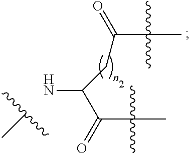
and
more preferably, Z₀ is 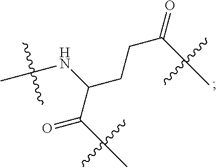 [SMILES: *NC(CCC(*)=O)C(*)=O ; ";"]
Q is —N-AC;
N is 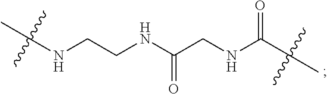 [SMILES: *NCCNC(=O)CNC(*)=O ; ";"]
AC is an anticancer drug;
preferably, AC is selected from AXT, PCB, 5FU, DXM, SRM, IMQ or LNL; and
more preferably, AC is 5FU;
V₁ is 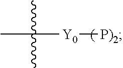
Y₀ is 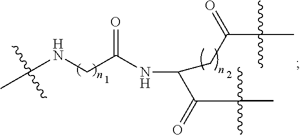
preferably, Y₀ is 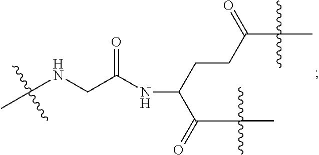 [SMILES: *NCC(=O)NC(CCC(*)=O)C(*)=O]
P is -Lᵥ-T;
Lᵥ is 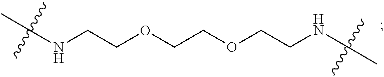 [SMILES: *NCCOCCOCCN*]
T is FA; and
n₁, n₂ and n₅ are each independently selected from 1, 2, 3, 4, 5, 6, 7 or 8;
preferably, n₁ is selected from 1, 2 or 3;
more preferably, n₁ is 1;
preferably, n₂ is selected from 1, 2 or 3;
more preferably, n₂ is 2;
preferably, n₅ is selected from 4, 5 or 6; and
more preferably, n₅ is 5.

8. The polyethylene glycol-drug conjugate, the stereoisomer thereof or the pharmaceutically acceptable salt thereof according to claim 1, wherein the polyethylene glycol-drug conjugate is selected from:
No. Structure [symbol] 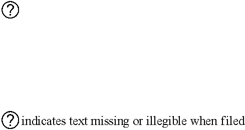
wherein, a number-average molecular weight of 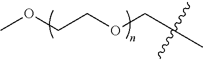
is 10k [symbol] 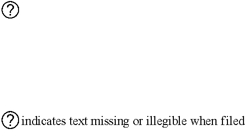
wherein, a number-average molecular weight of 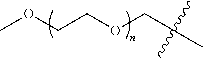
is 10k [symbol] 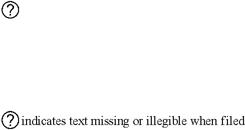
wherein, a number-average molecular weight of 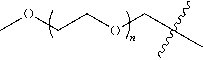
is 10k [symbol] 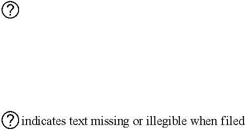
wherein, a number-average molecular weight of 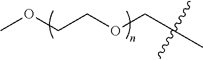
is 10k [symbol] 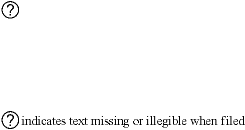
wherein, a number-average molecular weight of 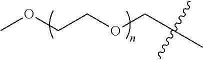
is 10k [symbol] 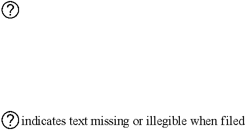
wherein, a number-average molecular weight of 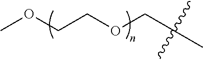
is 10k [symbol] 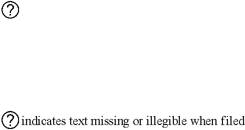
wherein, a number-average molecular weight of 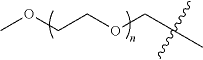
is 10k [symbol] 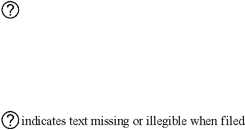
wherein, a number-average molecular weight of 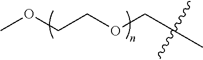
is 10k [symbol] 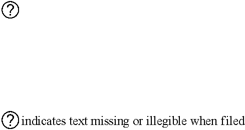
wherein, a number-average molecular weight of 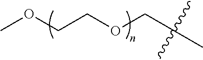
is 10k [symbol] 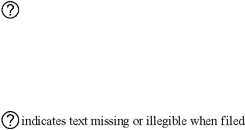
wherein, a number-average molecular weight of 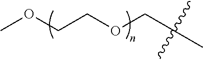
is 10k [symbol] 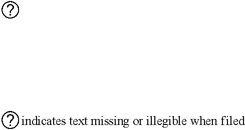
wherein, a number-average molecular weight of 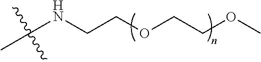
is 10k [symbol] 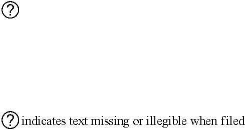
wherein, a number-average molecular weight of 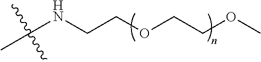
is 10k.

9. An intermediate for use in preparing the polyethylene glycol-drug conjugate, the stereoisomer thereof or the pharmaceutically acceptable salt thereof according to claim 1, wherein the intermediate is selected from:

[symbol]

| No. | Structure |
|---|---|
| 48-97 | 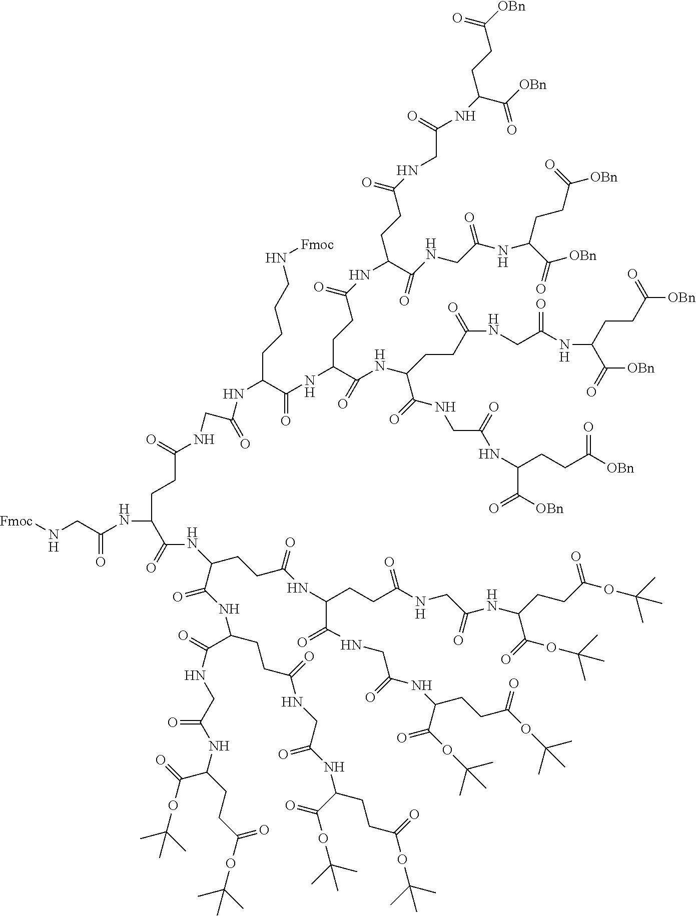 |
| 39-176 | 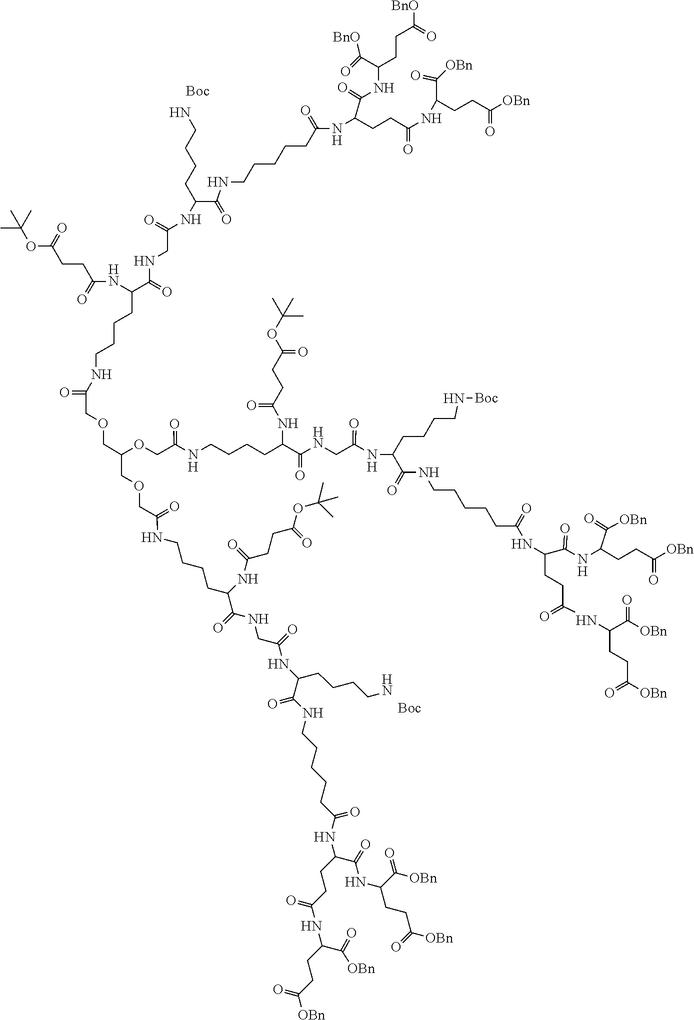 |
| 51-51 | 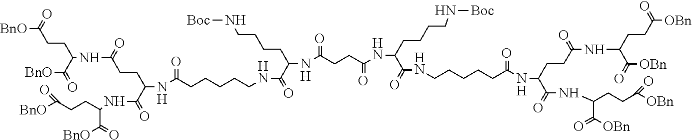 [SMILES: CC(C)(C)OC(=O)NCCCCC(NC(=O)CCC(=O)NC(CCCCNC(=O)OC(C)(C)C)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1] |
| 51-109 | 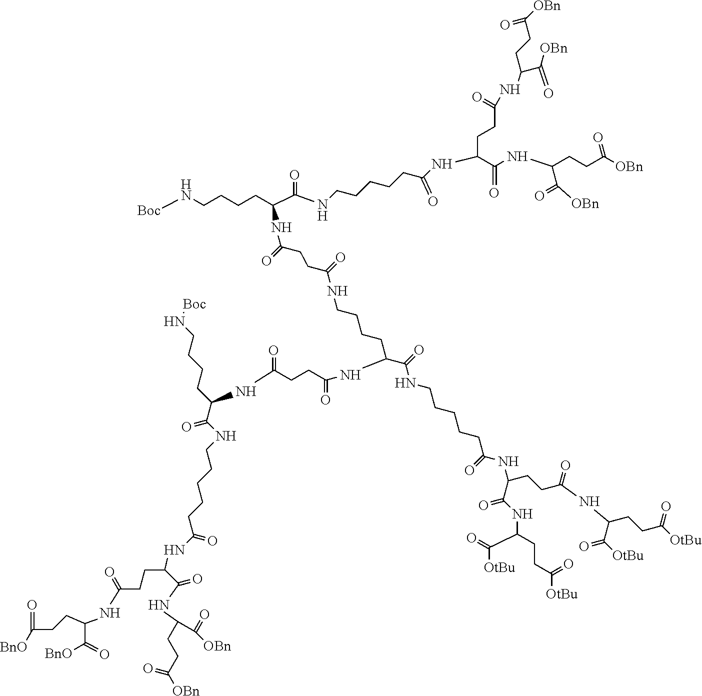 [SMILES: CC(C)(C)OC(=O)CCC(NC(=O)CCC(NC(=O)CCCCCNC(=O)C(CCCCNC(=O)CCC(=O)N[C@@H](CCCCNC(=O)OC(C)(C)C)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1)NC(=O)CCC(=O)N[C@H](CCCCNC(=O)OC(C)(C)C)C(=O)NCCCCCC(=O)NC(CCC(=O)NC(CCC(=O)OCc1ccccc1)C(=O)OCc1ccccc1)C(=O)NC(CCC(=O)OCc1ccccc1)C(=O)OCc1ccccc1)C(=O)NC(CCC(=O)OC(C)(C)C)C(=O)OC(C)(C)C)C(=O)OC(C)(C)C] |
| 43-228 | 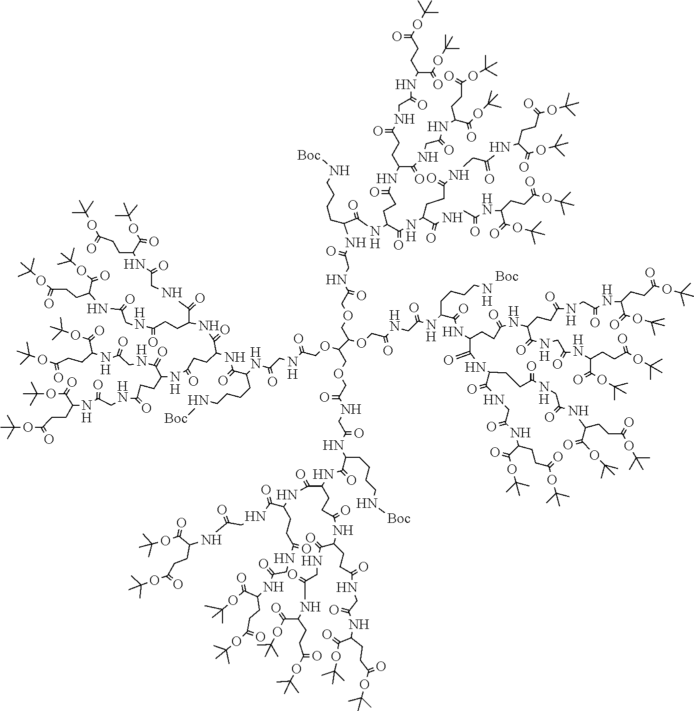 |
| 41-196 | 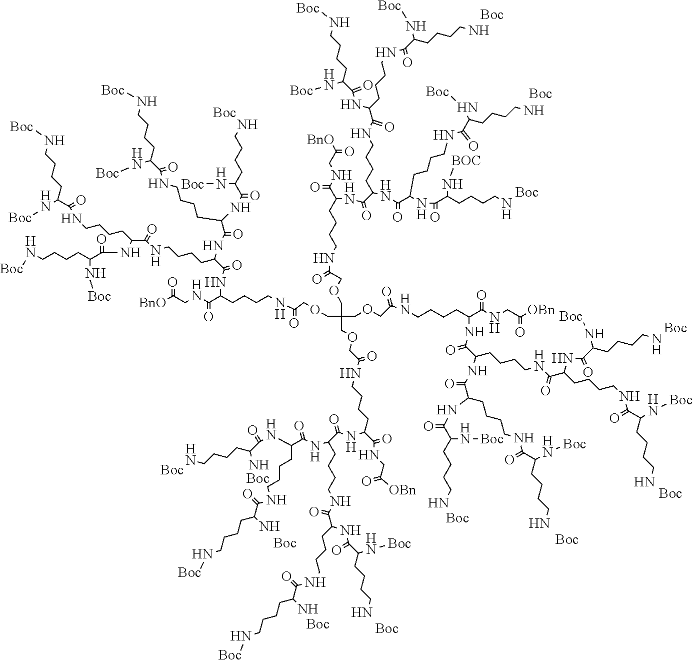 |
| 43-122 | 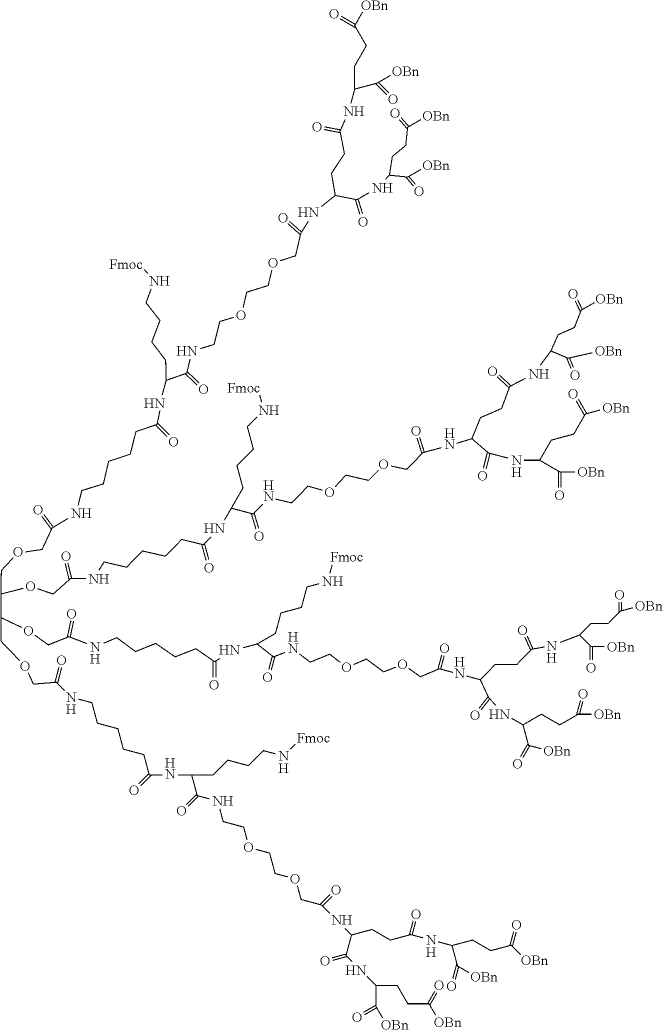 |
| 61-123 | 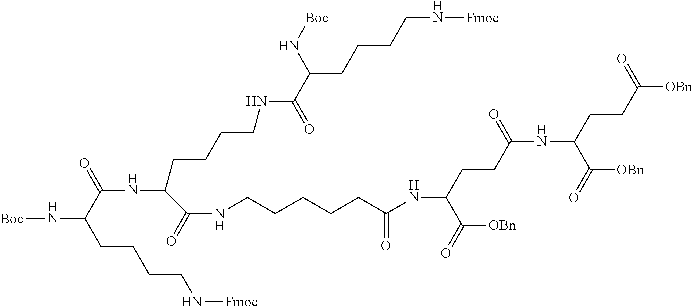 |
| 57-49 | 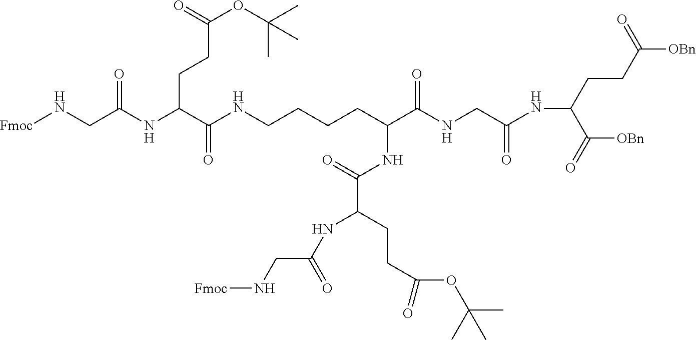 |
| 56-48 | 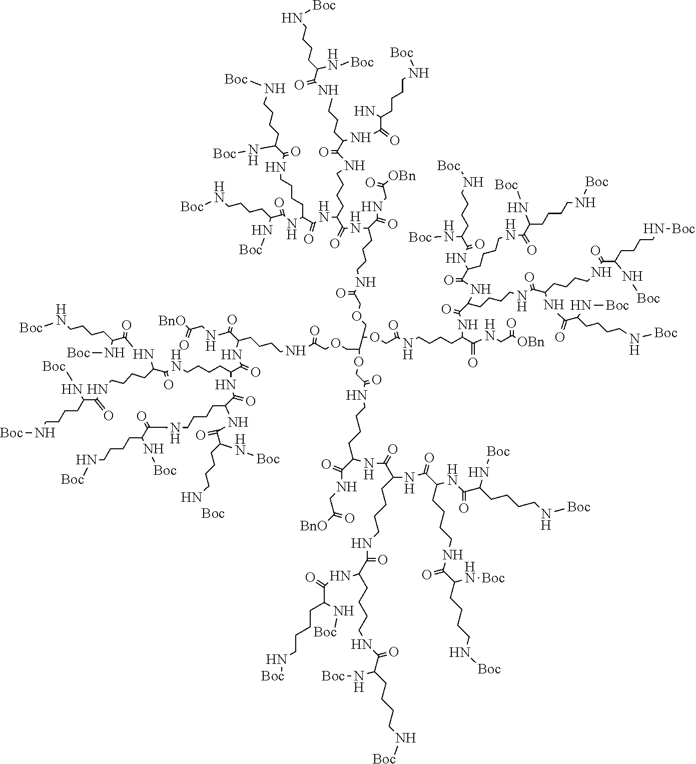 |
| 68-21 | 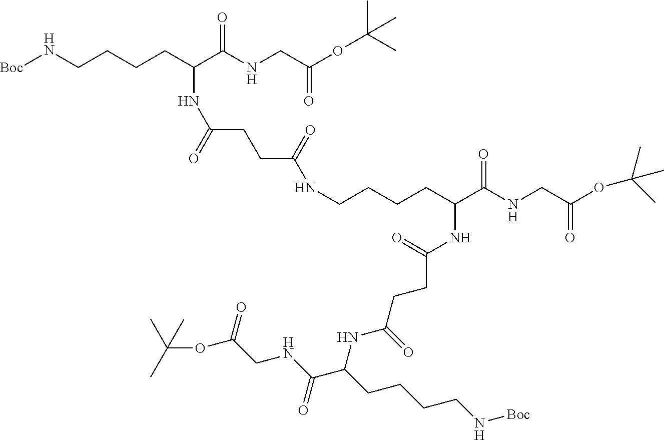 [SMILES: CC(C)(C)OC(=O)CNC(=O)C(CCCCNC(=O)OC(C)(C)C)NC(=O)CCC(=O)NCCCCC(NC(=O)CCC(=O)NC(CCCCNC(=O)OC(C)(C)C)C(=O)NCC(=O)OC(C)(C)C)C(=O)NCC(=O)OC(C)(C)C] |

10. A pharmaceutical composition comprising the polyethylene glycol-drug conjugate, the stereoisomer thereof or the pharmaceutically acceptable salt thereof according to claim 1; optionally, the composition further comprises one or more pharmaceutically acceptable auxiliary materials.

11. A method for treating and/or preventing a disease using the polyethylene glycol-drug conjugate, the stereoisomer thereof or the pharmaceutically acceptable salt thereof according to claim 1, wherein the disease is to be treated by an active ingredient in the polyethylene glycol-drug conjugate;
preferably, the disease is an ophthalmic disease;
more preferably, the disease is a disease associated with choroidal neovascularization (CNV);
most preferably, the disease is selected from a group consisting of choroidal neovascularization (CNV), diabetic retinopathy, central exudative retinochoroiditis, macular degeneration (for example, AMD), and high myopia retinopathy;
preferably, the disease is cancer selected from a group consisting of colon cancer, leukemia, lymphoma, bladder cancer, bone cancer, brain tumor, medulloblastoma, glioma, breast cancer, adenoma/carcinoid, adrenocortical carcinoma, islet cell carcinoma, cervical cancer, endometrial cancer, ovarian cancer, colorectal cancer, skin cancer, esophageal cancer, eye cancer, gallbladder cancer, gastric cancer, head and neck cancer, liver cancer, melanoma, Kaposi's sarcoma, kidney cancer, oral cancer, lung cancer, nasopharyngeal cancer, neuroblastoma, ovarian cancer, pancreatic cancer, thyroid cancer, parathyroid/penile cancer, prostate cancer, urethral cancer, vaginal cancer, vulvar cancer, anal cancer, sarcoma, and metastasis of the cancer; or
more preferably, the disease is cancer, and the cancer is eye cancer.
